(19) United States
(12) Patent Application Publication
(10) Pub. No.: US 2026/0256936 A1
(43) Pub. Date: Sep. 3, 2026

(54) BIOACTIVE CONJUGATES, PREPARATION METHOD AND USE THEREOF

(71) Applicant: BeOne Medicines I GmbH, Basel (CH)

(72) Inventors: Charng-Sheng Tsai, Beijing (CN); Mei-Hsuan Tsai, Beijing (CN); Xiaodong Wei, Beijing (CN); Wei Luo, Beijing (CN); Liming Wu, Beijing (CN)

(73) Assignee: BeOne Medicines I GmbH, Basel (CH)

(21) Appl. No.: 19/431,917

(22) Filed: Dec. 23, 2025

Related U.S. Application Data
(63) Continuation of application No. PCT/CN2024/102431, filed on Jun. 28, 2024.

(30) Foreign Application Priority Data
Jun. 29, 2023 (WO) PCT/CN2023/104058

(51) Int. Cl. A61K 47/68; A61K 31/353; A61K 31/4745; A61K 31/5517; A61K 31/706; A61K 38/05; A61K 38/06; C07K 16/00

(52) U.S. Cl. CPC A61K 47/6811; A61K 31/353; A61K 31/4745; A61K 31/5517; A61K 31/706; A61K 38/05; A61K 38/06; A61K 47/6807; A61K 47/6889; C07K 16/00; C07K 2317/21; C07K 2317/24

(57) ABSTRACT

Compounds of formula (Ia) are provided, wherein Conjugator is a group capable of bonding a BA and joining the BA to the rest of the compound, Cleavable and Linker together include at least one site for enzymatic cleavage and provide distance between Conjugator and Payload, Payload is a cytotoxic agent, and BA is a binding agent.

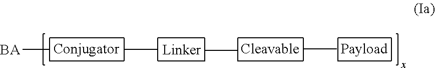

### CROSS REFERENCE TO RELATED APPLICATIONS

This application is a continuation of and claims priority under 35 U.S.C. §§ 120 and 365(c) to International Application No. PCT/CN2024/102431, filed Jun. 28, 2024, which claims priority to International Application No. PCT/CN2023/104058, filed Jun. 29, 2023, each of which is incorporated herein by reference in its entirety.

### FIELD

Provided herein are antibody drug conjugate platforms and antibody drug conjugates (ADCs) comprising the platforms and an antibody, or antigen-binding fragment thereof, as well as uses of the ADC platforms and ADCs.

### SEQUENCE LISTING

The contents of the electronic sequence listing (B176370004US00-SEQ-PJH.xml; Size: 8,224 bytes; and Date of Creation: Dec. 23, 2025) are herein incorporated by reference in their entirety.

### BACKGROUND

Antibody-drug conjugates (ADC) include an antibody against a tumor antigen linked to a biologically active small molecule such as a toxin or payload (i.e., a drug). ADCs selectively deliver the payload to cells expressing the tumor antigen. The payload monomethyl auristatin E (MMAE) is an antimitotic agent that inhibits cell division. ADCs including MMAE can be highly hydrophobic, leading to aggregation of ADCs with high drug-to-antibody ratios (DAR) and non-specific uptake of the ADCs. ADCs including MMAE can be unstable, leading to deconjugation, premature payload release, poor pharmacokinetic profiles, and off-target effects. ADCs employing other payloads can suffer from the same deficiencies.

The linker component of ADCs, including the portion that directly conjugates with an antibody, is one important feature in developing optimized therapeutic agents that are highly active at well-tolerated doses. The electrophilic maleimide functional group has been used to join linkers with a free thiol of an antibody. In vivo, the conjugation product is subject to slow elimination, thus reversing the conjugation reaction and leaving the maleimide of an ADC free to transfer to any other available thiol, including those from serum albumin in plasma.

There is an ongoing need for the development of new linkers for use with MMAE and other payloads in ADCs that would resist deconjugation and premature payload release, and allow for increased DAR, increased stability in circulation, improved pharmacokinetics, and improved efficacy. The present disclosure satisfies these needs.

### BRIEF SUMMARY

Provided herein are antibody drug conjugate platforms and antibody drug conjugates (ADCs). Also provided are uses of the ADC platforms to prepare ADCs.

In some embodiments, provided herein are ADC compounds of formula (Ia):

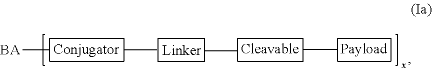

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

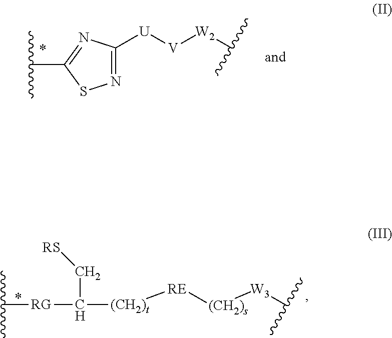

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

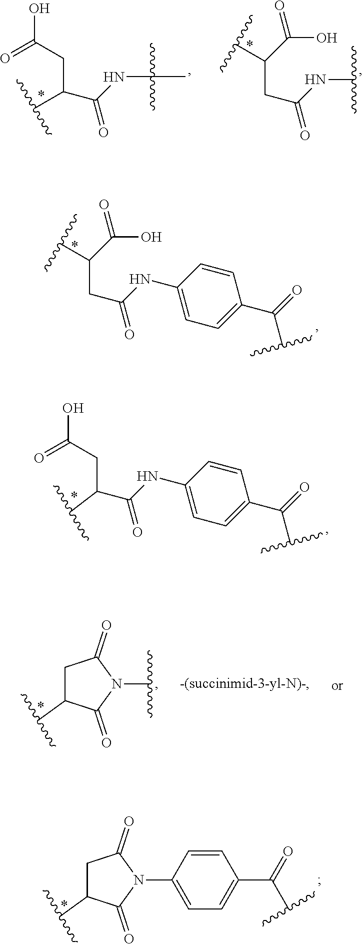 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b}:
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2; [symbol]
indicates a point of covalent attachment within the compound, and
* marks the bond where Conjugator connects to BA;
Linker has formula (IV):

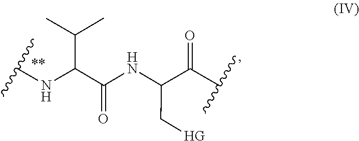

wherein:
HG is a hydrophilic residue, and
** marks the bond where Linker connects to Conjugator;
Cleavable has formula (V):

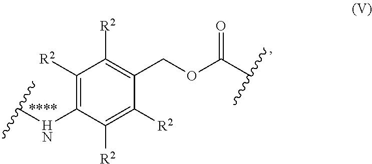

wherein:
each R² is independently hydrogen, a halogen, substituted or unsubstituted C₁₋₄ alkyl, —CN, or —NO₂, and
**** marks the bond where Cleavable connects to Linker;
Payload is a payload residue; and
x is from 1 to 15 inclusive.

In some embodiments, provided herein are ADC compounds of formula (Ib):

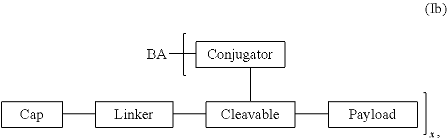

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

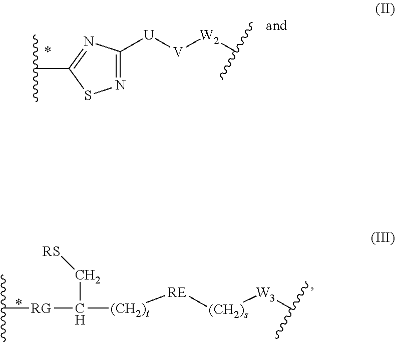

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

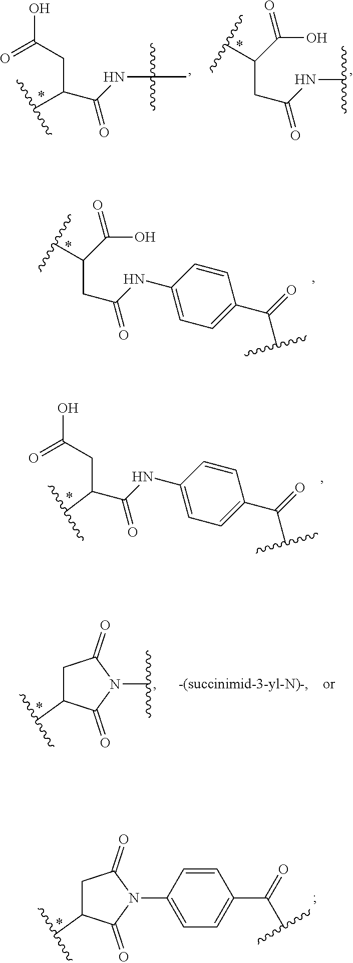 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Cleavable has formula (Va) (Vb), or (Vc):

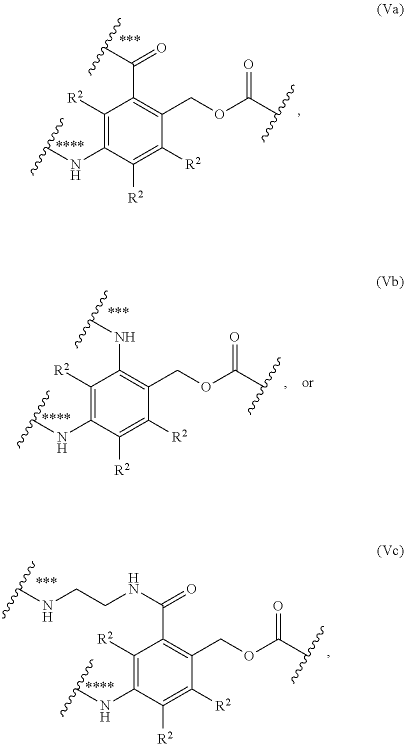

wherein
each R² is, independently, hydrogen, halogen, substituted or unsubstituted C₁₋₄ alkyl, —CN, or —NO₂.
*** marks the bond where Cleavable connects to Conjugator, and
**** marks the bond where Cleavable connects to Linker;
Linker has formula (IV′):

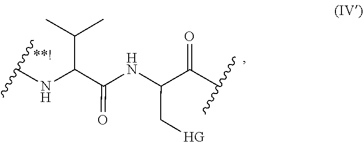

wherein
HG is a hydrophilic residue, and
**! marks the bond where Linker connects to Cap;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive;
Payload is a payload residue; and
x is from 1 to 15 inclusive.

In some embodiments, provided herein are ADC compounds of formula (Ic):

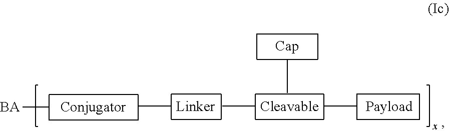

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

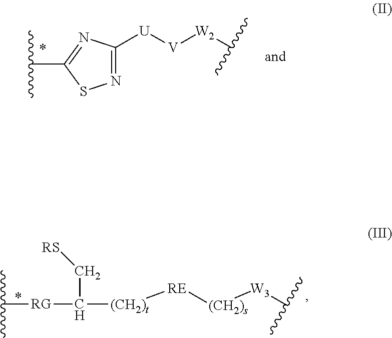

wherein;
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

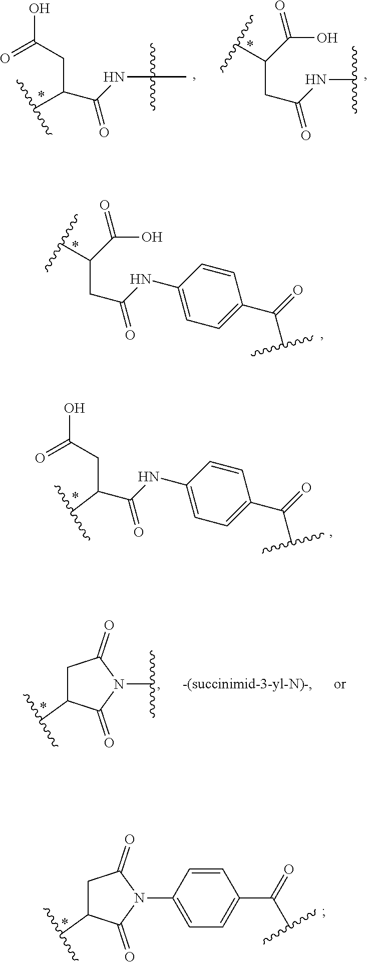 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b}.
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA:
Linker has formula (IV):

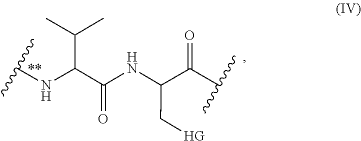

wherein:
HG is a hydrophilic residue, and;
** marks the bond where Linker connects to Conjugator;
Cleavable has formula (Va′), (Vb′), or (Vc′):

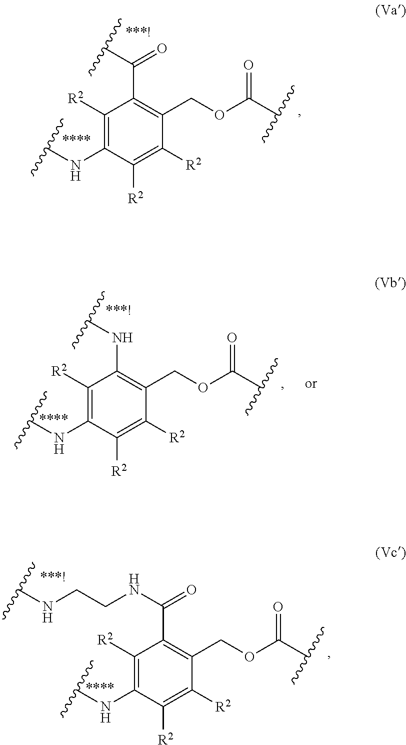

wherein:
each R² is, independently, hydrogen, halogen, substituted or unsubstituted C₁₋₄ alkyl, —CN, or —NO₂,
***! marks the bond where Cleavable connects to Cap, and
**** marks the bond where Cleavable connects to Linker:
Cap is CH₃C(═O)—, CH₃—(CH₂)_{b}—O—(CH₂CH₂O)ₐ—(CH₂CH₂)_{d}—C(═O)—, —C(═O)—(CH₂CH₂O)ₐ—(CH₂)_{b}CH₃, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂;
a is an integer between 1 and 18 inclusive;
each of b and d, independently, is 0, 1, or 2;
c is an integer between 1 and 4 inclusive;
Payload is a payload residue; and
x is from 1 to 15 inclusive.

In some embodiments, provided herein are ADC compounds of formula (Id):

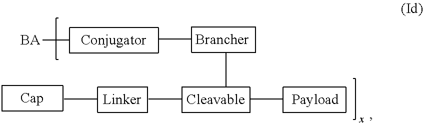

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

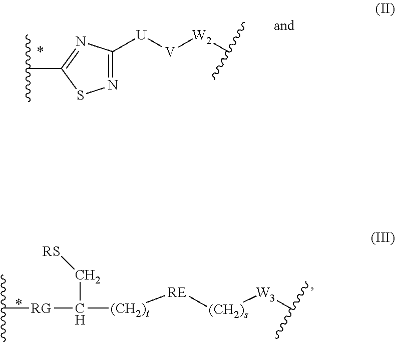

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

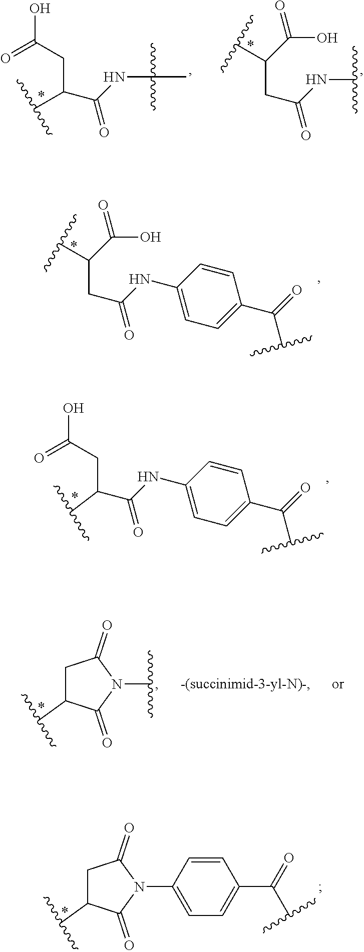 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Linker has formula (IV′):

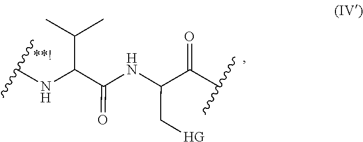

wherein:
HG is a hydrophilic residue, and
**! marks the bond where Linker connects to Cap;
Cleavable has formula (Va″), (Vb″) or (Vc″):

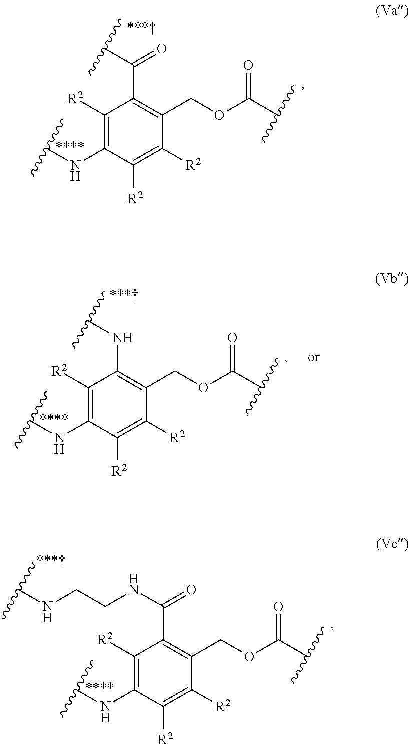

wherein:
each R² is, independently, hydrogen, halogen, substituted or unsubstituted C₁₋₄ alkyl, —CN, or —NO₂,
***† marks the bond where Cleavable connects to Brancher, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, —C(═O)—(CH₂CH₂O)ₐ—(CH₂)_{b}CH₃, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein
a is an integer between 1 and 18 inclusive;
each of b and d is, independently, 0, 1, or 2, and
c is an integer between 1 and 4 inclusive;
Brancher has formula (XIIa), (XIIa1), (XIIb) or (XIIb1):

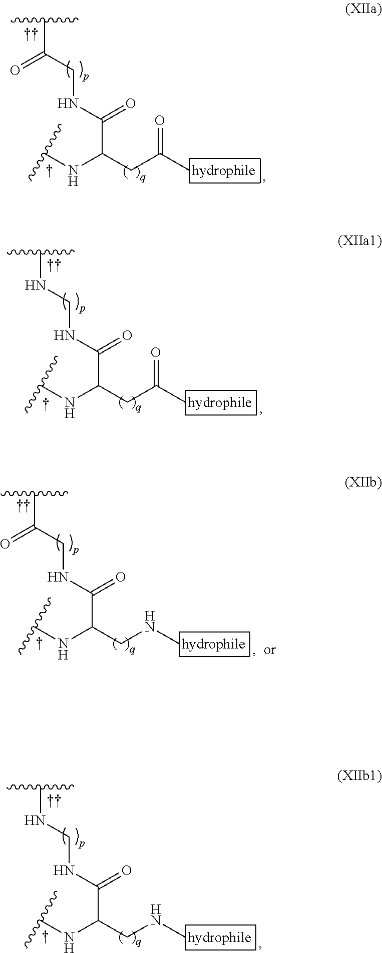

wherein:
hydrophile is —NH—(CH₂CH₂O)ₐ₁—(CH₂)_{b1}CH₃, —C(═O)NH₂, —C(═O)—(CH₂CH₂O)ₐ₁—(CH₂)_{b1}CH₃—, or —(N(CH₃)(CH₂)_{c1}C(═O)))ₐNH₂,
† marks the bond where Brancher connects to Conjugator,
†† marks the bond where Brancher connects to Cleavable,
a1 is an integer between 1 and 18 inclusive,
b1 is 0, 1, or 2; and
each of c1, p, and q is, independently, an integer between 1 and 4 inclusive,
Payload is a payload residue; and
x is from 1 to 15.

In some embodiments, the platform is a conjugator-linker-payload compound of formula (Ia′):

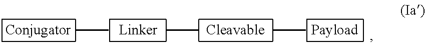

or a pharmaceutically acceptable salt, tautomer, solvate, or stereoisomer thereof, wherein:
Conjugator is selected from formula (II′) and (III′):

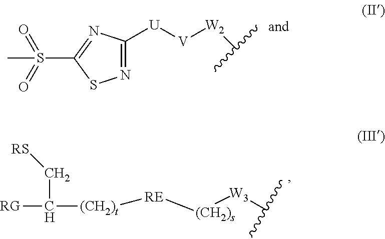

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

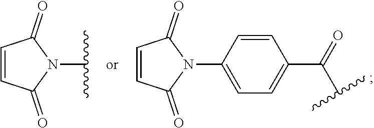 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
indicates a point of covalent attachment within the compound;
Linker has formula (IV):

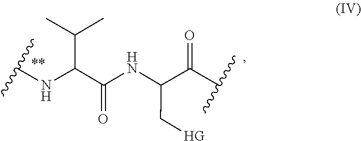

wherein:
HG is a hydrophilic residue, and
** marks the bond where Linker connects to Conjugator;
Cleavable has formula (V):

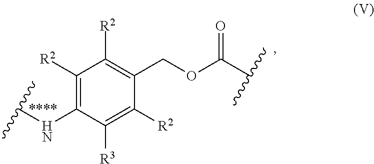

wherein:
each R² is independently hydrogen, a halogen, substituted or unsubstituted C₁₋₄ alkyl, —CN, or —NO₂, and
**** marks the bond where Cleavable connects to Linker; and
Payload is a payload residue.

In some embodiments, the platform is a conjugator-linker-payload compound of formula (Ib′):

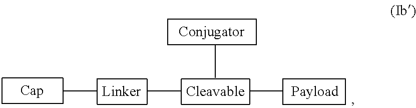

or a pharmaceutically acceptable salt, tautomer, solvate, or stereoisomer thereof, wherein:
Conjugator is selected from formula (II′) and (III′):

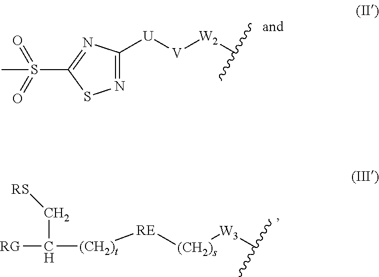

wherein
U is a bond, heteroarylene, or arylene;
V is a bond or —C≡C—(CH₂)ₙ—;
n is an integer between 0 and 10 inclusive;
W₂ is —C(O)—, —NH—, or —O—;
RG is or

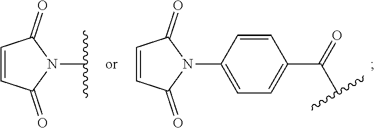 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
w indicates a point of covalent attachment within the compound;
Cleavable has formula (Va), (Vb), or (Vc):

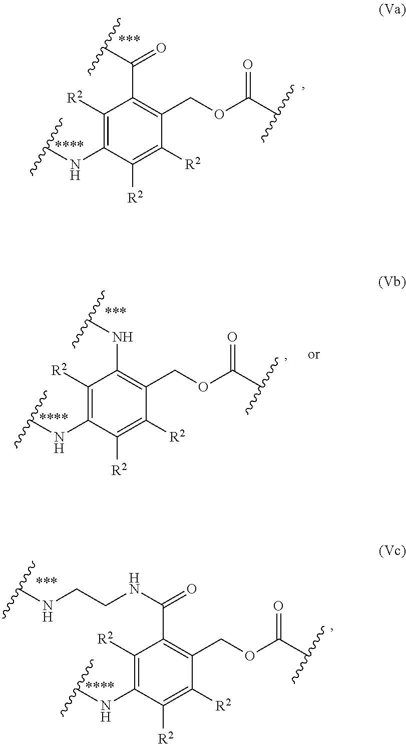

wherein:
each R² is, independently, hydrogen, halogen, substituted or unsubstituted C₁₋₄ alkyl, —CN, or —NO₂,
*** marks the bond where Cleavable connects to Conjugator, and
**** marks the bond where Cleavable connects to Linker;
Linker has formula (IV′):

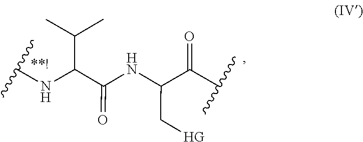

wherein
HG is a hydrophilic residue, and
**! marks the bond where Linker connects to Cap;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive; and
Payload is a payload residue.

In some embodiments, the platform is a conjugator-linker-payload compound of formula (Ib′):

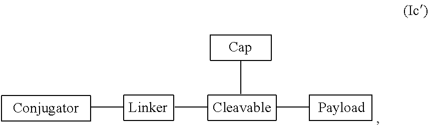

or a pharmaceutically acceptable salt, tautomer, solvate, or stereoisomer thereof, wherein:
Conjugator is selected from formula (II′) and (III′):

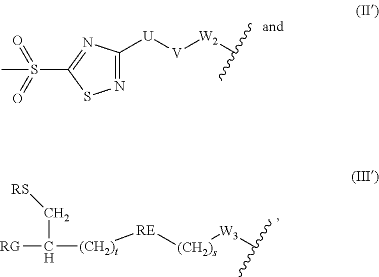

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

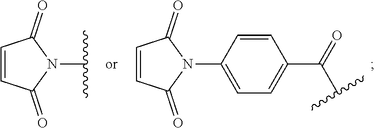 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—.
R⁶ is H or substituted or unsubstituted C₁₋₄ alkyl;
W₃ is —C(═O)—, —NH—, or —O—;
each of s and t is, independently. 1 or 2, and [symbol]
indicates a point of covalent attachment within the compound;
Linker has formula (IV):

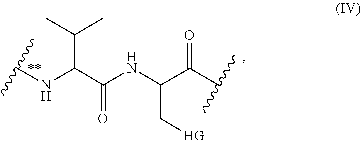

wherein:
HG is a hydrophilic residue, and
** marks the bond where Linker connects to Conjugator;
Cleavable has formula (Va′), (Vb′), or (Vc′):

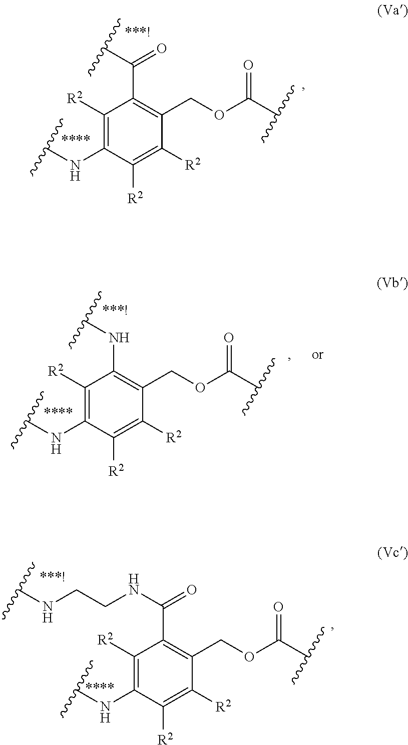

wherein:
each R² is, independently, hydrogen, halogen, substituted or unsubstituted C₁₋₄ alkyl, —CN, or —NO₂,
***! marks the bond where Cleavable connects to Cap, and
**** marks the bond where Cleavable connects to Linker:
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—, —(CH₂CH₂O)—(CH₂CH₂)ₐ—NH—C(═O)—, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive; and
Payload is a payload residue.

In some embodiments, the platform is a conjugator-linker-payload compound of formula (Id′):

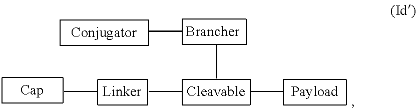

or a pharmaceutically acceptable salt, tautomer, solvate, or stereoisomer thereof, wherein:
Conjugator is selected from formula (II′) and (III′):

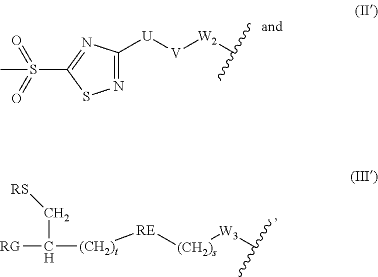

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

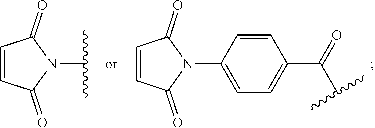 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
indicates a point of covalent attachment within the compound;
Linker has formula (IV′):

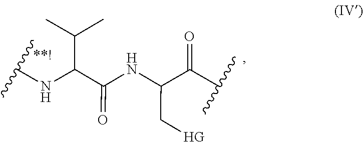

wherein:
HG is a hydrophilic residue, and
**! marks the bond where Linker connects to Cap;
Cleavable has formula (Va″), (Vb″), or (Vc″):

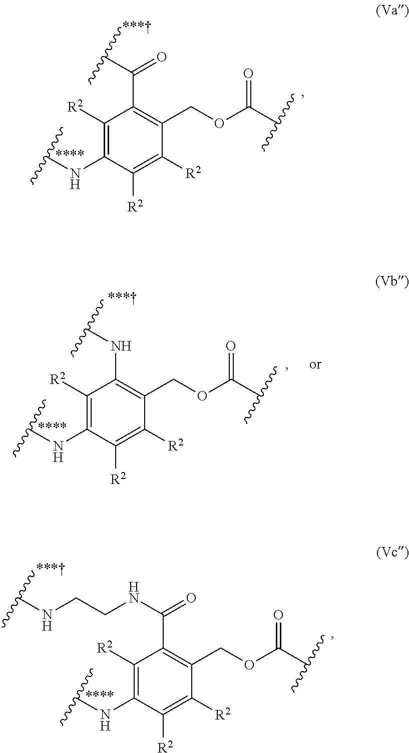

wherein:
each R² is, independently, hydrogen, halogen, substituted or unsubstituted C₁₋₄ alkyl, —CN, or —NO₂,
***^{†} marks the bond where Cleavable connects to Brancher, and
**** marks the bond where Cleavable connects to Linker:
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(═O)N(CH₃)CH₂)ₐC(═O)—, NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive; and
Brancher has formula (XIIa), (XIIa1), (XIIb) or (XIIb1):

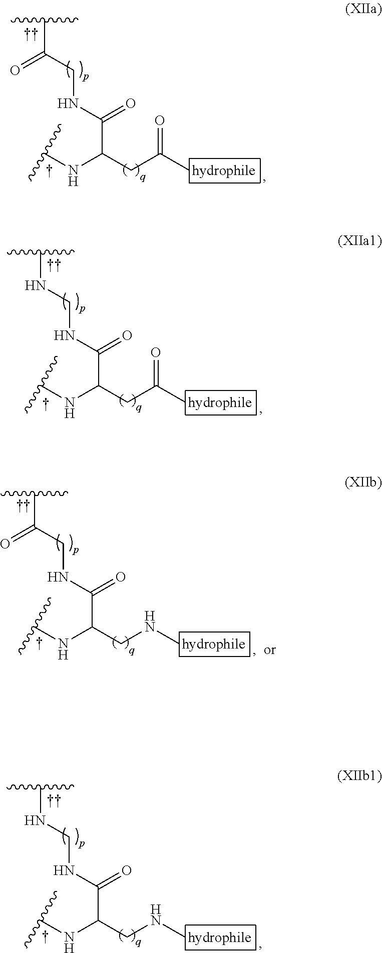

wherein:
hydrophile is —NH—(CH₂CH₂O)ₐ₁—(CH₂)_{b1}CH₃, —C(═O)NH₂, —C(═O)—(CH₂CH₂O)ₐ₁—(CH₂)_{b1}CH₃, or —(N(CH₃)(CH₂)_{c1}C(═O))ₐ₁NH₂,
† marks the bond where Brancher connects to Conjugator,
†† marks the bond where Brancher connects to Cleavable,
a1 is an integer between 1 and 18 inclusive,
b1 is 0, 1, or 2, and
each of c1, p, and q is, independently, an integer between 1 and inclusive; and
Payload is a payload residue.

Additional objects and advantages will be set forth in part in the description that follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate embodiments and together with the description serve to explain the principles described herein.

### BRIEF DESCRIPTION OF FIGURES

FIGS. 1-3 show direct cellular killing by ADC3-1, ADC3-2, ADC3-3, and ADC3-4 on U937 (FIG. 1), HL60 (FIG. 2), and TF1 (FIG. 3) cells.

FIGS. 4-6 show direct cellular killing by ADC3-5 and ADC3-6 on U937 (FIG. 4), HL60 (FIG. 5), and TF1 (FIG. 6) cells.

FIGS. 7-9 show direct cellular killing by ADC3-7, ADC3-8, and ADC3-9 on U937 (FIG. 7), HL60 (FIG. 8), and TF1 (FIG. 9) cells.

FIGS. 10-12 show direct cellular killing by ADC3-10, ADC3-11, and ADC3-12 on U937 (FIG. 10), HL60 (FIG. 11), and TF1 (FIG. 12) cells.

FIGS. 13-15 show direct cellular killing by ADC3-15 on U937 (FIG. 13), HL60 (FIG. 14), and TF1 (FIG. 15) cells.

FIGS. 16-18 show direct cellular killing by ADC3-B on NCI-H358 (FIG. 16), NCI-H11048 (FIG. 17), and MDA-MB-453 (FIG. 18) cells.

FIGS. 19-21 show direct cellular killing by ADC4-1, ADC4-2, and ADC4-3 on NCI-H358 (FIG. 19), NCI-H1048 (FIG. 20), and MDA-MB-453 (FIG. 21) cells.

FIG. 22 shows ADC, payload, and total antibody (“TAB”) PK profiles in mice.

FIG. 23 shows anti-tumor efficacy of ADCs in an NCI-H1650 xenograft model.

### DETAILED DESCRIPTION

Provided herein are antibody drug conjugates (ADCs) and covalent linkers and conjugator-linker-payloads (platforms) for making such ADCs. The ADCs may be used to treat a disease or disorder, such as cancer, such as by providing a composition comprising an ADC. The presently disclosed ADCs can better resist deconjugation and premature payload release, and allow for increased DAR, increased stability in circulation, improved pharmacokinetics, and improved efficacy compared to known ADCs, including those in which the payload is MMAE. The conjugates include linkers with at least one moiety that improves their performance, such as by increasing serum stability compared to conjugates without the moiety. The linkers comprise a hydrophilic moiety (group) that can be cleaved by a glycosidase, such as a lysosomal-specific glycosidase.

### Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise,

When a trade name is used herein, reference to the trade name also refers to the product formulation, the generic drug, and the active pharmaceutical ingredient(s) of the trade name product, unless otherwise indicated by context.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5^{th} Ed., Garland Publishing, New York). An antibody can be of any type (e.g., IgG, IgE, IgM, IgD, and IgA), class (e.g., IgG1, IgG2, IgG3, IgG4, IgA1, and IgA2) or subclass. The antibody can be derived from any suitable species. In some embodiments, the antibody is of human or murine origin. An antibody can be, for example, human, humanized, or chimeric.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH₁, CH₂, CH₃, and CH₄, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The term “cytotoxic activity” refers to a cell-killing, a cytostatic or an anti-proliferative effect of a drug-linker-ligand conjugate compound or an intracellular metabolite of a drug-linker-ligand conjugate. Cytotoxic activity may be expressed as the IC50 value, which is the concentration (molar or mass) per unit volume at which half the cells survive.

The term “cytotoxic agent” as used herein refers to a substance that inhibits the function of cells and/or causes destruction of cells. The term is intended to include radioactive isotopes (e.g., 211At, 131I, 125I, 90Y, 186Re, 188Re, 153Sm, 212Bi, 32P, 60C, and radioactive isotopes of Lu), chemotherapeutic agents, and toxins such as small molecule toxins or enzymatically active toxins of bacterial, fungal, plant, or animal origin, including synthetic analogs and derivatives thereof

The terms “cancer” and “cancerous” refer to or describe the physiological condition or disorder in mammals that is typically characterized by unregulated cell growth. A “tumor” comprises one or more cancerous cells.

An “autoimmune disease” herein is a disease or disorder arising from and directed against an individual's own tissues or proteins.

Examples of a “patient” or “subject” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6% 5%, 4%, 3%, 2%, 1.5%, 1%, 0.5%, or 0.25% of the recited value or range of values. For example, in some embodiments, the value of an XRPD peak position may vary by up to ±0.2° 2θ while still describing the particular XRPD peak.

As used herein, the term “inclusive,” when used in reference to range, includes the endpoints of the range. For example, if n is an integer from 0 to 4, n may be any of 0, 1, 2, 3, or 4.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl. 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H4-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, inidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthrax.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R, is as defined above.

An “aralkoxyamine” group is a radical of the formula N(R^{#})O-aryl or NHOaryl, wherein R is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R^{#} are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R^{#})C(═O)(R^{#}) or N((C═O)(R^{#}))₂, wherein each R^{#} is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#}), —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#}), —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, or —S(═O)₂NH(R^{#}), or —S(═O)₂NH₂, wherein each R^{#} is independently as defined above.

A “phosphonate” group is a radical of the formula —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L., Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

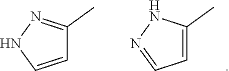

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (t-amino-1-deoxy-D-glucitol) linked to the rest of molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic”is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, orithine, and citrulline.

As used herein “peptide,” in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“Aho” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab. In one embodiment, trastuzumab can be made and used as described in U.S. Pat. Nos. 6,407,213 and 5,821,337, the entire disclosures of which are incorporated herein by reference.

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference.

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference.

As used herein, the term “ifnatamab” refers to an antibody selectively binding to the B7H3 receptor, i.e., an anti-human B7H3 antibody. In one embodiment, ifinatamab can be made and used as described in U.S. Pat. No. 10,117,952 or WO2022102695, the entire disclosures of which are incorporated herein by reference.

As used herein, the term “6E7” refers to a CLL1 monoclonal antibody.

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### Conjugates

In embodiments, a conjugate, or a pharmaceutically acceptable salt, tautomer, solvate, or stereoisomer thereof, includes a protein linked to at least one payload or payload residue (also referred to herein as a Drug unit) and linked to at least one hydrophilic moiety via a covalent linker. The covalent linker is bonded directly or indirectly to each of the protein, the payload residue, and the hydrophilic moiety. In some embodiments, the protein is a binding agent, such as an antibody or antigen-binding fragment thereof.

In some embodiments, the protein is bonded directly to a covalent linker, such as a linker set forth herein. In such cases, the binding agent is one bond position away from the covalent linker. The covalent linker may also be bonded directly to a payload residue such that the covalent linker is one bond position away from a payload residue. The payload may be any payload set forth herein, such as MMAE. In some embodiments, the covalent linker is also bonded directly or indirectly to a hydrophilic moiety such that the covalent linker is one bond position, or more than one bond position, away from a hydrophilic moiety. The hydrophilic moiety may be any hydrophilic moiety set forth herein.

In some embodiments, the binding agent is bonded indirectly to a covalent linker such that the binding agent is more than one bond position away from the covalent linker. In such cases, the binding agent is bonded through another moiety to the covalent linker. For example, the binding agent may be bonded to a maleimide group which is bonded to a polyethylene glycol group which is bonded to the covalent linker.

In some examples, the covalent linker is also bonded indirectly to a payload residue such that the covalent linker is more than one bond position away from a payload residue. The covalent linker is bonded through another moiety to the payload. For example, the covalent linker may be bonded to a dipeptide, such as but not limited to Val-Ala or Val-Cit, which may be bonded to a p-aminobenzyl alcohol (PAB)-derived unit, which may be bonded to the payload residue. An example of a PAB-derived unit is —NH—(C6H4)-CH2-O—C(═O)—, and the phenylene portion of the PAB unit may be substituted with —C1-C8 alkyl, —O—(C1-C8 alkyl), -halogen, -nitro, or -cyano.

The compounds disclosed herein include at least a Conjugator, Linker, Cleavable, and Payload. In some embodiments, compounds include a Cap. In some embodiments, compounds include a Brancher. In some embodiments, such as of conjugates, compounds include a binding agent (BA). Conjugator is a group capable of bonding a BA and joining the BA to the rest of the compound. Cleavable and Linker together include at least one site for enzymatic cleavage and provide distance between Conjugator and Payload. Linker may include a hydrophilic moiety Linker may join Conjugator to Cleavable or Cleavable to Cap, if present. Brancher is a group that extends the framework of the linker and includes a hydrophile. Linker, Cleavable, Cap, and Brancher may form the linker portion of a conjugate. Payload (also referred to herein as a Drug unit) is a cytotoxic agent or residue thereof

### Aspect 1

Provided herein are compounds of formula (Ia):

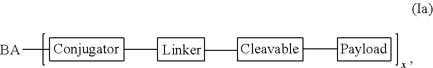

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

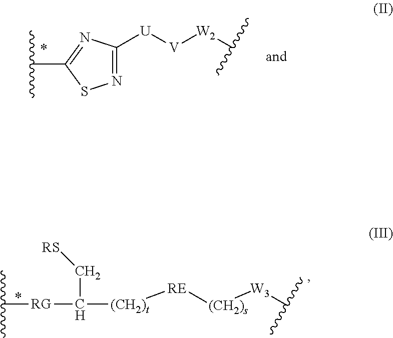

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

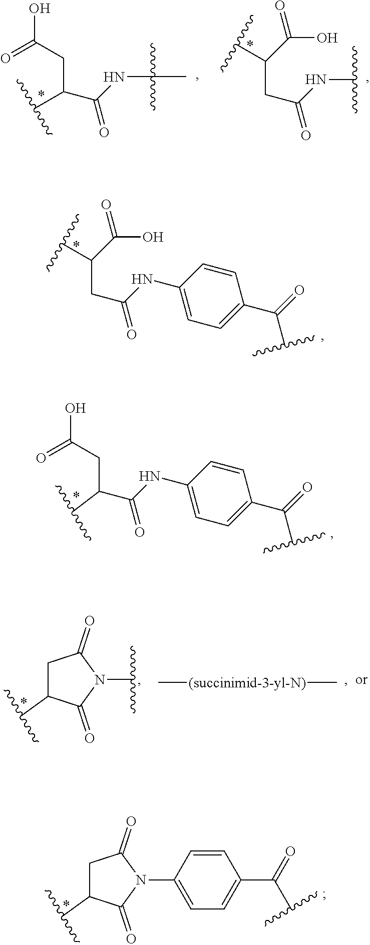

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2; [symbol]
indicates a point of covalent attachment within the compound, and
* marks the bond where Conjugator connects to BA;
Linker has formula (IV):

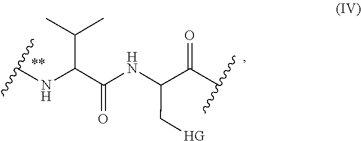

wherein:
HG is a hydrophilic residue, and
** marks the bond where Linker connects to Conjugator;
Cleavable has formula (V):

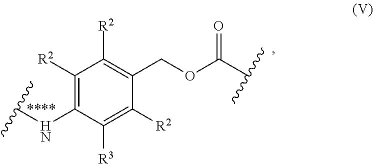

wherein:
each R² is independently hydrogen, a halogen, substituted or unsubstituted C₁₋₄ alkyl, —CN, or —NO₂, and
**** marks the bond where Cleavable connects to Linker;
Payload is a payload residue; and
x is from 1 to 15 inclusive.

In some embodiments of Aspect 1, Cleavable has the following formula:

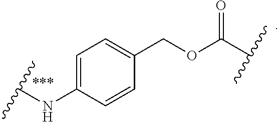 [SMILES: *Nc1ccc(COC(*)=O)cc1]

### Aspect 2

Provided herein are compounds of formula (Ib):

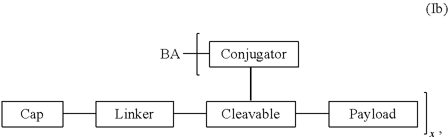

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

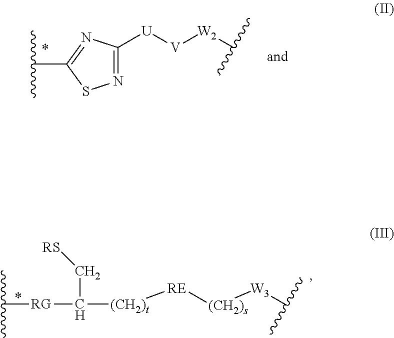

wherein:
U is a bond, heteroarylene, or arylene;
V is a bond or —C≡C—(CH₂)ₙ—;
n is an integer between 0 and 10 inclusive;
W₂ is —C(═O)—, —NH— or —O—;
RG is

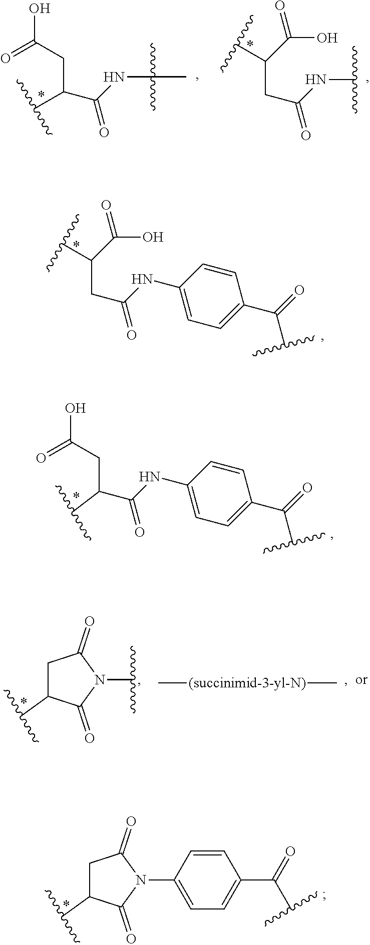

RS is —NR^{1a}R^{1b};
each of R^{1a} and R^{1b} is, independently, H or substituted or unsubstituted C₁₋₄ alkyl
RE is a bond, —O—, —OC(═O)—, —OC(═O)NR⁶—, —NHC(═O)NR⁶—. —OS(═O)NR⁶—, —NHS(═O)₂NR⁶—, or —OC(═O)NHS(═O)₂NR⁶—;
RP is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Cleavable has formula (Va), (Vb), or (Vc):

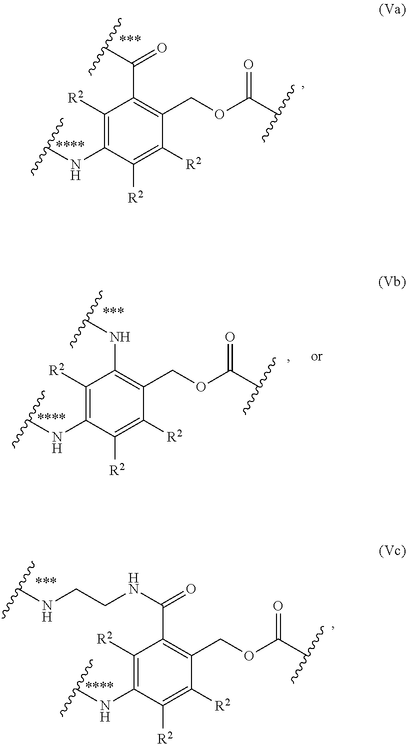

wherein:
each R² is, independently, hydrogen, halogen, substituted or unsubstituted C₁₋₄ alkyl, —CN, or —NO₂,
*** marks the bond where Cleavable connects to Conjugator, and
**** marks the bond where Cleavable connects to Linker;
Linker has formula (IV′):

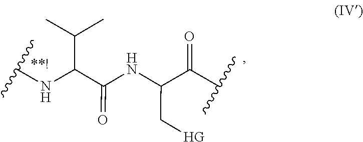

wherein
HG is a hydrophilic residue, and
**! marks the bond where Linker connects to Cap;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—, —(CH₂CH₂O)ₐ—(CH₂CH₂)—NH—C(═O)—, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive:
Payload is a payload residue; and
x is from 1 to 15 inclusive.

In some embodiments of Aspect 2, Cleavable has one of the following formulas:

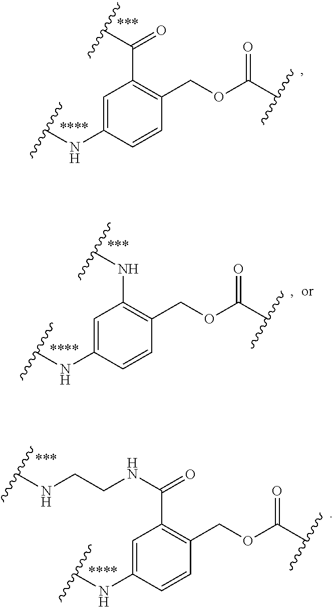

In some embodiments of Aspect 2, Cap is CH₃C(═O)—, CH₃—(CH₂)_{b}—O—(CH₂CH₂O)ₐ—(CH₁₂C₁₂)_{d}—C(═O)—, or CH₃(C(═O)N(CH₃)CH₂)ₐC(═O)—.

In some embodiments of Aspect 2, Cap is CH₃C(═O)—CH₃—O—(CH₂CH₂O)₁₁—(CH₂CH₂)—C(═O)—, or —CH₃(C(═O)N(CH₃)CH₂)₁₂C(═O)—.

### Aspect 3

Provided herein are compounds of formula (Ic):

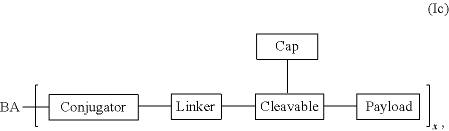

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

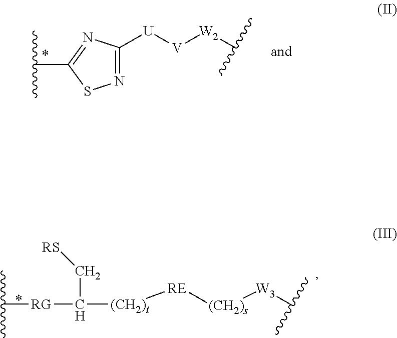

wherein;
U is a bond, heteroarylene, or arylene;
V is a bond or —C═—C—(CH₂)ₙ—;
n is an integer between 0 and 10 inclusive;
W₂ is —C(O)—, —NH—, or —O—;
RG is

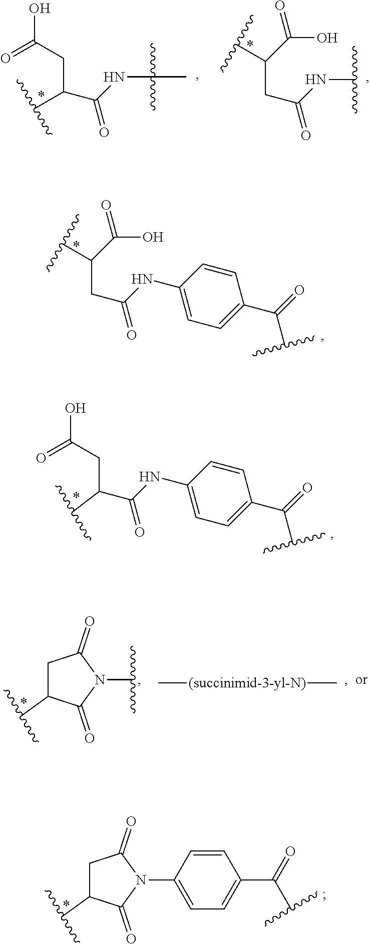

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Linker has formula (IV):

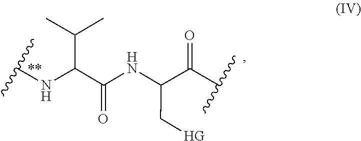

wherein:
HG is a hydrophilic residue, and;
** marks the bond where Linker connects to Conjugator;
Cleavable has formula (Va′), (Vb′), or (Vc′):

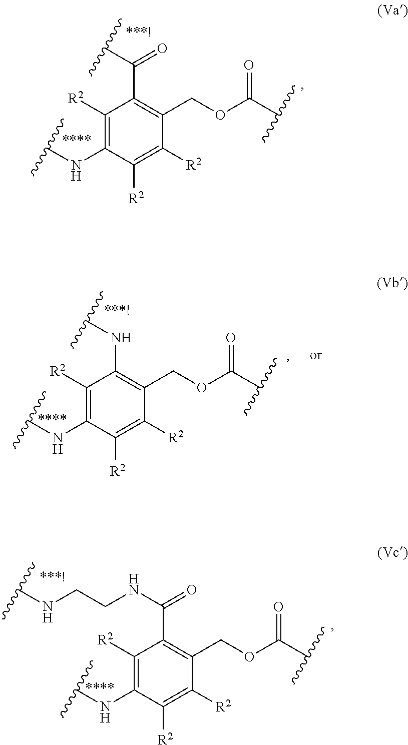

wherein:
each R² is, independently, hydrogen, halogen, substituted or unsubstituted C₁₋₄ alkyl, —CN, or —NO₂,
***! marks the bond where Cleavable connects to Cap, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, —C(═O)—(CH₂CH₂O)ₐ—(CH₂)_{b}CH₃, CH₃(C(═O)N(CH₃)CH₂)ₐC(═O)—, —NH—(CH₂CH₂O)ₐ—(CH₂)_{b}CH₃, or —(N(CH₃)(CH₂)₄C(═O))ₐNH₂;
a is an integer between 1 and 18 inclusive;
each of b and d, independently, is 0, 1, or 2;
c is an integer between 1 and 4 inclusive;
Payload is a payload residue; and
x is from 1 to 15 inclusive.

In some embodiments of Aspect 3, Cleavable has one of the following formulas:

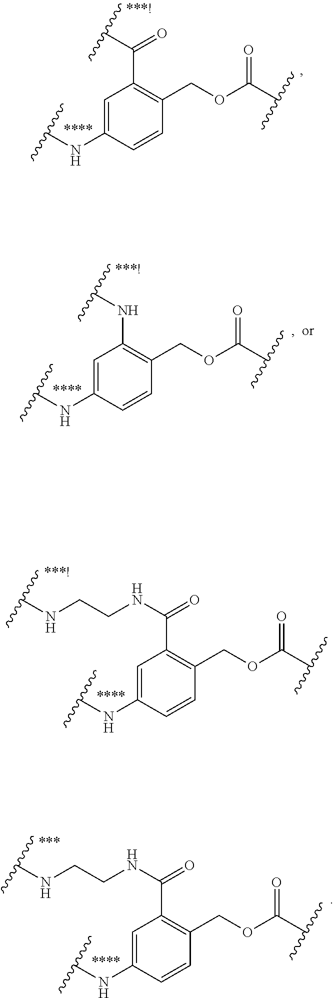 [SMILES: *Nc1ccc(COC(*)=O)c(C(*)=O)c1 ; *Nc1ccc(COC(*)=O)c(N*)c1 ; *NCCNC(=O)c1cc(N*)ccc1COC(*)=O ; *NCCNC(=O)c1cc(N*)ccc1COC(*)=O]

In some embodiments of Aspect 3, Cap is —NH—(CH₂CH₂O)ₐ—(CH₂)_{b}CH₃, or —(N(CH₃)(CH₂)_{c}C(═O))ₐNH₂.

In some embodiments of Aspect 3, Cap is —NH—(CH₂CH₂O)₁₂—CH₃ or —(N(CH₃)CH₂C(═O))₁₂NH₂.

### Aspect 4

Provided herein are ADC compounds of formula (Id):

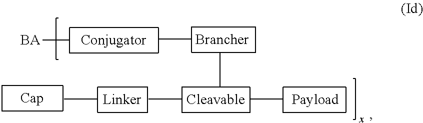

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

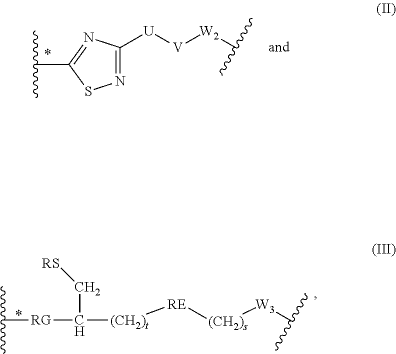

wherein:
U is a bond, heteroarylene, or arylene;
V is a bond or —C═—C—(CH₂)ₙ—;
n is an integer between 0 and 10 inclusive;
W₂ is —C(═O)—, —NH—, or —O—;
RG is

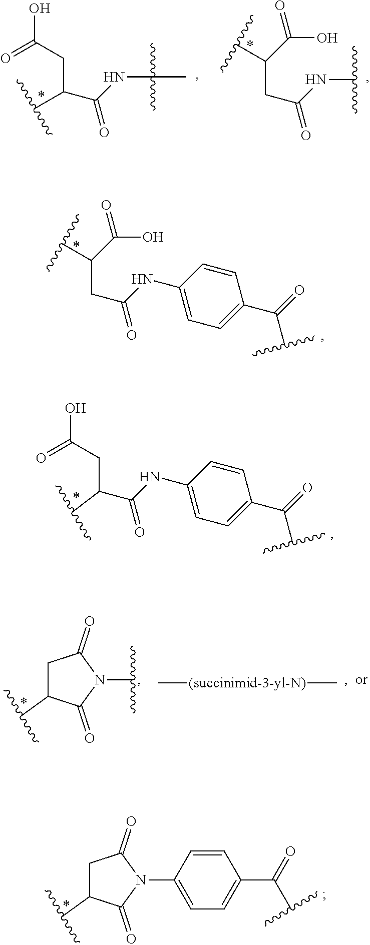

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Linker has formula (IV′):

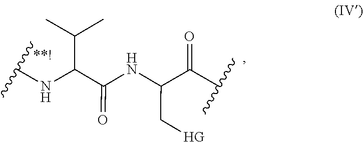

wherein:
HG is a hydrophilic residue, and
**! marks the bond where Linker connects to Cap;
Cleavable has formula (Va″), (Vb″), or (Vc″):

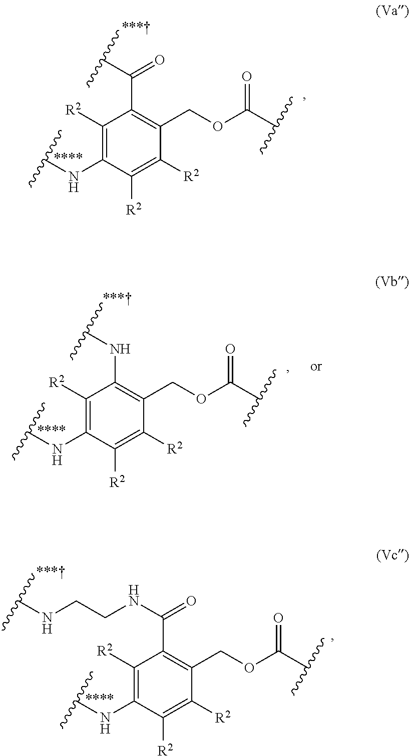

wherein:
each R² is, independently, hydrogen, halogen, substituted or unsubstituted C₁₄ alkyl, —CN, or —NO₂,
***^{†} marks the bond where Cleavable connects to Brancher, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, —C(═O)—(CH₂CH₂O)ₐ—(CH₂)_{b}CH₃, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein
a is an integer between 1 and 18 inclusive;
each of b and d is, independently, 0, 1, or 2, and
c is an integer between 1 and 4 inclusive;
Brancher has formula (XIIa), (XIIa1), (XIIb) or (XIIb1):

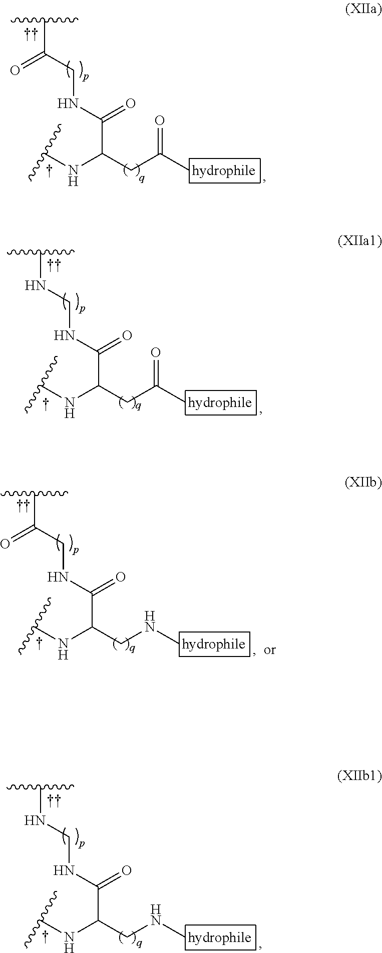

wherein:
hydrophile is —NH—(CH₂CH₂O)ₐ₁—(CH₂)₁CH₃, —C(═O)NH₂, —C(═O)—(CH₂CH₂O)ₐ₁—(CH₂)_{b1}CH₃, or —(N(CH₃)(CH₂)_{c1}C(═O))ₐ₁NH₂,
† marks the bond where Brancher connects to Conjugator,
†† marks the bond where Brancher connects to Cleavable,
a1 is an integer between 1 and 18 inclusive,
b1 is 0, 1, or 2; and
each of c1, p, and q is, independently, an integer between 1 and 4 inclusive,
Payload is a payload residue; and
x is from 1 to 15.

In some embodiments of Aspect 4, Cleavable has one of the following formulas:

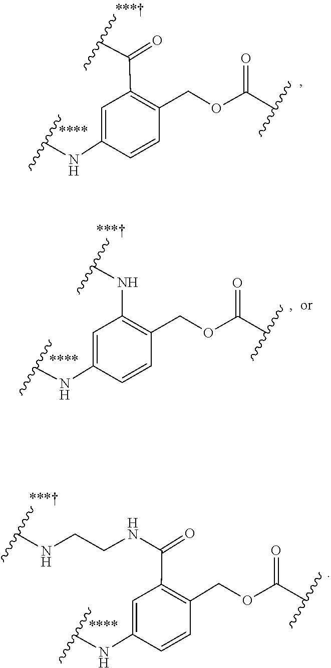 [SMILES: *Nc1ccc(COC(*)=O)c(C(*)=O)c1 ; *Nc1ccc(COC(*)=O)c(N*)c1 ; *NCCNC(=O)c1cc(N*)ccc1COC(*)=O]

In some embodiments of Aspect 4, a1 is 12, b1 is 0, c1 is 1, p is 2, and q is 2 or 4.

In some embodiments of Aspect 4, Brancher has one of the following formulas:

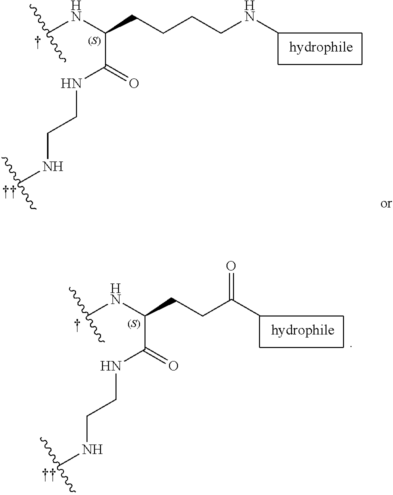

In some embodiments of Aspect 4, hydrophile is —C(═O)—(CH₂CH₂O)ₐ₁—CH₃ or —(N(CH₃)—CH₂—C(═O))ₐ₁NH₂, and a1 is an integer between 10 to 16 inclusive.

In some embodiments of Aspect 4, a1 is 12.

In some embodiments of Aspect 4, Cap is CH₃C(═O)—.

### Aspects 1, 2, 3, and 4

The foregoing ADC compounds of formula (Ia), (Ib), (Ic), and/or (Id) have a drug-to-antibody ratio (DAR) (x) and include a Conjugator, Linker, Payload, and BA (binding agent), each as described below.

In embodiments of Aspects 1 to 4, x is from 1 to 15 inclusive. In embodiments, x is from 1 to 12. In embodiments, x is from 1 to 10. In embodiments, x is from 2 to 10. In embodiments, x is from 3.5 to 10. In embodiments, x is from 3.5 to 8. In embodiments, x is from 3.5 to 6. In one embodiment, x is from 3.5 to 4.5.

### Binding Agents

Provided herein are binding agents (BA or Ab), e.g., for use in an ADC described herein.

Compounds of formula (I) may include any BA described herein.

In some embodiments, BA is an antibody or antigen-binding fragment thereof, e.g., a humanized, chimeric, or human antibody or an antigen-binding fragment thereof.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)₂ fragment.

In some embodiments, BA is an antibody or an antigen-binding fragment thereof that binds to one or more of B7H3, cytokeratin 15, PTK7, HER3, HER2, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, ECFR, ECFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, Tn, B7H3, BCMA, MUC1, cMet, ROR1 MSLn, FRa, CLDN18.2, CLDN6, PTK7, Ax1, FGFR2b, CLL1, CCR7, GPC1, CPC3, ISAC, CDCP1, ITGB6, ADAM9, or CD45-iADC.

In some embodiments, BA is an anti-human B7H3 antibody, an anti-CLL1 antibody, an anti-PTK7 antibody, an anti-HER3 antibody, an anti-HER2 antibody, or an antigen-binding fragment of any of the foregoing. In some embodiments, the anti-human B7H3 antibody is ifinatamab. In some embodiments, the anti-CLL1 antibody is 6E7. In some embodiments, the anti-PTK7 antibody is cofetuzumab. In some embodiments, the anti-HER3 antibody is patritumab. In some embodiments, the anti-HER2 antibody is trastuzumab.

In some embodiments, the antibody or antigen-binding fragment thereof specifically binds human B7H3. In some embodiments, the antibody or antigen-binding fragment thereof is ifinatamab.

In some embodiments, the antibody or antigen-binding fragment thereof specifically binds CLL1. In some embodiments, the antibody or antigen-binding fragment thereof is 6E7.

In some embodiments, the antibody or antigen-binding fragment thereof specifically binds PTK7. In some embodiments, the antibody or antigen-binding fragment thereof is cofetuzumab.

In some embodiments of any of Aspects 1 to 4, Conjugator comprises formula (II).

In some embodiments of any of Aspects 1 to 4, U is arylene.

In some embodiments of any of Aspects 1 to 4, U is phenylene.

In some embodiments of any of Aspects 1 to 4. U is

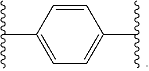 [SMILES: *c1ccc(*)cc1]

In some embodiments of any of Aspects 1 to 4, U is heteroarylene.

In some embodiments of any of Aspects 1 to 4, L is a bivalent pyrimidine ring.

In some embodiments of any of Aspects 1 to 4, U is

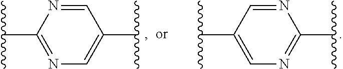 [SMILES: *c1cnc(*)nc1 ; *c1cnc(*)nc1]

In some embodiments of any of Aspects 1 to 4. U is a bond.

In some embodiments of any of Aspects 1 to 4, V is a bond.

In some embodiments of any of Aspects 1 to 4, V is —C≡C—(CH₂)ₙ—.

In some embodiments of any of Aspects 1 to 4, V is —C≡C—(CH₂)₃—.

In some embodiments of any of Aspects 1 to 4, W₂ is —C(═O)—.

In some embodiments of any of Aspects 1 to 4, Conjugator has formula (III).

In some embodiments of any of Aspects 1 to 4, RS is —N(CH₃)₂.

In some embodiments of any of Aspects 1 to 4, RS is —NH₂.

In some embodiments of any of Aspects 1 to 4, RE is —OC(═O)NH—.

In some embodiments of any of Aspects 1 to 4, RG is

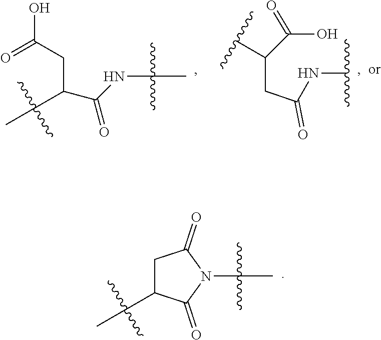

Certain RG described herein, e.g.,

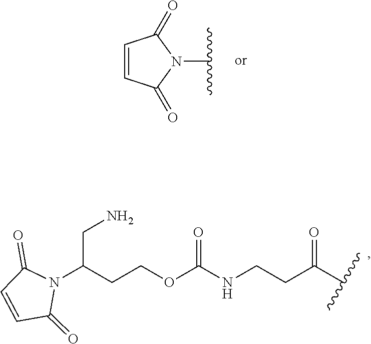 [SMILES: *N1C(=O)C=CC1=O ; *C(=O)CCNC(=O)OCCC(CN)N1C(=O)C=CC1=O]

may, upon conjugation with BA, be induced to undergo a ring opening process. During such process the maleimide structure formed upon conjugation of the RG with BA, e.g.,

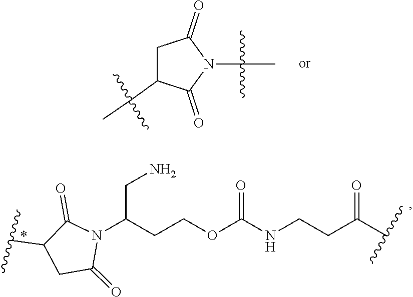 [SMILES: *C1CC(=O)N(*)C1=O ; *C(=O)CCNC(=O)OCCC(CN)N1C(=O)CC(*)C1=O]

undergoes ring opening. It will be appreciated that there are two possible regioisomers that may result from the ring opening of such maleimide structures. For example,

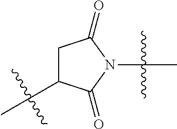 [SMILES: *C1CC(=O)N(*)C1=O]

may undergo ring opening to form

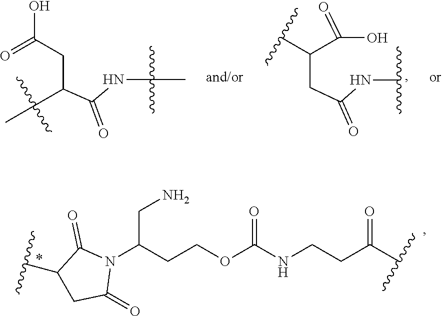 [SMILES: *NC(=O)C(*)CC(=O)O ; *NC(=O)CC(*)C(=O)O ; *C(=O)CCNC(=O)OCCC(CN)N1C(=O)CC(*)C1=O]

may undergo ring opening to form

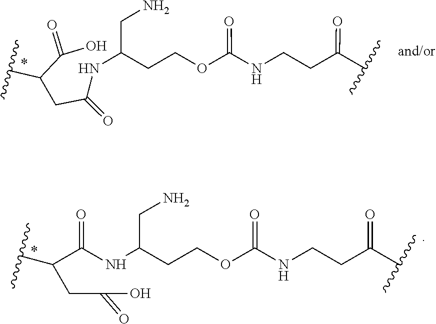 [SMILES: *C(=O)CCNC(=O)OCCC(CN)NC(=O)CC(*)C(=O)O ; *C(=O)CCNC(=O)OCCC(CN)NC(=O)C(*)CC(=O)O]

For simplicity, unless indicated otherwise, both possible ring-open regioisomers are encompassed by a depiction of a single ring-open regioisomer herein. Thus, unless indicated otherwise, depiction of the ring-open regioisomer

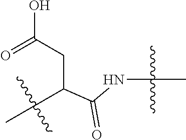 [SMILES: *NC(=O)C(*)CC(=O)O]

encompasses

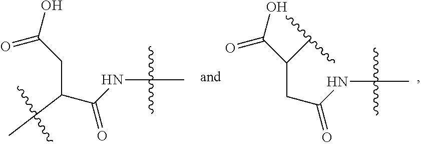 [SMILES: *NC(=O)C(*)CC(=O)O ; *NC(=O)CC(*)C(=O)O]

and likewise, depiction of the ring-open regioisomer

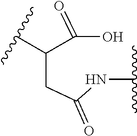 [SMILES: *NC(=O)CC(*)C(=O)O]

encompasses

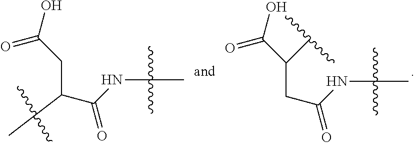 [SMILES: *NC(=O)C(*)CC(=O)O ; *NC(=O)CC(*)C(=O)O]

In some embodiments, a mixture of ring-open regioisomers is present. In some embodiments, a single ring-open regioisomer is present.

In some embodiments of any of Aspects 1 to 4, each of s and t is 2.

In some embodiments of any of Aspects 1 to 4, Conjugator has formula (IIa1), (IIa2), (IIa3), or (IIIa):

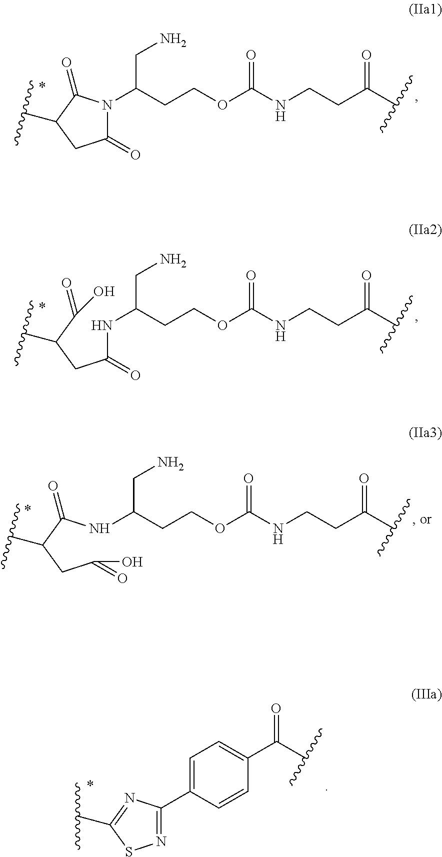 [SMILES: *C(=O)CCNC(=O)OCCC(CN)N1C(=O)CC(*)C1=O ; *C(=O)CCNC(=O)OCCC(CN)NC(=O)CC(*)C(=O)O ; *C(=O)CCNC(=O)OCCC(CN)NC(=O)C(*)CC(=O)O ; *C(=O)c1ccc(-c2nsc(*)n2)cc1]

In some embodiments of any of Aspects 1 to 4, the Linker has the following formula:

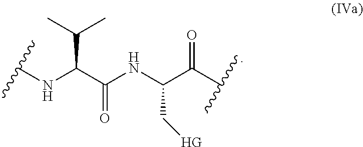

In some embodiments of any of Aspects 1 to 4.
HG is

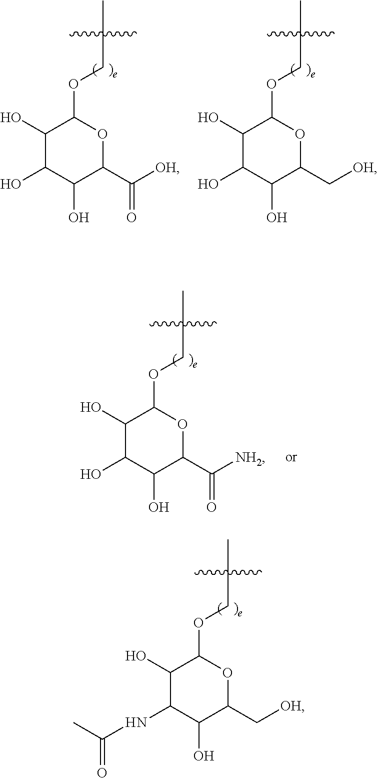

wherein e is 0, 1, or 2; or
HG is —(CH₂CH₂)_{z}NR^{a}C(O)NR^{b}R^{c}, wherein z is 1, 2, 3, or 4, and each of R^{a}, R^{b}, and R^{c} is, independently, H, or substituted or unsubstituted C₁₋₄ alkyl.

In some embodiments of any of Aspects 1 to 4. Linker has one of the following formulas:

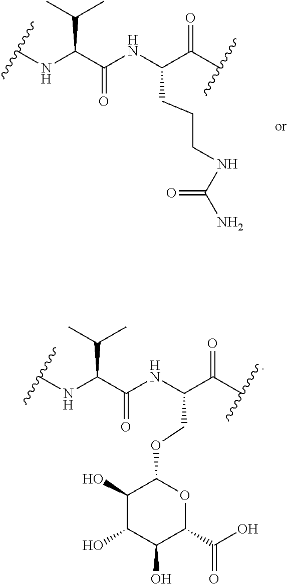 [SMILES: *N[C@H](C(=O)N[C@@H](CCCNC(N)=O)C(*)=O)C(C)C ; *N[C@H](C(=O)N[C@@H](CO[C@@H]1O[C@H](C(=O)O)[C@@H](O)[C@H](O)[C@H]1O)C(*)=O)C(C)C]

in some embodiments of any of Aspects 1 to 4, Payload is a residue of one of the following formulas:

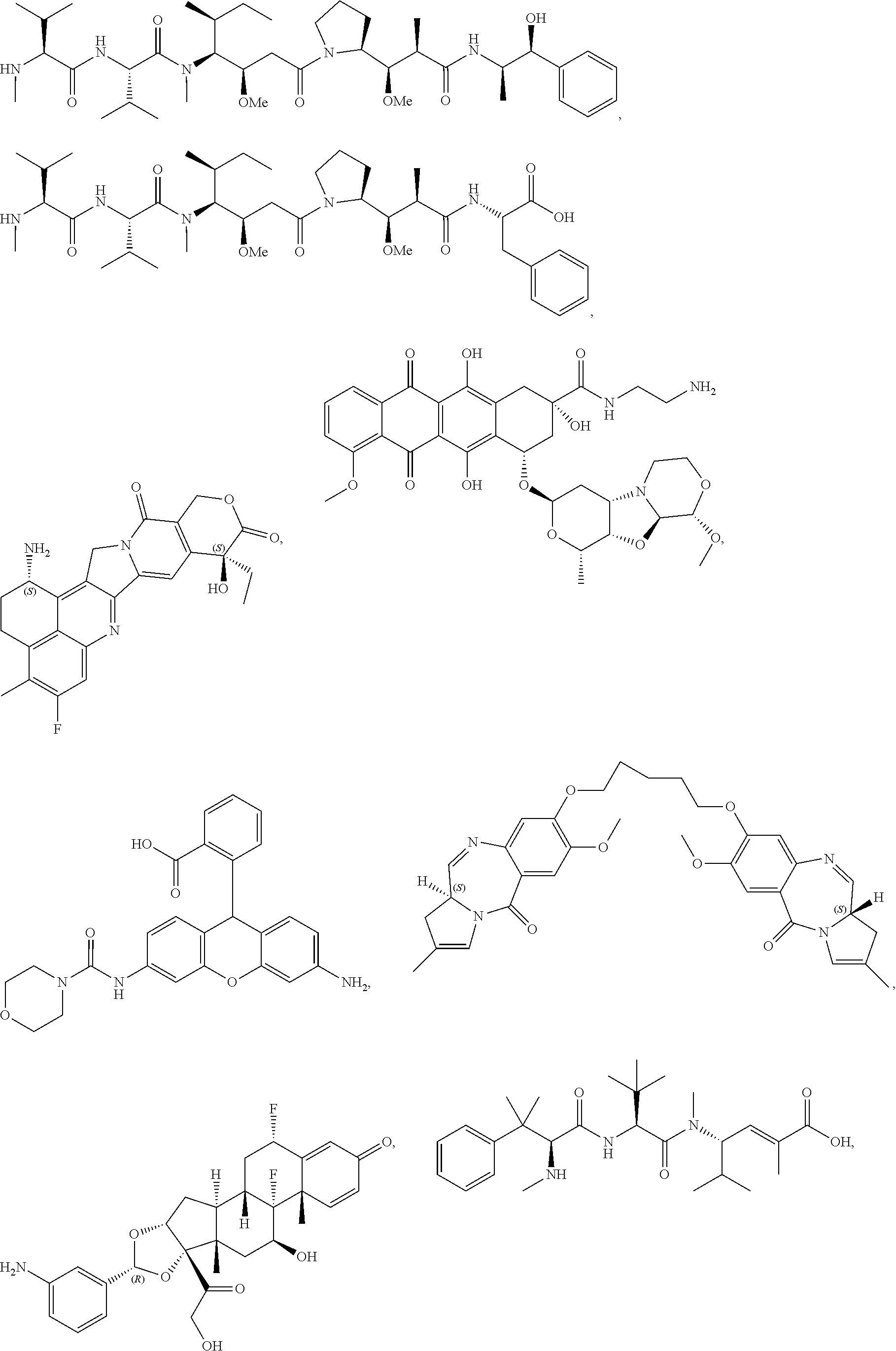

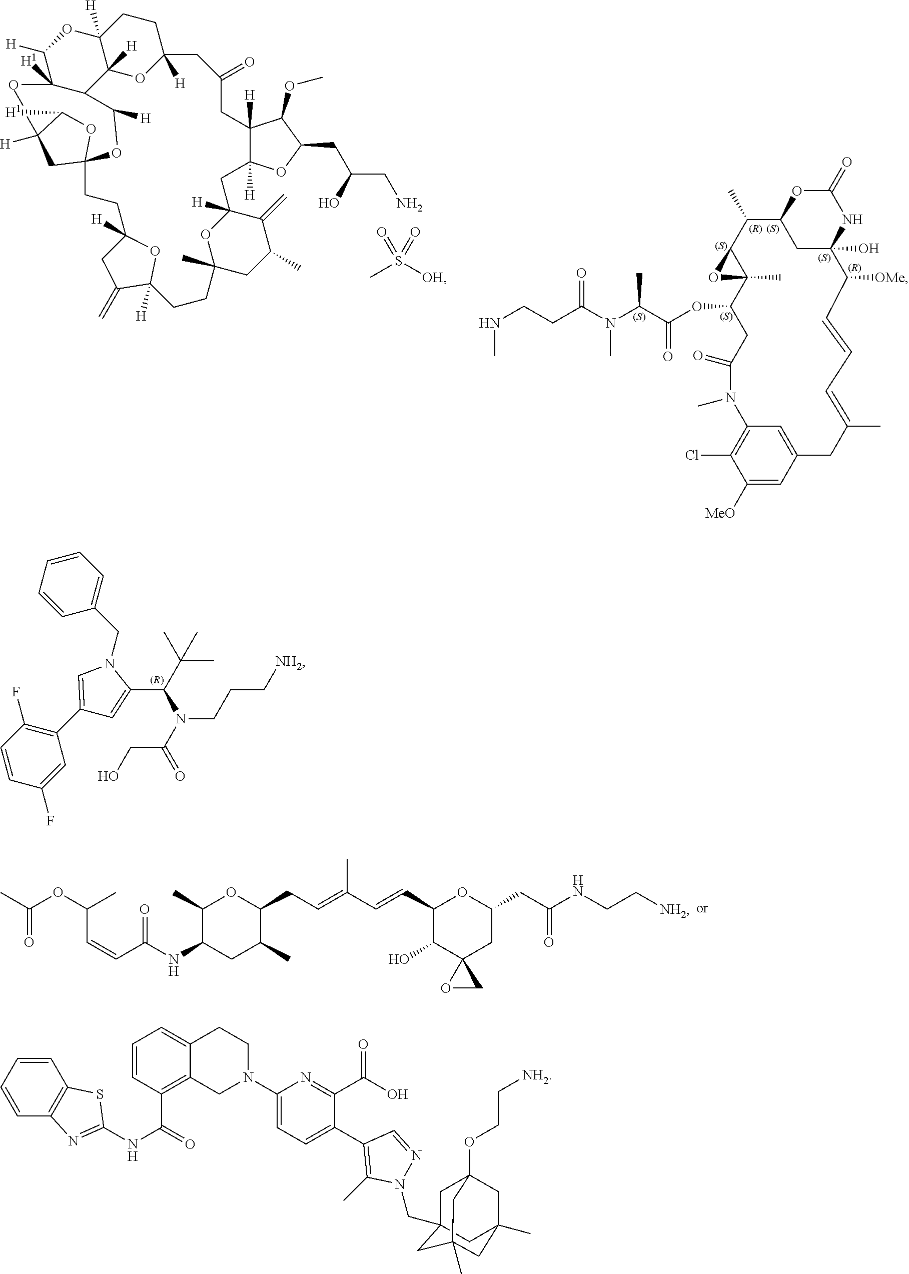

In some embodiments of any of Aspects 1 to 4, Payload is

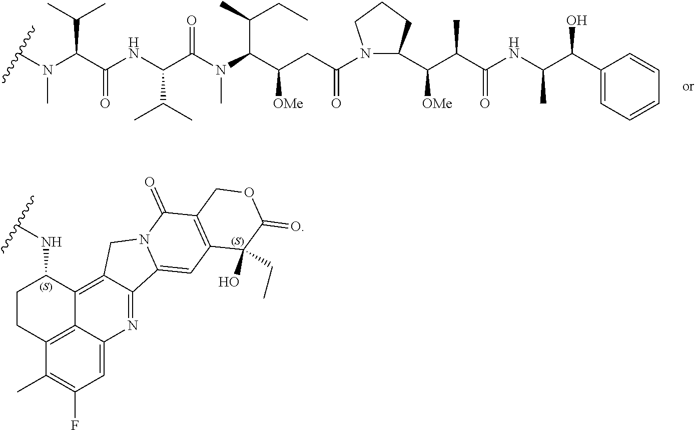

In some embodiments of Aspect 1, the compound is

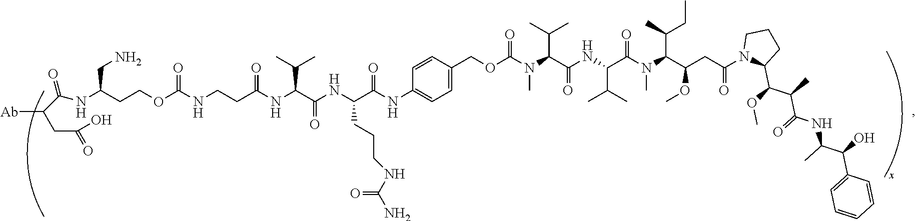

wherein Ab is 6E7 or an antigen-binding fragment thereof; or

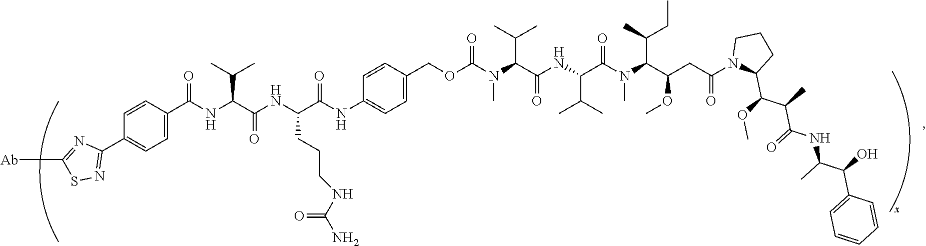

wherein Ab is 6E7 or an antigen-binding fragment thereof, or a pharmaceutically acceptable salt, tautomer, solvate, or stereoisomer of any of the foregoing.

In some embodiments of Aspect 1, the compound is the compound is

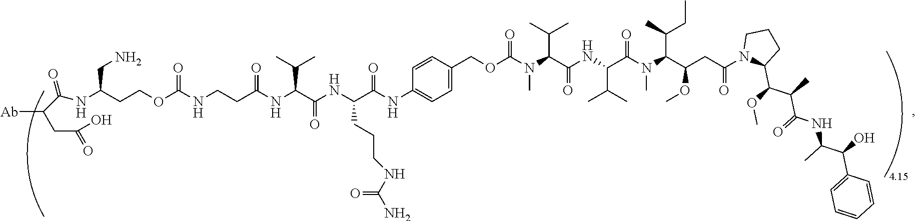

wherein Ab is 6E7 or an antigen-binding fragment thereof; or

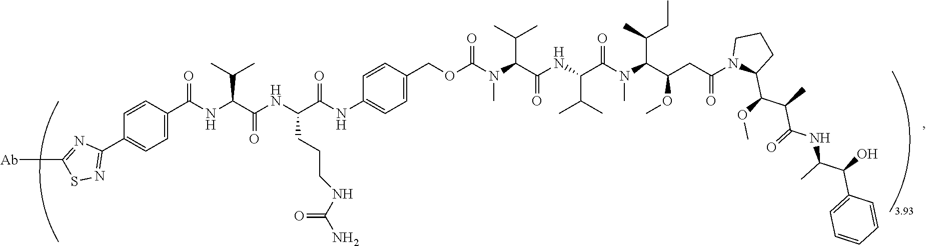

wherein Ab is 6E7 or an antigen-binding fragment thereof, or a pharmaceutically acceptable salt, tautomer, solvate, or stereoisomer of any of the foregoing.

In some embodiments of Aspect 2, the compound is

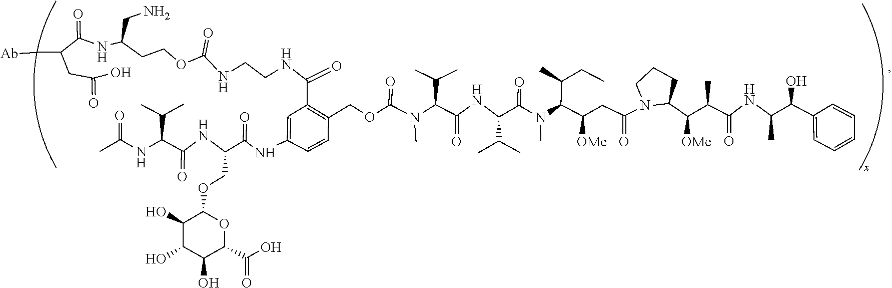

wherein Ab is 6E7 or an antigen-binding fragment thereof:

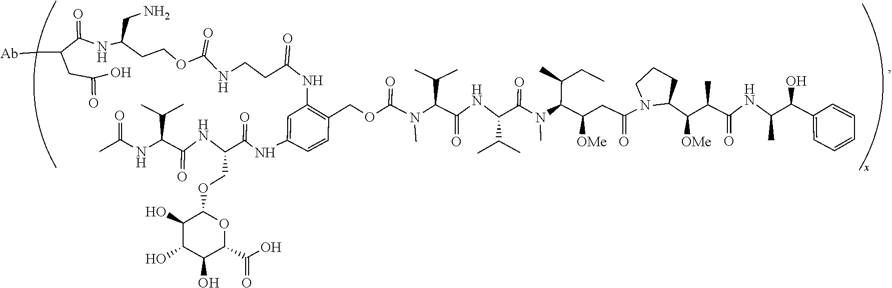

wherein Ab is 6E7 or an antigen-binding fragment thereof;

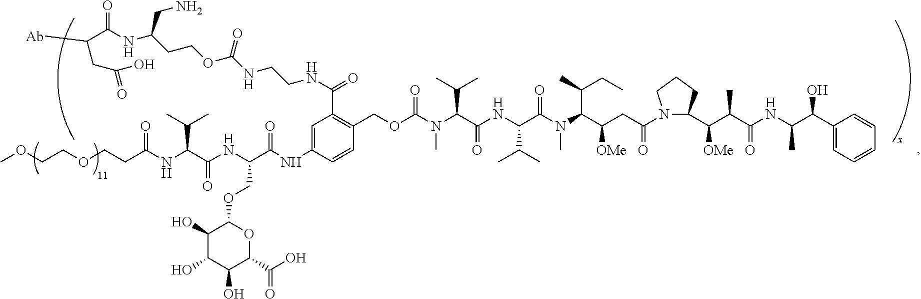

wherein Ab is 6E7 or an antigen-binding fragment thereof;

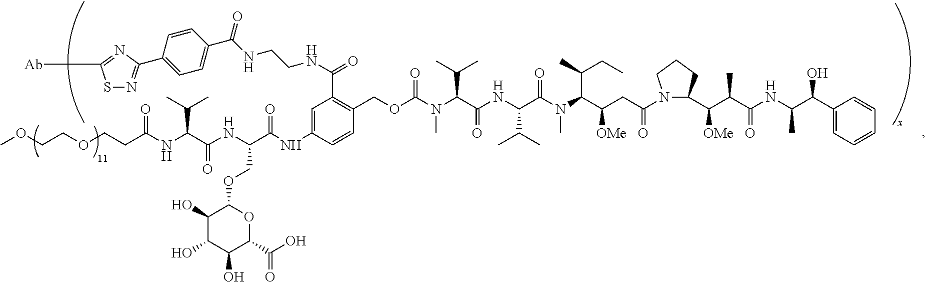

wherein Ab is 6E7 or an antigen-binding fragment thereof;

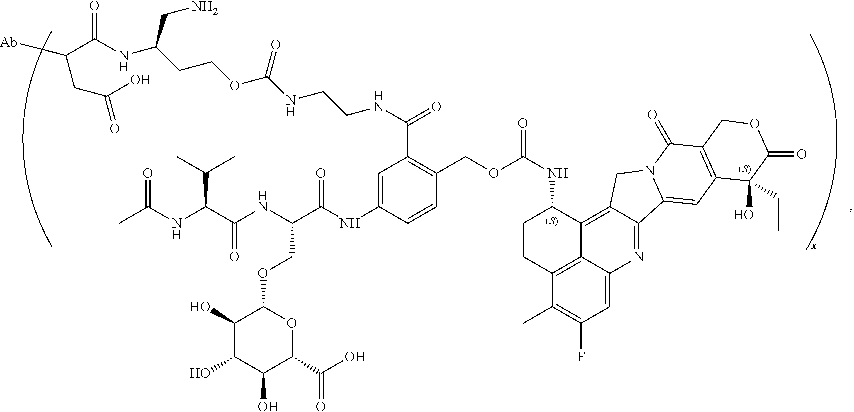

wherein Ab is ifinatamab or an antigen-binding fragment thereof;

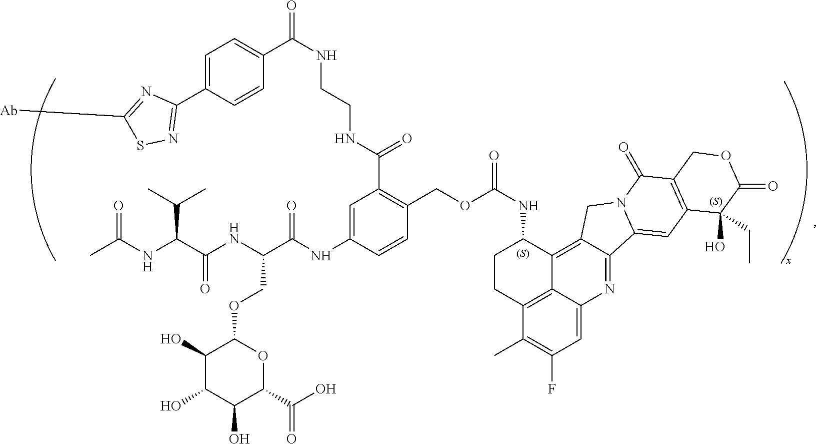

wherein Ab is ifinatamab or an antigen-binding fragment thereof;

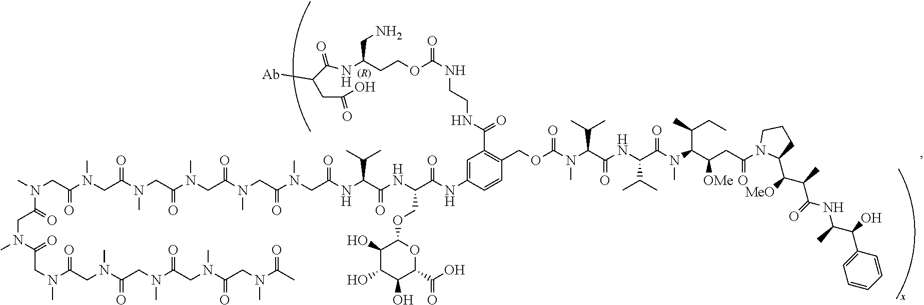

wherein Ab is 6E7 or an antigen-binding fragment thereof; or

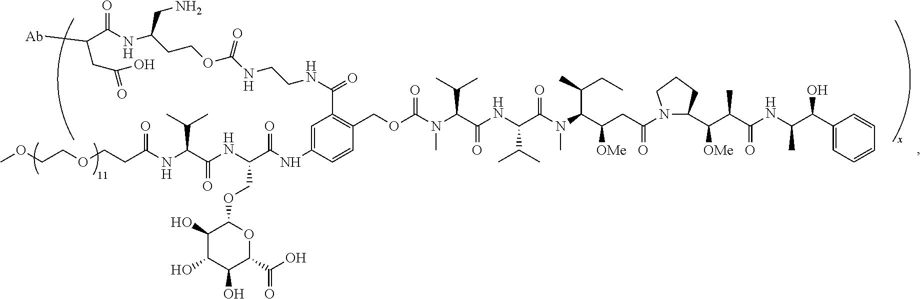

wherein Ab is cofetuzumab or an antigen-binding fragment thereof, or a pharmaceutically acceptable salt, tautomer, solvate, or stereoisomer of any of the foregoing.

In some embodiments of Aspect 2, the compound is

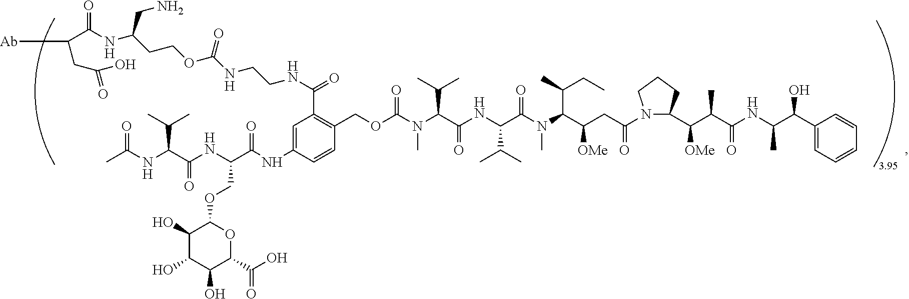

wherein Ab is 6E7 or an antigen-binding fragment thereof;

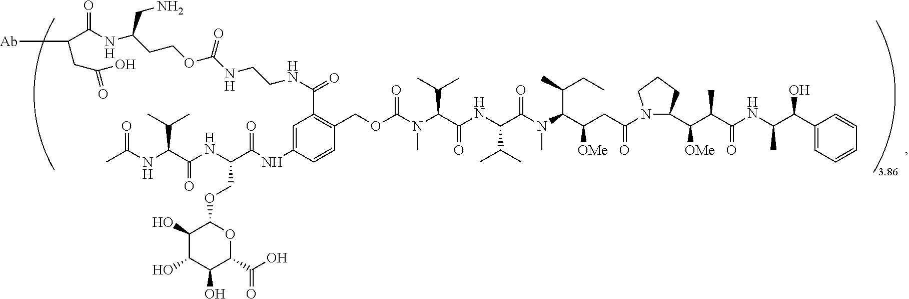

wherein Ab is 6E7 or an antigen-binding fragment thereof;

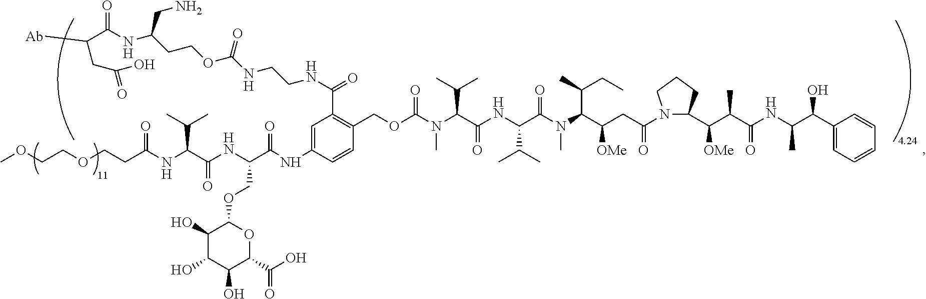

wherein Ab is 6E7 or

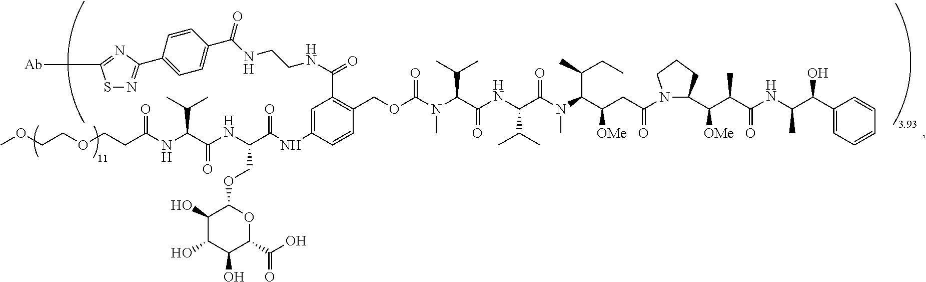

an antigen-binding fragment thereof;

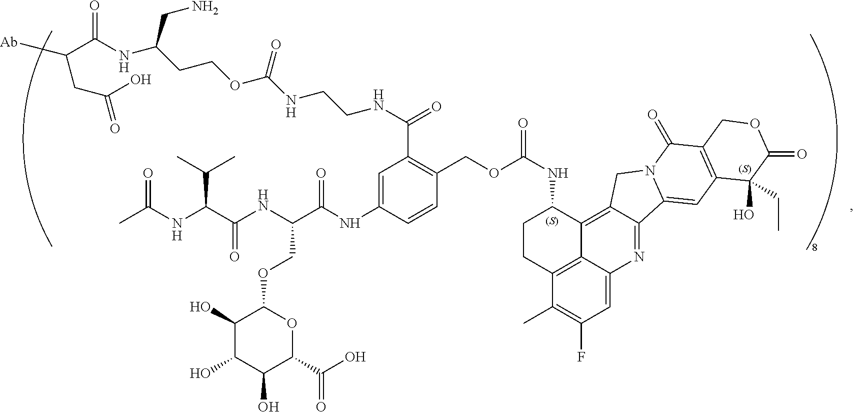

wherein Ab is ifinatamab or an antigen-binding fragment thereof;

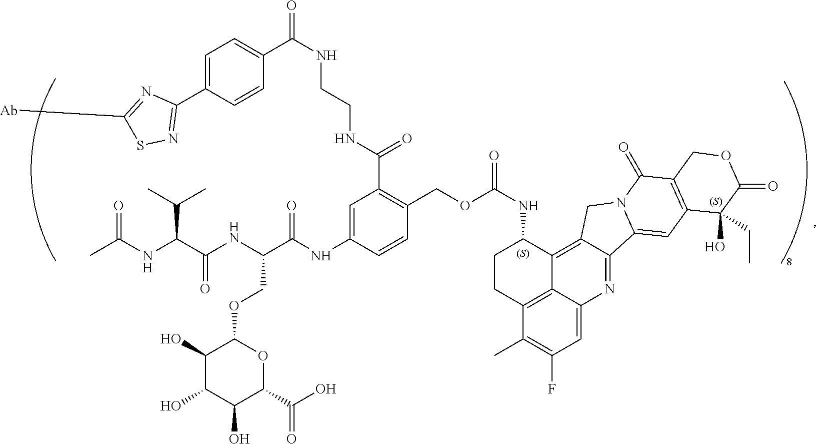

wherein Ab is ifinatamab or an antigen-binding fragment thereof;

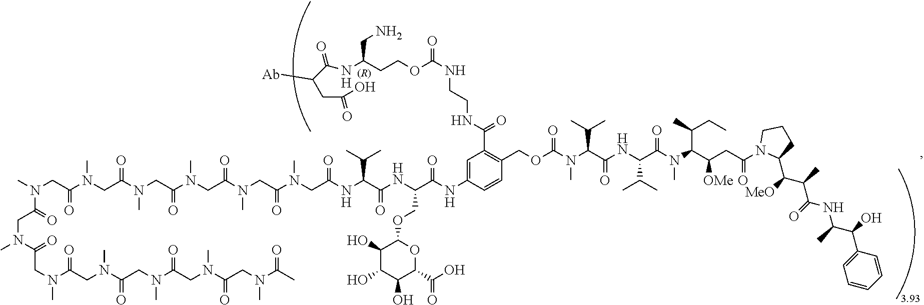

wherein Ab is 6E7 or an antigen-binding fragment thereof; or

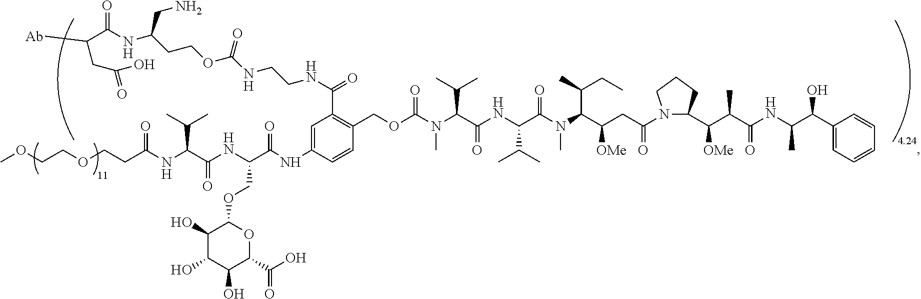

wherein Ab is cofetuzumab or an antigen-binding fragment thereof or a pharmaceutically acceptable salt, tautomer, solvate, or stereoisomer of any of the foregoing.

In some embodiments of Aspect 3, the compound is

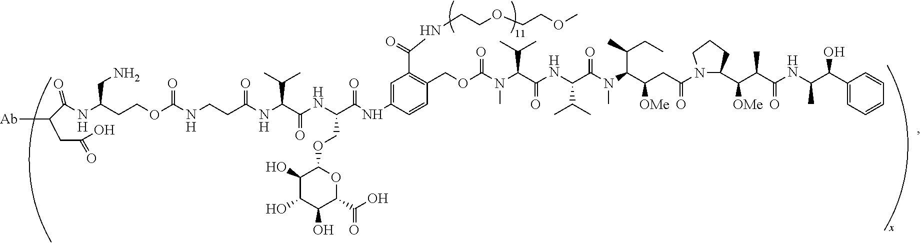

wherein Ab is 6E7 or an antigen-binding fragment thereof:

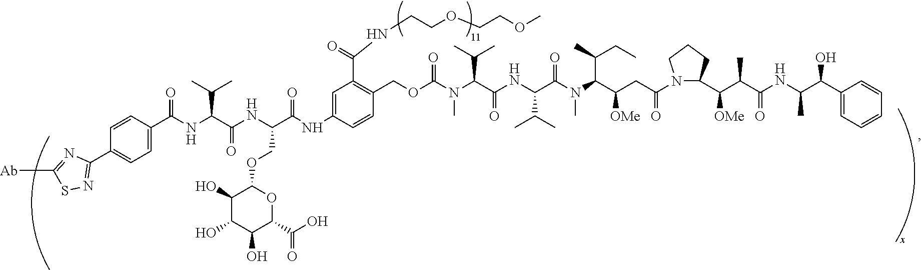

wherein Ab is 6E7 or an antigen-binding fragment thereof:

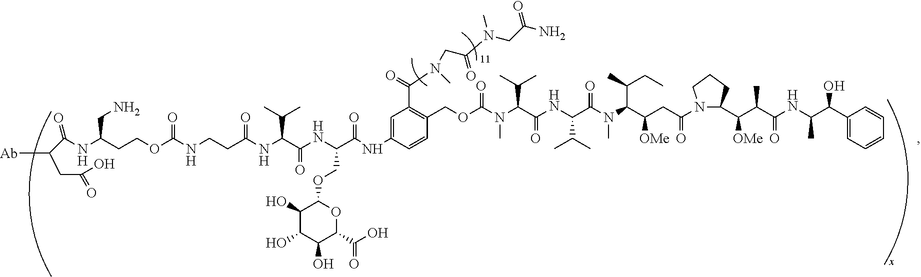

wherein Ab is 6E7 or an antigen-binding fragment thereof;

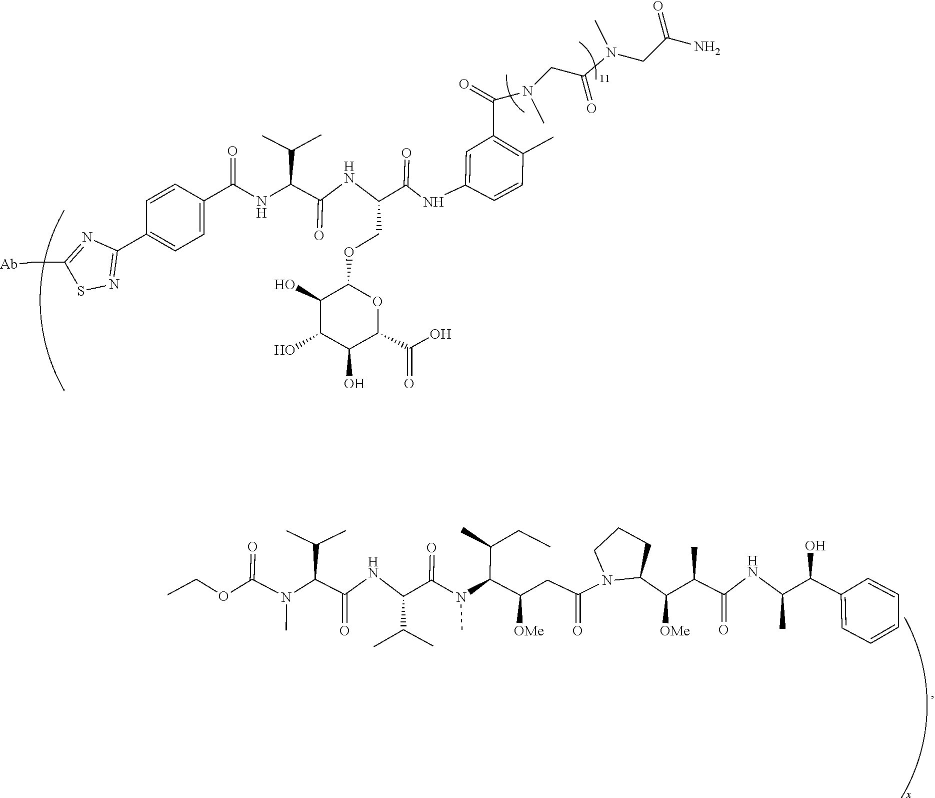

wherein Ab is 6E7 or an antigen-binding fragment thereof:

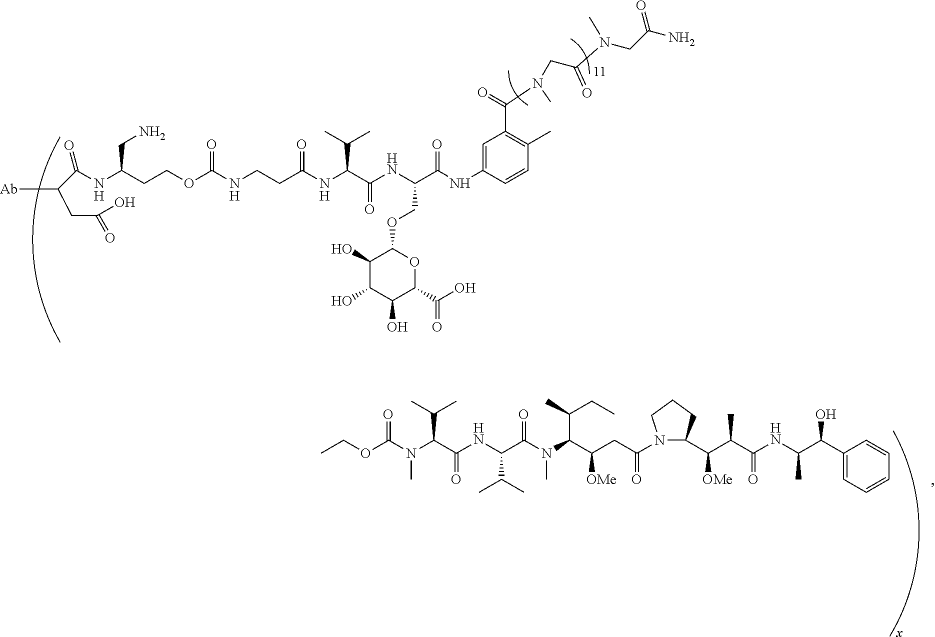

wherein Ab is ifinatamab or an antigen-binding fragment thereof:

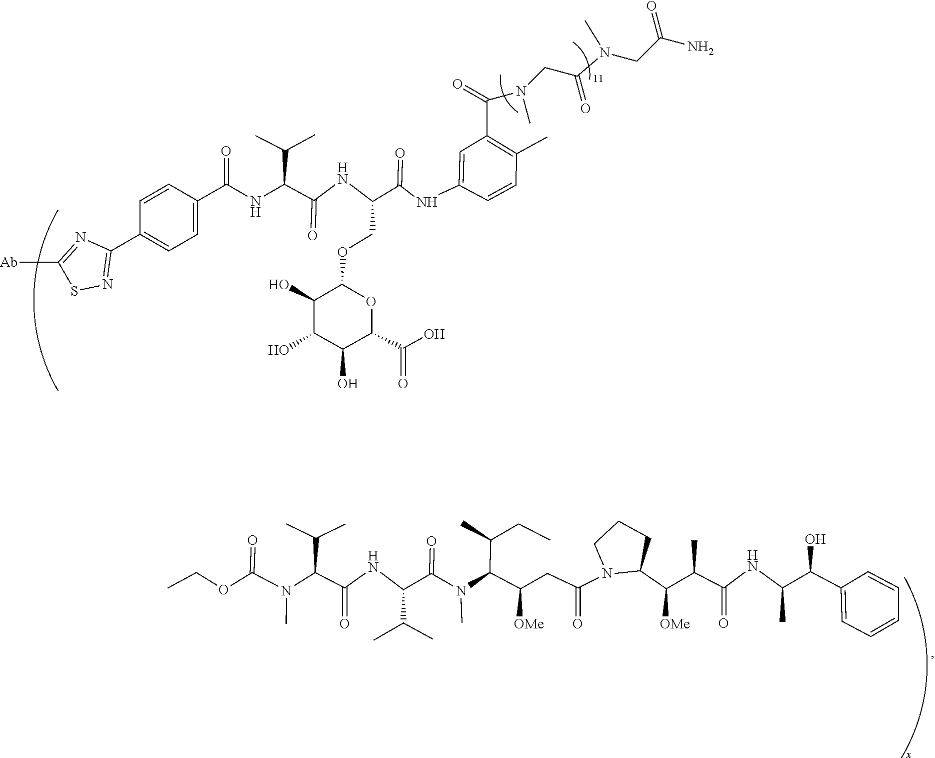

wherein Ab is ifinatamab or an antigen-binding fragment thereof; or

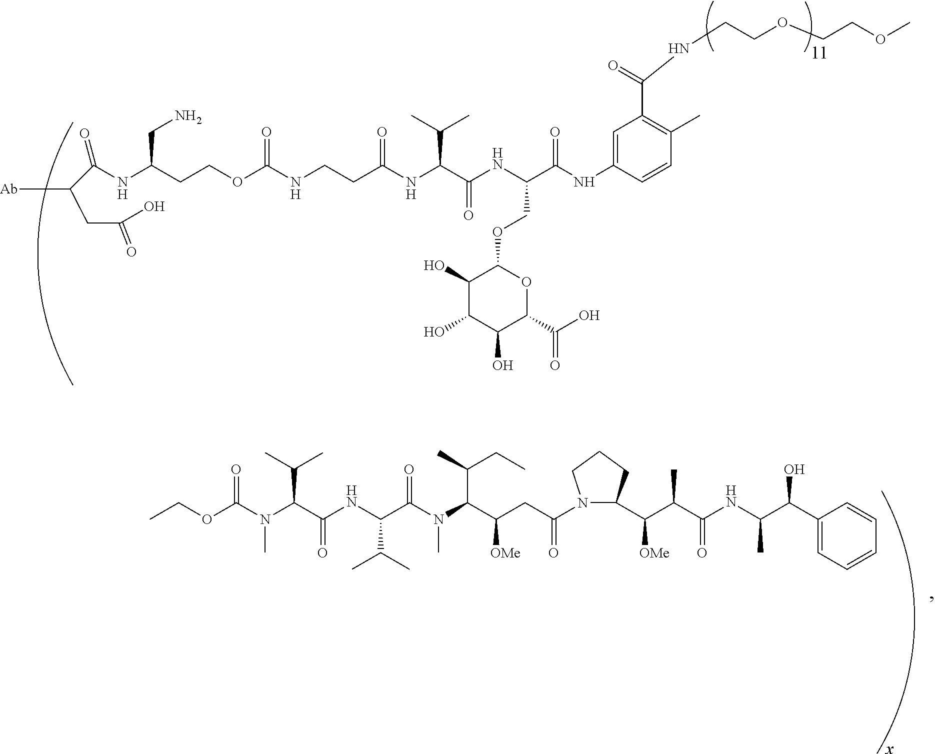

wherein Ab is cofetuzumab or an antigen-binding fragment thereof, or a pharmaceutically acceptable salt, tautomer, solvate, or stereoisomer of any of the foregoing.

In some embodiments of Aspect 3, the compound is

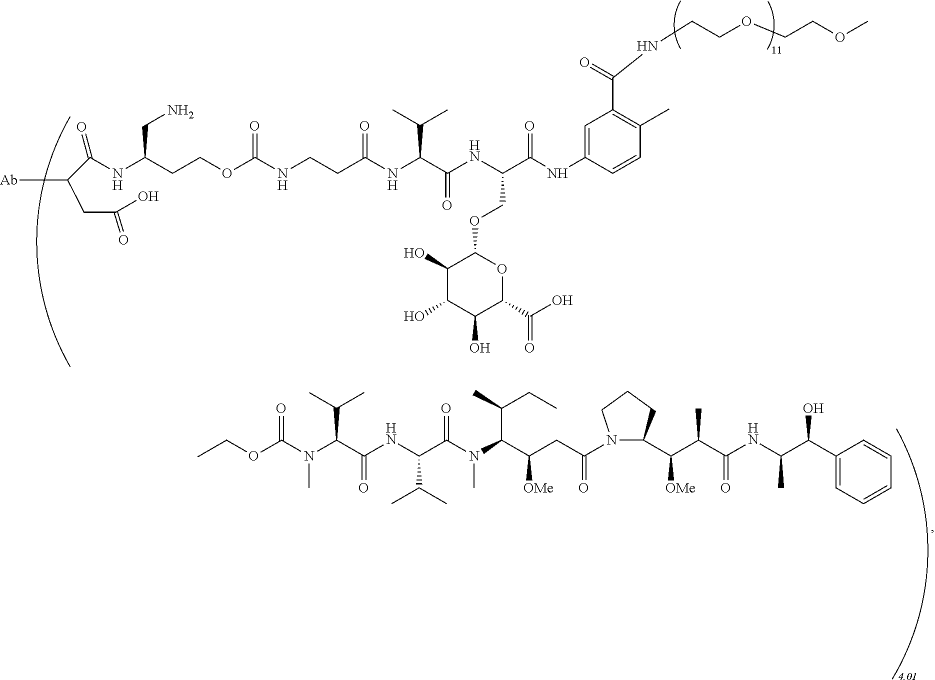

wherein Ab is 6E7 or an antigen-binding fragment thereof;

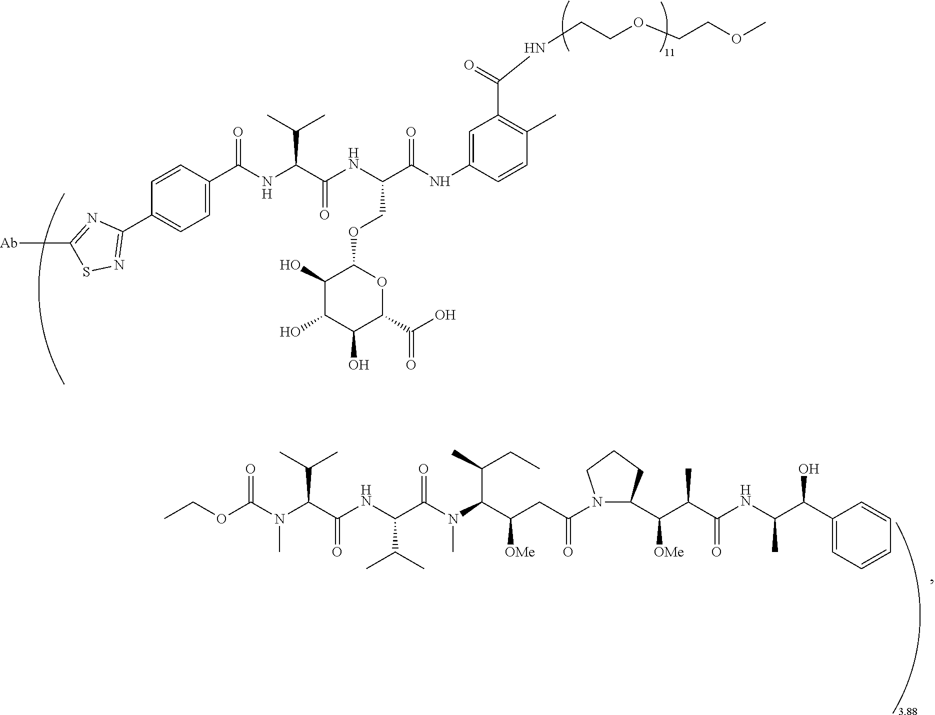

wherein Ab is 6E7 or an antigen-binding fragment thereof;

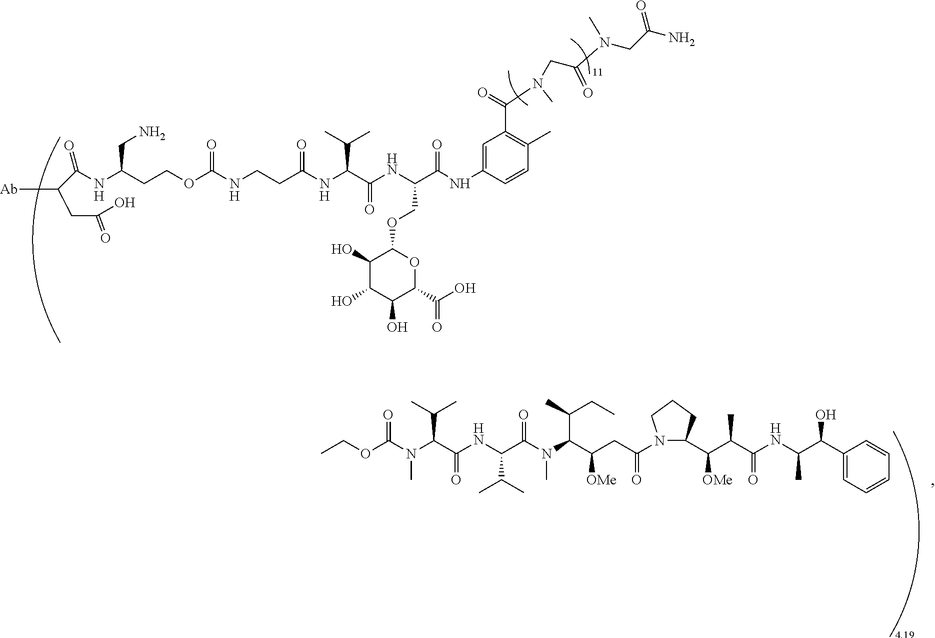

wherein Ab is 6E7 or an antigen-binding fragment thereof;

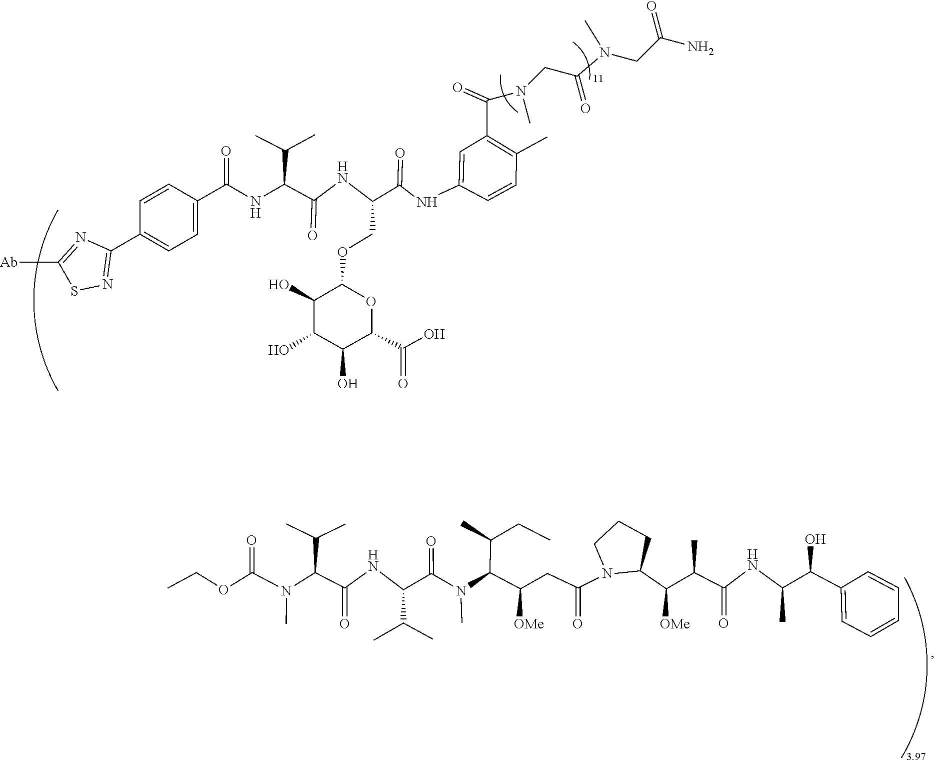

wherein Ab is 6E7 or an antigen-binding fragment thereof;

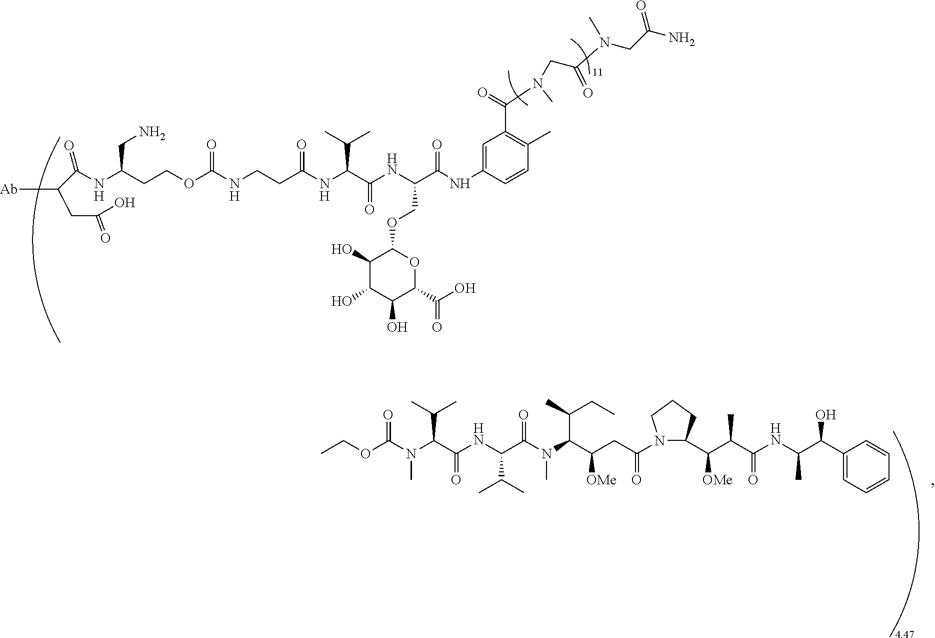

wherein Ab is ifinatamab or an antigen-binding fragment thereof;

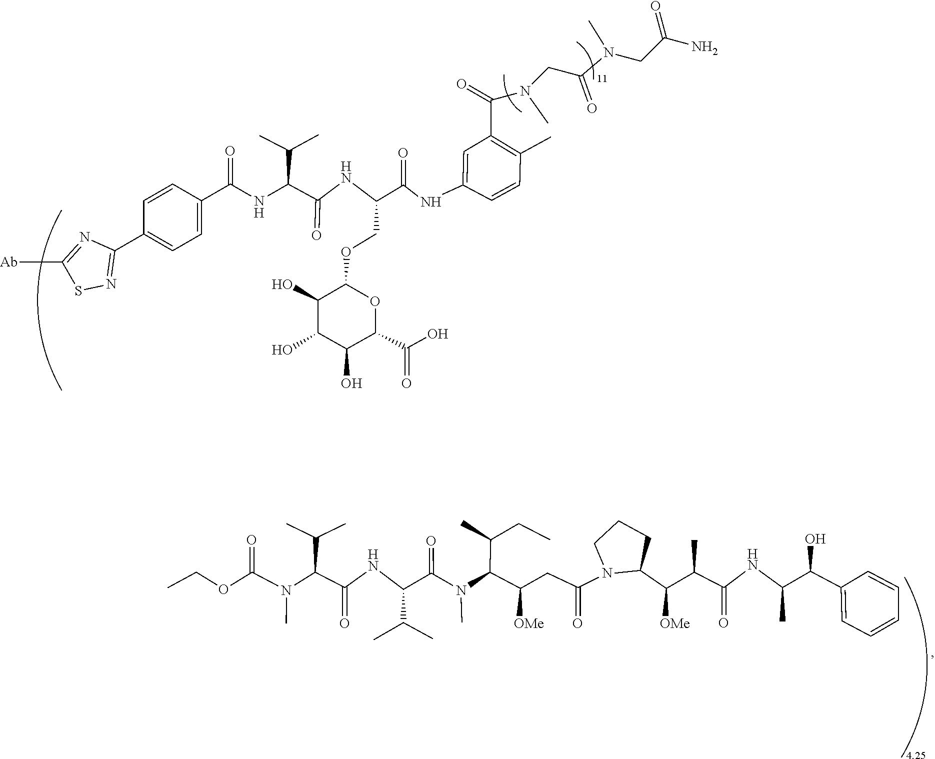

wherein Ab is ifinatamab or an antigen-binding fragment thereof or

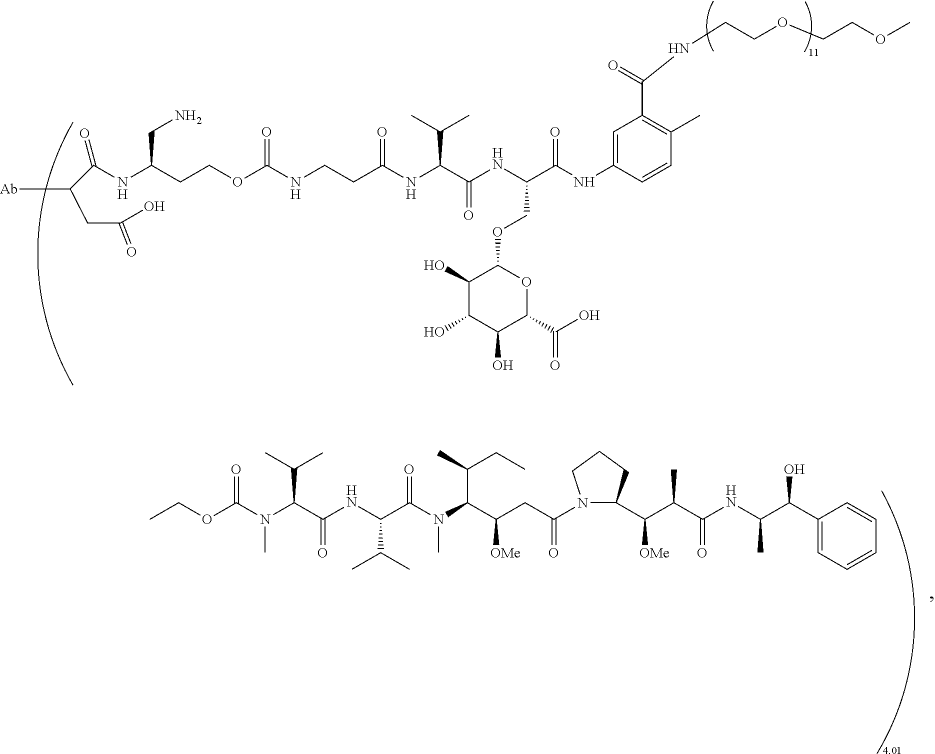

wherein Ab is cofetuzumab or an antigen-binding fragment thereof, or a pharmaceutically acceptable salt, tautomer, solvate, or stereoisomer of any of the foregoing.

In some embodiments of Aspect 4, the compound is

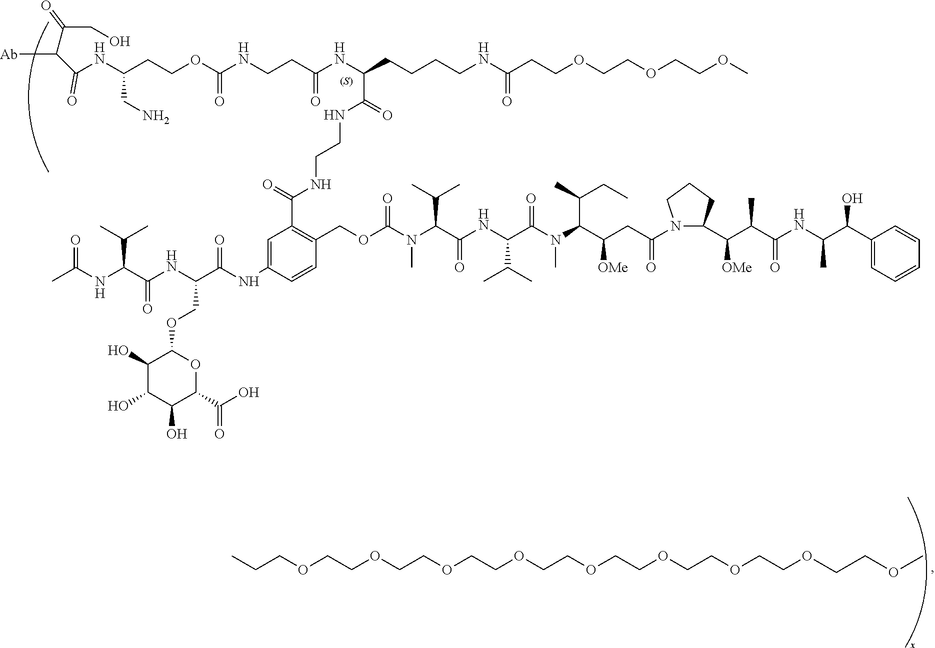

wherein Ab is 6E7 or an antigen-binding fragment thereof;

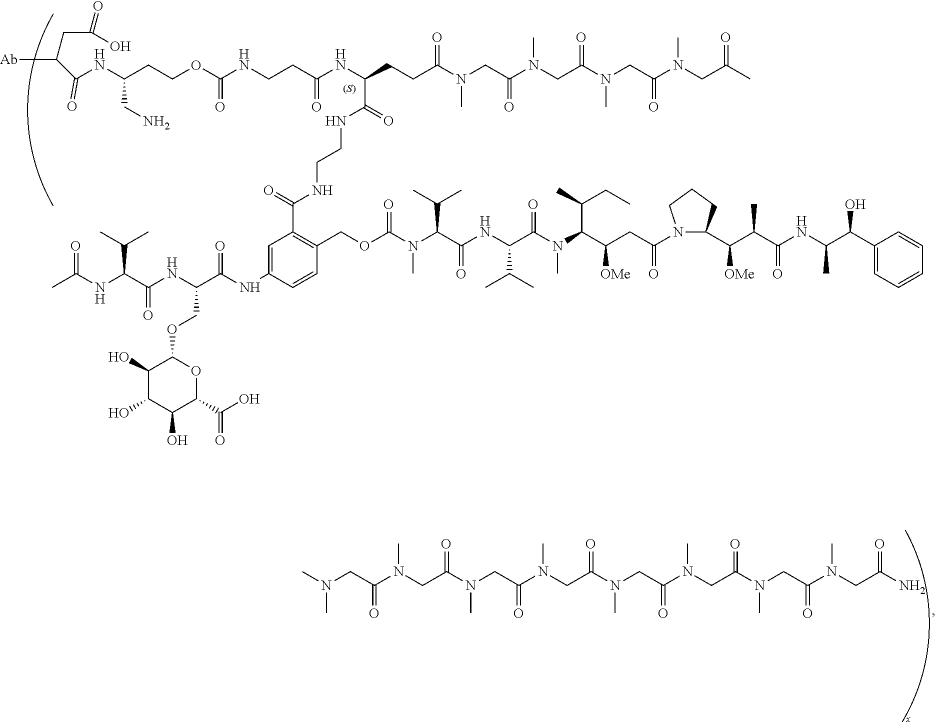

wherein Ab is 6E7 or an antigen-binding fragment thereof or

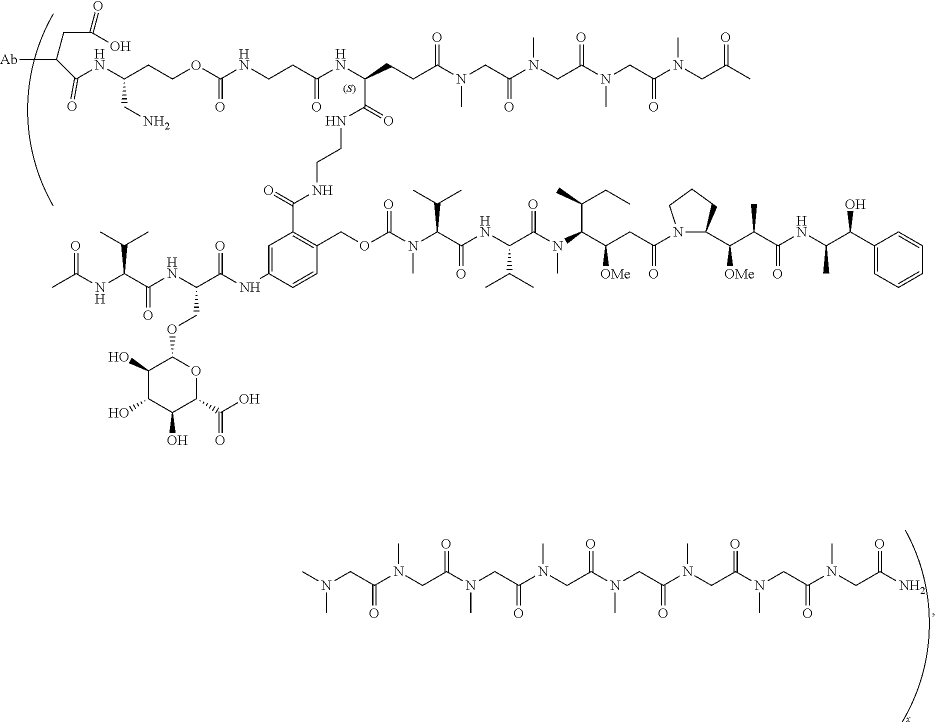

wherein Ab is ifinatamab or an antigen-binding fragment thereof, or a pharmaceutically acceptable salt, tautomer, solvate, or stereoisomer of any of the foregoing.

In some embodiments of Aspect 4, the compound is

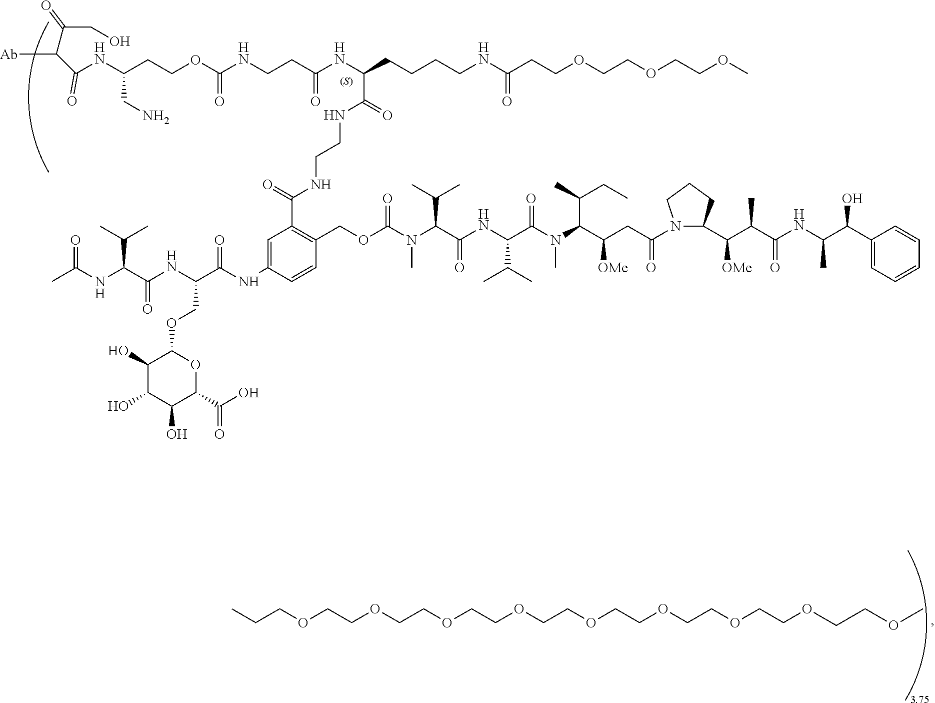

wherein Ab is 6E7 or an antigen-binding fragment thereof;

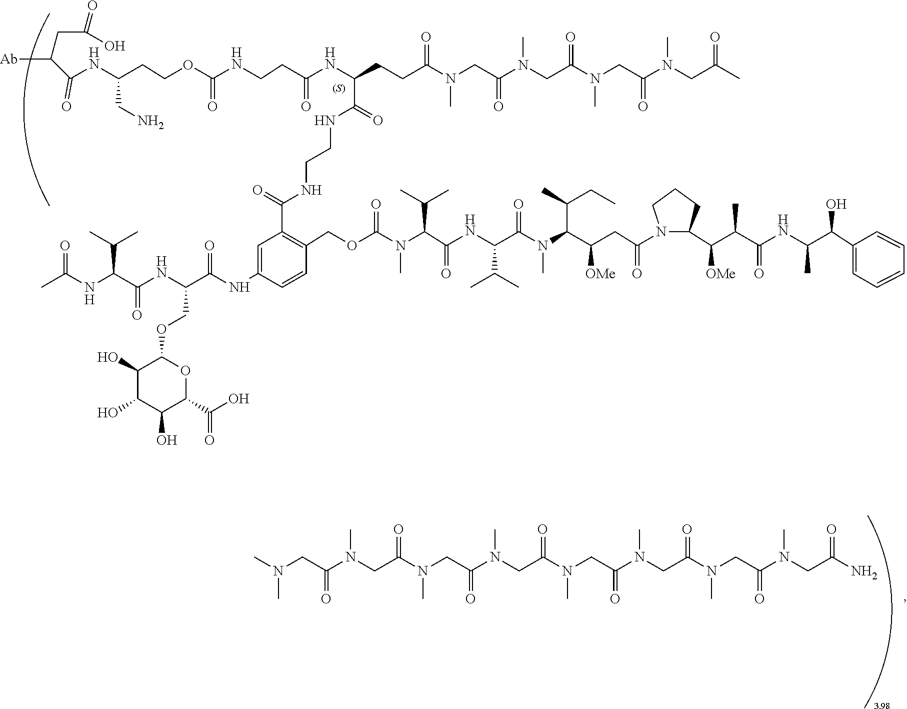

wherein Ab is 6E7 or an antigen-binding fragment thereof; or

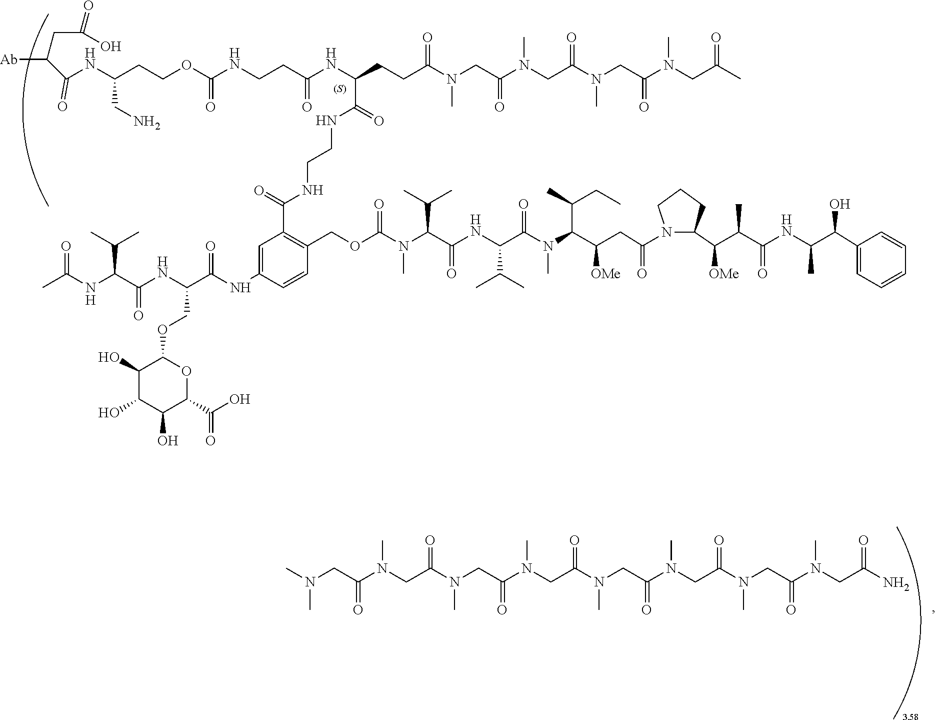

wherein Ab is ifinatamab or an antigen-binding fragment thereof, or a pharmaceutically acceptable salt, tautomer, solvate, or stereoisomer of any of the foregoing.

### Aspect 5

Provided herein are compounds of formula (Ia′):

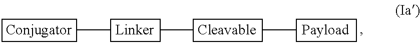

or a pharmaceutically acceptable salt, tautomer, solvate, or stereoisomer thereof, wherein:
Conjugator is selected from formula (II′) and (III′):

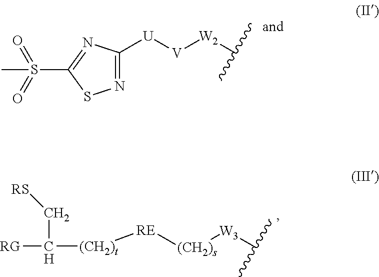

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

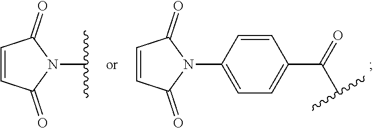 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
indicates a point of covalent attachment within the compound;
Linker has formula (IV):

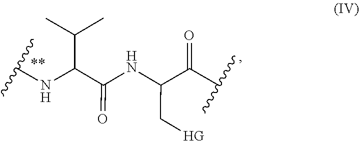

wherein:
HG is a hydrophilic residue, and
** marks the bond where Linker connects to Conjugator;
Cleavable has formula (V):

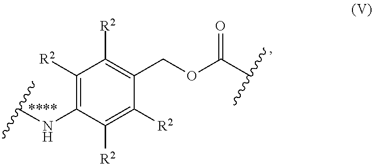

wherein:
each R² is independently hydrogen, a halogen, substituted or unsubstituted C₁₋₄ alkyl, —CN, or —NO₂, and
**** marks the bond where Cleavable connects to Linker; and
Payload is a payload residue.

In some embodiments of Aspect 5, Cleavable has the following formula:

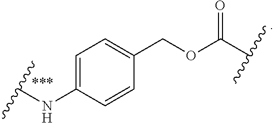 [SMILES: *Nc1ccc(COC(*)=O)cc1]

### Aspect 6

Provided herein are compounds of Formula (Ib′):

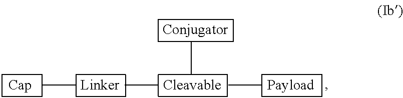

or a pharmaceutically acceptable salt, tautomer, solvate, or stereoisomer thereof, wherein:
Conjugator is selected from formula (II′) and (III′):

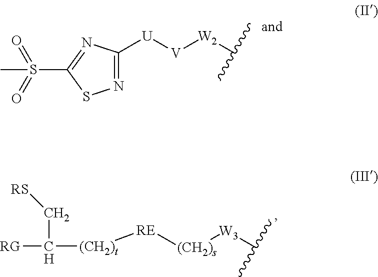

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

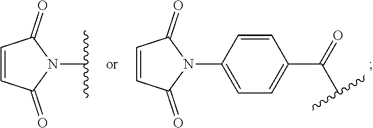 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
indicates a point of covalent attachment within the compound;
Cleavable has formula (Va), (Vb), or (Vc):

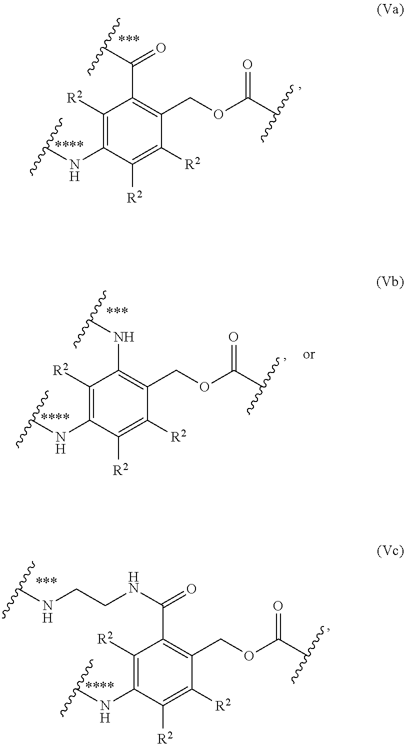

wherein:
each R² is, independently, hydrogen, halogen, substituted or unsubstituted C₁₋₄ alkyl, —CN, or —NO₂,
*** marks the bond where Cleavable connects to Conjugator. and
**** marks the bond where Cleavable connects to Linker:
Linker has formula (IV′):

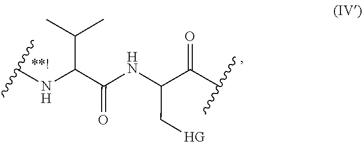

Wherein
HG is a hydrophilic residue, and
**! Marks the bond where Linker connects to Cap;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—, —(CH₂CH₂O)ₐ—(CH₂CH₂)_{d}—NH—C(═O)—, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive; and
Payload is a payload residue.

In some embodiments of Aspect 6, Cleavable has one of the following formulas:

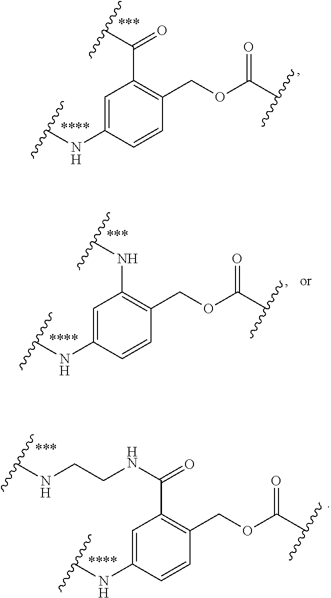

In some embodiments of Aspect 6, Cap is CH₃C(═O)—, CH₃—(CH₂)_{b}—O—(CH₂CH₂O)ₐ—(CH₂CH₂)_{d}—C(═O)—, or CH₃(C(═O)N(CH₃)CH₂)C(═O)—.

In some embodiments of Aspect 6, Cap is CH₃C(═O)— CH₃—O—(CH₂CH₂O)₁₁—(CH₂CH₂)—C(═O)—, or —CH₃(C(═O)N(CH₃)CH₂)₁₂C(═O)—.

### Aspect 7

Provided herein are compounds of Formula (Ic′):

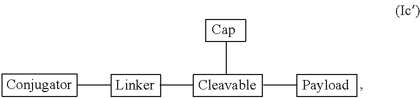

or a pharmaceutically acceptable salt, tautomer, solvate, or stereoisomer thereof, wherein:
Conjugator is selected from formula (II′) and (III′):

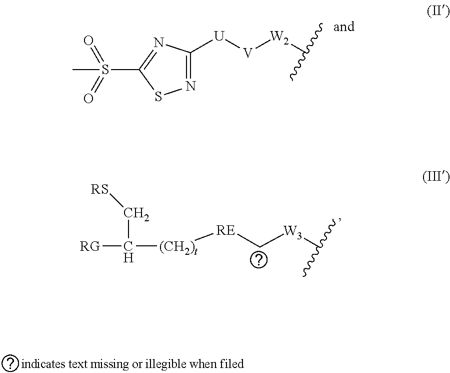

wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is

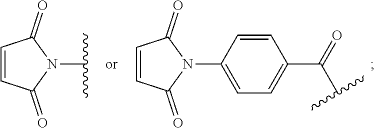 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂—NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2, and [symbol]
indicates a point of covalent attachment within the compound;
Linker has formula (IV):

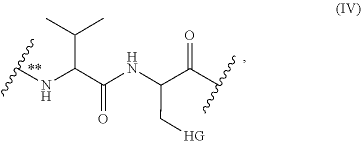

wherein.
HG is a hydrophilic residue, and
** marks the bond where Linker connects to Conjugator;
Cleavable has formula (Va′), (Vb′), or (Vc′):

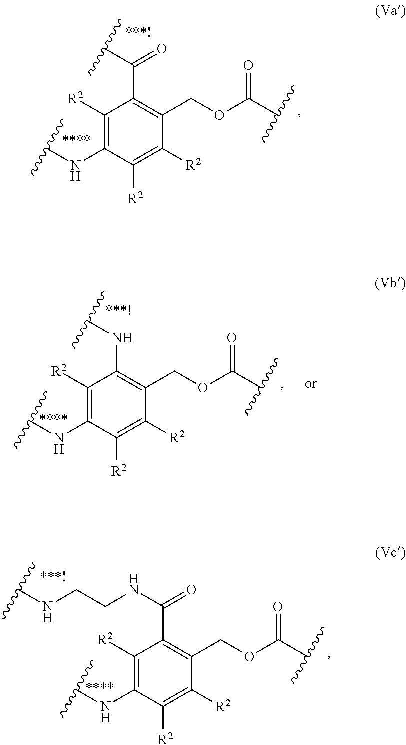

wherein:
each R² is, independently, hydrogen, halogen, substituted or unsubstituted C₁₋₄ alkyl, —CN, or —NO₂,
***! Marks the bond where Cleavable connects to Cap, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(═O)N(CH₃)CH₂)ₐC(═O)—, —NH—(CH₂CH₂O)—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive; and
Payload is a payload residue.

In some embodiments of Aspect 7, Cleavable has one of the following formulas:

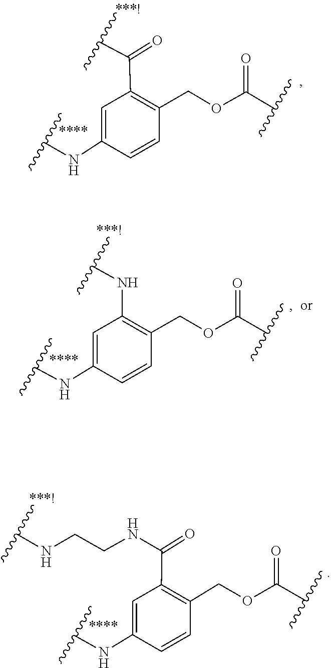 [SMILES: *Nc1ccc(COC(*)=O)c(C(*)=O)c1 ; *Nc1ccc(COC(*)=O)c(N*)c1 ; *NCCNC(=O)c1cc(N*)ccc1COC(*)=O]

In some embodiments of Aspect 7, Cap is —NH—(CH₂CH₂O)ₐ—(CH₂)_{b}CH₃, or —(N(CH₃)(CH₂)_{c}C(═O))ₐNH₂.

In some embodiments of Aspect 7, Cap is —NH—(CH₂CH₂O)₁₂—CH₃ or —(N(CH₃)CH₂C(═O))₁₂NH₂.

### Aspect 8

Provided herein are compounds of Formula (Id):

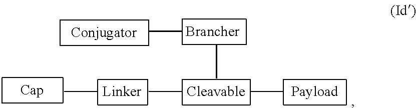

or a pharmaceutically acceptable salt, tautomer, solvate, or stereoisomer thereof, wherein:
Conjugator is selected from formula (II′) and (III′):

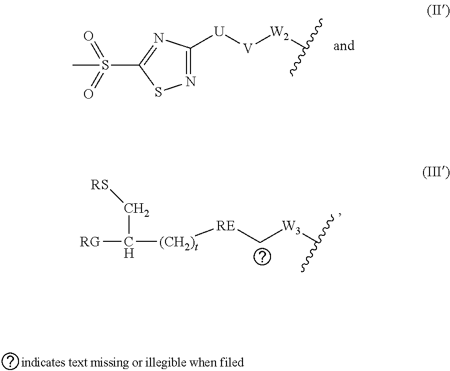

wherein:
U is a bond, heteroarylene, or arylene:
V is a bond or —C≡C—(CH₂)ₙ—;
n is an integer between 0 and 10 inclusive;
W₂ is —C(═O)—, —NH—, or —O—;
RG is

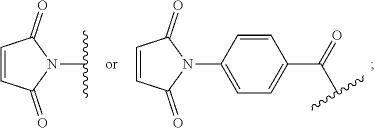 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently. 1 or 2, and [symbol]
indicates a point of covalent attachment within the compound;
Linker has formula (IV′):

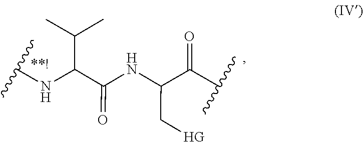

wherein:
HG is a hydrophilic residue, and
**! Marks the bond where Linker connects to Cap:
Cleavable has formula (Va″) (Vb″), or (Vc″):

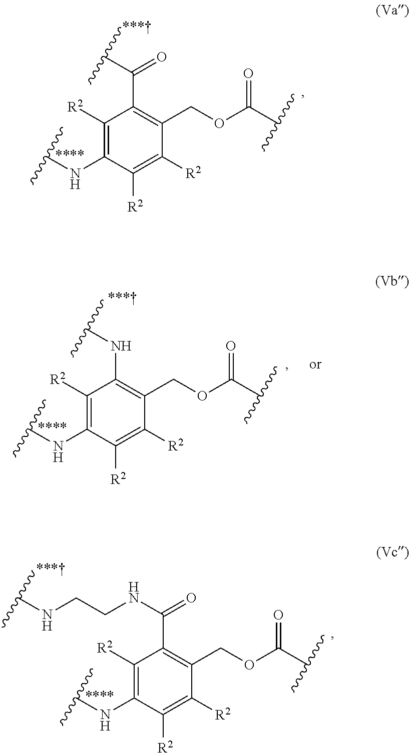

wherein:
each R² is, independently, hydrogen, halogen, substituted or unsubstituted C₁₋₄ alkyl, —CN, or —NO₂,
***^{†} marks the bond where Cleavable connects to Brancher, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(═O)N(CH₃)CH₂)ₐC(═O)—, NH—(CH₂CH₂O)ₐ—(CH₂)_{b}CH₃, or —(N(CH₃)(CH₂)ₐC(═O))ₐNH₂, wherein:
a is an integer between 1 and 18 inclusive,
each of b and d is, independently, 0, 1, or 2, and
c is an integer between 1 and 4 inclusive; and
Brancher has formula (XIIa), (XIIa1), (XIIb) or (XIIb1):

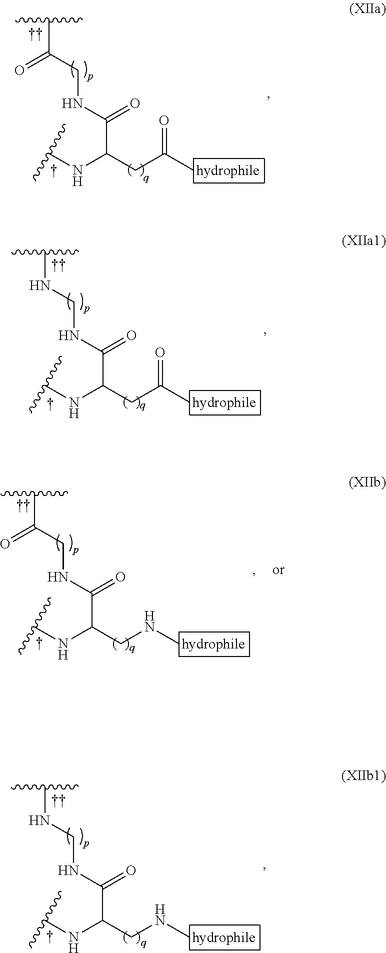

wherein
hydrophile is —NH—(CH₂CH₂O)ₐ₁—(CH₂)_{b1}—CH₃, —C(═O)NH₂, —C(═O)—(CH₂CH₂O)ₐ₁—(CH₂)_{b1}CH, or —(N(CH₃)(CH₂)_{c1}C(═O))ₐ₁NH₂,
† marks the bond where Brancher connects to Conjugator,
†† marks the bond where Brancher connects to Cleavable,
a1 is an integer between 1 and 18 inclusive,
b1 is 0, 1, or 2, and
each of c1, p, and q is, independently, an integer between 1 and inclusive; and
Payload is a payload residue.

In some embodiments of Aspect 8, Cleavable has one of the following formulas:

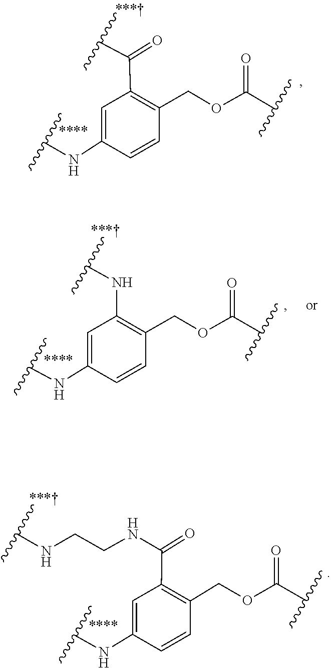 [SMILES: *Nc1ccc(COC(*)=O)c(C(*)=O)c1 ; *Nc1ccc(COC(*)=O)c(N*)c1 ; *NCCNC(=O)c1cc(N*)ccc1COC(*)=O]

In some embodiments of Aspect 8, a1 is 12, b1 is 0, c1 is 1, p is 2, and q is 2 or 4.

In some embodiments of Aspect 8, Brancher has one of the following formulas:

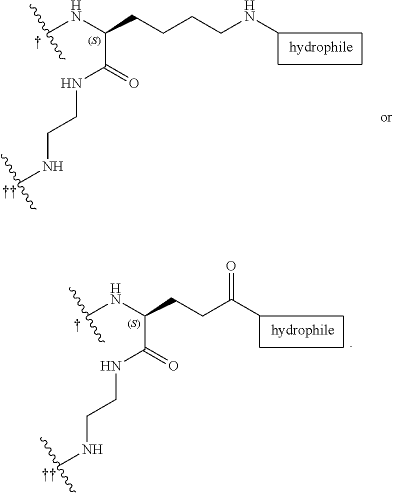

In some embodiments of Aspect 8, hydrophile is —C(═O)—(CH₂CH₂O)ₐ₁—CH₃ or —(N(CH₃)—CH₂—C(═O))ₐ₁NH₂, and a1 is an integer between 10 to 16 inclusive.

In some embodiments of Aspect 8, a1 is 12.

In some embodiments of Aspect 8, Cap is CH₃C(═O)—.

### Aspects 5, 6, 7, and 8

The foregoing platform compounds of formula (Ia′), (Ib′), (Ic′), and/or (Id′) include a Conjugator, Linker, and Payload, each as described below.

In some embodiments of any of Aspects 5 to 8, Conjugator has formula (II′).

In some embodiments of any of Aspects 5 to 8, U is arylene.

In some embodiments of any of Aspects 5 to 8, U is phenylene.

In some embodiments of any of Aspects 5 to 8, U is

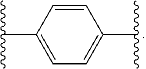 [SMILES: *c1ccc(*)cc1]

In some embodiments of any of Aspects 5 to 8, Li is heteroarylene.

In some embodiments of any of Aspects 5 to 8, U is a bivalent pyrimidine ring.

In some embodiments of any of Aspects 5 to 8, U is

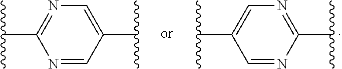 [SMILES: *c1cnc(*)nc1 ; "or" ; *c1cnc(*)nc1 ; "."]

In some embodiments of any of Aspects 5 to 8. U is a bond.

In some embodiments of any of Aspects 5 to 8, V is a bond.

In some embodiments of any of Aspects 5 to 8, V is —C≡C—(CH₂)—.

In some embodiments of any of Aspects 5 to 8, V is —C≡C—(CH₂)₃—.

In some embodiments of any of Aspects 5 to 8, W₂ is —C(═O)—.

In some embodiments of any of Aspects 5 to 8. Conjugator has formula (III′).

In some embodiments of any of Aspects 5 to 8, RS is —N(CH₃)₂.

In some embodiments of any of Aspects 5 to 8, RS is —NH₂.

In some embodiments of any of Aspects 5 to 8, RE is —OC(═O)NH—.

In some embodiments of any of Aspects 5 to 8, RG is

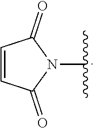 [SMILES: *N1C(=O)C=CC1=O]

In some embodiments of any of Aspects 5 to 8, each of s and t is 2.

In some embodiments of any of Aspects 5 to 8, Conjugator has formula (Iia1), or (IIIa1):

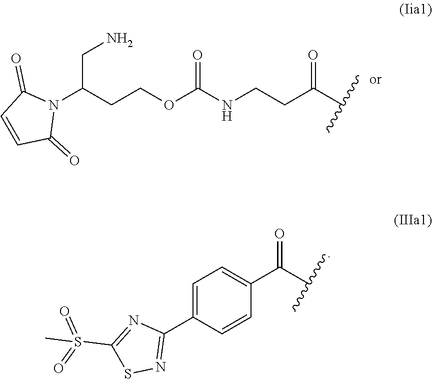 [SMILES: *C(=O)CCNC(=O)OCCC(CN)N1C(=O)C=CC1=O ; *C(=O)c1ccc(-c2nsc(S(C)(=O)=O)n2)cc1]

In some embodiments of any of Aspects 5 to 8, Linker has the following formula:

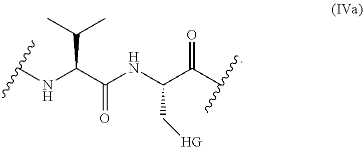

In some embodiments of any of Aspects 5 to 8.

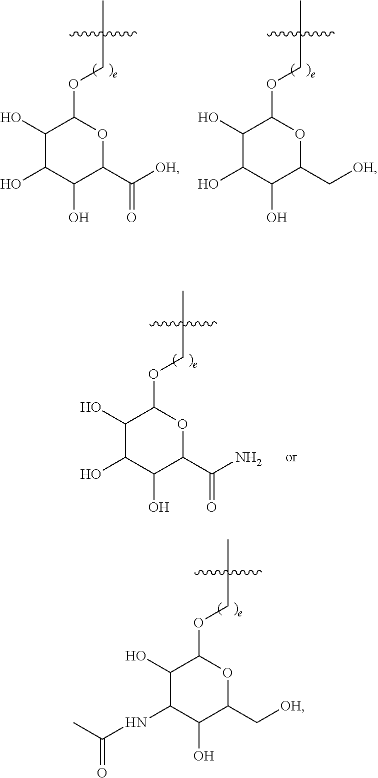

wherein e is 0, 1, or 2; or
HG is —(CH₂CH₂)_{z}NR^{a}C(O)NR^{b}R^{c}, wherein z is 1, 2, 3, or 4, and each of R^{a}, R^{b}, and R^{c} is, independently, H, or substituted or unsubstituted C₁₋₄ alkyl.

In some embodiments of any of Aspects 5 to 8, Linker has one of the following formulas:

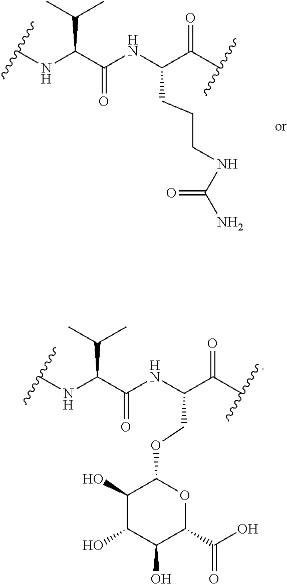 [SMILES: *N[C@H](C(=O)N[C@@H](CCCNC(N)=O)C(*)=O)C(C)C ; *N[C@H](C(=O)N[C@@H](CO[C@@H]1O[C@H](C(=O)O)[C@@H](O)[C@H](O)[C@H]1O)C(*)=O)C(C)C]

In some embodiments of any of Aspects 5 to 8, Payload is a residue of one of the following formulas:

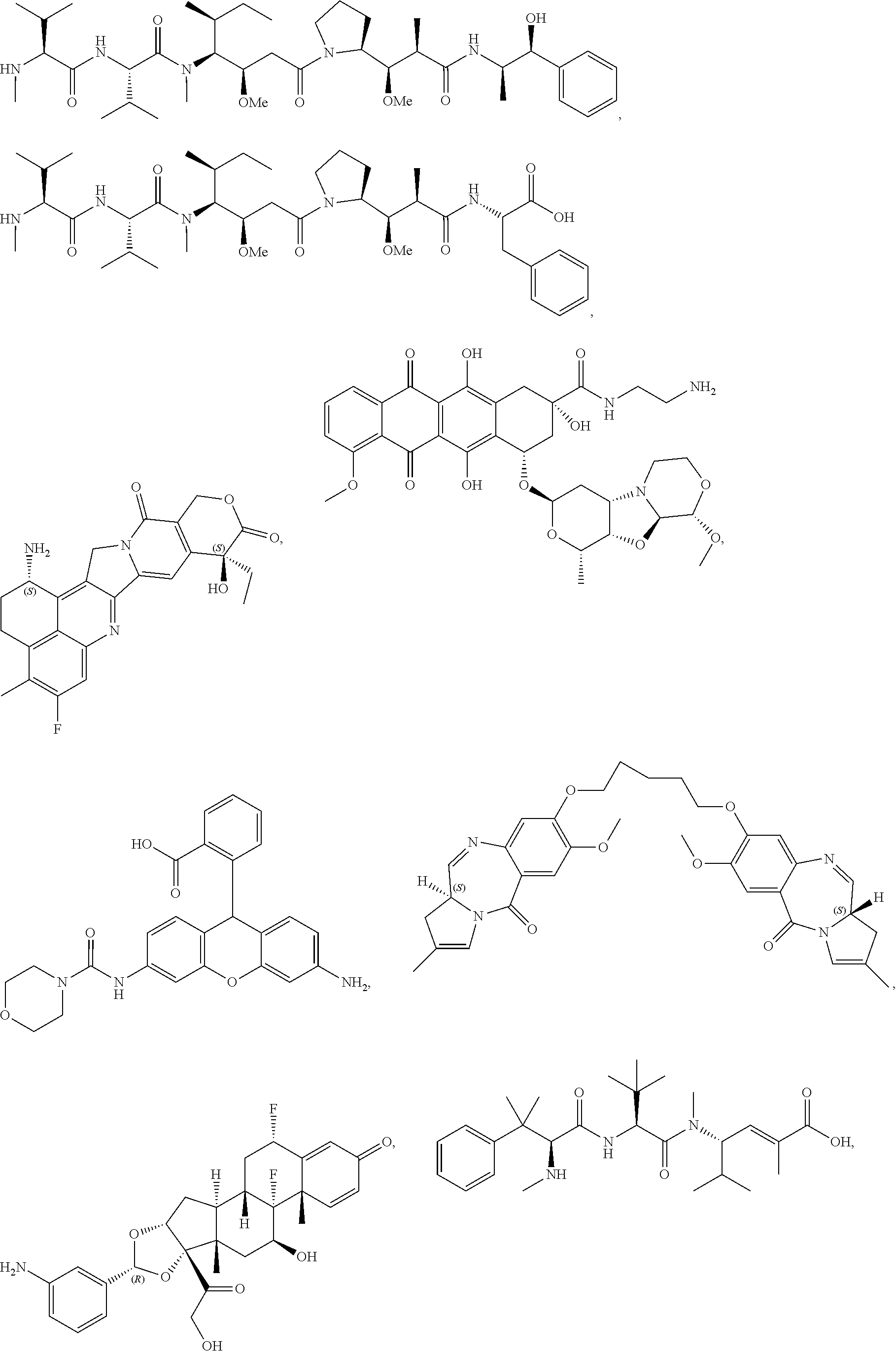

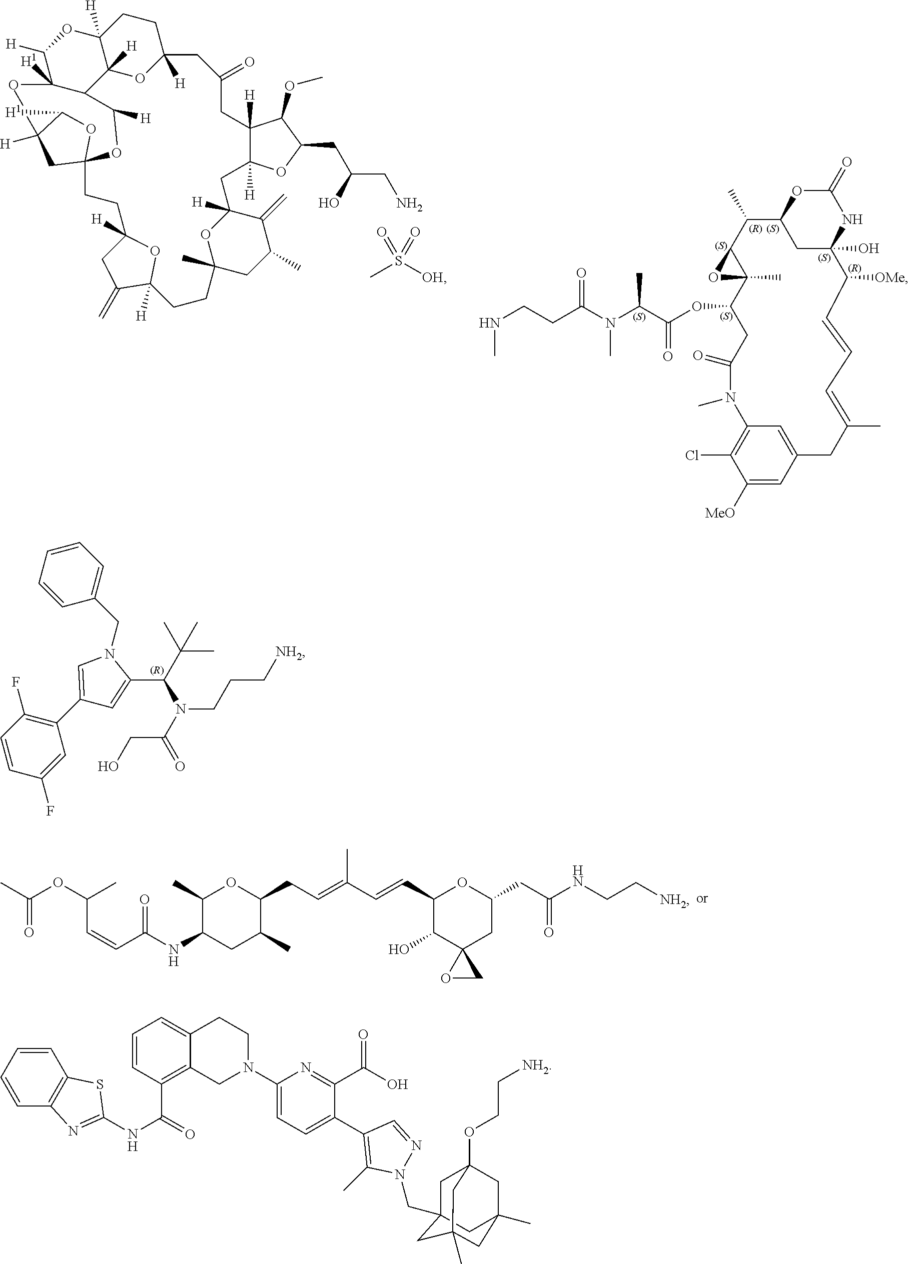

In some embodiments of any of Aspects 5 to 8, Payload is

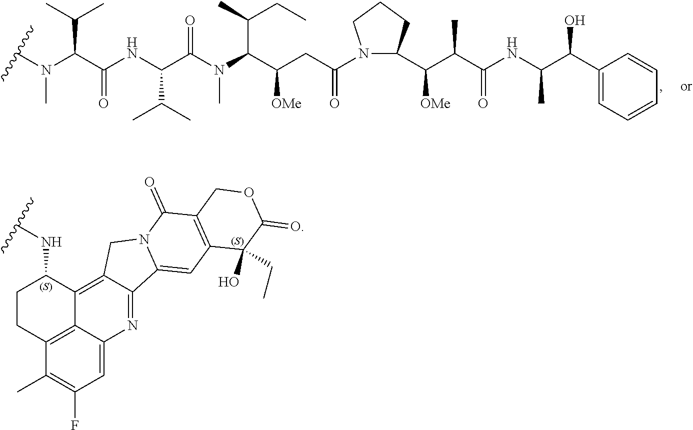

In some embodiments of Aspect 5, the compound is

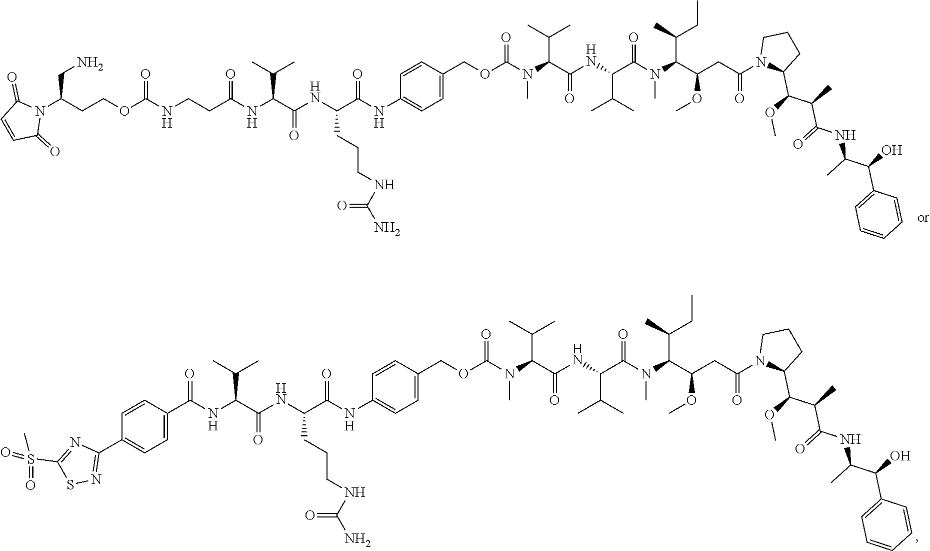 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCNC(=O)OCC[C@H](CN)N2C(=O)C=CC2=O)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2ccc(-c3nsc(S(C)(=O)=O)n3)cc2)C(C)C)cc1)C(C)C]

or a pharmaceutically acceptable salt, tautomer, solvate, or stereoisomer of any of the foregoing.

In some embodiments of Aspect 6, the compound is

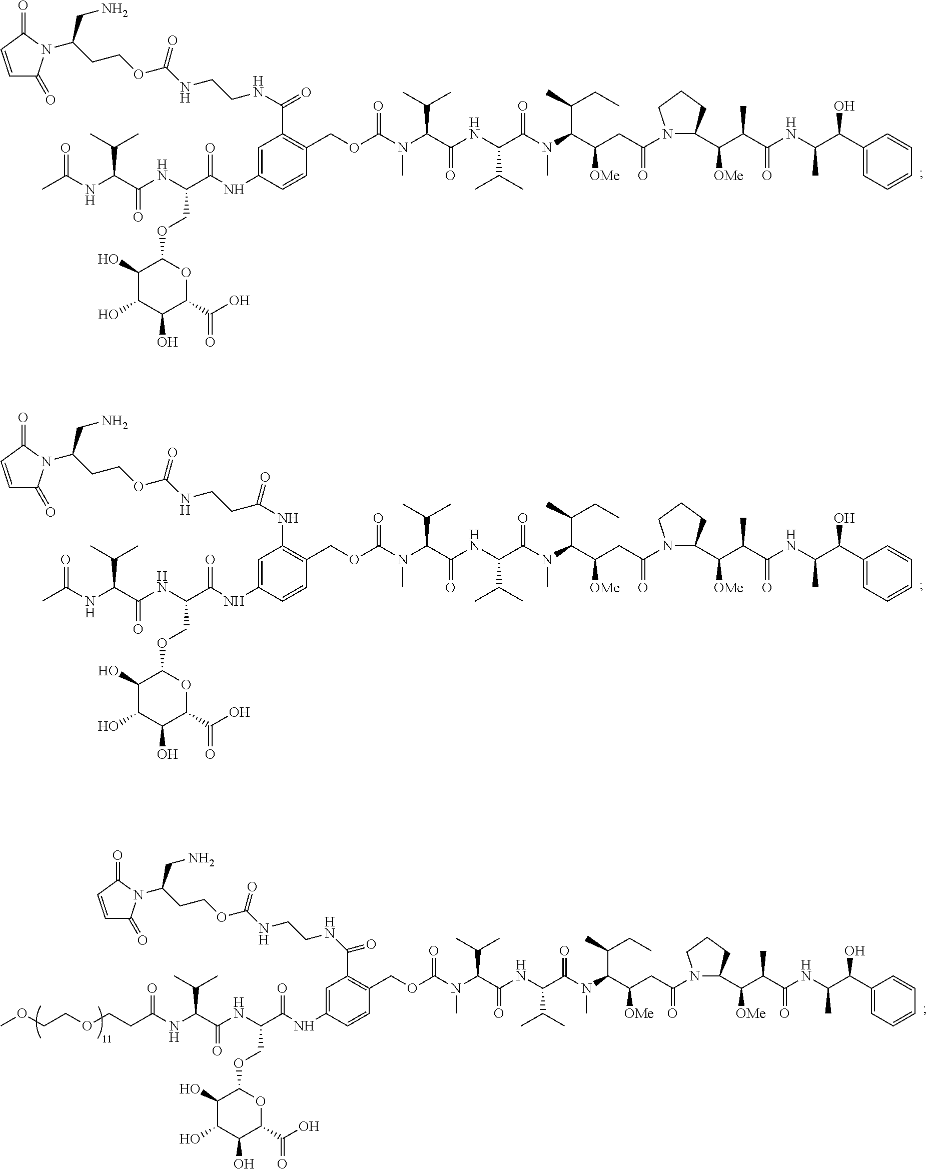

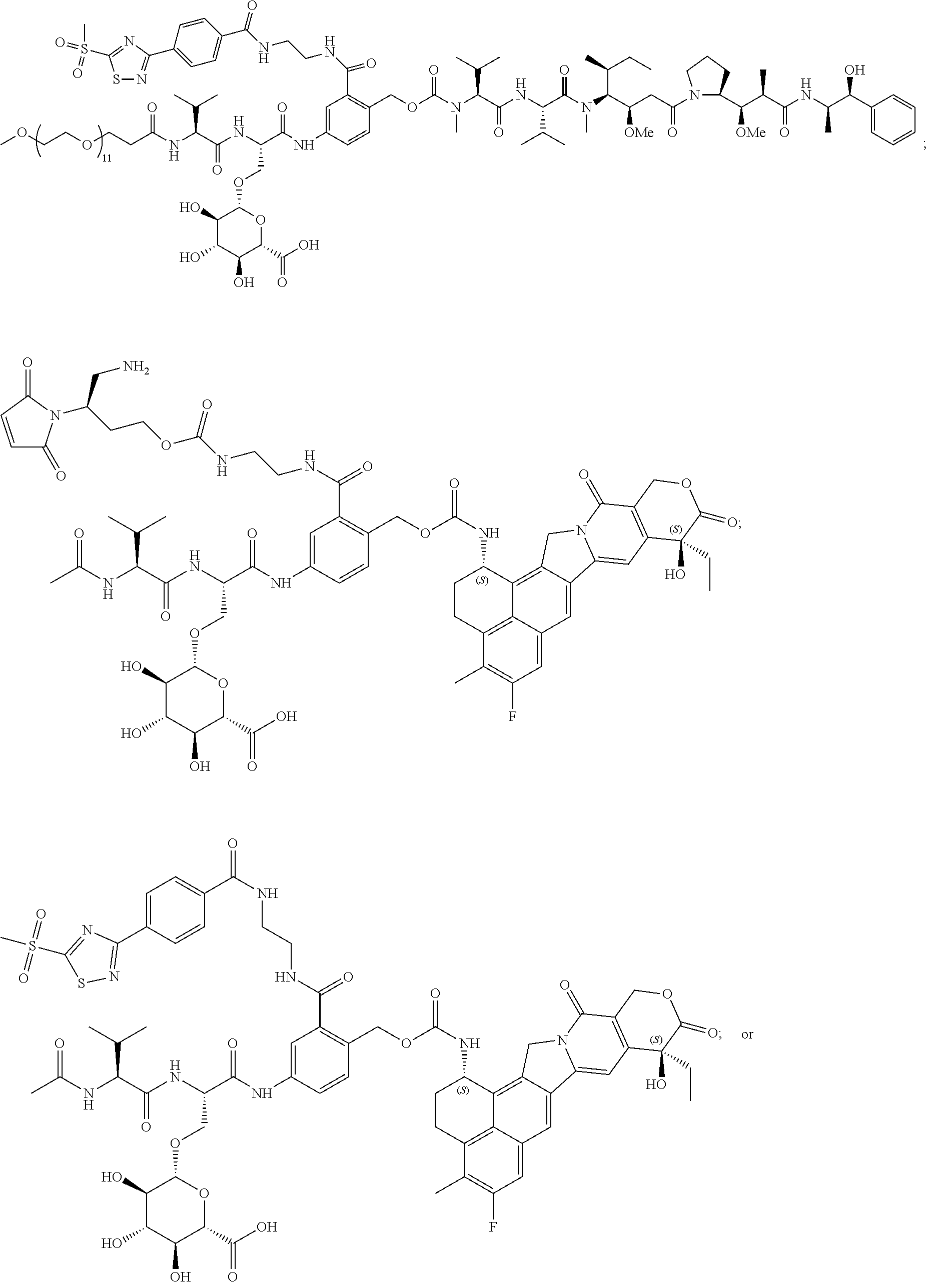

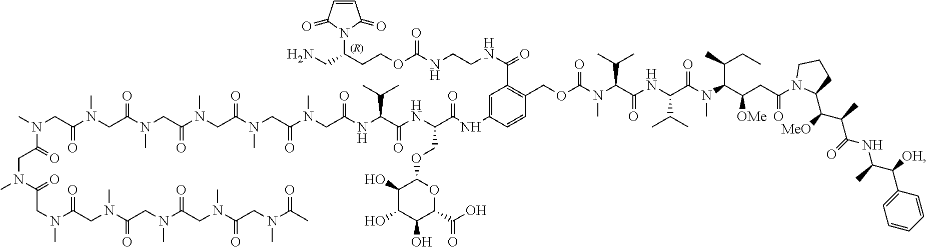

or a pharmaceutically acceptable salt, tautomer, solvate, or stereoisomer of any of the foregoing.

In some embodiments of Aspect 7, the compound is

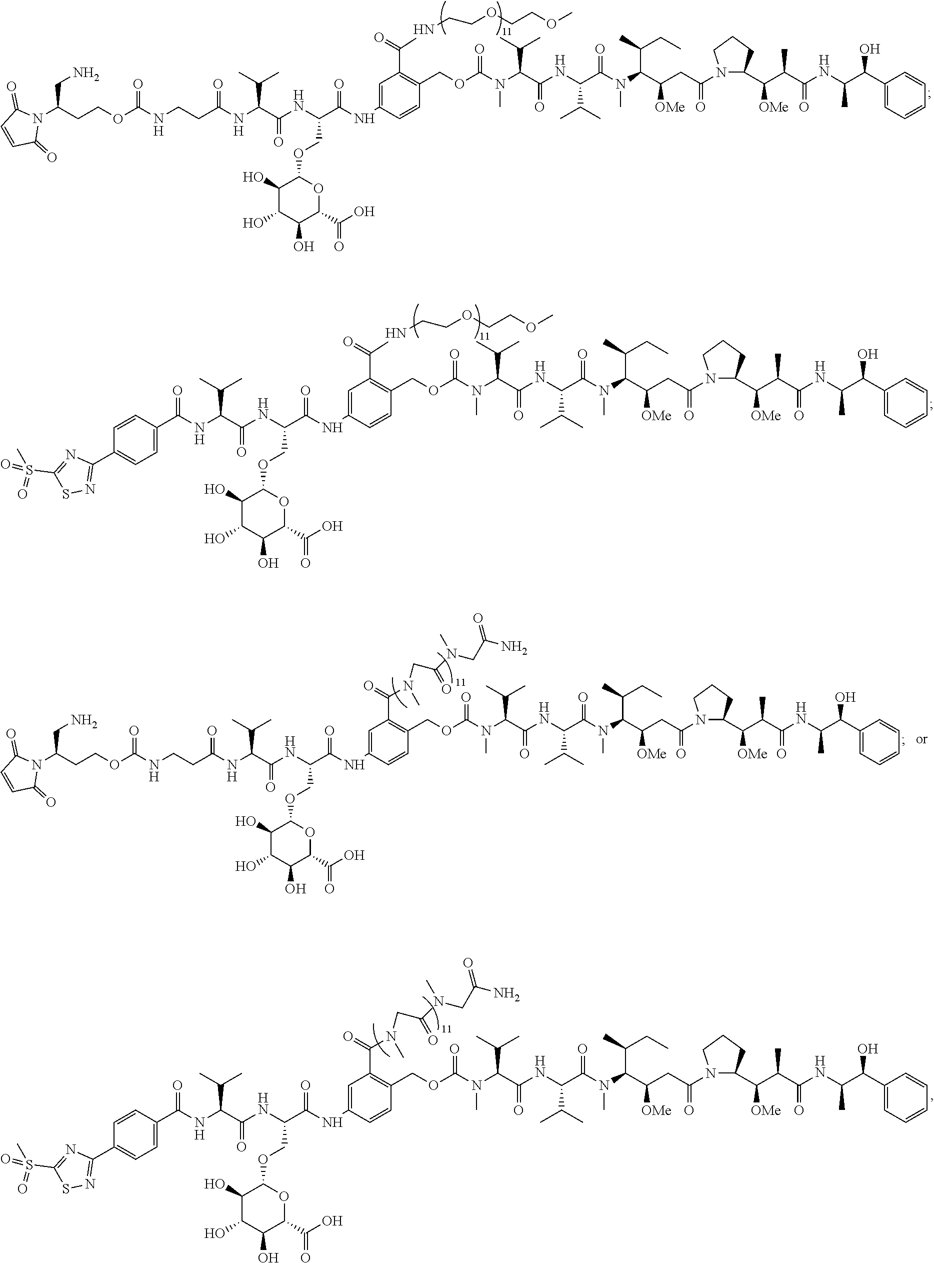

or a pharmaceutically acceptable salt, tautomer, solvate, or stereoisomer of any of the foregoing.

In some embodiments of Aspect 8 the compound is

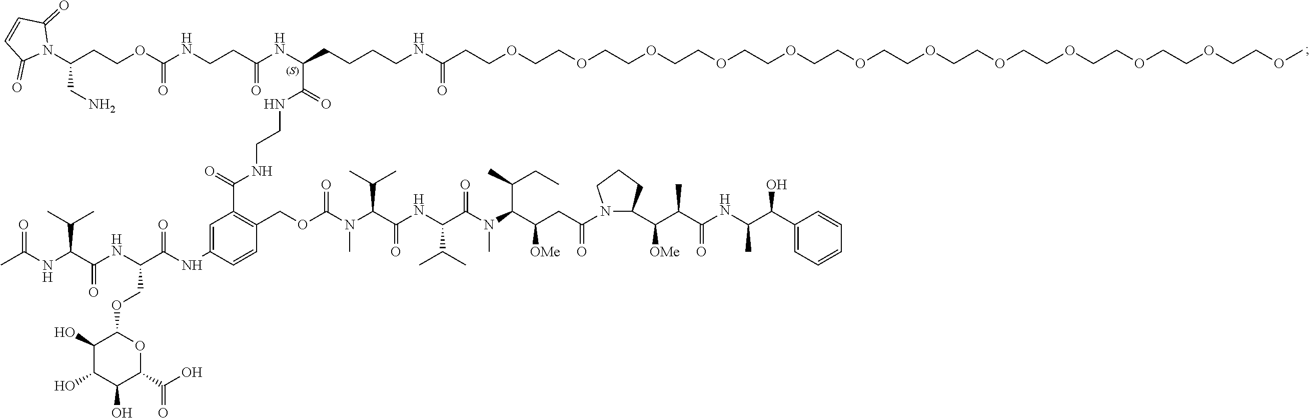

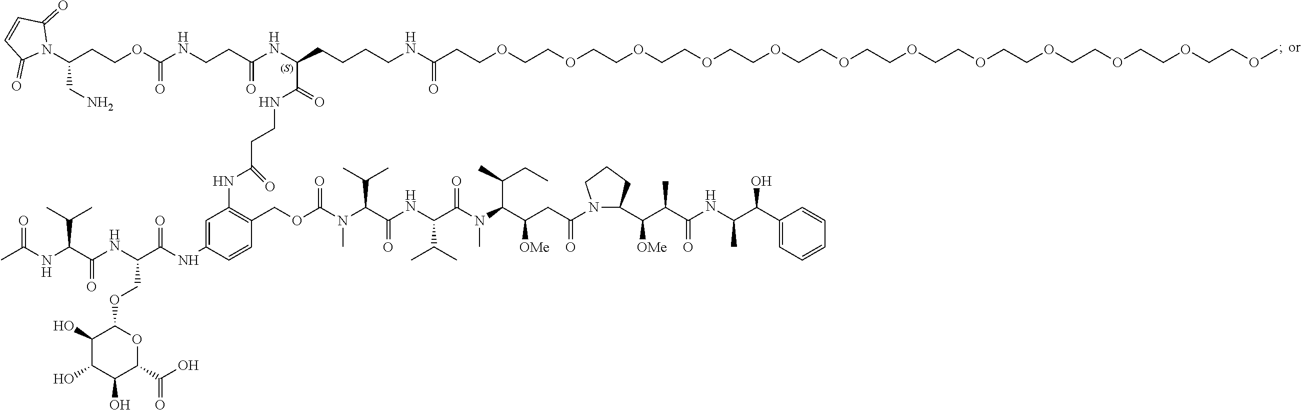

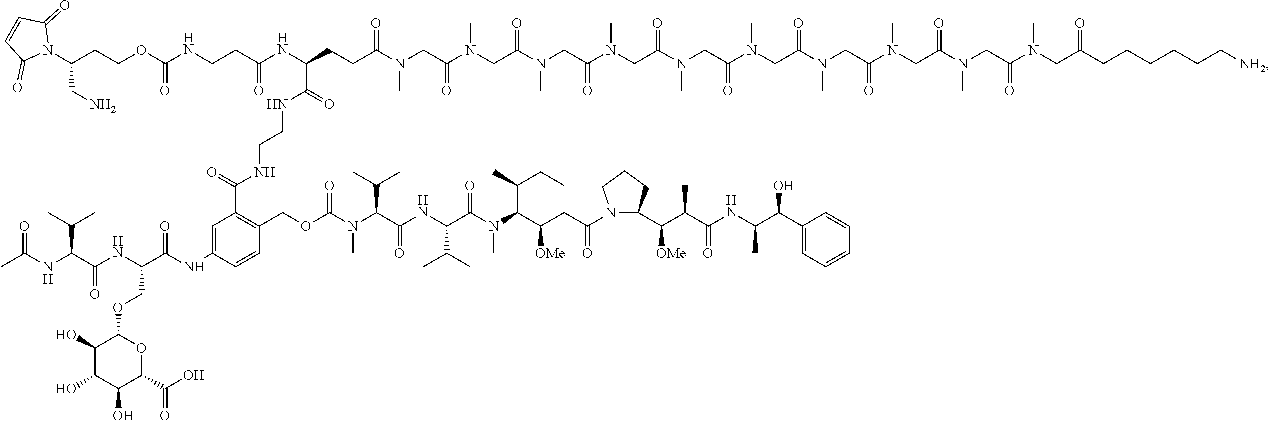

or a pharmaceutically acceptable salt, tautomer, solvate, or stereoisomer of any of the foregoing.

### Methods or Processes of Making the Conjugates

Provided herein are methods of preparing a conjugate by contacting a binding agent (BA) with a conjugator-linker-payload compound under conditions suitable for forming a bond between the binding agent and the conjugator-linker-payload compound. The reaction conditions may be any suitable reaction conditions known in the art. The binding agent may be an antibody an d the bond may form an antibody-drug conjugate.

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 1^{6t}h edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

### Methods of Using the Conjugates

In some embodiments, set forth herein is a method of treating a disease or disorder (e.g., a cancer) in a subject (e.g., patient) in need thereof, comprising administering to the patient a therapeutically effective amount of a conjugate disclosed herein.

The conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (in), or a combination thereof; br stands for broad. LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent an amino acid. The amino acids and their corresponding three letter and single letter abbreviations are as follows:

| | | |
|---|---|---|
| alanine | Ala | (A) |
| arginine | Argcan) | |
| asparagine | Asn | (N) |
| aspartic acid | Asp | (D) |
| cysteine | can | can |
| glutamic | | |
| acicanGcan(E) | | |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| TEA | Triethyl amine |
| THF | Tetrahydrofuran |
| MeOH | Methanol |
| PhI(OAc)₂ | (Diacetoxyiodo)benzene |
| PyBOP | Benzotriazol-1-yl-oxytripyrrolidino-phosphonium |
| | Hexafluorophosphate |
| HOBt | 1-Hydroxybenzotriazole |
| DIEA | N,N-Diisopropylethylamine |
| DMF | N,N-Dimethylformamide |
| DCM | Dichloromethane |
| NH₄Cl | Ammonium chloride |
| LiOH | Lithium hydroxide |
| HCl | Hydrochloric acid |
| NPC | Bis(4-nitrophenyl)carbonate |
| TFA | Trifluoroacetic acid |
| K₂CO₃ | Potassium carbonate |
| MeI | Methyl iodide |
| Na₂SO₄ | Sodium sulfate |
| EtOAc | Ethyl acetate |
| MeONa | Sodium methoxide |
| Prep-HPLC | Preparative high performance liquid chromatography |
| TBSCl | t-Butyldimethylchlorosilane |
| Cu(OAc)₂ | Anhydrous Cupric Acetate |
| Pb(OAc)₄ | Lead tetraacetate |
| TSTU | 2-Succinimido-1,1,3,3-tetra-methyluronium tetrafluor |
| Et₂N | Diethylamine |
| r.t. | room temperature |
| MS | Mass spectrometry |
| ESI | Electron spray ionization |
| FA | Formic acid |
| NHS | N-hydroxysuccinimide |
| MTBE | Methyl tert-butyl ether |
| NHS | N-hydroxysuccinimide |
| Fmoc | 9-fluorenylmethoxycarbonyl protecting group |
| DBU | 1,8-diazabicyclo[5.4.0]undec-7-ene |
| DCC | Dicyclohexylcarbodiimide |
| HPLC | High Performance Liquid Chromatography |
| Mc-OSu | Maleimidocaproyl N-hydroxysuccimidyl ester |
| DMSO | Dimethylsulfoxide |

### UPLC Analysis Methods

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm.
Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC) BEH C18 1.7 μm.
Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

### Example 1: Synthesis of Conjugator-Linker-Payloads

### Example 1-1

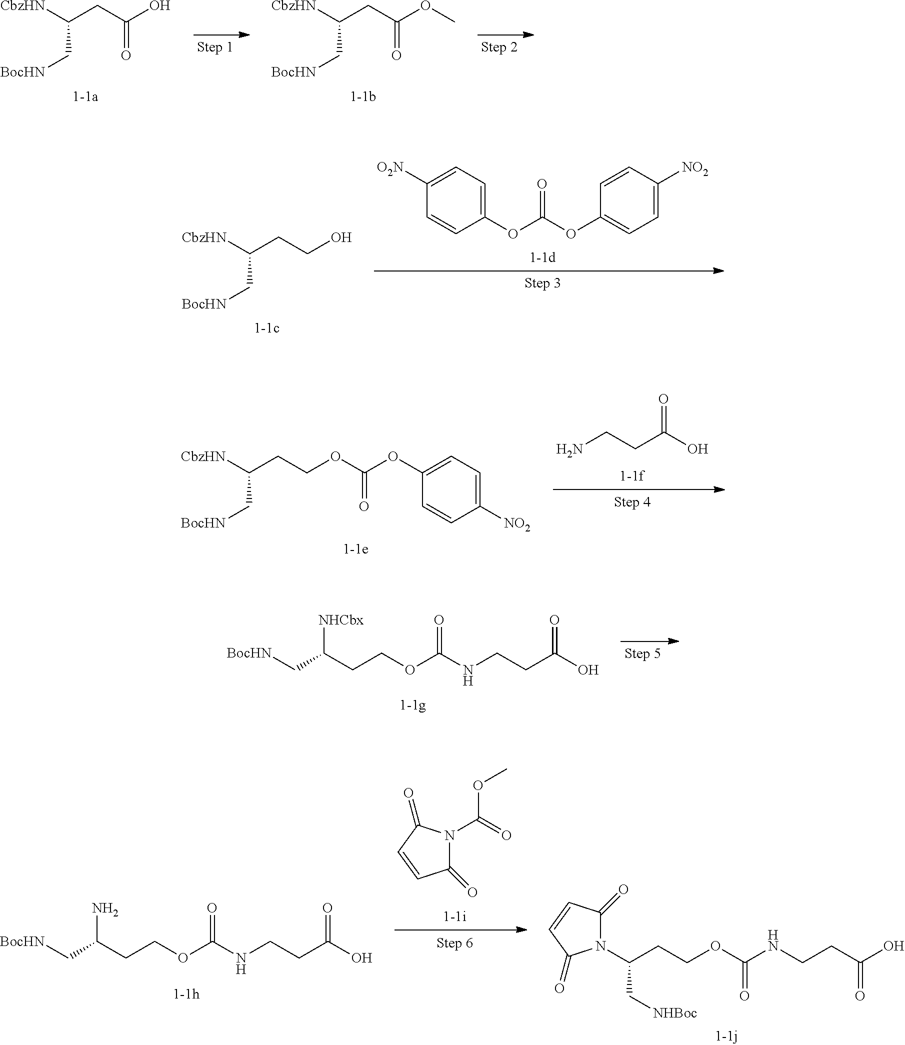

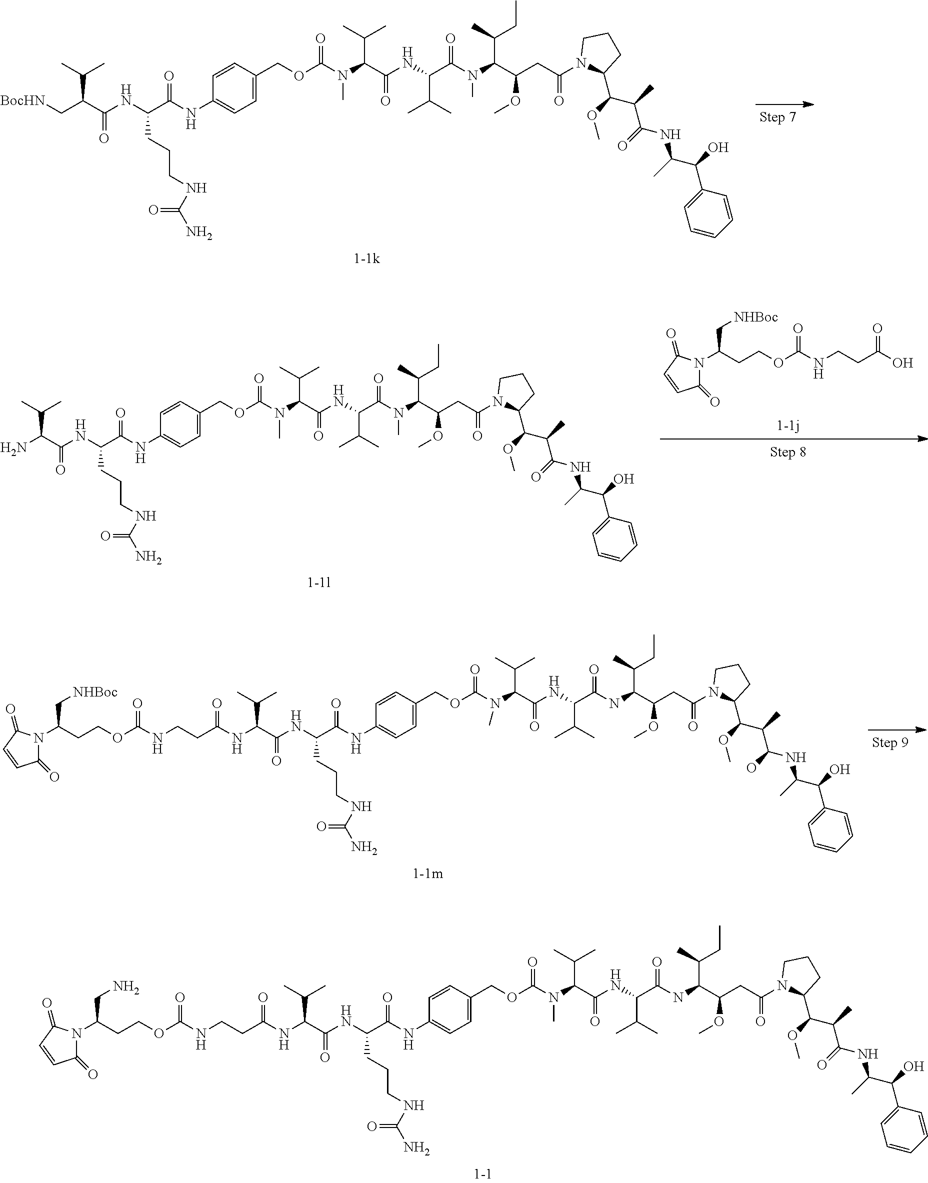

### Step 1: Methyl (R)-3-(((benzyloxy)carbonyl)amino)-4-((tert-butoxycarbonyl)amino)butanoate (1-1b)

1-1a (2.00 g, 5.68 mmol) and K₂CO₃ (863 mg, 6.24 mmol) were added into DMF (10 mL) followed by the dropwise addition of CH₃I (1.61 g, 11.35 mmol) at 0° C. The resulting mixture was stirred at 0° C. for 20 min and allowed to warm to 25° C. and further stirred at 25° C. for 60 min. The reaction process was monitored by TLC (PE/EA) and LCMS. After complete reaction, the reaction mixture was diluted with EA (80 mL) and washed with brine (30 mL*3) and H₂O (30 mL*2). The organic layer was dried over anhydrous Na₂SO₄, filtered, and concentrated under reduced pressure to afford the methyl ester of 1-1b (2.08 g, quant.) as a light-yellow solid. MS (ESI) m/z: 267.2 [M-Boc+H]⁺

### Step 2: Benzyl tert-butyl (4-hydroxybutane-1,2-diyl)(R)-dicarbamate (1-1c)

1-1b (1.00 g, 2.73 mmol) was dissolved in MeOH (15 mL) followed by the addition of LiBH₄ (2 M stock solution in THF, 6.80 mL) at 0° C. The resulting mixture was stirred at 25° C. for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EA (50 mL*3). The combined organic layers were washed with brine (40 mL*2) and water (40 mL*2), dried over anhydrous Na₂SO₄, filtered, and concentrated under reduced pressure and further purified by flash column chromatography (PE/EA) to afford 1-1c (760 mg, 82.3% yield) as a white solid. MS (ESI) m/z: 239.2 [M-Boc+H]⁺

### Step 3: Benzyl tert-butyl (4-(((4-nitrophenoxy)carbonyl)oxy) butane-1,2-diyl)(R)-dicarbamate (1-1e)

1-1c (300 mg, 0.89 mmol) and 1-1d (405 mg, 1.33 mmol) were dissolved in DMF (5 mL) followed by the ad6868uccn of DIEA (229 mg, 1.77 mmol). The resulting mixture was stirred at 25° C. for 1.5 h. After complete reaction. The reaction mixture was diluted with EA (100 mL) and washed with brine (35 mL*2) and water (35 mL*2). The organic layer was dried over anhydrous Na₂SO₄, filtered, and concentrated under reduced pressure to afford 1-1e as a white solid (371 mg, 83.1% yield). MS (ESI) m/z: 404.4 [M-Boc+H]⁺

### Step 4: (9H-fluoren-9-yl)methyl tert-butyl (4-(((3-(dimethylamino)-3-oxopropyl)carbamoyl)oxy) butane-1,3-diyl)(S)-dicarbamate (1-1g)

1-1e (420 mg, 0.83 mmol) and 1-1f (149 mg, 1.67 mmol) were dissolved in DMF (5 mL) followed by the addition of aqueous NaHCO₃ (1M, 5 mL). The resulting mixture was stirred at 25° C. for 2.5 h. After complete reaction, the reaction mixture was concentrated and purified by flash column chromatography (DCM/MeOH) to afford 1-1g as a pale-yellow solid (365 mg, 96.5% yield). MS (ESI) m/z: 354.4 [M-Boc+H]⁺

### Step 5: (R)-7-(2,5-dioxo-2,5-dihydro-1H-pyrrol-1-yl)-2,2-dimethyl-4,11-dioxo-3,10-dioxa-5,12-diazapentadecan-15-oic acid (1-h)

1-1g (360 mg, 0.79 mmol) was dissolved in MeOH (18 mL) followed by the addition of Pd/C (wet base, 108 mg). The resulting mixture was stirred at r.t. under H₂ (15 psi) for 2 h. After complete reaction, the reaction mixture was filtered and concentrated under reduced pressure to afford 1-1 h as a clear syrup (252 mg, 99.4% yield). The crude product was used directly in next step without purification. MS (ESI) m/z: 320.3 [M+H]⁺

### Step 6: (R)-7-(2,5-dioxo-2,5-dihydro-1H-pyrrol-1-yl)-2,2-dimethyl-4,11-dioxo-3,10-dioxa-5,12-diazapentadecan-15-oic acid (1-j)

1-1h (250 mg, 0.78 mmol) and 1-1i (243 mg, 1.57 mmol) were dissolved in a mixed solvent of ACN (8 mL) and aqueous NaHCO₃ (1M, 16 mL). The resulting mixture was stirred at 0° C. for 1 h and further stirred at 25° C. until the reaction was complete. Then the reaction mixture was acidified with aq. KHSO₄ (20 mL) and extracted with EA (35 mL*3). The combined organic layers were dried over anhydrous Na₂SO₄, filtered, and concentrated under reduced pressure to afford a yellow oil as the crude product which was purified by flash column chromatography to afford 1-1j (280 mg, 89.6% yield) as a white solid. MS (ESI) m/z: 422.3 [M+Na]⁺

¹H NMR (400 MHz, d₆-DMSO) δ 12.48 (s, 1H), 7.03-7.01 (m, 2H), 6.99 (s, 2H), 4.08-4.03 (m, 3H), 3.86-3.83 (m, 2H), 3.14-3.11 (m, 2H), 2.35 (t, J=7.2 Hz, 2H), 2.16-2.09 (m, 1H), 1.91-1.84 (m, 1H), 1.32 (s, 9H).

### Step 7: 4-((S)-2-((S)-2-amino-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1-1l)

1-1k (126 mg, 0.10 mmol, purchased from ChemExpress) was added into DCM (6 mL) followed by the addition of TFA (1.5 mL). The resulting mixture was stirred at 25° C. until complete consumption of 1-1k. Then the resulting mixture was concentrated under reduced pressure to afford 1-11 (116 mg, quant.) as a white solid. The crude product was used directly in next step without purification. MS (ESI) m/z: 1123.9 [M+H]⁺

### Step 8: (R)-4-((tert-butoxycarbonyl)amino)-3-(2,5-dioxo-2,5-dihydro-1H-pyrrol-1-yl)butyl (3-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-3-oxopropyl)carbamate (1-1m)

1-1j (20 mg, 0.050 mmol), 1-11 (51 mg, 0.045 mmol) and HATU (22 mg, 0.058 mmol) were dissolved in DMF (3 mL) followed by the addition of DIEA (13 mg, 0.10 mmol). The resulting mixture was stirred at r.t. until 1-11 was completely consumed. After complete reaction, the reaction mixture was purified by Prep-HPLC (Method: column: XBridge Prep C18 OBD 5 μm 19*250 mm; Mobile phase: A-water (0.1% formic acid): B-acetonitrile; Flow rate: 20 mL/min). After lyophilization, 1-1m (35 mg, 51.6% yield) was obtained as a white solid. MS (EST) m/z: 1504.9 [M+H]⁺

### Step 9: 4-((2S,5S,15R)-16-amino-15-(2,5-dioxo-2,5-dihydro-1H-pyrrol-1-yl)-5-isopropyl-4,7,11-trioxo-2-(3-ureidopropyl)-12-oxa-3,6,10-triazahexadecanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1-1)

1-1m (32 mg, 0.021 mmol) was added into DCM (2 mL) followed by the addition of TFA (0.5 mL) at 0° C. The resulting mixture was stirred at 25° C. until the reaction was complete. Then the reaction mixture was concentrated and purified by Prep-HPLC (Method: column: XBridge Prep C18 OBD 5 μm 19*250 mm; Mobile phase: A-water (0.1% trifluoroacetic acid): B-acetonitrile; Flow rate: 20 mL/min). After lyophilization, 1-1 (23 mg, 72.0% yield) was obtained as a white solid. MS (ESI) m/z: 1406.0 [M+H]⁺

### Example 1-2

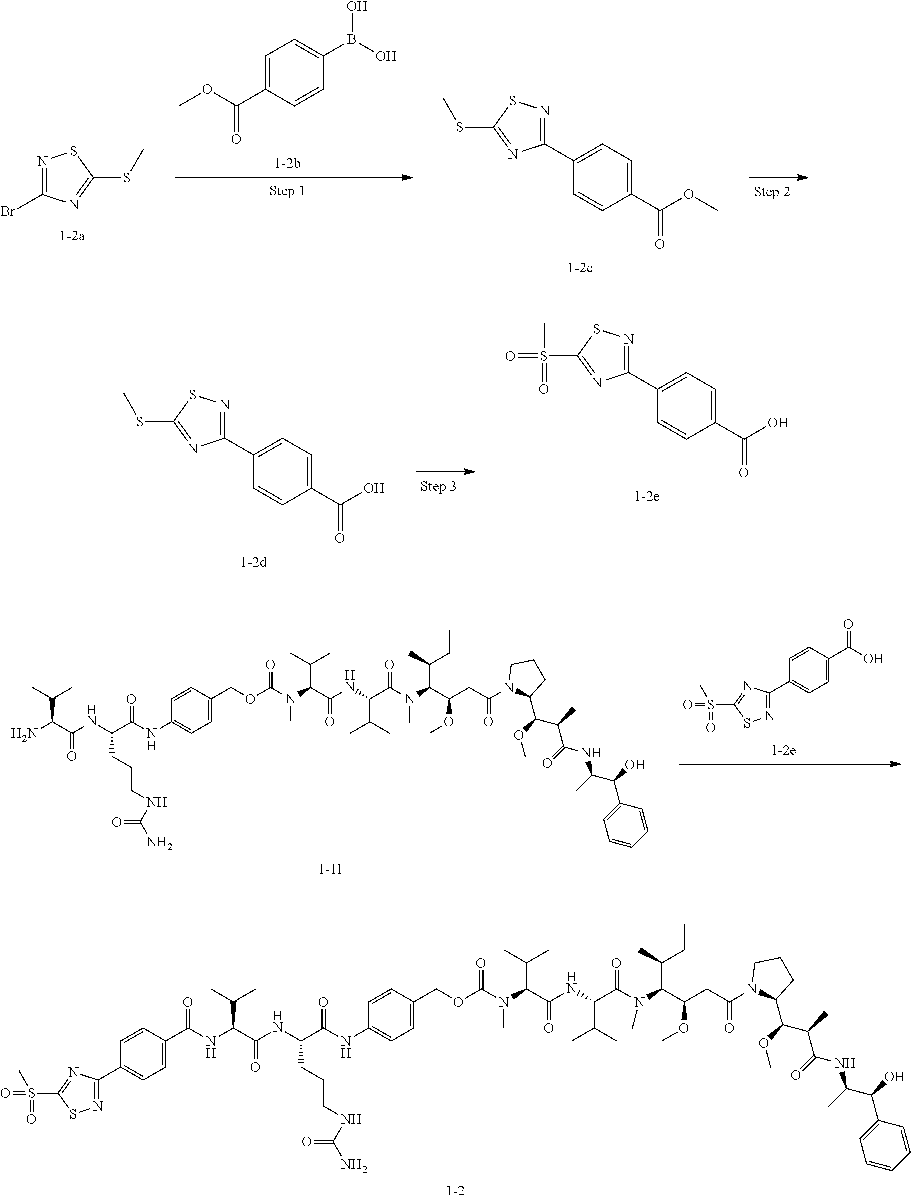

### Step 1: Methyl 4-(5-(methylthio)-1,2,4-thiadiazol-3-yl)benzoate (1-2c)

To a solution of compound 1-2a (100 mg, 0.47 mmol) in toluene (4 mL) and H₂O (1 mL) were added compound 1-2b (110 mg, 0.57 mmol), K₂CO₃ (168 mg, 0.95 mmol) and Pd(dppf)Cl₂. (35 mg, 0.047 mmol). The mixture was stirred at 110° C. for 3 h under N₂ atmosphere. Then the mixture was filtered through a pad of celite, diluted with EA (100 mL), washed by brine (50 mL*4). The organic layer was dried over anhydrous Na₂SO₄, filtered and concentrated. The crude was purified by flash column chromatography (eluted with PE:EA=100/0~60/40). Compound 1-2c (56 mg, 44.4% yield) was obtained as an off-white solid. MS (ESI) m/z: 267.1 [M+H]⁺

### Step 2:4-(5-(methylthio)-1,2,4-thiadiazol-3-yl)benzoic acid (1-2d)

To a solution of compound 1-2c (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (Method: column: XBridge Prep C18 OBD 5 μm 19*250 mm; Mobile phase: A-water (0.1% formic acid): B-acetonitrile; Flow rate: 20 mL/min) to give compound 1-2d (36 mg, 70.3% yield) as a white solid. MS (ESI) m/z: 253.1 [M+H]⁺

### Step 3:4-(5-(methylsulfonyl)-1,2,4-thiadiazol-3-yl)benzoic acid (1-2e)

To a solution of compound 1-2d (35 mg, 0.14 mmol) in DCM (3 mL) and THF (3 mL) was added m-CPBA (96 mg, 0.55 mmol). The mixture was stirred at room temperature for 16 h. The mixture was concentrated and purified by prep-HPLC (Method: column: XBridge Prep C18 OBD 5 um 19*250 mm; Mobile phase: A-water (0.1% TFA): B-acetonitrile; Flow rate: 20 mL/min). Compound 1-2e (12 mg, 99% purity) was obtained as a white solid. MS (ESI) m/z: 284.8 [M+H]⁺

### Step 4: 4-((S)-2-((S)-3-methyl-2-(4-(5-(methylsulfonyl)-1,2,4-thiadiazol-3-yl)benzamido) butanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1-2)

1-2 (13 mg, 31.8% yield) was synthesized according to the procedure of step 8 of example 1-1. MS (ESI) m/z: 1389.8 [M+H]⁺

### Example 1-3

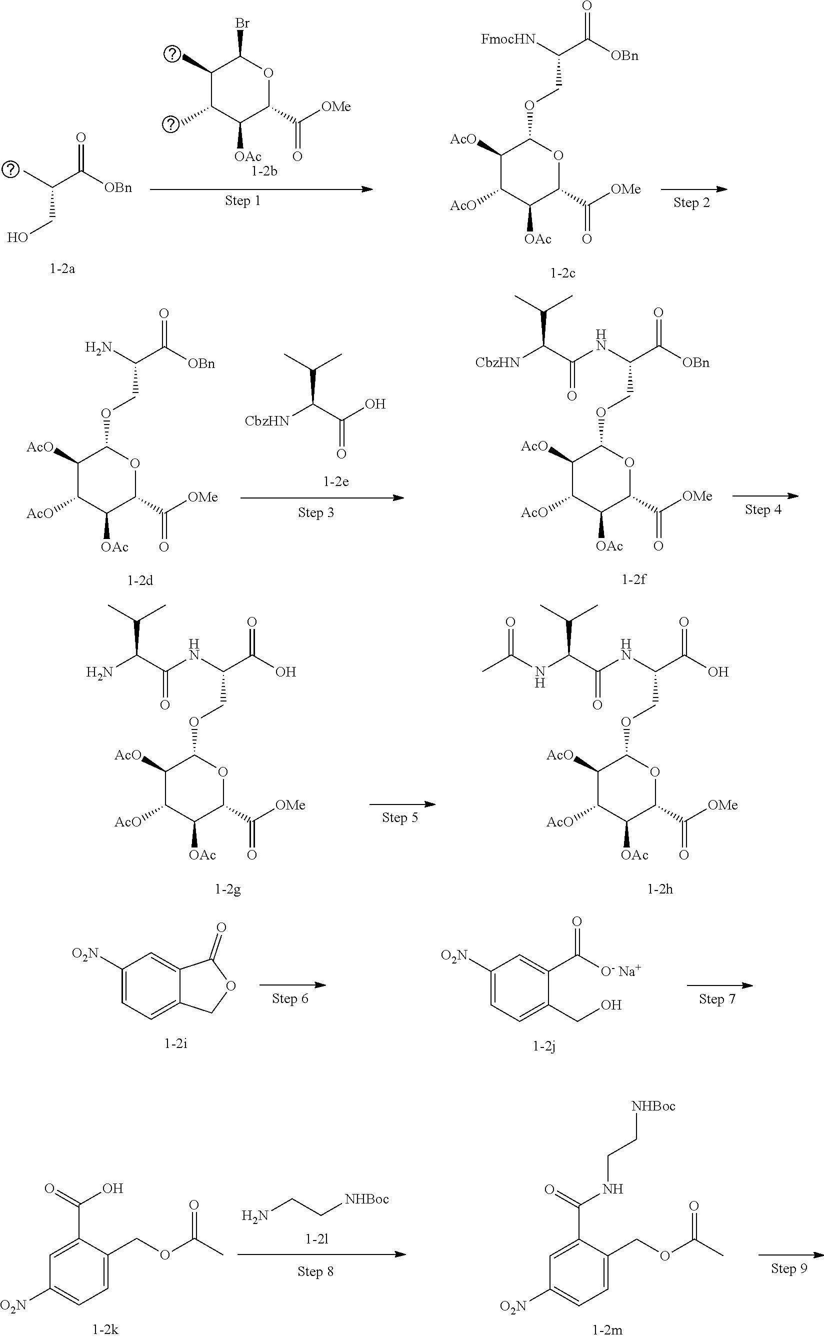

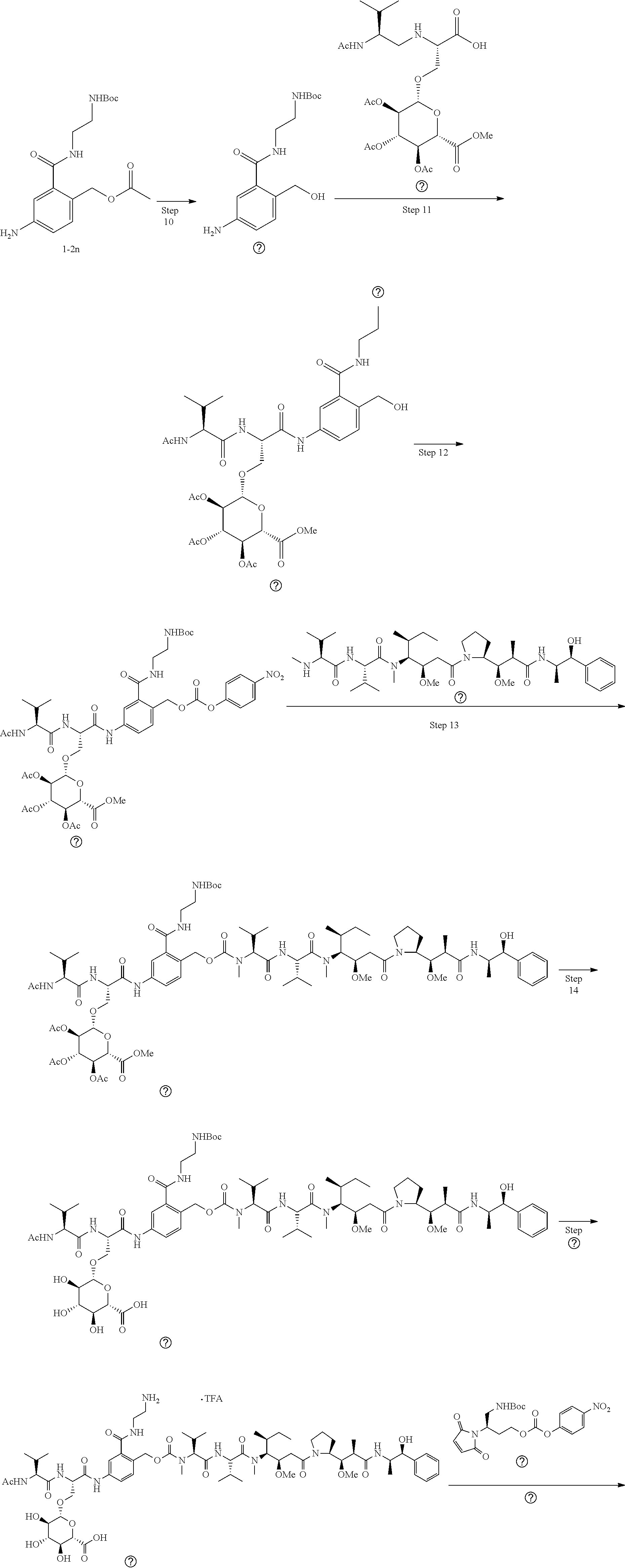

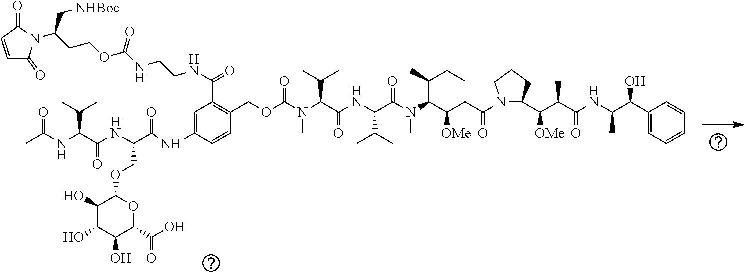 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1C(=O)NCCNC(=O)OCC[C@H](CNC(=O)OC(C)(C)C)N1C(=O)C=CC1=O)C(C)C]

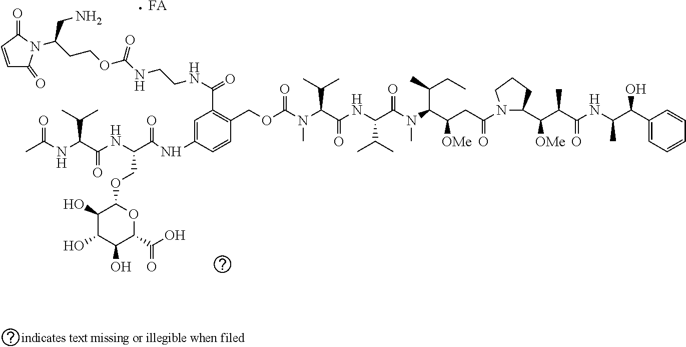

### Step 1:(2R,3R,4S,5S,6S)-2-((S)-2-((((9H-fluoren-9-yl)methoxy)carbonyl)amino)-3-(benzyloxy)-3-oxopropoxy)-6-(methoxy carbonyl)tetrahydro-2H-pyran-3,4,5-triyl triacetate (1-3c)

To a solution of compound 1-3a (1.00 g, 2.41 mmol) in DCM (30 mL) was added 1-3b (1.14 g, 2.87 mmol) and 4 Å molecular sieve (4.00 g). The mixture was stirred at room temperature for 30 min. The mixture was cooled to −20° C. Then AgOTf (0.74 g, 2.87 mmol) was added into the mixture in portion at −20° C. The mixture was stirred at −20° C. for 4 h. The mixture was filtered, the filtrate was diluted with EA (150 mL), washed by sat. NaHCO₃ (100 mL*3), brine (100 mL*3), respectively. The organic layer was dried over anhydrous Na₂SO₄, filtered and concentrated. The residue was purified by flash column chromatography (eluted with Petroleum ether:EtOAc=0%-40%) to give the crude product. The crude was further purified by isopropanol to give compound 1-3c (340 mg, 19.3% yield) was obtained as white solid. MS (ESI) m/z: 734.4 [M+H]⁺

### Step 2: (2R,3R,4S,5S,6S)-2-((S)-2-amino-3-(benzyloxy)-3-oxopropoxy)-6-(methoxycarbonyl)tetrahydro-2H-pyran-3,4,5-triyl triacetate (1-3d)

To a solution of compound 1-3c (250 mg, 0.34 mmol) in DMF (1 mL) was added Et2NH (125 mg, 1.70 mmol). The mixture was stirred at r.t. for 30 min. The mixture was concentrated under high vacuum, and co-evaporated with toluene (3 mL*3) to give compound 1-3d (174 mg, crude) as brown oil, which was used directly without further purification. MS (ESI) m/z: 512.3 [M+H]⁺

### Step 3: (2R,3R,4S,5S,6S)-2-((S)-3-(benzyloxy)-2-((S)-2-(((benzyloxy)carbonyl)amino)-3-methylbutanamido)-3-oxopropoxy)-6-(methoxycarbonyl)tetrahydro-2H-pyran-3,4,5-triyl triacetate (1-3f)

To a solution of compound 1-3d (174 mg, 0.34 mmol) in DMF (4 mL) were added compound 1-3e (103 mg, 0.41 mmol), HATU (168 mg, 0.44 mmol) and DIEA (88 mg, 0.68 mmol). The mixture was stirred at r.t. for 4 h. The reaction was diluted with EA (50 mL), washed by sat. NaHCO₃ solution (30 mL*3), brine (30 mL*3). The organic layer was dried over anhydrous Na₂SO₄, filtered, concentrated to give the residue. The residue was purified by flash column chromatography (eluted with CH₂Cl₂:EtOAc=100/0~70/30). Compound 1-3f (230 mg, 91% yield) was obtained as a white solid. MS (ESI) m/z: 745.4 [M+H]⁺

### Step 4: N-(L-valyl)-O-((2R,3R,4S,5S,6S)-3,4,5-triacetoxy-6-(methoxycarbonyl)tetrahydro-2H-pyran-2-yl)-L-serine (1-3g)

To a solution of compound 1-3f (230 mg, 0.31 mmol) in MeOH (3 mL) was added Pd/C (10%, 23 mg). The mixture was stirred at r.t. under H₂ (15 psi) for 1 h. H₂O (3 mL) was added into the mixture, filtered, concentrated to give crude product 1-3g (161 mg, crude) as a white solid, which was used directly without further purification. MS (ESI) m/z: 521.3 [M+H]⁺

### Step 5: N-(acetyl-L-valyl)-O-((2R,3R,4S,5S,6S)-3,4,5-triacetoxy-6-(methoxycarbonyl)tetrahydro-2H-pyran-2-yl)-L-serine (1-3h)

To a solution of compound 1-3g (161 mg, 0.31 mmol) in CH₃COOH (3 mL) was added Ac₂O) (316 mg, 3.09 mmol) at 0° C. The mixture was stirred at room temperature for 1 h. The mixture was concentrated and purified by prep-HPLC (Method: column: XBridge Prep C18 OBD 5 μm 19*250 mm; Mobile phase: A-water (0.1% TFA): B-acetonitrile; Flow rate: 20 mL/min). Compound 1-3h (145 mg, 83.3% yield) was obtained as a white solid. MS (ESI) m/z: 563.4 [M+H]⁺

### Step 6: Sodium 2-(hydroxymethyl)-5-nitrobenzoate (1-3j)

A solution of compound 1-3i (5.00 g, 27.9 mmol) in NaOH (1 N, 27.9 mL, 27.9 mmol) was refluxed for 2 hours. The mixture was cooled to room temperature and lyophilized to give compound 1-3j (6.12 g, crude) for next step directly without further purification. MS (ESI) m/z: 195.9 [M−H]⁻

### Step 7:2-(acetoxymethyl)-5-nitrobenzoic acid (1-3k)

To a solution of compound 1-3j (5.00 g, 22.8 mmol) in DMF (100 mL) were added Ac₂O (11.60 g, 114 mmol) and Et₃N (11.5 g, 114 mmol) at 0° C. The mixture was stirred at 25° C. for 16 h. The mixture diluted with EtOAc (500 mL) washed by brine (200 mL*4). The organic layer was dried over Na₂SO₄ and concentrated under reduced pressure to give the residue. The crude product was purified by silica gel column chromatography (CH₂Cl₂/MeOH=100/0~10/90) to give compound 1-3k (2.30 g, 42.1% yield) as a light-yellow solid. MS (ESI) m/z: 239.2 [M+H]⁺

### Step 8: 2-((2-((tert-butoxycarbonyl)amino)ethyl)carbamoyl)-4-nitrobenzyl acetate (1-3m)

To a solution of compound 1-3k (1.60 g, 6.69 mmol) in DMF (10 mL) were added compound 1-31 (1.29 g, 8.03 mmol), HATU (3.82 g, 10.03 mmol) and DIEA (1.73 g, 13.38 mmol). The mixture was stirred at r.t. for 30 min. The reaction was diluted with EA (150 mL), washed by saturated NaHCO₃ solution (50 mL*3), brine (50 mL*3). The organic layer was dried over anhydrous Na₂SO₄, filtered, and concentrated to give the residue. The residue was purified by silica gel column chromatography (eluted with petroleum ether:EtOAc=100/0~70/30). Compound 1-3m (1.20 g, 47.1% yield) was obtained as white solid. MS (ESI) m/z: 404.3 [M+Na]⁺

### Step 9:4-amino-2-((2-((tert-butoxycarbonyl)amino)ethyl)carbamoyl)benzyl acetate (1-3n)

To a solution of compound 1-3m (1.20 g, 3.15 mmol) in EtOAc (15 mL) was added PtO₂ (120 mg). The mixture was stirred at r.t. under H₂ for 2 h. The mixture was filtered and concentrated to give crude product 1-3n (1110 mg, crude) as an off-white solid, which was used directly without further purification. MS (ESI) m/z: 374.3 [M+Na]⁺

### Step 10: tert-butyl(2-(5-amino-2-(hydroxymethyl)benzamido)ethyl)carbamate (1-30)

To a solution of compound 1-3n (1.10 g, 3.13 mmol) in MeOH (10 mL) was added K₂CO₃ (216 mg). The mixture was stirred at r.t. for 1 h. The mixture was filtered and concentrated to give crude. The residue was purified by silica gel column chromatography (eluted with CH₂Cl₂/MeOH=100/0~90/10) to give compound 1-30 (783 mg, 80.8% yield). MS (ESI) m/z: 292.3 [M−H₂O+H]⁺

### Step 11

### (2R,3R,4S,5S,6S)-2-((S)-2-((S)-2-acetamido-3-methylbutanamido)-3-((3-((2-((tert-butoxycarbonyl)amino)ethyl) carbamoyl)-4-(hydroxymethyl)phenyl)amino)-3-oxopropoxy)-6-(methoxycarbonyl)tetrahydro-2H-pyran-3,4,5-triyl triacetate (1-3p)

To a solution of compound 1-30 (780 mg, 1.39 mmol) in CH₂Cl₂ (6 mL) were added compound 1-3h (515 mg, 1.66 mmol), EEDQ (446 mg, 1.80 mmol). The mixture was stirred at r.t. for 2 h. The reaction was diluted with EtOAc (150 mL), washed by sat. NaHCO₃ solution (50 mL*3), brine (50 mL*3). The organic layer was dried over anhydrous Na₂SO₄, filtered and concentrated to give the residue. The residue was purified by silica gel column chromatography (eluted with petroleum ether=100/0~70/30). Compound 1-3p (650 mg, 55.1% yield) was obtained as a white solid. MS (ESI) m/z: 876.5 [M+Na]⁺

### Step 12

(2R,3R,4S,5S,6S)-2-((S)-2-((S)-2-acetamido-3-methylbutanamido)-3-((3-((2-((tert-butoxycarbonyl)amino)ethyl) carbamoyl)-4-((((4-nitrophenoxy)carbonyl)oxy)methyl)phenyl)amino)-3-oxopropoxy)-6-(methoxycarbonyl)tetrahydro-2H-pyran-3,4,5-triyl triacetate (1-3q)

To a solution of compound 1-3p (600 mg, 0.70 mmol) in DMF (10 mL) was added bis(4-nitrophenyl)carbonate (641 mg, 2.11 mmol) and DIEA (272 mg, 2.11 mmol). The mixture was stirred at r.t. for 3 h. The solvent was evaporated under high vacuum. The crude was purified by silica gel column chromatography (eluted with CH₂Cl₂:MeOH=100/0~10/90) to give compound 1-3q (476 mg, 66.5% yield) as a light-yellow oil. MS (ESI) m/z: 1041.6 [M+Na]⁺

### Step 13

### (2R,3R,4S,5S,6S)-2-((S)-2-((S)-2-acetamido-3-methylbutanamido)-3-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-6-(methoxycarbonyl)tetrahydro-2H-pyran-3,4,5-triyl triacetate (1-3s)

To a solution of compound 1-3q (353 mg, 0.35 mmol) in DMF (6 mL) were added 1-3r (261 mg, 0.36 mmol), HOBt (47 mg, 0.35 mmol) and DIEA (90 mg, 0.69 mmol). The mixture was stirred at r.t. for 16 h. The solvent was evaporated under high vacuum. The crude was purified by silica gel column chromatography (eluted with CH₂Cl₂:MeOH=100/0~90/10) to give compound 1-3s (455 mg, 82.3% yield) as a light-yellow oil. MS (ESI) m/z: 1598.3 [M+H]⁺

### Step 14

### (2S,3S,4S,5R,6R)-6-((S)-2-((S)-2-acetamido-3-methylbutanamido)-3-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-3,4,5-trihydroxytetrahydro-2H-pyran-2-carboxylic acid (1-3t)

To a solution of compound 1-3s (100 mg, 0.063 mmol) in MeOH (15 mL) was added NaOMe (1.25 mL, 2 N, 0.13 mmol) at 0° C. The mixture was stirred at r.t. for 2 h. The mixture was cooled down to 0° C., then NaOH (63 μL, 2N, 0.13 mmol) was added into the mixture dropwise. The mixture was stirred at 0° C. for 1 hr. The reaction was quenched by CH₃COOH (1 mL), concentrated. The crude was purified using Prep-HPLC (Method: column: XBridge Prep C18 OBD 5 μm 19*150 mm; Mobile phase: A-water (0.1% formic acid): B-acetonitrile; Flow rate: 20 mL/min) to give compound 1-3t (71 mg, 77.8% yield) as a white solid. MS (ESI) m/z: 1458.2 [M+H]⁺

### Step 15

### (2S,3S,4S,5R,6R)-6-((S)-2-((S)-2-acetamido-3-methylbutanamido)-3-((3-((2-aminoethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-3,4,5-trihydroxytetrahydro-2H-pyran-2-carboxylic acid-2,2,2-trifluoroacetaldehyde (1/1) (1-3u) [335]

To a solution of compound 1-3t (71 mg, 0.049 mmol) in CH₂Cl₂ (2 mL) were added TFA (1 mL, 20% in DCM). The mixture was stirred at r.t. for 1 h. The mixture was concentrated to give compound 1-3u (70.9 mg, crude) as an off-white solid, which was used for next step without further purification. MS (ESI) m/z: 1358.8 [M+H]⁺

### Step 16

### (2S,3S,4S,5R,6R)-6-((S)-2-((S)-2-acetamido-3-methylbutanamido)-3-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-3-(((R)-8-(2,5-dioxo-2,5-dihydro-1H-pyrrol-1-yl)-13,13-dimethyl-4,11-dioxo-5,12-dioxa-3,10-diazatetradecyl)carbamoyl)phenyl)amino)-3-oxopropoxy)-3,4,5-trihydroxytetrahydro-2H-pyran-2-carboxylic acid (1-3w)

To a solution of compound 1-3u (35 mg, 0.024 mmol) in DMF (2 mL) were added compound 1-3v (21 mg, 0.048 mmol) and DIEA (6.2 mg, 0.048 mmol). The mixture was stirred at r.t. for 2 h. The reaction was purified using prep-HPLC (Method: column: XBridge Prep C18 OBD 5 μm 19*150 mm; Mobile phase: A-water (0.1% formic acid): B-acetonitrile; Flow rate: 20 mL/min) to give compound 1-3w (19 mg, 47.5% yield) as a white solid. MS (ESI) m/z: 1668.9 [M+H]⁺

### Step 17

### (2S,3S,4S,5R,6R)-6-((S)-2-((S)-2-acetamido-3-methylbutanamido)-3-((3-((2-((((R)-4-amino-3-(2,5-dioxo-2,5-dihydro-1H-pyrrol-1-yl)butoxy)carbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-3,4,5-trihydroxytetrahydro-2H-pyran-2-carboxylic acid—formic acid (1/1) (1-3)

To a solution of compound 1-3w (19 mg, 0.011 mmol) in CH₂Cl₂ (2 mL) were added TFA (1 mL, 20% in CH₂Cl₂). The mixture was stirred at r.t. for 1 h. The mixture was concentrated and purified using prep-HpLC (Method: column: XBridge Prep C18 OBD 5 μm 19*150 mm;

### Mobile Phase:

A-water (0.1% formic acid): B-acetonitrile; Flow rate: 20 mL/min) to give compound 1-3 (12 mg, 65.8% yield) as a white solid. MS (ESI) m/z: 1568.5 [M+H]⁺

### Example 1-4

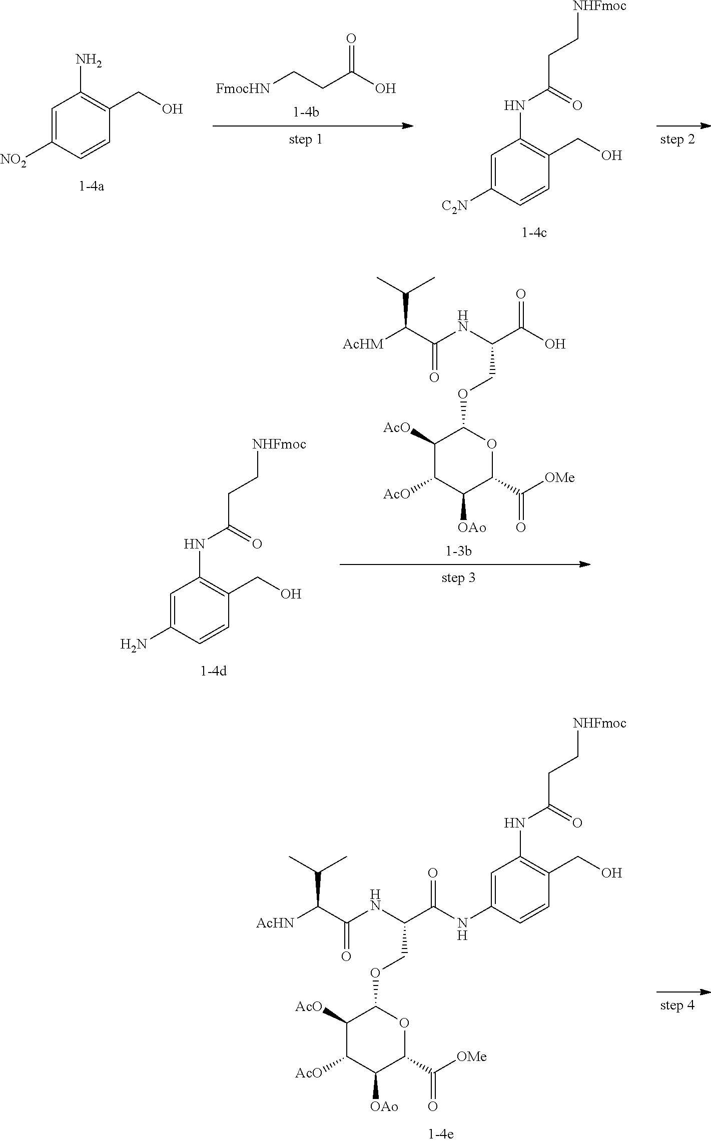

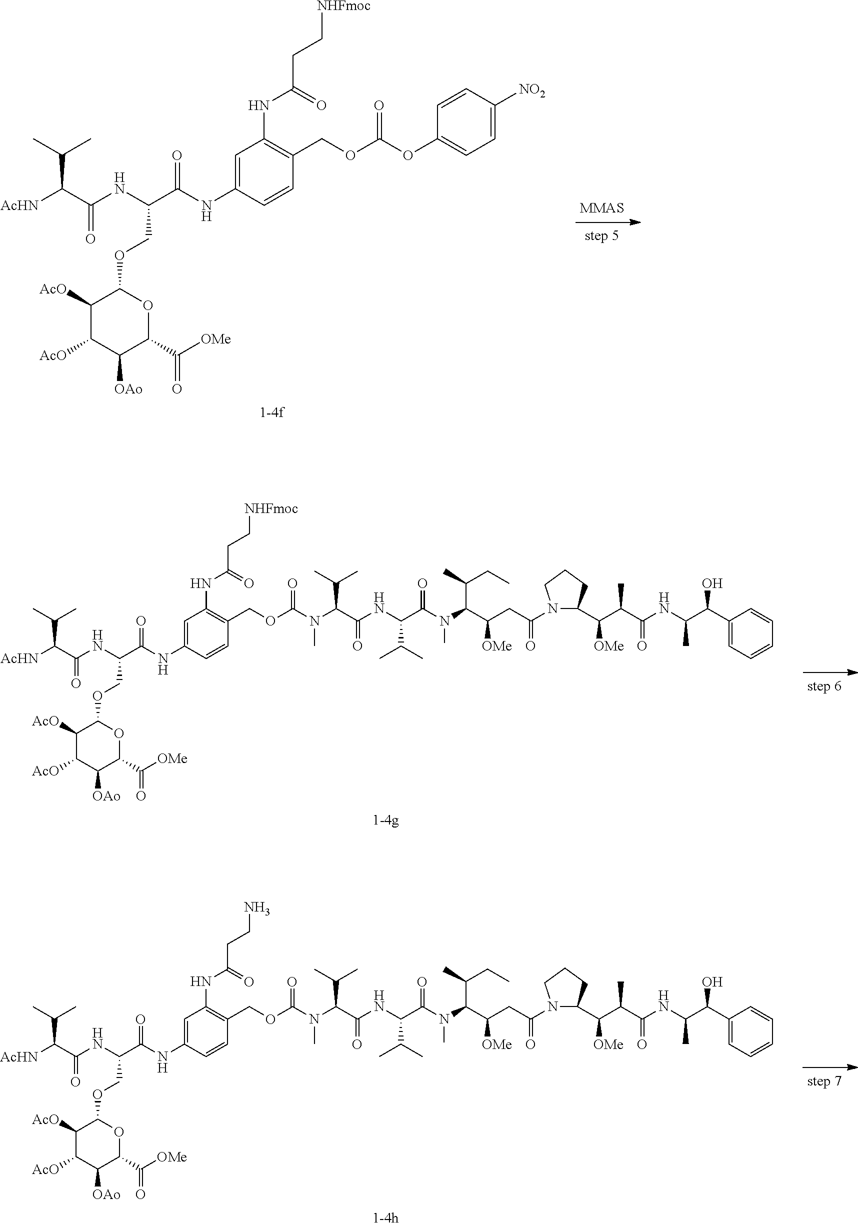

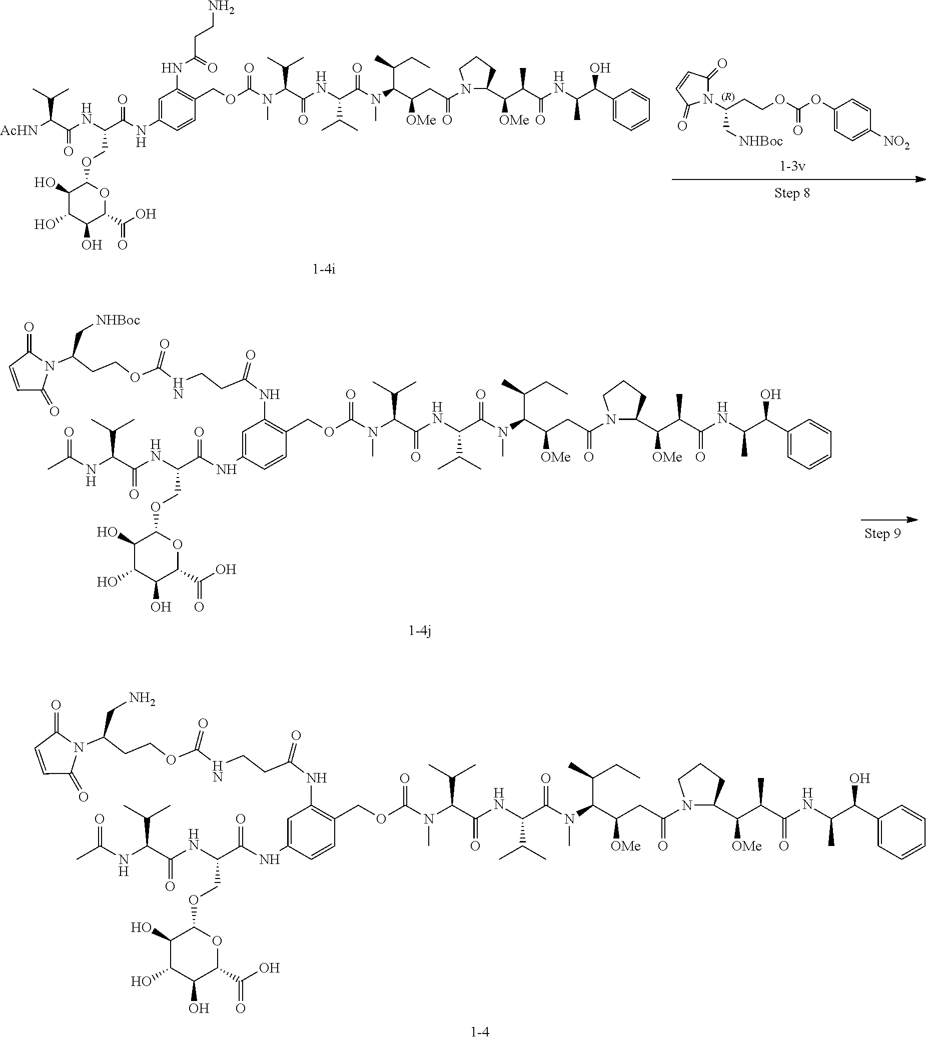

1-4 (8.5 mg, 77.4% yield) was synthesized according to the procedures of example 1-3.

### Example 1-5

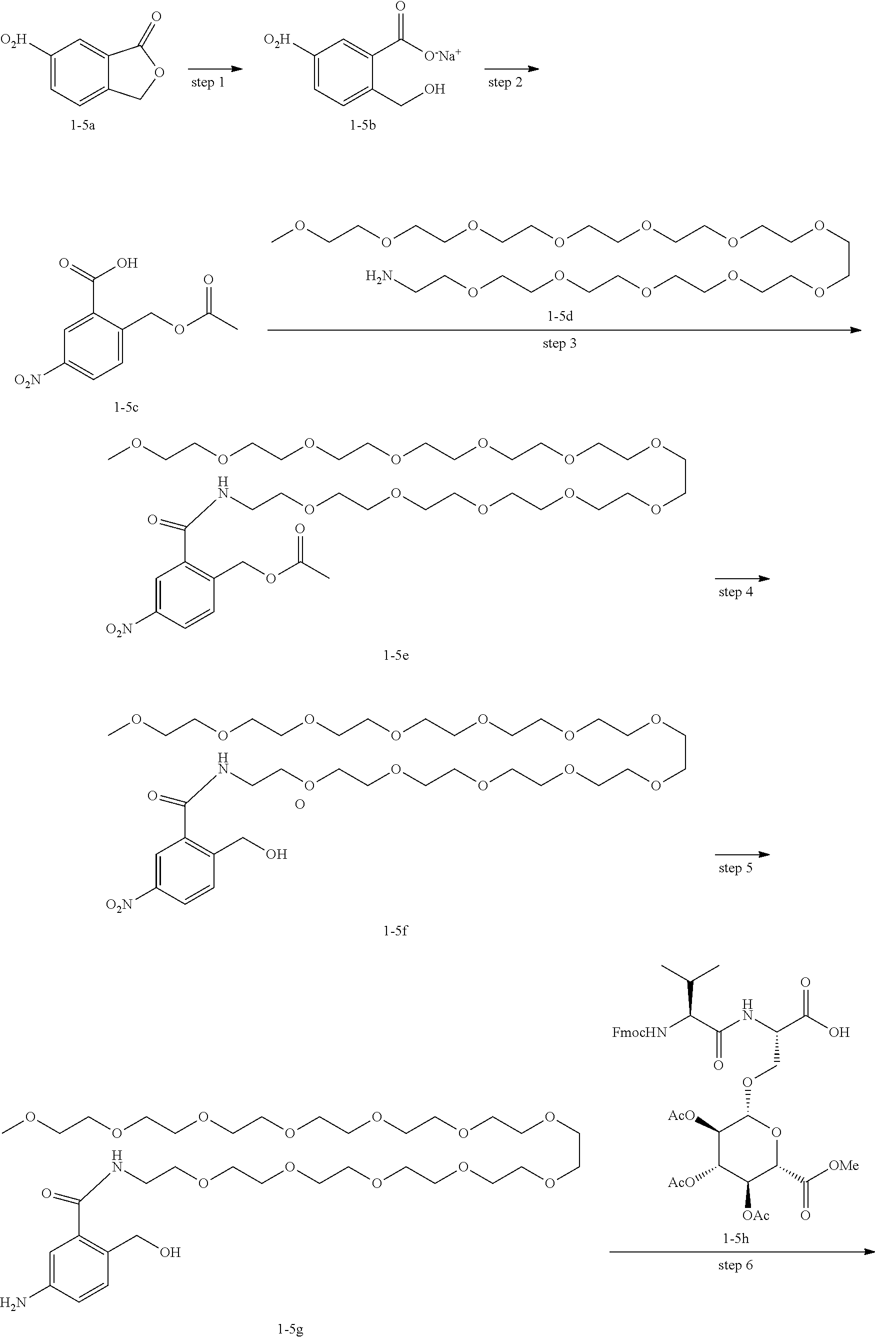

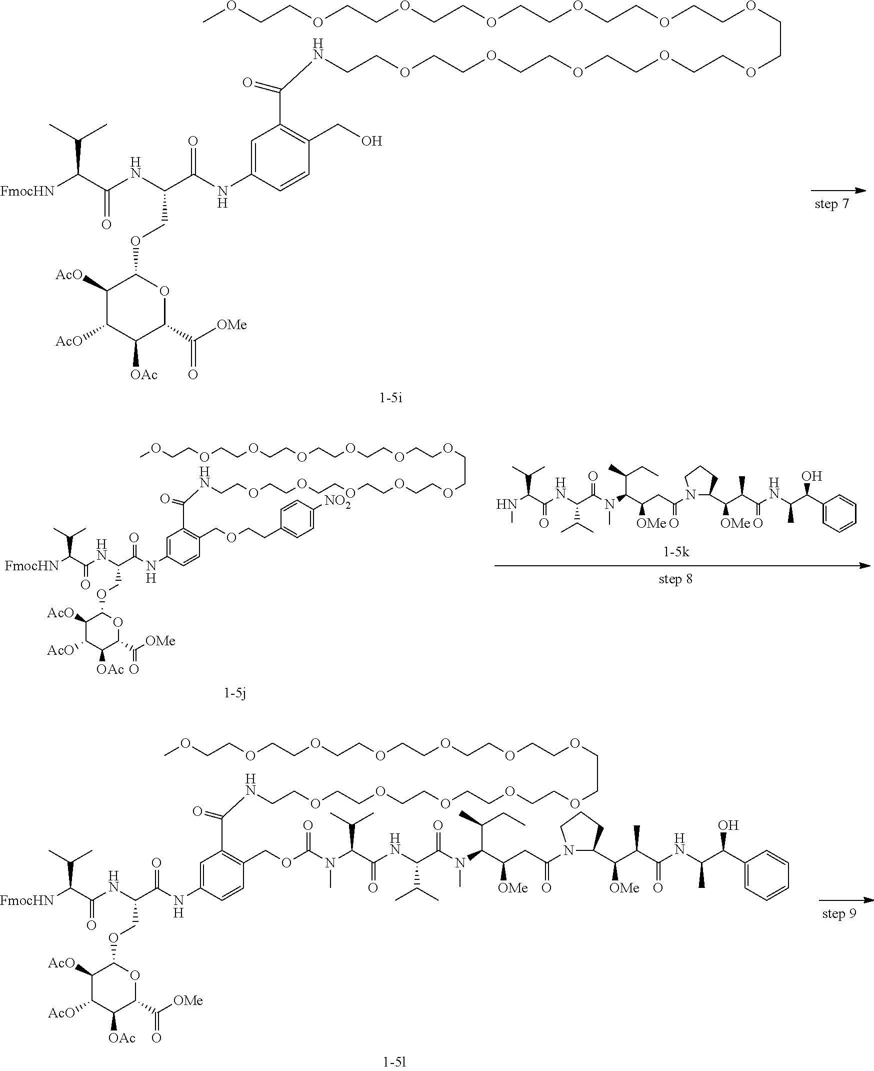

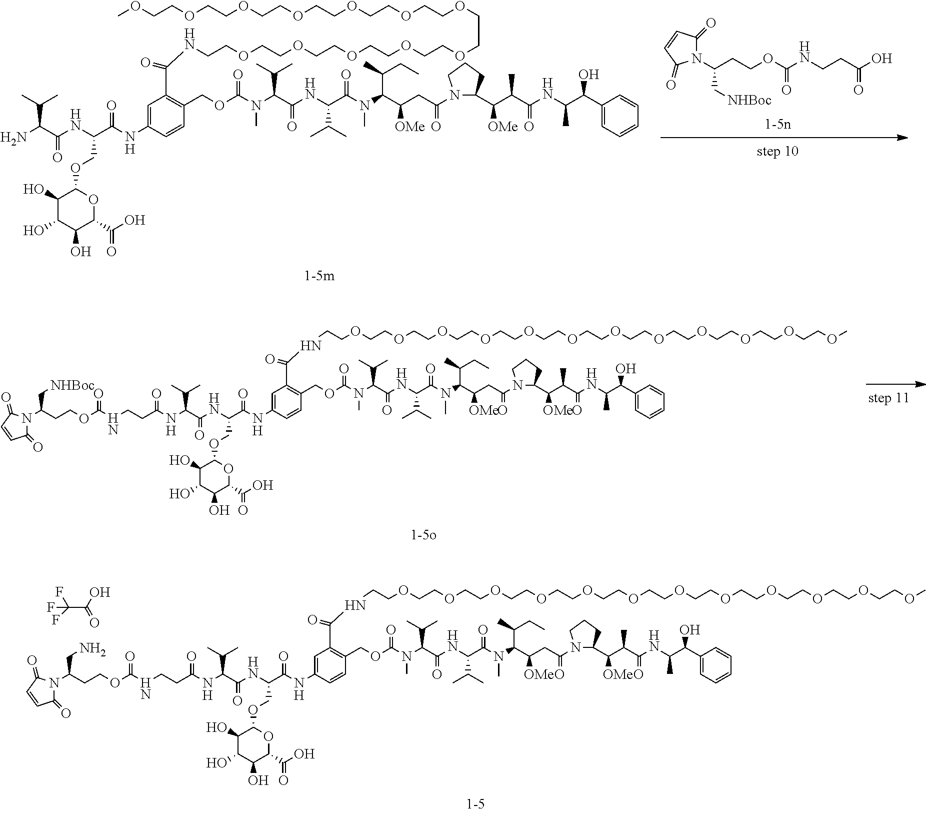

### Step 1~Step 5: 5-amino-N-(2,5,8,11,14,17,20,23,26,29,32,35-dodecaoxaheptatriacontan-37-yl)-2-(hydroxymethyl)benzamide (1-5g)

1-5g (281 mg, 98.7% yield) was synthesized according to the synthetic procedures of 1-3o of example 1-3. MS (ESI) m/z: 709.8 [M+H]⁺

### Step 6: (2R,3R,4S,5S,6S)-2-((S)-3-((3-((2,5,8,11,14,17,20,23,26,29,32,35-dodecaoxaheptatriacontan-37-yl)carbamoyl)-4-(hydroxymethyl)phenyl)amino)-2-((S)-2-((((9H-fluoren-9-yl)methoxy)carbonyl)amino)-3-methylbutanamido)-3-oxopropoxy)-6-(methoxycarbonyl)tetrahydro-2H-pyran-3,4,5-triyl triacetate (1-5i)

To a mixture of 1-5g (281 mg, 0.40 mmol) and 1-5h (295 mg, 0.40 mmol) in THF (5 mL) was added EEDQ (108 mg, 0.44 mmol). The mixture was stirred at RT for 9 hr. TLC showed the reaction was consumed. The mixture was concentrated under vacuum to give a crude product, purified by silica gel column chromatography ((MeOH:DCM=1:5)/DCM)=0/100~30/70), fraction was concentrated under vacuum to give 1-5i (377 mg, 66.3% yield) as a white solid. MS (ESI) m/z: 1435.2 [M+H]⁺

### Step 7: (2R,3R,4S,5S,6S)-2-((S)-3-((3-((2,5,8,11,14,17,20,23,26,29,32,35-dodecaoxaheptatriacontan-37-yl)carbamoyl)-4-((((4-nitrophenoxy)carbonyl)oxy)methyl)phenyl)amino)-2-((S)-2-((((9H-fluoren-9-yl)methoxy)carbonyl)amino)-3-methylbutanamido)-3-oxopropoxy)-6-(methoxycarbonyl)tetrahydro-2H-pyran-3,4,5-triyl triacetate (1-5j)

To a mixture of 1-5i (375 mg, 0.26 mmol) and NPC (88 mg, 0.29 mmol) in DMF (3.5 mL) was added DIEA (100 μL, 74 mg, 0.575 mmol). The resulted light-yellow mixture was stirred at RT for 12 hr. The mixture was quenched by saturated KHSO₄ (5 mL)/H₂O (20 mL), extracted with EA (20 mL*2). After separation the combined organic layers were washed with Brine (20 mL), dried over Na₂SO₄, filtered and the filtrate was concentrated under vacuum to give 1-5j (206 mg, 49.2% yield) as a pale-yellow solid. MS (ESI) m/z: 1600.2 [M+H]⁺

### Step 8: (2R,3R,4S,5S,6S)-2-((S)-3-((3-((2,5,8,11,14,17,20,23,26,29,32,35-dodecaoxaheptatriacontan-37-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-2-((S)-2-((((9H-fluoren-9-yl)methoxy)carbonyl)amino)-3-methylbutanamido)-3-oxopropoxy)-6-(methoxycarbonyl)tetrahydro-2H-pyran-3,4,5-triyl triacetate (1-51)

To a mixture of 1-5j (190 mg, 0.12 mmol), 1-5k (128 mg, 0.19 mmol) and HOBt (1.6 mg, 0.012 mmol) in DMF (1 mL) was added Pyridine (19 μL, 19 mg, 0.24 mmol). The mixture was stirred at RT for overnight. The mixture was purified by prep-HpLC (Method: column: XBridge Prep C18 OBD 5 μm 19*150 mm; Mobile phase: A-water (0.1% formic acid): B-acetonitrile; Flow rate: 20 mL/min) to give 1-51 (130 mg, 50.2% yield) as a white solid. MS (ESI) m/z: 2178.0 [M+H]⁺

### Step 9: (2S,3S,4S,5R,6R)-6-((S)-3-((3-((2,5,8,11,14,17,20,23,26,29,32,35-dodecaoxaheptatriacontan-37-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-2-((S)-2-amino-3-methylbutanamido)-3-oxopropoxy)-3,4,5-trihydroxytetrahydro-2H-pyran-2-carboxylic acid (1-5m)

To a solution of 1-51 (130 mg, 0.06 mmol) in THF (8 mL) were added IN LiOH (2.6 mL). The mixture was stirred at 0° C. for 30 min. Then the mixture was acidified with AcOH to pH=5. The mixture was concentrated under vacuum to give crude product, which was dissolved in DMF (2 mL). Diethylamine (309 μL, 2.99 mmol) was added and reacted at r.t. for 1 hr. Finally the mixture was purified by prep-HpLC (Method: column: XBridge Prep C18 OBD 5 μm 19*150 mm; Mobile phase: A-water (0.1% formic acid): B-acetonitrile; Flow rate: 20 mL/min), fraction was concentrated under vacuum to give 1-5m (60 mg, 55.4% yield) as a white solid. MS (ESI) m/z: 1816.1 [M+H]⁺

### Step 10: (2S,3S,4S,5R,6R)-6-(((7R,17S,20S)-20-((3-((2,5,8,11,14,17,20,23,26,29,32,35-dodecaoxaheptatriacontan-37-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-7-(2,5-dioxo-2,5-dihydro-1H-pyrrol-1-yl)-17-isopropyl-2,2-dimethyl-4,11,15,18-tetraoxo-3,10-dioxa-5,12,16,19-tetraazahenicosan-21-yl)oxy)-3,4,5-trihydroxytetrahydro-2H-pyran-2-carboxylic acid (1-50)

To a mixture of 1-5n (9.7 mg, 0.024 mmol) and HATU (9.2 mg, 0.024 mmol) in DMF (2 mL) was added DIPEA (8 μL, 5.70 mg, 0.044 mmol). The mixture was stirred at r.t. for 10 min. 1-5m (40 mg, 0.022 mmol) was added. The mixture was stirred at r.t. for 1 hr. It was purified by prep-HpLC

(Method: column: XBridge Prep C18 OBD 5 μm 19*150 mm; Mobile phase: A-water (0.1% formic acid): B-acetonitrile; Flow rate: 20 mL/min), fraction was freeze-dried to give 1-50 (24 mg, 48.5% yield) as a white solid. MS (ESI) m/z: 2196.8 [M+H]⁺

### Step 11

### (2S,3S,4S,5R,6R)-6-(((2S,5S,15R)-2-((3-((2,5,8,11,14,17,20,23,26,29,32,35-dodecaoxaheptatriacontan-37-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-16-amino-15-(2,5-dioxo-2,5-dihydro-1H-pyrrol-1-yl)-5-isopropyl-4,7,11-trioxo-12-oxa-3,6,10-triazahexadecyl)oxy)-3,4,5-trihydroxytetrahydro-2H-pyran-2-carboxylic acid (1-5)

A mixture of 1-50 (24 mg, 0.011 mmol) in TFA/DCM (v/v=1:4, 2 mL) was stirred at 0° C. for 1 hr. Bubble with nitrogen atmosphere to remove DCM then diluted with acetonitrile (2 mL) then purified by prep-HpLC (Method: column: XBridge Prep C18 OBD 5 μm 19*150 mm; Mobile phase: A-water (0.1% formic acid): B-acetonitrile; Flow rate: 20 mL/min) to give 1-5 (16 mg, 71.8% yield) as a white solid. MS (ESI) m/z: 2096.8 [M+H]⁺

### Example 1-6

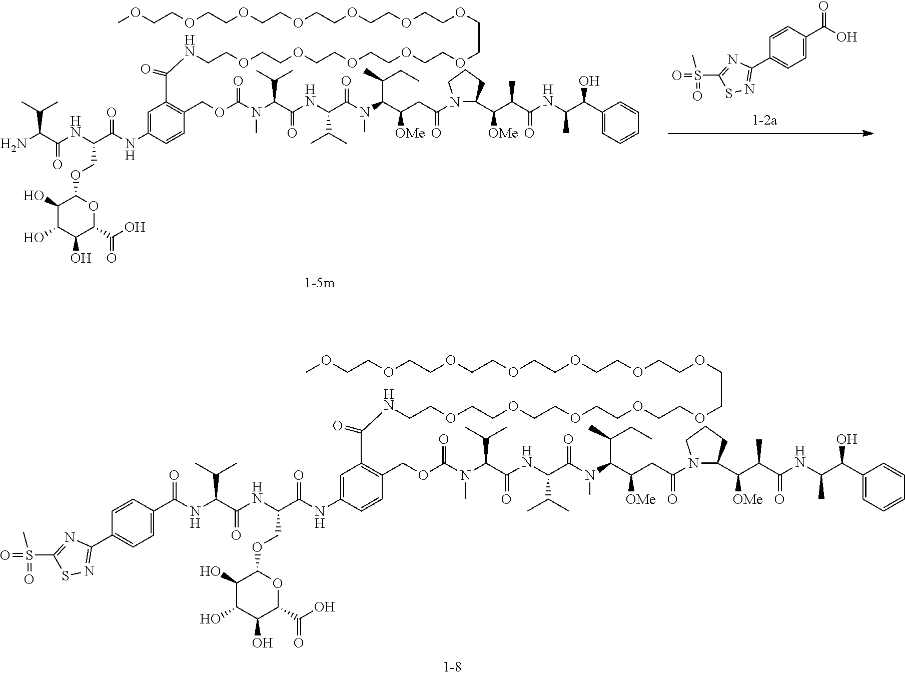

### (2S,3S,4S,5R,6R)-6-((S)-3-((3-((2,5,8,11,14,17,20,23,26,29,32,35-dodecaoxaheptatriacontan-37-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-2-((S)-3-methyl-2-(4-(5-(methylsulfonyl)-1,2,4-thiadiazol-3-yl)benzamido) butanamido)-3-oxopropoxy)-3,4,5-trihydroxytetrahydro-2H-pyran-2-carboxylic acid (1-6)

Compound 1-6 (8.9 mg, 38.9% yield) was synthesized according to the synthetic procedure of step 10 of example 1-5. MS (ESI) m/z: 2082.1 [M+H]⁺

### Example 1-7

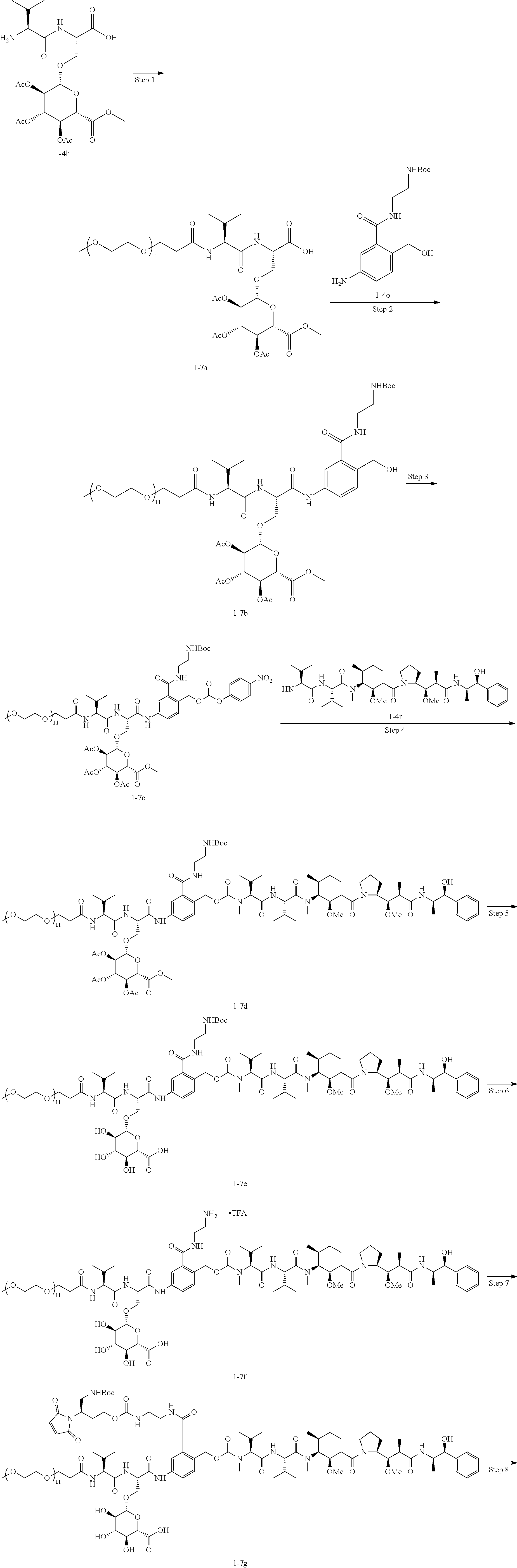

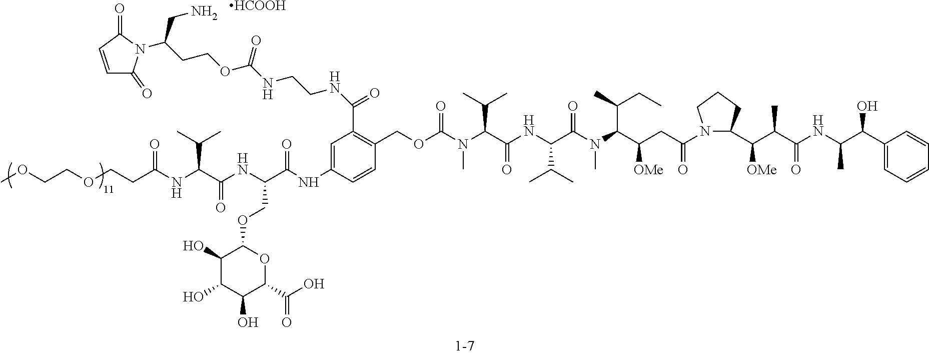

### Step 1: N-((2,5,8,11,14,17,20,23,26,29,32,35-dodecaoxaoctatriacontan-38-oyl)-L-valyl)-O-((2R,3R,4S,5S,6S)-3,4,5-triacetoxy-6-(methoxycarbonyl)tetrahydro-2H-pyran-2-yl)-L-serine (1-7a)

Compound 1-7a (380 mg, 51.8% yield) was synthesized according to synthetic procedure of step 5 of example 1-3. MS (ESI) m/z: 1091.7 [M+H]⁺

### Step 2: (2R,3R,4S,5S,6S)-2-(((40S,43S)-43-((3-((2-((tert-butoxycarbonyl)amino)ethyl) carbamoyl)-4-(hydroxymethyl)phenyl)carbamoyl)-40-isopropyl-38,41-dioxo-2,5,8,11,14,17,20,23,26,29,32,35-dodecaoxa-39,42-diazatetratetracontan-44-yl)oxy)-6-(methoxycarbonyl)tetrahydro-2H-pyran-3,4,5-triyl triacetate (1-7b)

Compound 1-7b (294 mg, 51.7% yield) was synthesized according to synthetic procedure of step 11 of example 1-3. MS (ESI) m/z: 1383.2 [M+H]⁺

### Step 3: (2R,3R,4S,5S,6S)-2-(((40S,43S)-43-((3-((2-((tert-butoxycarbonyl)amino)ethyl) carbamoyl)-4-((((4-nitrophenoxy)carbonyl)oxy)methyl)phenyl)carbamoyl)-40-isopropyl-38,41-dioxo-2,5,8,11,14,17,20,23,26,29,32,35-dodecaoxa-39,42-diazatetratetracontan-44-yl)oxy)-6-(methoxycarbonyl)tetrahydro-2H-pyran-3,4,5-triyl triacetate (1-7c)

Compound 1-7c (289 mg, 87.8% yield) was synthesized according to synthetic procedure of step 12 of example 1-3. MS (ESI) m/z: 1548.4 [M+H]⁺

### Step 4: (2R,3R,4S,5S,6S)-2-(((40S,43S)-43-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-40-isopropyl-38,41-dioxo-2,5,8,11,14,17,20,23,26,29,32,35-dodecaoxa-39,42-diazatetratetracontan-44-yl)oxy)-6-(methoxycarbonyl)tetrahydro-2H-pyran-3,4,5-triyl triacetate (1-7d)

Compound 1-7d (290 mg, 73.0% yield) was synthesized according to synthetic procedure of step 13 of example 1-3. MS (ESI) m/z: 2126.8 [M+H]⁺

### Step 5: (2S,3S,4S,5R,6R)-6-(((40S,43S)-43-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-40-isopropyl-38,41-dioxo-2,5,8,11,14,17,20,23,26,29,32,35-dodecaoxa-39,42-diazatetratetracontan-44-yl)oxy)-3,4,5-trihydroxytetrahydro-2H-pyran-2-carboxylic acid (1-7e)

Compound 1-7e (243 mg, crude) was synthesized according to synthetic procedure of step 14 of example 1-3. MS (ESI) m/z: 1987.2 [M+H]⁺

### Step 6: (2S,3S,4S,5R,6R)-6-(((40S,43S)-43-((3-((2-aminoethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-40-isopropyl-38,41-dioxo-2,5,8,11,14,17,20,23,26,29,32,35-dodecaoxa-39,42-diazatetratetracontan-44-yl)oxy)-3,4,5-trihydroxytetrahydro-2H-pyran-2-carboxylic acid (1-7f)

Compound 1-7f (63 mg, 27.3% for two steps) was synthesized according to synthetic procedure of step 15 of example 1-3. MS (ESI) m/z: 1887.1 [M+H]⁺

### Step 7: (2S,3S,4S,5R,6R)-6-(((40S,43S)-43-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-3-(((R)-8-(2,5-dioxo-2,5-dihydro-1H-pyrrol-1-yl)-13,13-dimethyl-4,11-dioxo-5,12-dioxa-3,10-diazatetradecyl)carbamoyl)phenyl)carbamoyl)-40-isopropyl-38,41-dioxo-2,5,8,11,14,17,20,23,26,29,32,35-dodecaoxa-39,42-diazatetratetracontan-44-yl)oxy)-3,4,5-trihydroxytetrahydro-2H-pyran-2-carboxylic acid (1-7g)

Compound 1-7g (28 mg, 80.0% yield) was synthesized according to synthetic procedure of step 16 of example 1-3. MS (ESI) m/z: 2197.2 [M+H]⁺

### Step 8: (2S,3S,4S,5R,6R)-6-(((40843S)-43-((3-((2-((((R)-4-amino-3-(2,5-dioxo-2,5-dihydro-1H-pyrrol-1-yl)butoxy)carbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-40-isopropyl-38,41-dioxo-2,5,8,11,14,17,20,23,26,29,32,35-dodecaoxa-39,42-diazatetratetracontan-44-yl)oxy)-3,4,-trihydroxytetrahydro-2-pyran-2-carboxylic acid—formic acid (1-7)

Compound 1-7 (8.2 mg, 30.8% yield) was synthesized according to synthetic procedure of step 17 of example 1-3. MS (ESI) m/z: 2097.1 [M+H]⁺

### Example 1-8

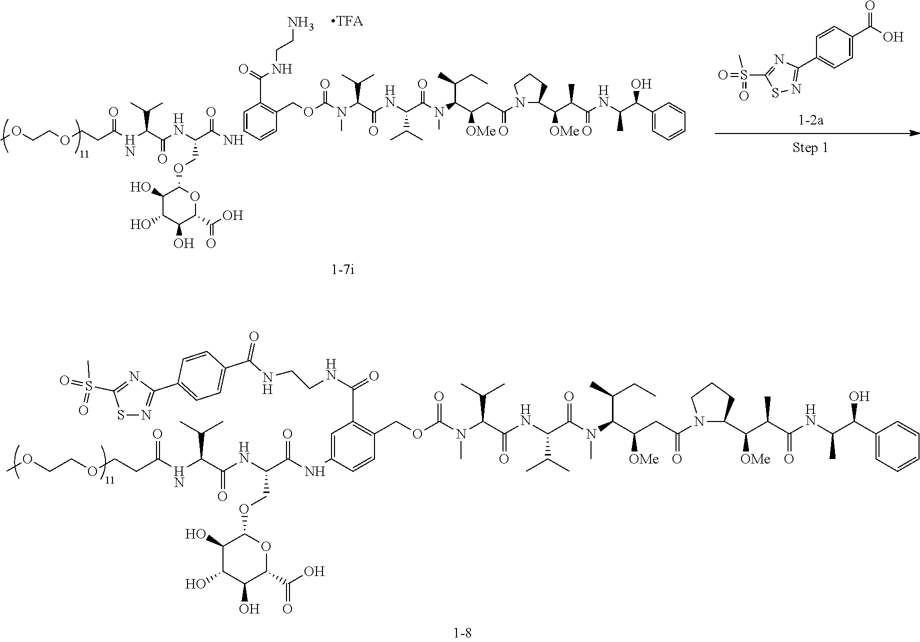

### Step 1:(2S,3S,4S,5R,6R)-6-(((40S,43S)-43-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-3-((2-(4-(5-(methylsulfonyl)-1,2,4-thiadiazol-3-yl)benzamido)ethyl)carbamoyl)phenyl)carbamoyl)-40-isopropyl-38,41-dioxo-2,5,8,11,14,17,20,23,26,29,32,35-dodecaoxa-39,42-diazatetratetracontan-44-yl)oxy)-3,4,5-trihydroxytetrahydro-2H-pyran-2-carboxylic acid (1-8)

To a solution of compound 1-2e (6.6 mg, 0.023 mmol) in DMF (1 mL) were added HATU (7.7 mg, 0.020 mmol) and DIEA (4.0 mg, 0.031 mmol). The mixture was stirred at r.t. for 10 min. Compound 1-7f (30 mg, 0.016 mmol) was added into the mixture and stirred at r.t. for 1 h. The mixture was purified using prep-HPLC (Method: column: XBridge Prep C18 OBD 5 μm 19*150 mm; Mobile phase: A-water (0.1% formic acid): B-acetonitrile; Flow rate: 20 mL/min) to give compound 1-8 (19 mg, 56.9% yield) as white solid. MS (ESI) m/z: 2152.7 [M+H]⁺

### Example 1-9

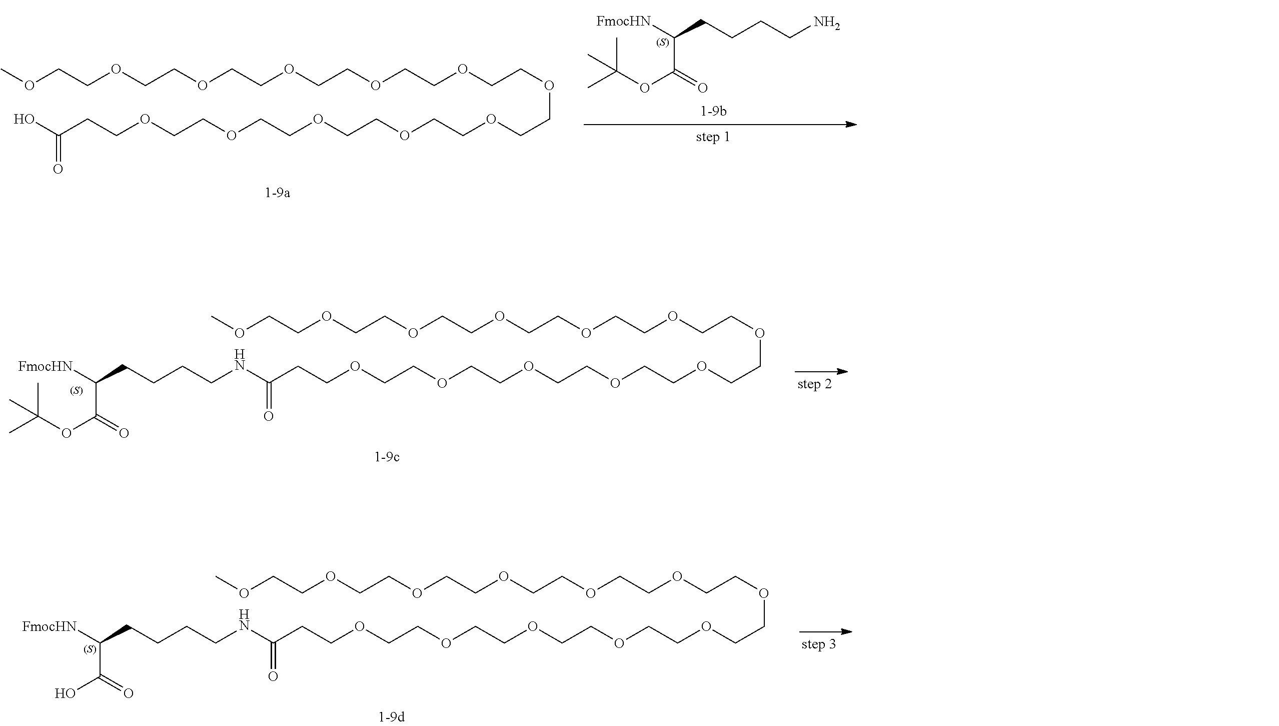

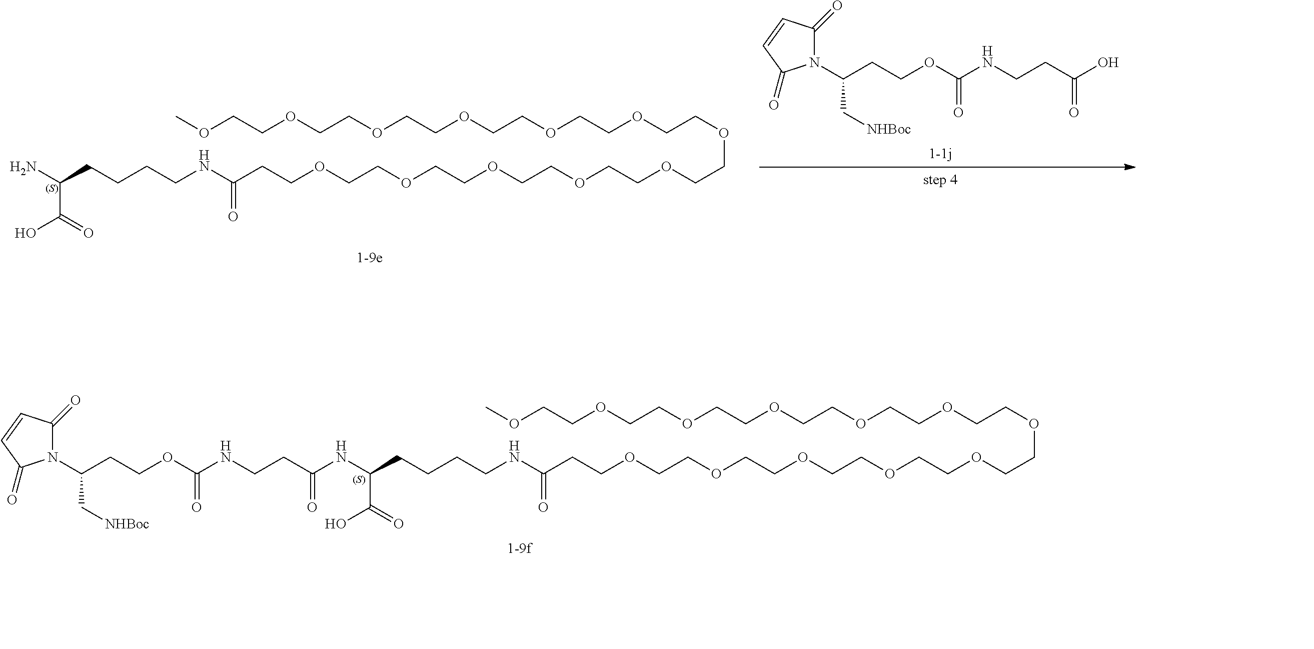

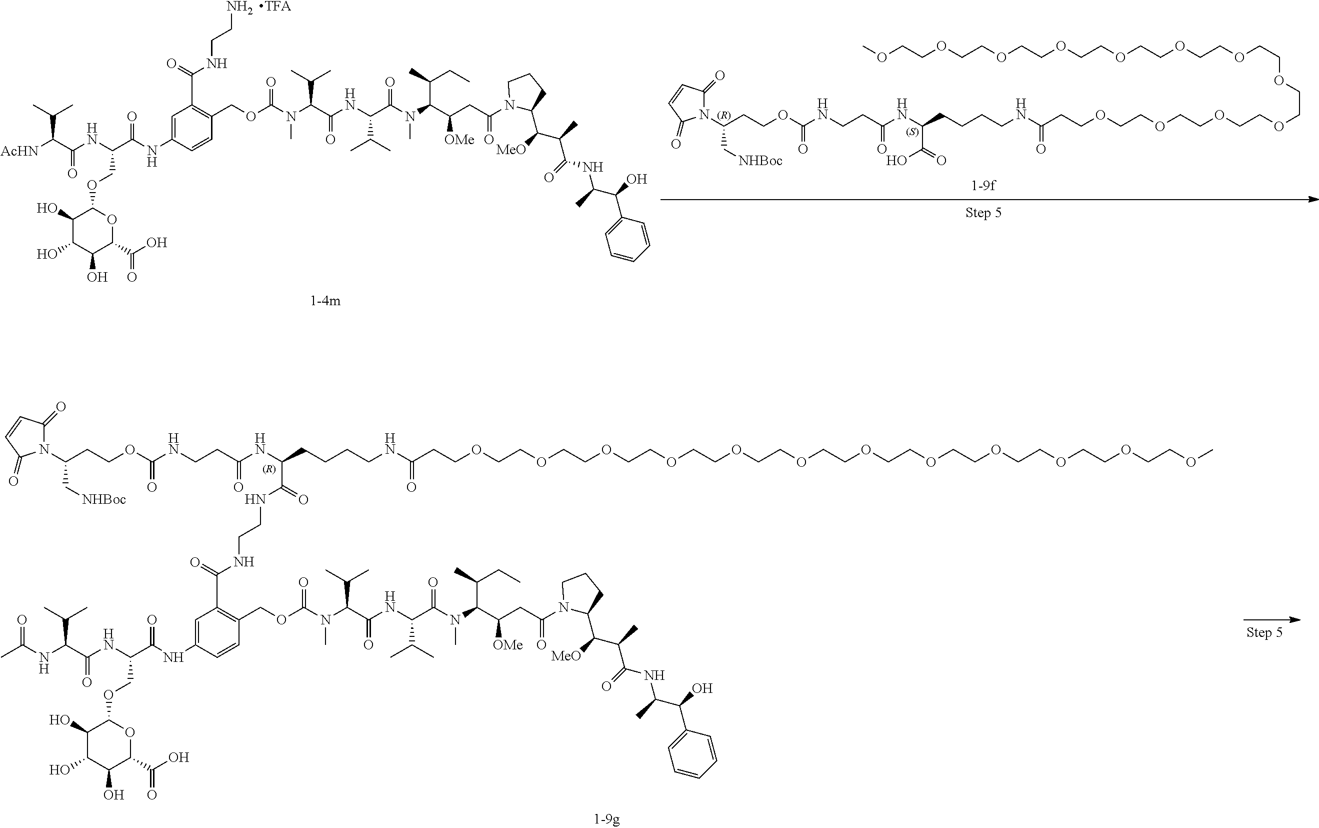

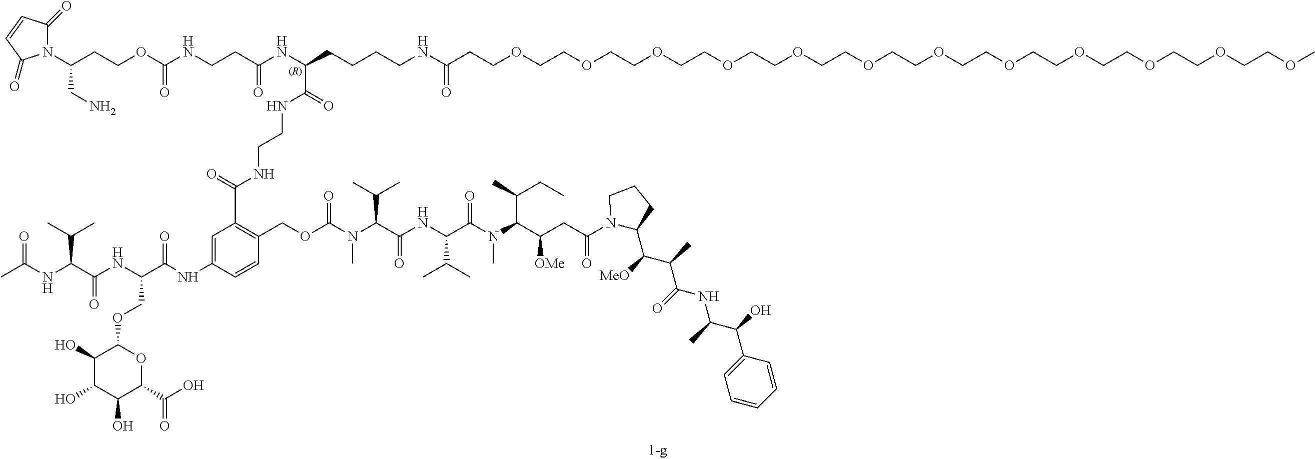

### Step 1: tert-butyl(S)-44-((((9H-fluoren-9-yl)methoxy)carbonyl)amino)-38-oxo-2,5,8,11,14,17,20,23,26,29,32,35-dodecaoxa-39-azapentatetracontan-45-oate (1-9c)

To a solution of 1-9a (580 mg, 0.99 mmol) in DMF (6 mL) were added HATU (375 mg, 0.99 mmol) and DIEA (343 μL, 255 mg, 1.97 mmol). The mixture was stirred at r.t. for 10 min. Then to the brown mixture was added 1-9b (454 mg, 0.99 mmol) and stirred at r.t. for 15 min. The mixture was quenched with water (20 mL), extracted with EtOAc (30 mL*2). After separation combined organic layers were washed with brine (30 mL), dried over Na₂SO₄, filtered, and purified by silica gel column chromatography (MeOH:DCM=0/100~10/90) to give 1-9c (1.08 g, quant.) as a brown oil. MS (ESI) m/z: 995.7 [M+H]⁺

### Step 2: (S)-44-((((9H-fluoren-9-yl)methoxy)carbonyl)amino)-38-oxo-2,5,8,11,14,17,20,23,26,29,32,35-dodecaoxa-39-azapentatetracontan-45-oic acid (1-9d)

A mixture of 1-9c (1.08 g, 0.99 mmol) in DCM/TFA (v/v=5 mL/5 mL) stirred at r.t. for 2 hr. The mixture was concentrated under vacuum and co-evaporated with Tol (10 mL) for three times to give crude product (1.20 g) as a brown oil then purified by silica gel column chromatography (MeOH/DCM=0/100~10/90) to give 1-9d (874 mg, 94.5% yield) as a yellow oil. MS (ESI) m/z: 939.7 [M+H]⁺

### Step 3: (S)-44-amino-38-oxo-2,5,8,11,14,17,20,23,26,29,32,35-dodecaoxa-39-azapentatetracontan-45-oic acid (1-9e)

To a solution of 1-9d (874 mg, 0.88 mmol) in DMF (13 mL) was added Et₂N (909 μL, 642 mg, 8.78 mmol). The mixture was stirred at r.t. for 40 min then concentrated under vacuum and co-evaporated with Tol (10 mL) for three times to give crude product and purified by prep-HPLC to give 1-9e (179 mg, 28.4% yield) as a white solid. MS (ESI) m/z: 717.5 [M+H]⁺

### Step 4: (S)-44-((R)-7-(2,5-dioxo-2,5-dihydro-1H-pyrrol-1-yl)-2,2-dimethyl-4,11-dioxo-3,10-dioxa-5,12-diazapentadecan-15-amido)-38-oxo-2,5,8,11,14,17,20,23,26,29,32,35-dodecaoxa-39-azapentatetracontan-45-oic acid (1-9f)

To a mixture of 1-1j (105 mg, 0.26 mmol) and HATU (100.0 mg, 0.26 mmol) in DMF (6 mL) was added DIEA (87 μL, 65 mg, 0.50 mmol). The mixture was stirred at r.t. for 10 min. 1-9e (179 mg, 0.25 mmol) was added and stirred at r.t. for 1 hr. The mixture was purified by prep-HPLC (Method: column: XBridge Prep C18 OBD 5 μm 19*150 mm; Mobile phase: A-water (0.1% formic acid): B-acetonitrile; Flow rate: 20 mL/min) to give 1-9f (153 mg, 55.7% yield) as a transparent glass solid. MS (ESI) m/z: 1098.8 [M+H]⁺

### Step 5: (2S,3S,4S,5R,6R)-6-((S)-2-((S)-2-acetamido-3-methylbutanamido)-3-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-3-(((S)-44-((R)-7-(2,5-dioxo-2,5-dihydro-1H-pyrrol-1-yl)-2,2-dimethyl-4,11-dioxo-3,10-dioxa-5,12-diazapentadecan-15-amido)-38,45-dioxo-2,5,8,11,14,17,20,23,26,29,32,35-dodecaoxa-39,46-diazaoctatetracontan-48-yl)carbamoyl)phenyl)amino)-3-oxopropoxy)-3,4,5-trihydroxytetrahydro-2H-pyran-2-carboxylic acid (1-9g)

Compound 1-9g (19 mg, 35.2% yield) was synthesized according to synthetic procedure of step 1 of example 1-8. MS (ESI) m/z: 2438.2 [M+H]⁺

### Step 6: (2S,3S,4S,5R,6R)-6-((S)-2-((S)-2-acetamido-3-methylbutanamido)-3-((3-(((S)-44-(3-((((R)-4-amino-3-(2,5-dioxo-2,5-dihydro-1H-pyrrol-1-yl)butoxy)carbonyl)amino) propanamido)-38,45-dioxo-2,5,8,11,14,17,20,23,26,29,32,35-dodecaoxa-39,46-diazaoctatetracontan-48-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-3,4,5-trihydroxytetrahydro-2H-pyran-2-carboxylic acid (1-9)

Compound 1-9 (8.4 mg, 31.2% yield) was synthesized according to synthetic procedure of step 17 of example 1-3. MS (ESI) m/z: 2338.0 [M+H]⁺

### Example 1-10

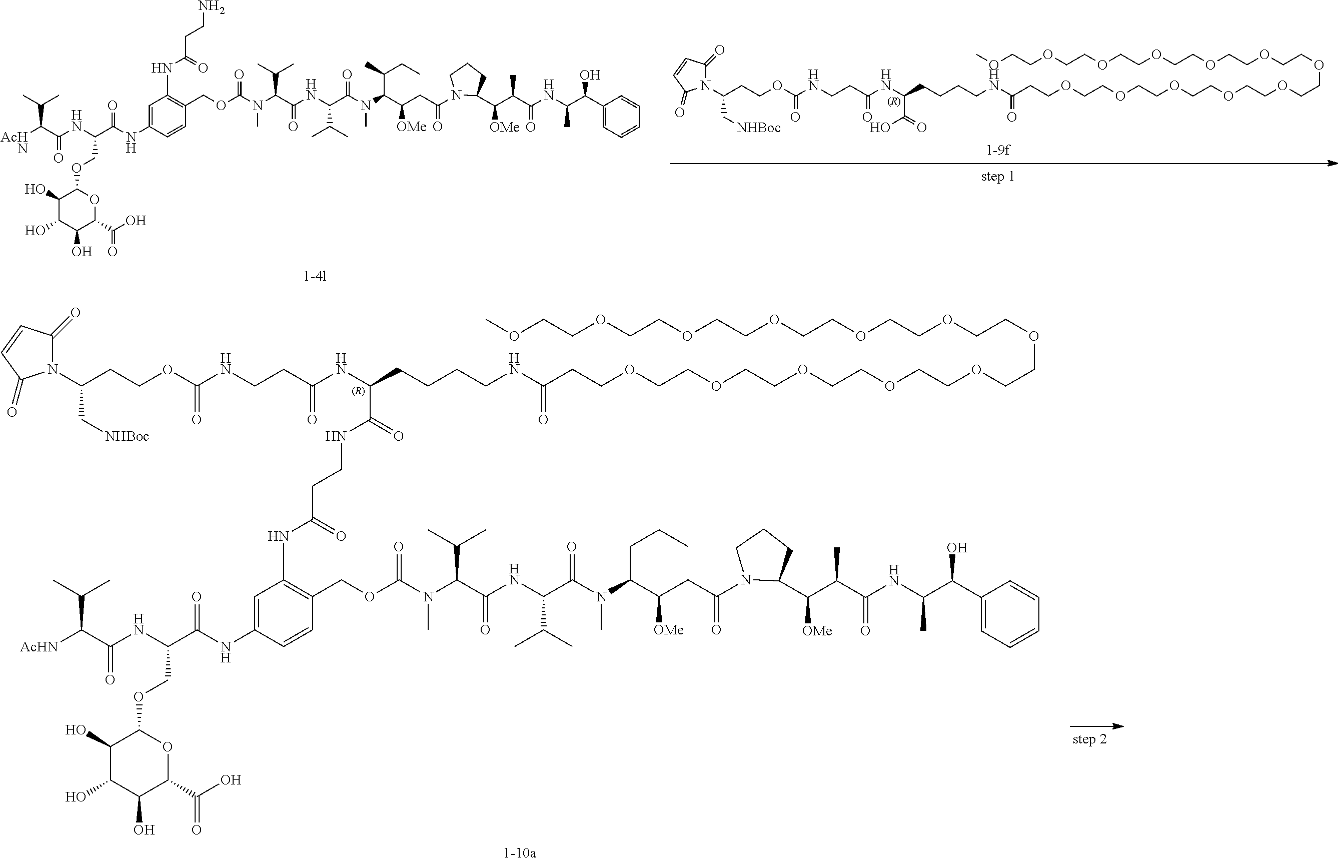

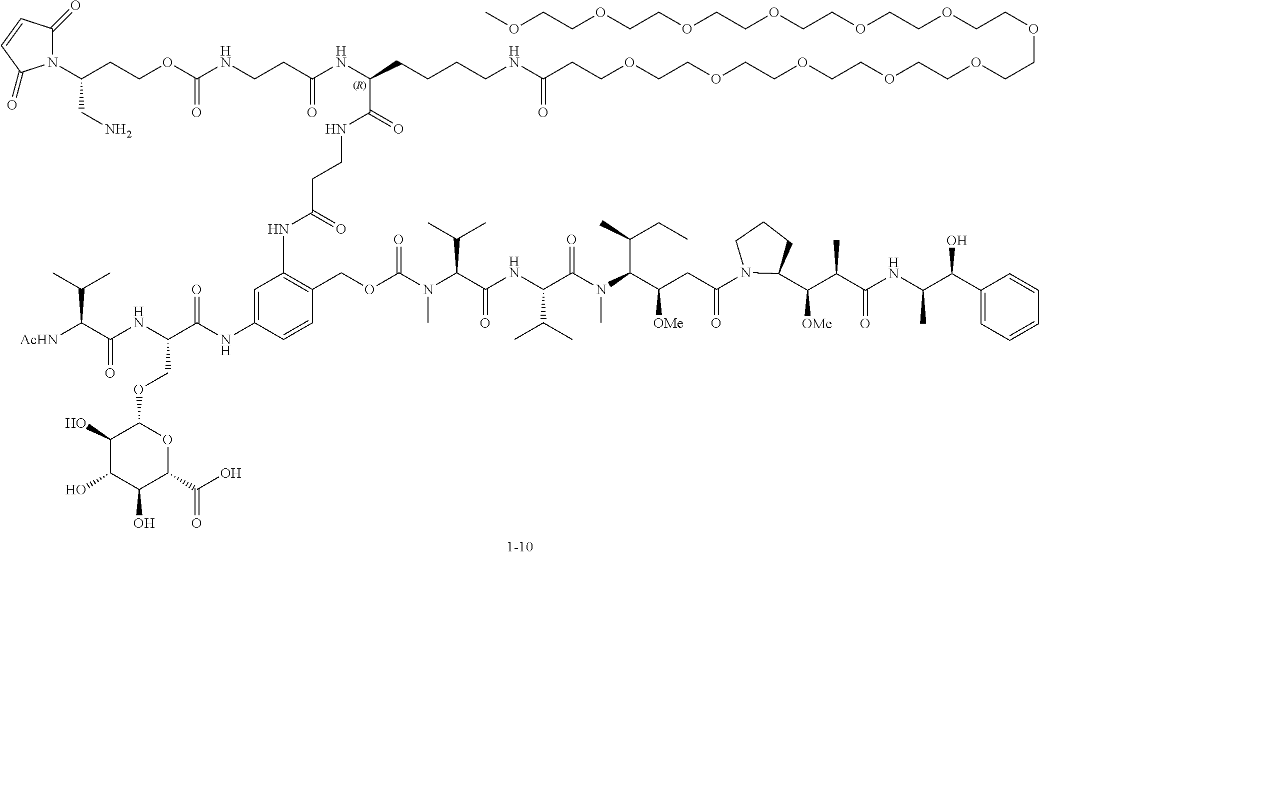

Compound 1-10 (2.6 mg, 88.0% yield) was synthesized according to the synthetic procedures of example 1-9.

### Example 1-11

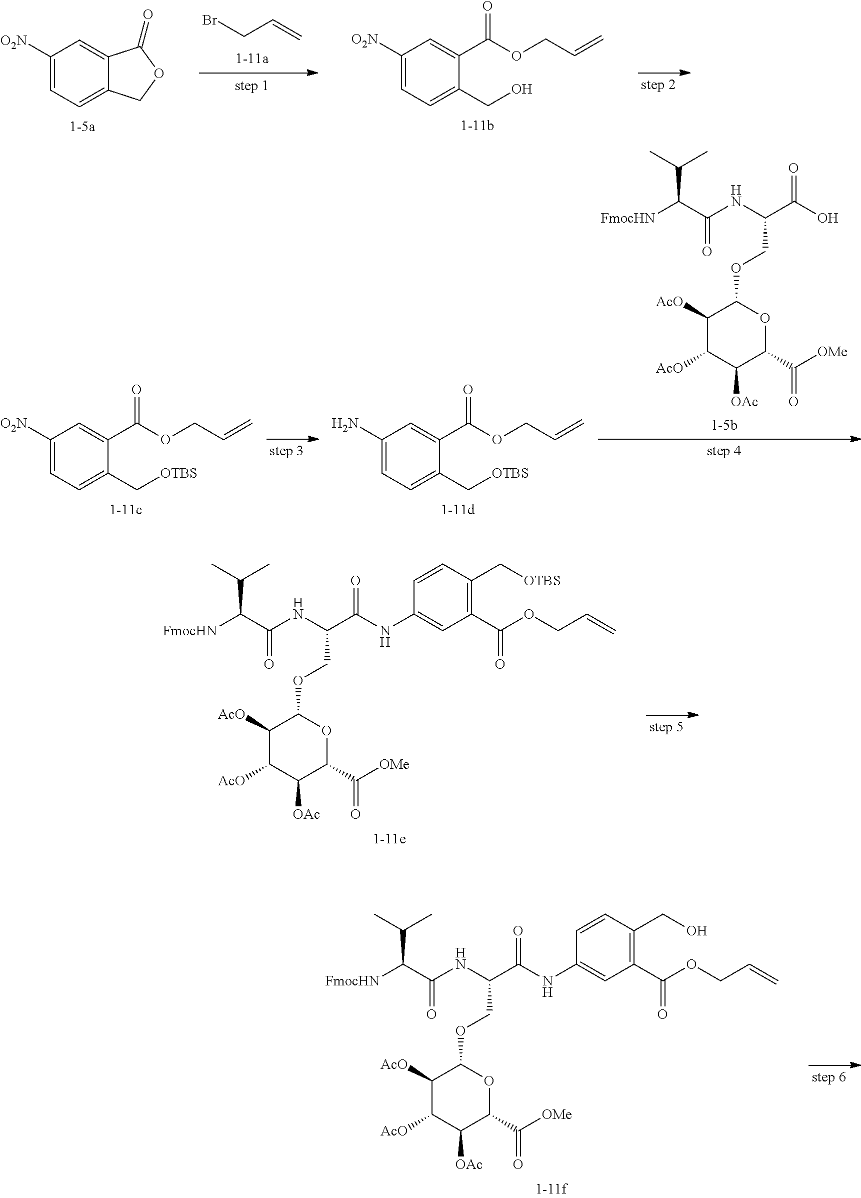

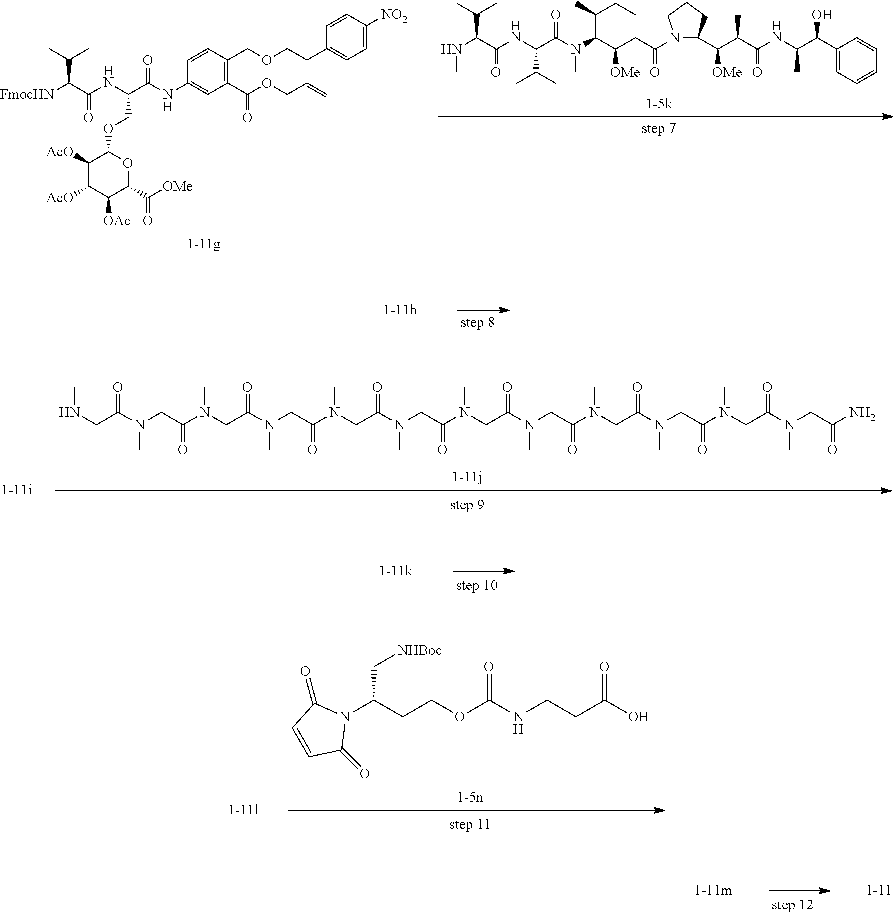

### Step 1: allyl 2-(hydroxymethyl)-5-nitrobenzoate (1-11b)

To a solution of Reactant 1 (3.0 g, 16.75 mmol) in H₂O (100 mL) was added NaOH (670 mg, 16.75 mmol). The reaction was stirred under reflux for 1 h. The reaction was concentrated in vacuo and directly used for allylation. The residue was dissolved in dry DMF (50 mL) and allyl bromide (2027 mg, 16.75 mmol) was added. The reaction was stirred for 16 hr before it was concentrated in vacuo. Silica gel flash column chromatography (30 g, EtOAc/Petroleum ether=0/100~30/70) to give 1-11b (3.0 g, 75.8% yield) as a white solid.

¹H NMR (400 MHz, CDCl₃) δ 8.87 (d, J=2.4 Hz, 1H), 8.39 (dd, J=8.4, 2.4 Hz, 1H), 7.79 (d, J=8.4 Hz, 1H), 6.15-5.98 (m, 1H), 5.52-5.43 (m, 1H), 5.38 (dd, J=10.4, 1.2 Hz, 1H), 4.97 (s, 3H), 4.89 (dt, J=5.9, 1.2 Hz, 2H).

### Step 2: allyl 2-(((tert-butyldimethylsilyl)oxy)methyl)-5-nitrobenzoate (1-11c)

To a solution of 1-11b (1000 mg, 4.22 mmol) in DCM (10 mL) were added TBS-Cl (763 mg, 5.06 mmol) and Imidazole (431 mg, 6.32 mmol). The mixture was stirred at r.t. for 2 hr. Quenched with IN NaHCO₃ (30 mL), extracted with DCM (30 mL*2). After separation the combined organic layers were washed with brine (30 mL), dried over Na₂SO₄, filtered and concentrated under vacuum then purified by silica gel column chromatography (EtO)Ac/Petroleum ether=0%~10%) to give P1 (1.01 g, 68.2% yield) as a colorless oil. MS (ESI) m/z: 352.5 [M+H]⁺

¹H NMR (400 MHz, CDCl₃) δ 8.85 (d, J=2.4 Hz, 1H), 8.41 (dd, J=8.8, 2.4 Hz, 1H), 8.07 (d, J=8.8 Hz, 1H), 6.06 (ddt, J=16.4, 10.4, 5.9 Hz, 1H), 5.45 (dq, J=17.2, 1.4 Hz, 1H), 5.40-5.29 (m, 1H), 5.20 (s, 3H), 4.85 (dt, J=5.8, 1.2 Hz, 2H), 1.01-0.95 (m, 9H), 0.17-0.13 (m, 6H).

### Step 3: allyl 5-amino-2-(((tert-butyldimethylsilyl)oxy)methyl)benzoate (1-11d)

To a mixture of 1-11c (1.0 g, 2.87 mmol) in MeOH (10 mL) and H₂O (3 mL) were added Fe (802 mg, 14.4 mmol) and NH₄Cl (1537 mg, 28.73 mmol). The mixture was heated to 75° C. for 4 hr. Then filtered through a pad of celite and washed with EtOAc (30 mL), combined organic layers was washed with brine (20 mL), dried over Na₂SO₄, filtered, and concentrated under vacuum to give a residue. It was purified by silica gel column chromatography (EtOAc/Petroleum ether=0/100~70/30) to give 1-11d (868 mg, 94% yield) as a colorless oil. MS (ESI) m/z: 322.5 [M+H]⁺

¹H NMR (400 MHz, CDCl₃) δ 7.57 (d, J=8.4 Hz, 1H), 7.35 (s, 1H), 6.93 (dd, J=8.4, 2.1 Hz, 1H), 6.02 (ddd, J=16.0, 10.8, 5.7 Hz, 1H), 5.40 (dd, J=17.2, 1.4 Hz, 1H), 5.31-5.25 (m, 1H), 5.01 (s, 2H), 4.77 (dt, J=5.7, 1.4 Hz, 2H), 4.31 (s, 2H), 1.04-0.86 (m, 10H), 0.21-0.01 (m, 6H).

### Step 4: (2R,3R,4S,5S,6S)-2-((S)-2-((S)-2-((((9H-fluoren-9-yl)methoxy)carbonyl)amino)-3-methylbutanamido)-3-((3-((allyloxy)carbonyl)-4-(((tert-butyldimethylsilyl)oxy)methyl)phenyl)amino)-3-oxopropoxy)-6-(methoxycarbonyl)tetrahydro-2H-pyran-3,4,5-triyl triacetate (1-11e)

To a solution of 1-5h (400 mg, 0.54 mmol) and 1-11d (173 mg, 0.54 mmol) in DCM (5 mL) was added EEDQ (133 mg, 0.539 mmol). The mixture was stirred at r.t. for 3 hr. The mixture was concentrated under vacuum to give a residue and then purified by silica gel column chromatography (EtOAc/Petroleum ether=0/100~70/30) to give 1-11e (392 mg, 69.6% yield) as a white solid. MS (ESI) m/z: 914.8 [M-OTBS]⁺

### Step 5: (2R,3R,4S,5S,6S)-2-((S)-2-((S)-2-((((9H-fluoren-9-yl)methoxy)carbonyl)amino)-3-methylbutanamido)-3-((3-((allyloxy)carbonyl)-4-(hydroxymethyl)phenyl)amino)-3-oxopropoxy)-6-(methoxycarbonyl)tetrahydro-2H-pyran-3,4,5-triyl triacetate (1-11f)

A mixture of 1-11e (375 mg, 0.36 mmol) in AcOH/THF/H₂O (1 mL/1 mL/0.5 mL) was stirred at r.t. for overnight. The mixture was concentrated under vacuum to give a residue then purified by silica gel column chromatography (EtOAc/Petroleum ether=0/100~100/0) to give 1-11f (326 mg, 97.6%) as a white solid. MS (ESI) m/z: 915.3 [M-OH]⁺

### Step 6: (2R,3R,4S,5S,6S)-2-((S)-2-((S)-2-((((9H-fluoren-9-yl)methoxy)carbonyl)amino)-3-methylbutanamido)-3-((3-((allyloxy)carbonyl)-4-((((4-nitrophenoxy)carbonyl)oxy)methyl)phenyl)amino)-3-oxopropoxy)-6-(methoxycarbonyl)tetrahydro-2H-pyran-3,4,5-triyl triacetate (1-11g)

To a mixture of 1-11f (326 mg, 0.35 mmol) and NPC (160 mg, 0.53 mmol) in DMF (5 mL) was added DIPEA (79 μL, 0.46 mmol). The resulted golden mixture was stirred at r.t. for 2 hr. The mixture was diluted with EtOAc (20 mL), washed with brine (20 mL*3), dried over Na₂SO₄, filtered and the filtrate was concentrated under vcauum to give a residue then purified by silica gel column chromatography (EtOAc/Petroleum ether=0/100~100/0), fraction was concentrated under vacuum to give 1-11g (266 mg, 69.3% yield) as a white solid. MS (ESI) m/z: 1097.8 [M+H]⁺

### Step 7: (2R,3R,4S,5S,6S)-2-((S)-2-((S)-2-((((9H-fluoren-9-yl)methoxy)carbonyl)amino)-3-methylbutanamido)-3-((3-((allyloxy)carbonyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-6-(methoxycarbonyl)tetrahydro-2H-pyran-3,4,5-triyl triacetate (1-11h)

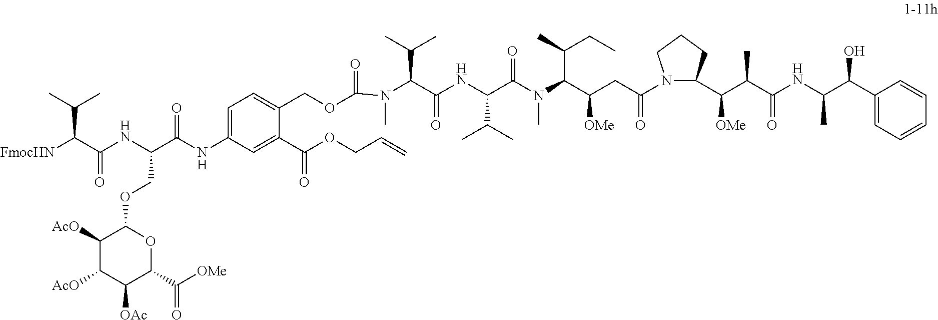

To a mixture of 1-11g (266 mg, 0.24 mmol) and 1-5k (183 mg, 0.26 mmol) in DMF (5 mL) was added HOBt (9.8 mg, 0.073 mmol) and Pyridine (39 μL, 0.49 mmol). The mixture was stirred at r.t. for overnight. The mixture was diluted with EtOAc (20 mL), washed with brine (10 mL), dried over Na₂SO₄, filtered and the filtrate was concentrated under vacuum to give a residue, which was purified by silica gel column chromatography (Methanol/DCM=0/100~10/90) to give 1-11h (369 mg, 90.8% yield) as a white solid. MS (ESI) m/z: 1676.9 [M+H]⁺

### Step 8: 5-((S)-2-((S)-2-((((9H-fluoren-9-yl)methoxy)carbonyl)amino)-3-methylbutanamido)-3-(((2R,3R,4S,5S,6S)-3,4,5-triacetoxy-6-(methoxycarbonyl)tetrahydro-2H-pyran-2-yl)oxy) propanamido)-2-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)benzoic acid (1-11i)

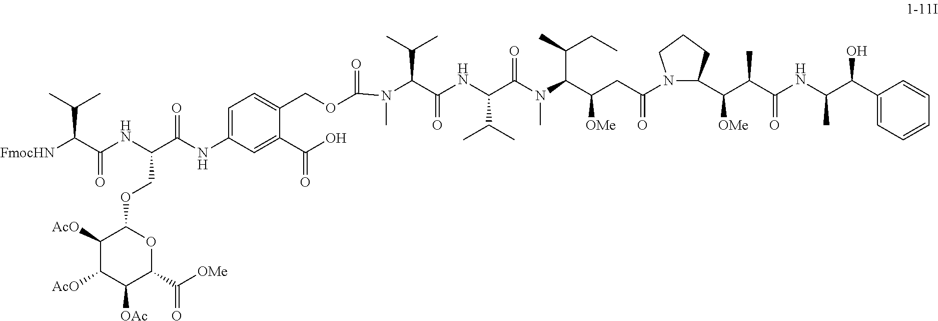

To a mixture of 1-11h (107 mg, 0.064 mmol) and 5,5-dimethylcyclohexane-1,3-dione (18 mg, 0.13 mmol) in DCM (1 ml) was added Pd(PPh₃)₄ (22 mg, 0.019 mmol). The resulted yellow mixture was degassed with nitrogen balloon for three times then stirred at r.t. for 2 hr under nitrogen atmosphere. The mixture was concentrated under vacuum and dissolved in MeCN (2 mL), filtered and purified by prep-HPlC (0.1% FA) (Method: column: XBridge Prep C18 OBD 5 um 19*250 mm; Mobile phase: A-water (0.1% formic acid): B-acetonitrile; Flow rate: 20 mL/min), fraction was concentrated under vacuum to give 1-11i (76 mg, 72.8% yield) as a white solid. MS (ESI) m/z: 1637.3 [M+H]

### Step 9: (2R,3R,4S,5S,6S)-2-((S)-2-((S)-2-((((9H-fluoren-9-yl)methoxy)carbonyl)amino)-3-methylbutanamido)-3-((3-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-6-(methoxycarbonyl)tetrahydro-2H-pyran-3,4,5-triyl triacetate (1-11k)

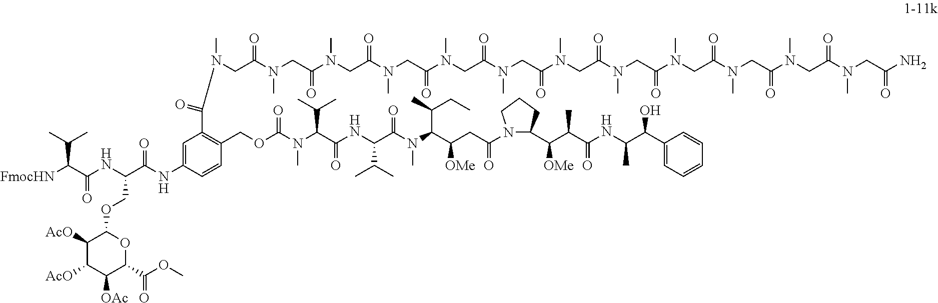

To a mixture of 1-11i (76 mg, 0.046 mmol) and HATU (5.3 mg, 0.014 mmol) in DMF (2 mL) was added DIPEA (16 μL, 0.093 mmol) then stirred at r.t. for 10 min, 1-11j (40 mg, 0.046 mmol, purchased from WuXi AppTec) was added. Finally, the mixture was stirred at r.t. for 1 hr. filtered and purified by prep-HPlC (0.1% FA) (Method: column: XBridge Prep C18 OBD Sum 19*250 mm; Mobile phase: A-water (0.1% formic acid): B-acetonitrile; Flow rate: 20 mL/min), fraction was concentrated under vacuum to give 1-11k (100 mg, 86.5% yield) as a white solid. MS (ESI) m/z: 2487.2 [M+H]⁺

### Step 10: (2S,3S,4S,5R,6R)-6-((S)-3-((3-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-2-((S)-2-amino-3-methylbutanamido)-3-oxopropoxy)-3,4,5-trihydroxytetrahydro-2H-pyran-2-carboxylic acid (1-11l)

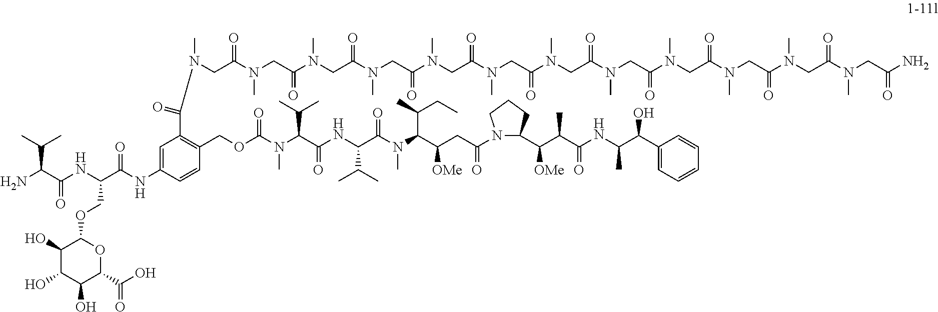 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](N)C(C)C)cc1C(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)C(C)C]

To a mixture of 1-11k (100 mg, 0.04 mmol) in THF (6 mL) was added IN LiOH (2 mL) at 0° C. and stirred at 0° C. for 60 min, then acidified with AcOH to pH=6 and concentrated under vacuum to give a crude product. It was dissolved in DMF (2 mL) and DEA (83 μL, 0.804 mmol) was added and reacted at r.t. for 1 hr. Finally, the mixture was purified by prep-HPlC (0.1% FA) (Method: column: XBridge Prep C18 OBD 5 um 19*250 mm; Mobile phase: A-water (0.1% formic acid): B-acetonitrile; Flow rate: 20 mL/min), fraction was lyophilized to give 1-111 (38 mg, 44.5% yield) as a white solid. MS (ESI) m/z: 2126.2 [M+H]⁺

### Step 11: (2S,3S,4S,5R,6R)-6-(((2S,5S,15S)-16-amino-butyl carbamate-2-((3-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-15-(2,5-dioxo-2,5-dihydro-1H-pyrrol-1-yl)-5-isopropyl-4,7,11-trioxo-12-oxa-3,6,10-triazahexadecyl)oxy)-3,4,5-trihydroxytetrahydro-2H-pyran-2-carboxylic acid (1-11m)

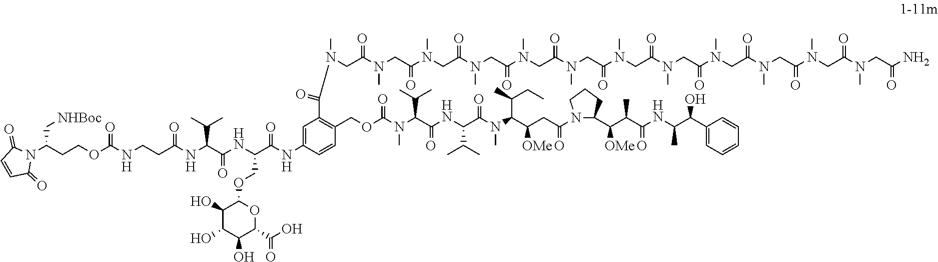 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(=O)CCNC(=O)OCC[C@@H](CNC(=O)OC(C)(C)C)N2C(=O)C=CC2=O)C(C)C)cc1C(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)C(C)C]

To a mixture of 1-5n (3.9 mg, 0.010 mmol) and HATU (3.7 mg, 0.01 mmol) in DMF (2 mL) was added DIPEA (3 μL, 2.3 mg, 0.018 mmol). The mixture was stirred at RT for 5 min. 1-11m (19 mg, 0.009 mmol) was added. The mixture was stirred at r.t. for 1 hr. It was purified by prep-HPlC (0.1% FA) (Method: column: XBridge Prep C18 OBD 5 um 19*250 mm; Mobile phase: A-water (0.1% formic acid): B-acetonitrile; Flow rate: 20 mL/min), fraction was lyophilized to give 1-11m (10.6 mg, 47.3% yield) as a white solid. MS (ESI) m/z: 2506.3 [M+H]⁺

### Step 12: (2S,3S,4S,5R,6R)-6-(((2S,5S,15S)-16-amino-2-((3-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-15-(2,5-dioxo-2,5-dihydro-1H-pyrrol-1-yl)-5-isopropyl-4,7,11-trioxo-12-oxa-3,6,10-triazahexadecyl)oxy)-3,4,5-trihydroxytetrahydro-2H-pyran-2-carboxylic acid (1-11)

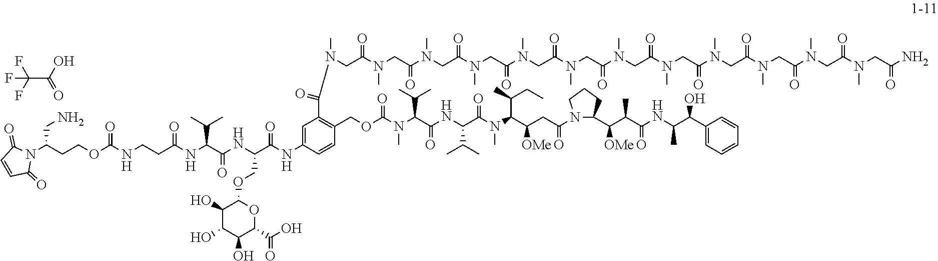

A mixture of 1-11m (10.6 mg, 0.011 mmol) in TFA/DCM (1:4, 2 mL) was stirred at 0° C. for 1 hr. Bubble with nitrogen atmosphere to remove DCM then diluted with ACN (2 mL) then purified by prep-HPlC (0.1% FA) (Method: column: XBridge Prep C18 OBD 5 um 19*250 mm; Mobile phase: A-water (0.1% formic acid): B-acetonitrile; Flow rate: 20 mL/min), fraction was lyophilized to give 1-11 (9 mg, 84.4% yield) in the form of trifluoroacetate salt. MS (ESI) m/z: 2406.9 [M+H]⁺

### Example 1-12

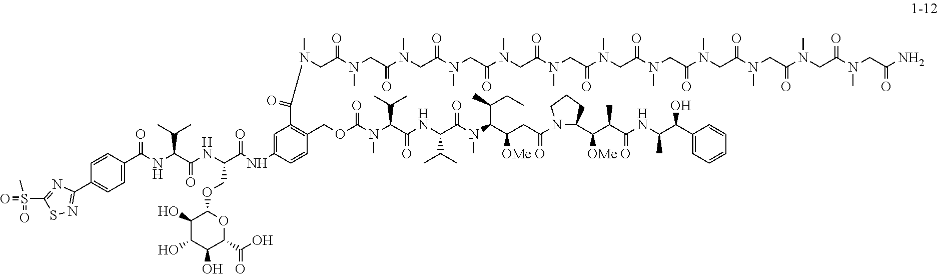 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(=O)c2ccc(-c3nsc(S(C)(=O)=O)n3)cc2)C(C)C)cc1C(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)C(C)C]

### Step 1:(2S,3S,4S,5R,6R)-6-((S)-3-((3-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-2-((S)-3-methyl-2-(4-(5-(methylsulfonyl)-1,2,4-thiadiazol-3-yl)benzamido) butanamido)-3-oxopropoxy)-3,4,5-trihydroxytetrahydro-2H-pyran-2-carboxylic acid (1-12)

To a mixture of 1-6m (5.6 mg, 0.020 mmol) and HATU (7.5 mg, 0.020 mmol) in DMF (2 mL) was added DIPEA (6 μL 4.6 mg, 0.036 mmol). The mixture was stirred at RT for 5 min. 1-111 (19 mg, 0.011 mmol) was added. The mixture was stirred at r.t. for 1 hr. It was purified by prep-HPLC (FA), fraction was freeze-dried to 1-12 (9.2 mg, 43.0% yield) as a white solid. MS (ESI) m/z: 2391.8 [M+H]⁺

### Example 1-13

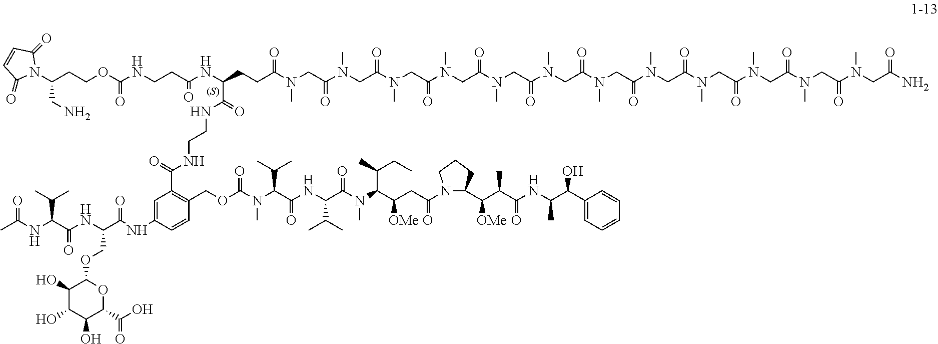

Compound 1-13 (9.9 mg, 51.2% yield) was synthesized according to the synthetic procedures of example 1-9. MS (ESI) m/z: 2618.6 [M+H]⁺

### Example 1-14

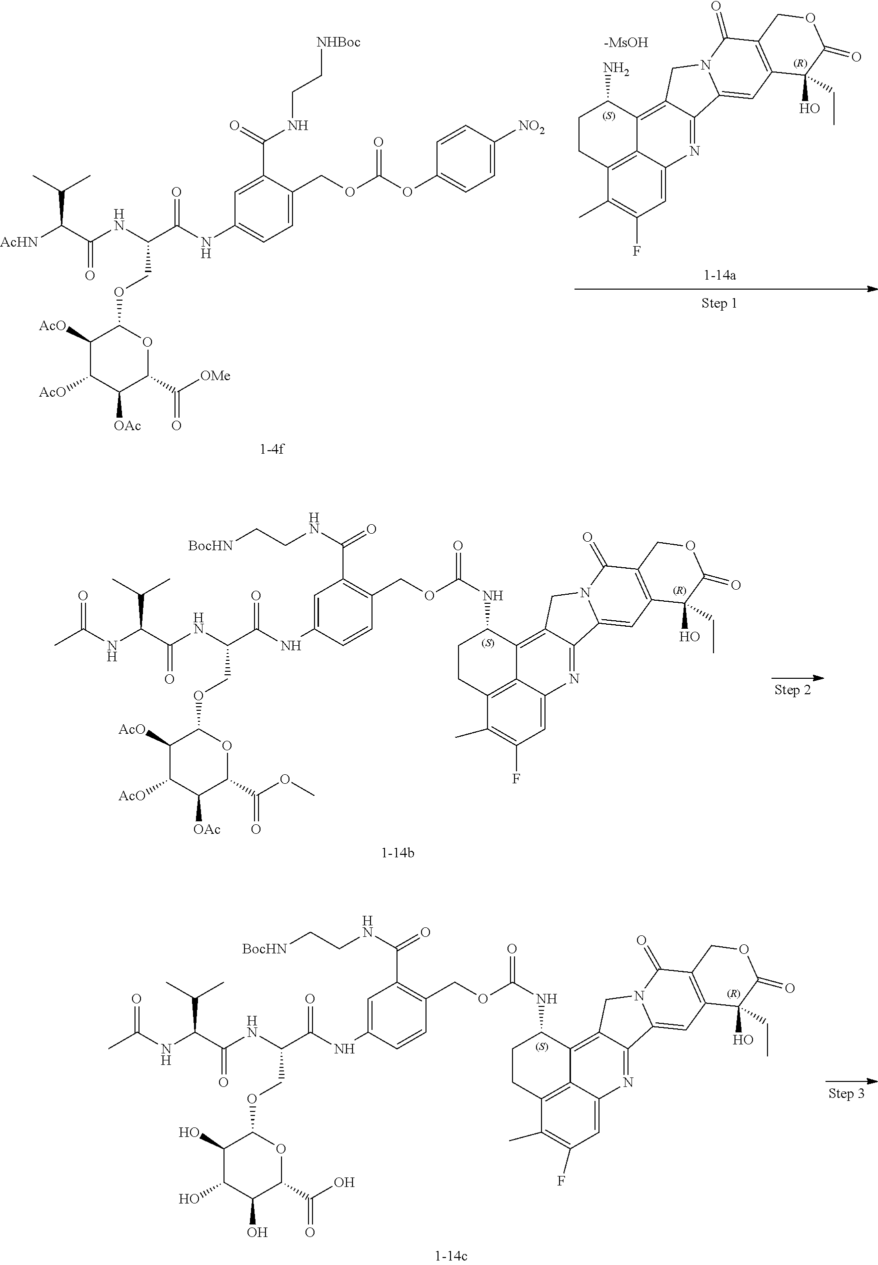

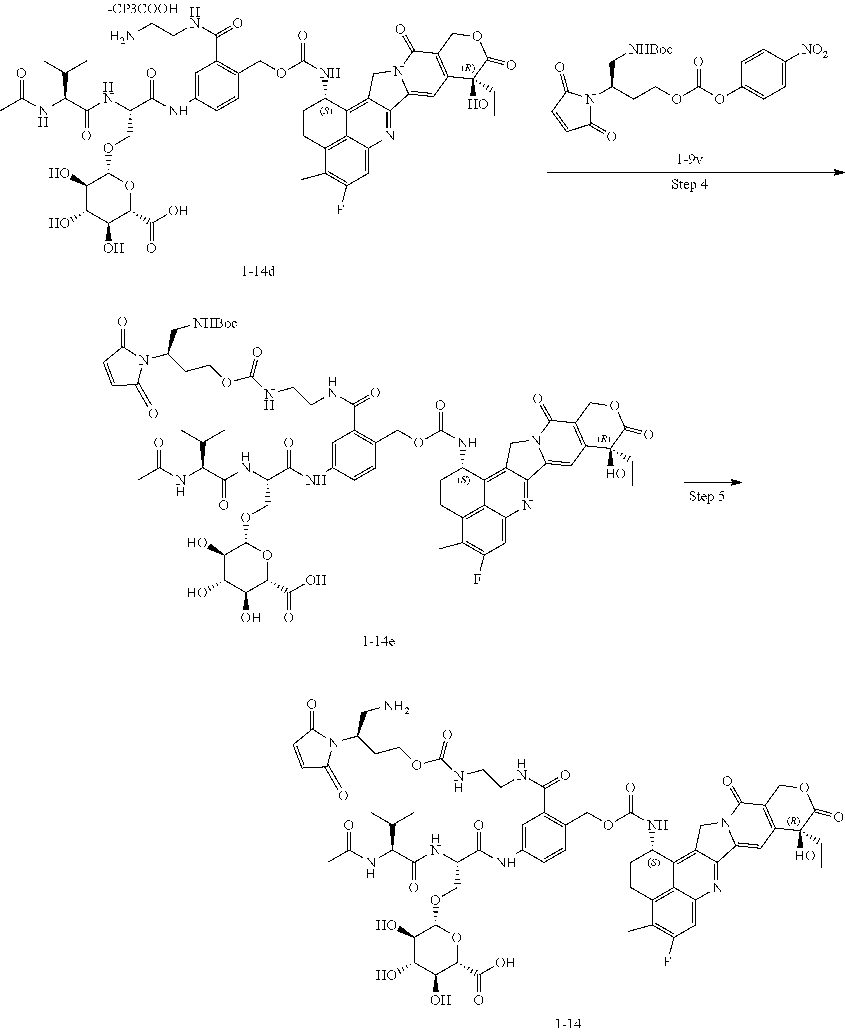

### Step 1:(2R,3R,4S,5S,6S)-2-((S)-2-((S)-2-acetamido-3-methylbutanamido)-3-((3-((2-((tert-butoxy carbonyl)amino)ethyl)carbamoyl)-4-(((((1S,9S)-9-ethyl-5-fluoro-9-hydroxy-4-methyl-10,13-dioxo-2,3,9,10,13,15-′ex′hydro-1H′12′-benzo[de]pyrano[3′,4′: 6,7]indolizino[1,2-b]quinolin-1-yl)carbamoyl)oxy)methyl)phenyl)amino)-3-oxopropoxy)-6-(methoxycarbonyl)tetrahydro-2H-pyran-3,4,5-triyl triacetate (1-14b)

Compound 1-14b (135 mg, 72.2% yield) was synthesized according to synthetic procedure of step 13 of example 1-4. MS (ESI) m/z: 1316.0 [M+H]⁺

### Step 2: (2S,3S,4S,5R,6R)-6-((S)-2-((S)-2-acetamido-3-methylbutanamido)-3-((3-((2-((tert-butoxycarbonyl)amino)ethyl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-3-oxopropoxy)-3,4,5-trihydroxytetrahydro-2H-pyran-2-carboxylic acid (1-14c)

Compound 1-14c (121 mg, crude) was synthesized according to synthetic procedure of step 14 of example 1-4. MS (ESI) m/z: 1176.0 [M+H]⁺

### Step 3: (2S,3S,4S,5R,6R)-6-((S)-2-((S)-2-acetamido-3-methylbutanamido)-3-((3-((2-aminoethyl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-1)carbamoyl)oxy)methyl)phenyl)amino)-3-oxopropoxy)-3,4,5-trihydroxytetrahydro-2H-pyran-2-carboxylic acid-2,2,2-trifluoroacetic acid (1/1) (1-14d)

Compound 1-14d (78 mg, 57.4% yield) was synthesized according to synthetic procedure of step 15 of example 1-4. MS (ESI) m/z: 1075.8 [M+H]⁺

### Step 4: (2S,3S,4S,5R,6R)-6-((S)-2-((S)-2-acetamido3-methylbutanamido)-3-((3-(((R)-8-(2,5-dioxo-2,5-dihydro-IH-pyrrol-1-yl)-13,13-dimethyl-4,11-dioxo-5,12-dioxa-3,10-diazatetradecyl)carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-3-oxopropoxy)-3,4,5-trihydroxytetrahydro-2H-pyran-2-carboxylic acid (1-14e)

Compound 1-14e (21 mg, 60.0% yield) was synthesized according to synthetic procedure of step 16 of example 1-4. MS (ESI) m/z: 1386.3 [M+H]⁺

### Step 5: (2S,3S,4S,5R,6R)-6-((S)-2-((S)-2-acetamido-3-methylbutanamido)-3-((3-((2-((((R)-4-amino-3-(2,5-dioxo-2,5-dihydro-1H-pyrrol-1-yl)butoxy)carbonyl)amino)ethyl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-3-oxopropoxy)-3,4,5-trihydroxytetrahydro-2H-pyran-2-carboxylic acid (1-14)

Compound 1-14 (13.6 mg, 64.5% yield) was synthesized according to synthetic procedure of step 17 of example 1-4. MS (ESI) m/z: 1286.1 [M+H]⁺

### Example 1-15

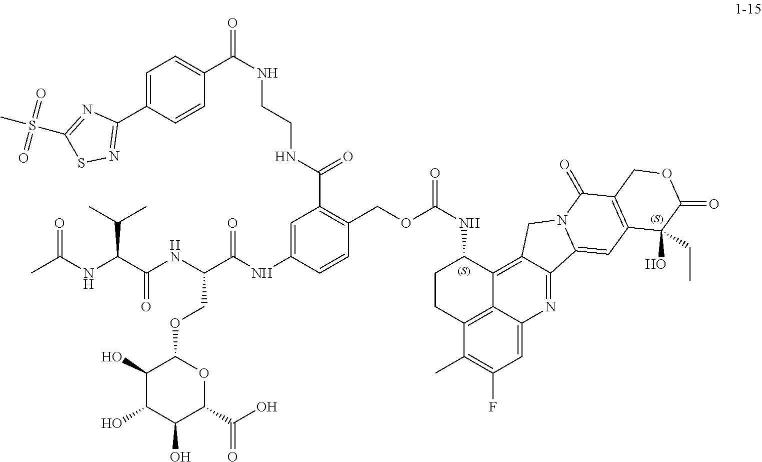

### (2S,3S,4S,5R,6R)-6-((S)-2-((S)-2-acetamido-3-methylbutanamido)-3-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)-3-((2-(4-(5-(methylsulfonyl)-1,2,4-thiadiazol-3-yl)benzamido)ethyl)carbamoyl)phenyl)amino)-3-oxopropoxy)-3,4,5-trihydroxytetrahydro-2H-pyran-2-carboxylic acid hydrate (1-15)

Compound 1-15 (7.3 mg, 49.7% yield) was synthesized according to synthetic procedure of step 1 of example 1-8. MS (ESI) m/z: 1341.9 [M+H]⁺

### Example 1-16

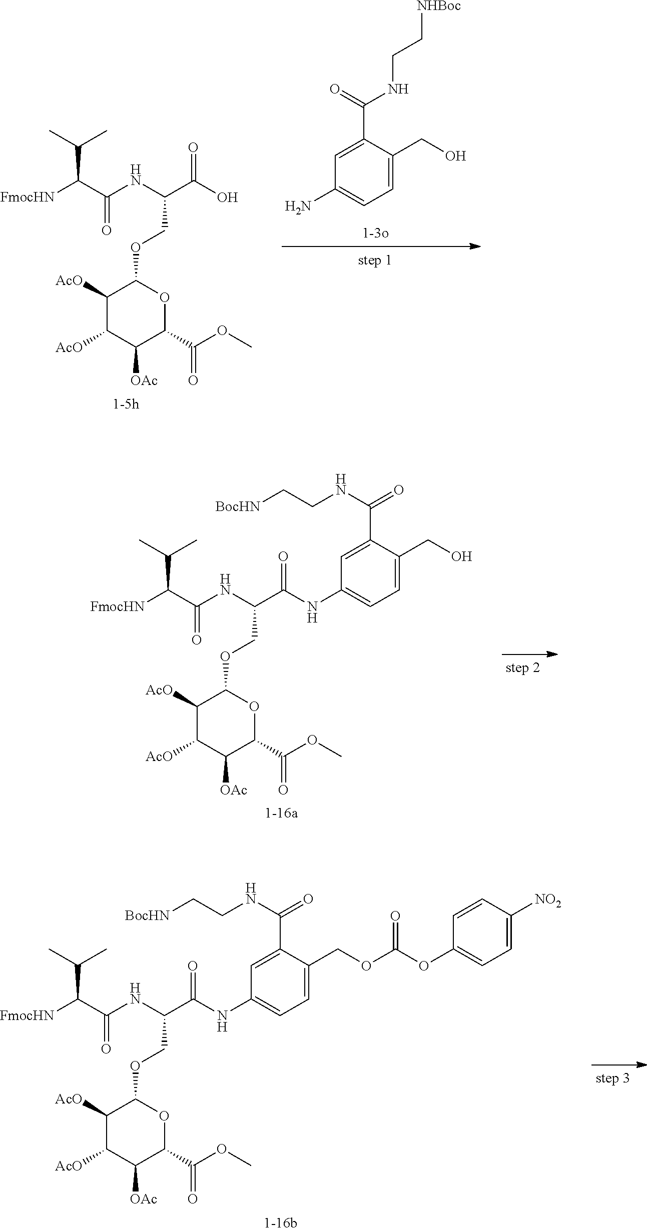

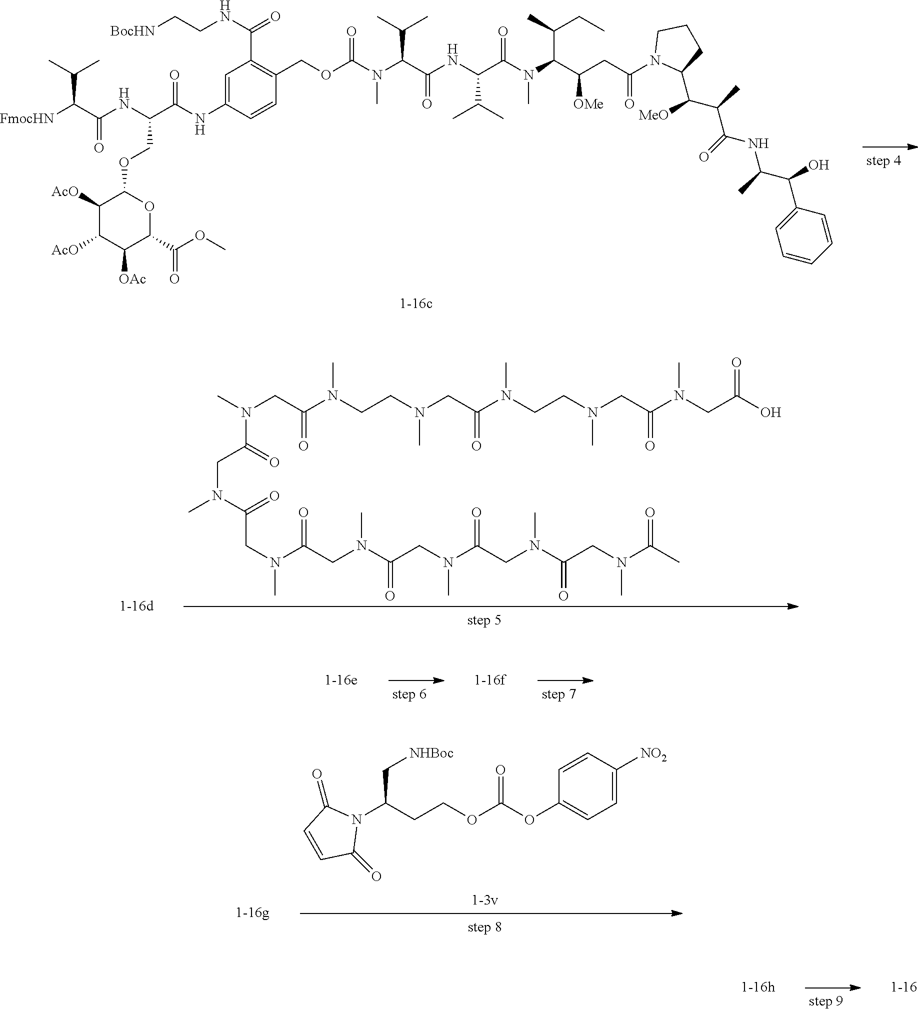

### Step 1:(2R,3R,4S,5S,6S)-2-((S)-2-((S)-2-((((9H-fluoren-9-yl)methoxy)carbonyl)amino)-3-methylbutanamido)-3-((3-((2-((tert-butoxycarbonyl)amino)ethyl)carbamoyl)-4-(hydroxymethyl)phenyl)amino)-3-oxopropoxy)-6-(methoxycarbonyl)tetrahydro-2H-pyran-3,4,5-triyl triacetate (1-16a)

To a mixture of 1-30 (416 mg, 1.34 mmol) and 1-5h (1000 mg, 1.34 mmol) in DCM (10 mL) was added EEDQ (397 mg, 1.60 mmol). The mixture was stirred at RT for 5 h. TLC showed the reaction was consumed. The mixture was concentrated under vacuum to give a crude product, purified by silica gel column chromatography (EA:DCM=0-10%) to give 1-16a (900 mg, 51.4% yield) as a white solid. MS (ESI) m/z: 1035.1 [M+H]⁺

### Step 2: (2R,3R,4S,5S,6S)-2-((S)-2-((S)-2-((((9H-fluoren-9-yl)methoxy)carbonyl)amino)-3-methylbutanamido)-3-((3-((2-((tert-butoxycarbonyl)amino)ethyl)carbamoyl)-4-((((4-nitrophenoxy)carbonyl)oxy)methyl)phenyl)amino)-3-oxopropoxy)-6-(methoxycarbonyl)tetrahydro-2H-pyran-3,4,5-triyl triacetate (1-16b)

To a mixture of 1-16a (300 mg, 0.29 mmol) and 4-Nitrophenyl chloroformate (11697ucc 097uccmmol) in DCM (3 mL) was added DIEA (202 μL, 1.16 mmol). The resulted light-yellow mixture was stirred at RT for 12 hr. The mixture was concentrated and purified by silica gel column chromatography (EA:DCM=0-10%) to give 1-16b (250 mg, 71.8% yield) as a pale-yellow solid. MS (ESI) m/z: 1200.2 [M+H]⁺

### Step 3: (2R,3R,4S,5S,6S)-2-((S)-2-((S)-2-((((9H-fluoren-9-yl)methoxy)carbonyl)amino)-3-methylbutanamido)-3-((3-((2-((tert-butoxycarbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-6-(methoxy carbonyl)tetrahydro-2H-pyran-3,4,5-triyl triacetate (1-16c)

To a solution of compound 1-16b (250 mg, 0.208 mmol) and MMAE (164 mg, 0.229 mmol) in DMF (3.0 mL) was added DIPEA (90 mL, 0.52 mmol) and HOBt (7 mg, 0.052 mmol) at 0° C. The mixture was stirred at r.t. for 24 h. The reaction was concentrated and purified by flash column chromatography (MeOH/DCM=0-5%), Compound 1-16c (100 mg, 27.0% yield) was obtained as a White solid. MS (ESI) m/z: 1779.2 [M+H]⁺.

### Step 4: (2R,3R,4S,5S,6S)-2-((S)-2-((S)-2-amino-3-methylbutanamido)-3-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-3-oxopropoxy)-6-(methoxycarbonyl)tetrahydro-2H-pyran-3,4,5-triyl triacetate (1-16d)

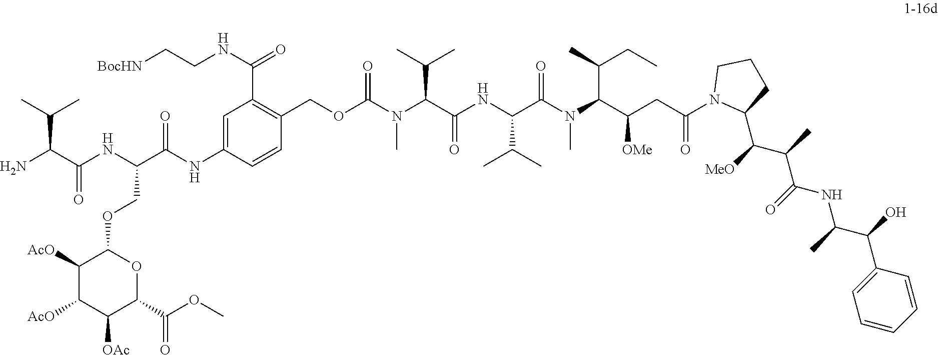 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)OC)[C@@H](OC(C)=O)[C@H](OC(C)=O)[C@H]2OC(C)=O)NC(=O)[C@@H](N)C(C)C)cc1C(=O)NCCNC(=O)OC(C)(C)C)C(C)C]

To a solution of compound 1-16c (100 mg, 0.0562 mmol) in DMF (2.0 mL) was added DEA (58 mL, 0.562 mmol) at 0° C. The mixture was stirred at r.t. for 1 h. The reaction was concentrated to give the crude product 1-16d. MS (ESI) m/z: 1156.3 [M+H]⁺.

### Step 5: (2R,3R,4S,5S,6S)-2-(((2S,5S)-2-((3-((2-((tert-butoxycarbonyl)amino)ethyl) carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-5-isopropyl-9,12,15,18,21,24,27,30,33,36,39,42-dodecamethyl-4,7,10,13,16,19,22,25,28,31,34,37,40,43-tetradecaoxo-3,6,9,12,15,18,21,24,27,30,33,36,39,42-tetradecaazatetratetracontyl)oxy)-6-(methoxycarbonyl)tetrahydro-2H-pyran-3,4,5-triyl triacetate (1-16e)

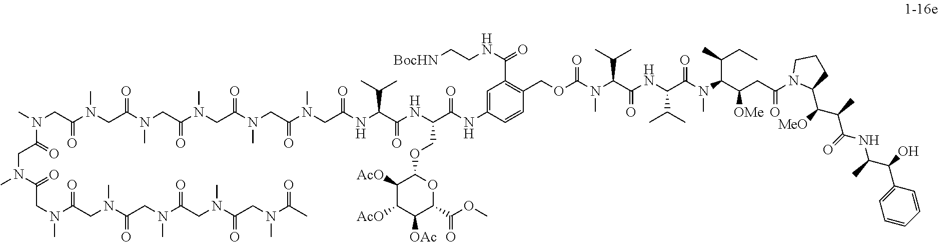 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)OC)[C@@H](OC(C)=O)[C@H](OC(C)=O)[C@H]2OC(C)=O)NC(=O)[C@@H](NC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C)cc1C(=O)NCCNC(=O)OC(C)(C)C)C(C)C]

To a solution of crude compound 1-16d (0.0562 mmol) and 3,6,9,12,15,18,21,24,27,30,33,36-dodecamethyl-4,7,10,13,16,19,22,25,28,31,34,37-dodecaoxo-3,6,9,12,15,18,21,24,27,30,33,36-dodecaazaoctatriacontanoic acid (51 mg, 0.0562 mmol) in DMF (2.0 mL) was added HATU (25 mg, 0.067 mmol) and DIPEA (19 mL, 0.11 mmol) at 0° C. The mixture was stirred at r.t. for 1 h. The reaction was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-16e (trifluoroacetate) (40 mg, 29.0% yield, two steps) as a white solid. MS (ESI) m/z: 2451.3 [M+H]⁺.

### Step 6: (2R,3R,4S,5S,6S)-2-(((2S,5S)-2-((3-((2-aminoethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-5-isopropyl-9,12,15,18,21,24,27,30,33,36,39,42-dodecamethyl-4,7,10,13,16,19,22,25,28,31,34,37,40,43-tetradecaoxo-3,6,9,12,15,18,21,24,27,30,33,36,39,42-tetradecaazatetratetracontyl)oxy)-6-(methoxycarbonyl)tetrahydro-2H-pyran-3,4,5-triyl triacetate (1-16f)

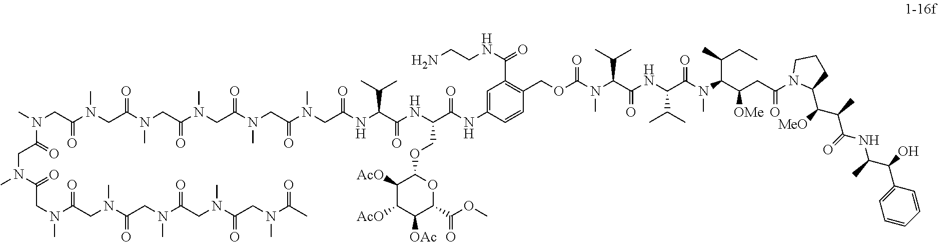 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)OC)[C@@H](OC(C)=O)[C@H](OC(C)=O)[C@H]2OC(C)=O)NC(=O)[C@@H](NC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C)cc1C(=O)NCCN)C(C)C]

To a solution of compound 1-16e (30 mg, 0.0122 mmol) in DCM (1.0 mL) was added TFA (0.2 mL) at 0° C. The mixture was stirred at 0° C. for 10 min. The reaction was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-16f (trifluoroacetate) (20 mg, 69.7% yield) as a white solid. MS (ESI) m/z: 2351.2 [M+H]⁺.

### Step 7: (2S,3S,4S,5R,6R)-6-(((2S,5S)-2-((3-((2-aminoethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-5-isopropyl-9,12,15,18,21,24,27,30,33,36,39,42-dodecamethyl-4,7,10,13,16,19,22,25,28,31,34,37,40,43-tetradecaoxo-3,6,9,12,15,18,21,24,27,30,33,36,39,42-tetradecaazatetratetracontyl)oxy)-3,4,5-trihydroxytetrahydro-2H-pyran-2-carboxylic acid (1-16g)

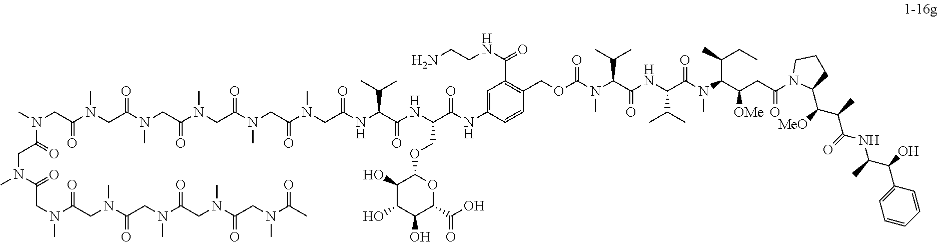 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)C(C)C)cc1C(=O)NCCN)C(C)C]

To a solution of compound 1-16f (20 mg, 0.0085 mmol) in saturated LiBr (MeCn/WATER 10/1) (1.0 mL) was added NEt₃ (7 mL, 0.0510 mmol) at 0° C. The mixture was stirred at r.t. for 4 h. The reaction was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-16g (trifluoroacetate) (10 mg, 53.2% yield) as a white solid. MS (ESI) m/z: 2211.27 [M+H]⁺.

### Step 8: (2S,3S,4S,5R,6R)-6-(((2S,5S)-2-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dcana-4,7,10-triazatetradecyl)-3-(((R)-8-(2,5-dioxo-2,5-dihydro-1H-pyrrol-1-yl)-13,13-dimethyl-4,11-dioxo-5,12-dioxa-3,10-diazatetradecyl)carbamoyl)phenyl)carbamoyl)-5-isopropyl-9,12,15,18,21,24,27,30,33,36,39,42-dodecamethyl-4,7,10,13,16,19,22,25,28,31,34,37,40,43-tetradecaoxo-3,6,9,12,15,18,21,24,27,30,33,36,39,42-tetradecaazatetratetracontyl)oxy)-3,4,5-trihydroxytetrahydro-2H-pyran-2-carboxylic acid (1-16h)

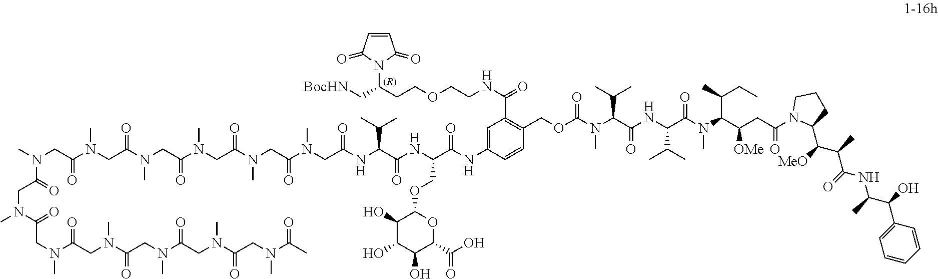

To a solution of compound 1-16g (10 mg, 0.0045 mmol) and 1-3v (2.4 mg, 0.0054 mmol) in DMF (1.0 mL) was added DIPEA (2.3 mL, 0.0135 mmol) at 0° C. The mixture was stirred at r.t. for 5 h. The reaction was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-16h (trifluoroacetate) (7 mg, 61.7% yield) as a white solid. MS (ESI) m/z: 2522.1 [M+H]⁺.

### Step 9: (2cancanS,5R,6R)-6-(((2S,5S)-2-((3-((2-((((R)-4-amino-3-(2,5-dioxo-2,5-dihydro-1H-pyrrol-1-yl)butoxy)carbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl)-5-isopropyl-9,12,15,18,21,24,27,30,33,36,39,42-dodecamethyl-4,7,10,13,16,19,22,25,28,31,34,37,40,43-tetradecaoxo-3,6,9,12,15,18,21,24,27,30,33,36,39,42-tetradecaazatetratetracontyl)oxy)-3,4,5-trihydroxytetrahydro-2H-pyran-2-carboxylic acid (1-16)

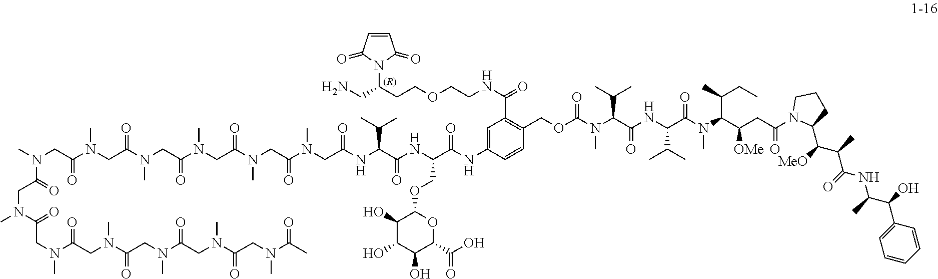

To a solution of crude compound 1-16h (7 mg, 0.00277 mmol) in DCM (1.0 mL) was added TFA (0.2 mL) at 0° C. The mixture was stirred at 0° C. for 10 min. The reaction was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-16 (trifluoroacetate) (3 mg, 44.7% yield) as a white solid. MS (ESI) m/z: 2421.94 [M+H]⁺.

The conjugator-linker-payload structures prepared herein are summarized below in Table

TABLE 1
Conjugator-linker-payloads

| Com- | |
|---|---|
| pound | |
| No. | Conjugator-Linker-Payload Structure |
| 1-1 | 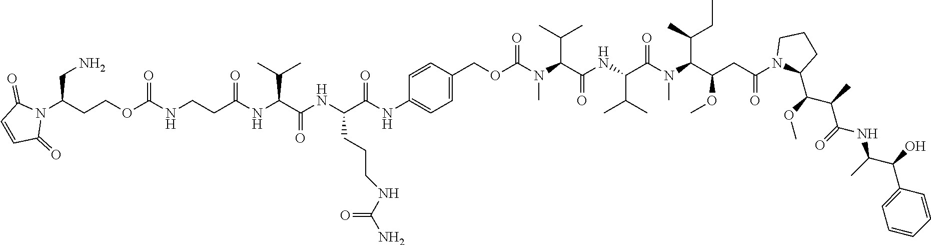 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCNC(=O)OCC[C@H](CN)N2C(=O)C=CC2=O)C(C)C)cc1)C(C)C] |
| 1-2 | 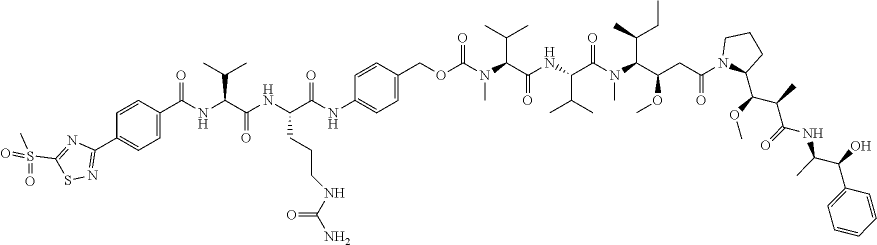 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2ccc(-c3nsc(S(C)(=O)=O)n3)cc2)C(C)C)cc1)C(C)C] |
| 1-3 | 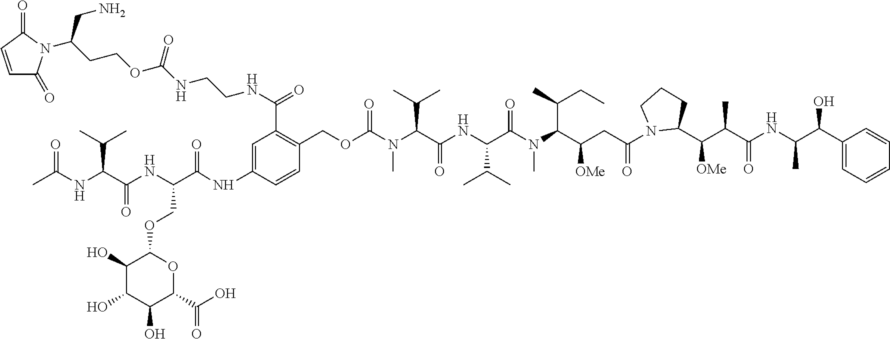 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1C(=O)NCCNC(=O)OCC[C@H](CN)N1C(=O)C=CC1=O)C(C)C] |
| 1-4 | 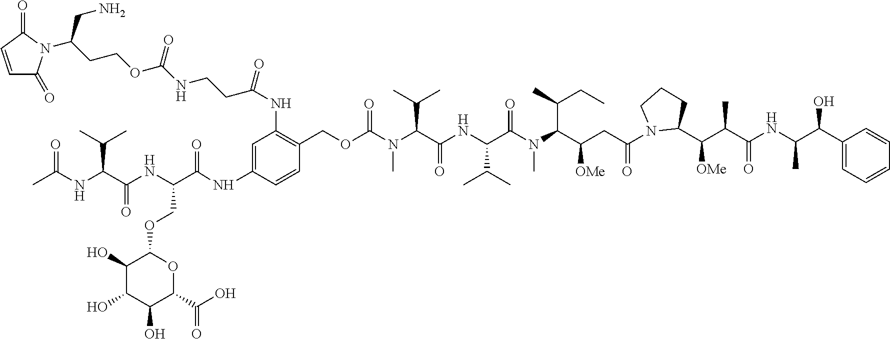 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1NC(=O)CCNC(=O)OCC[C@H](CN)N1C(=O)C=CC1=O)C(C)C] |
| 1-5 | 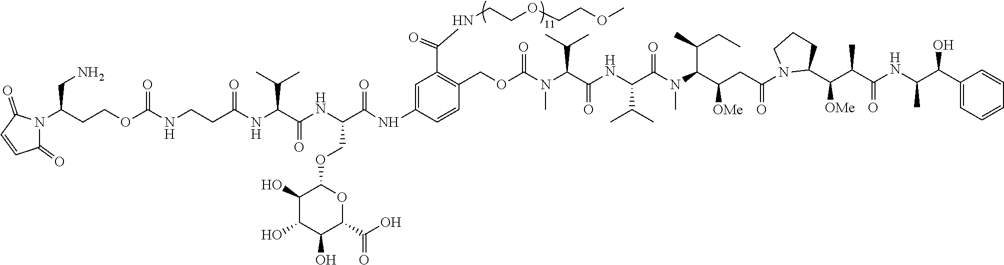 |
| 1-6 | 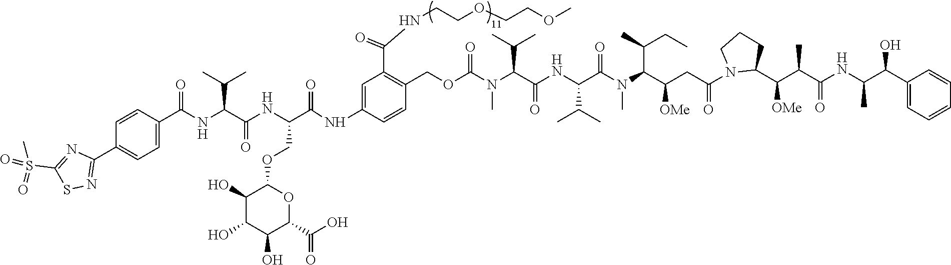 |
| 1-7 | 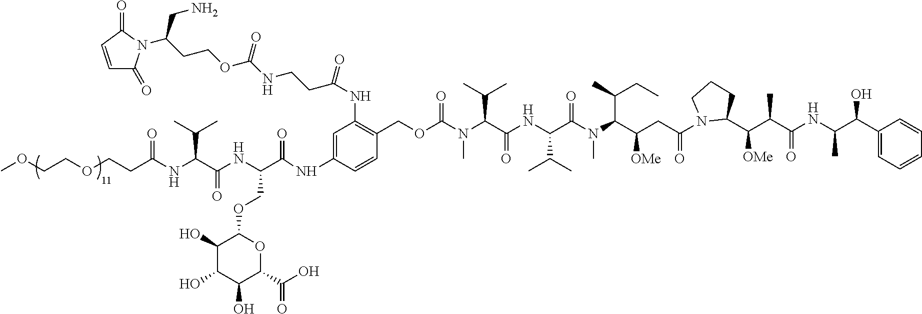 |
| 1-8 | 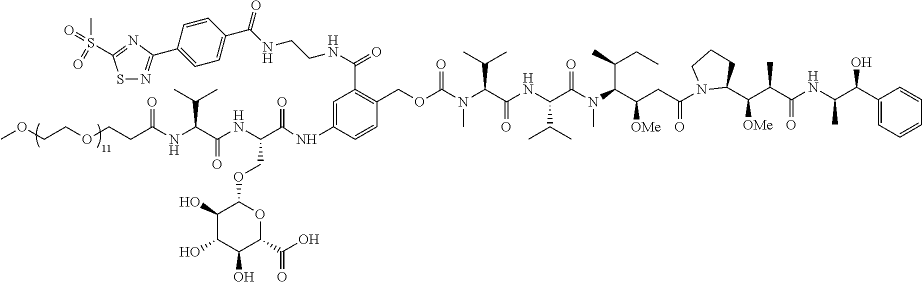 |
| 1-9 | 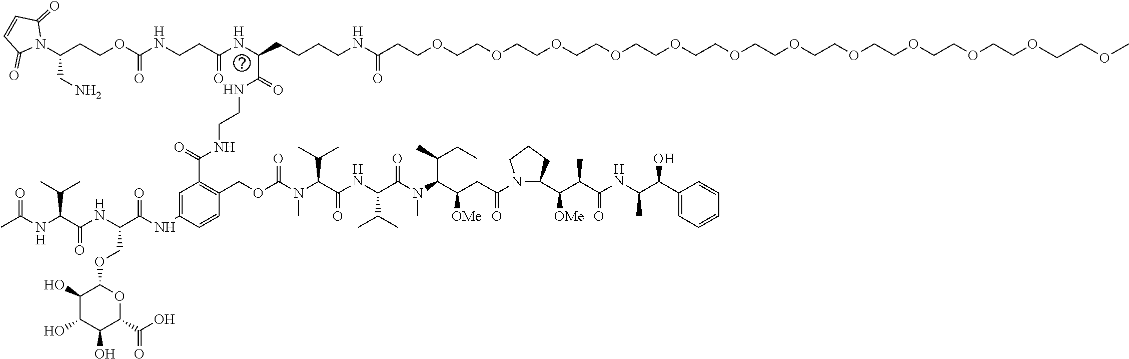 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1C(=O)NCCNC(=O)[C@H](CCCCNC(=O)CCOCCOCCOCCOCCOCCOCCOCCOCCOCCOCCOCCOC)NC(=O)CCNC(=O)OCC[C@H](CN)N1C(=O)C=CC1=O)C(C)C] |
| 1-10 | 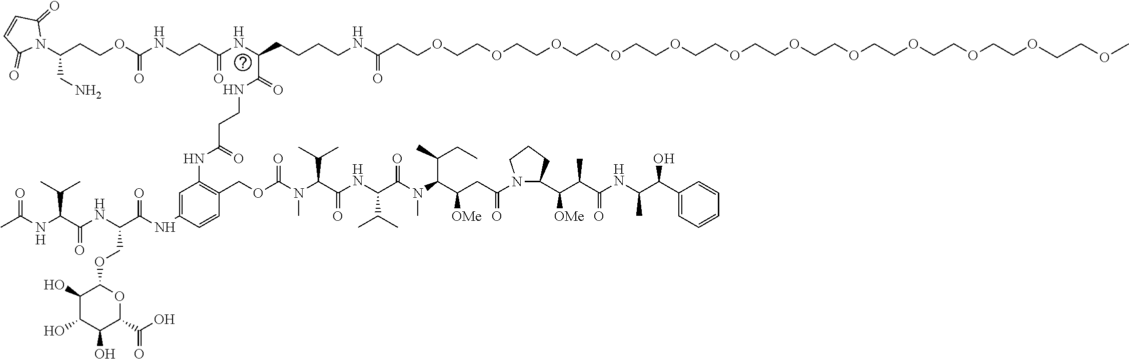 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1NC(=O)CCNC(=O)[C@H](CCCCNC(=O)CCOCCOCCOCCOCCOCCOCCOCCOCCOCCOCCOCCOC)NC(=O)CCNC(=O)OCC[C@H](CN)N1C(=O)C=CC1=O)C(C)C] |
| 1-11 | 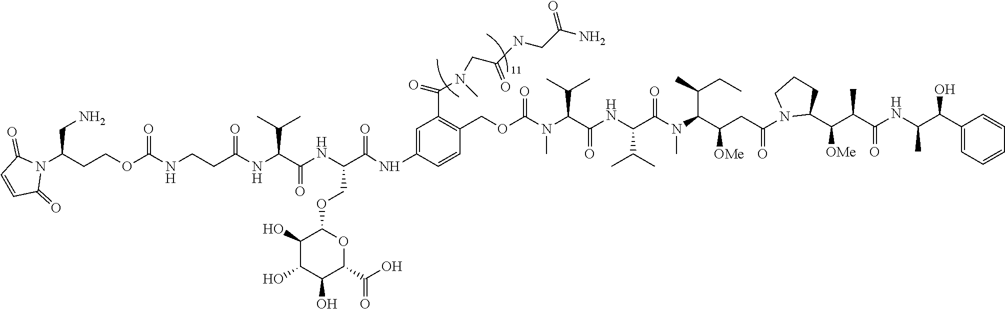 |
| 1-12 | 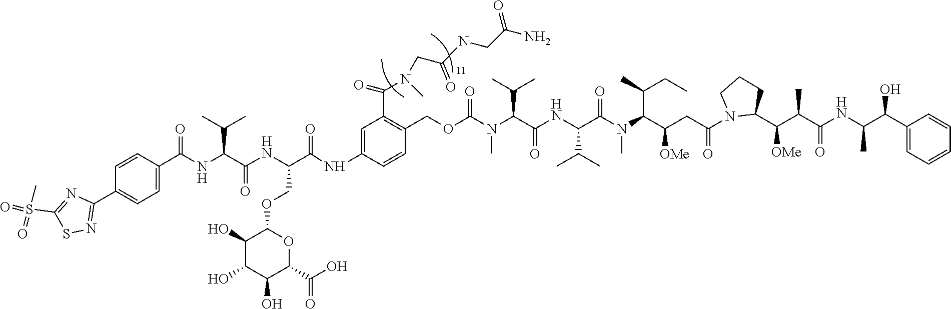 |
| 1-13 | 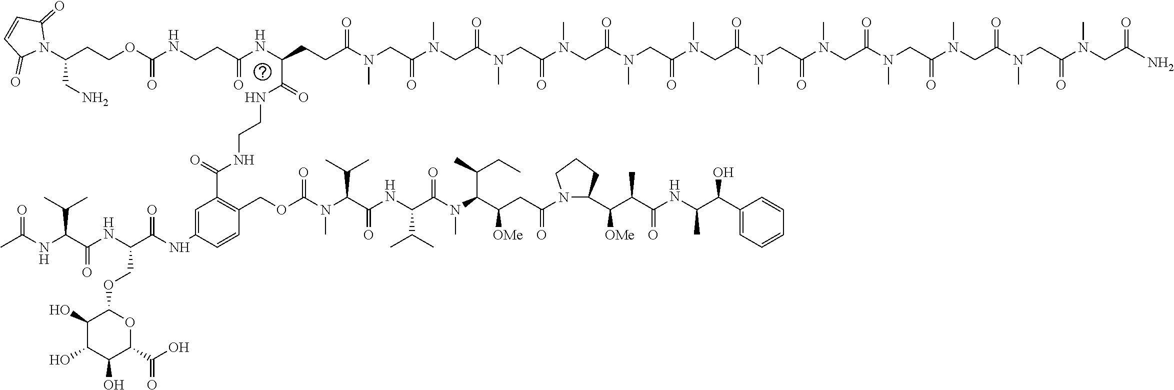 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CO[C@@H]2O[C@H](C(=O)O)[C@@H](O)[C@H](O)[C@H]2O)NC(=O)[C@@H](NC(C)=O)C(C)C)cc1C(=O)NCCNC(=O)[C@H](CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)NC(=O)CCNC(=O)OCC[C@H](CN)N1C(=O)C=CC1=O)C(C)C] |
| 1-14 | 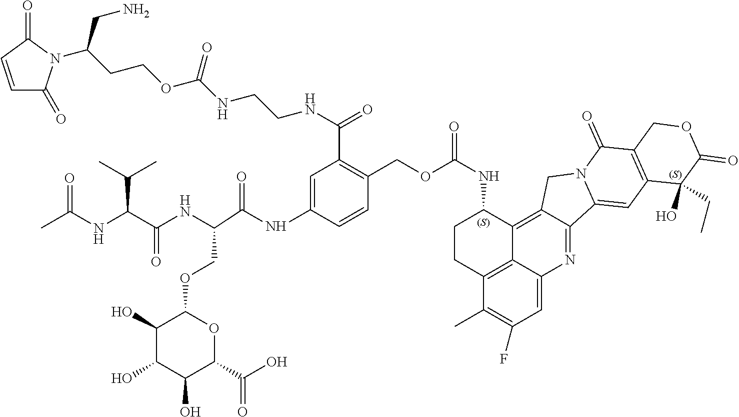 |
| 1-15 | 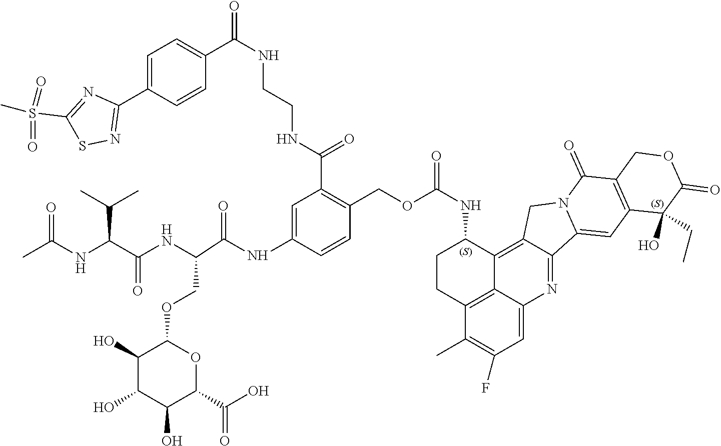 |
| 1-16 | 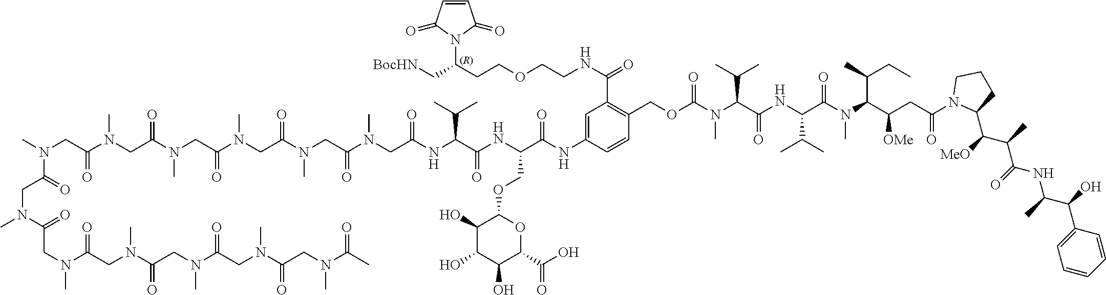 |

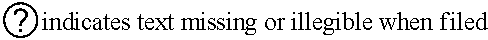

### Example 2: Antibody Drug Conjugate Preparation and Characterization

DAR 3-5 antibody drug conjugate preparation. An antibody in a conjugation buffer (with concentration 0.5-25 mg/mL, PBS buffer pH 6.0-8.5) was incubated under reduction temperature (0-40° C.) for 10 min. 2-10 eq. TECP solution (5 mM stock in PBS buffer) was added to the reaction mixture and the reduction reaction was left for 1-8 hours at reduction temperature. Organic solvent (e.g., DMSO, DMF, DMA, PG, acetonitrile, 0-25% v/v) and the conjugator-linker-payload stock prepared herein (6-25 eq, 10 mM stock in organic solvent) (see Tables 2A-2D) were added stepwise after the reduction mixture was cooled to 0-25′° C. Conjugation solution was left for 1-3 h at 0-25° C., and the reaction can be quenched with N-acetyl cysteine (1 mM stock). The solution was submitted to buffer exchange (spin desalting column, ultra filtration, and dialysis) into storage buffer (for example, pH 5.5-6.5 histidine acetate buffer, with optional additive such as sucrose, trehalose, tween 20, 60, 80).

After the conjugation step, the ADC was subjected to buffer exchange into ring opening buffer (pH 6.5~9.0, PBS, borate or tris buffer) and the solution was left at 22 or 37° C. for 1~48 h. The maleimide ring opening process was monitored via reduced LCMS. Once the conjugated maleimide hydrolysis was completed, the resulting ADCs were buffer exchanged into basic tris pH 8.0-8.5 buffer or acidic histidine-acetate pH 5.0-6.5 buffer via dialysis.

LCMS method to monitor and determine maleimide hydrolysis. LC-MS analysis was carried out under the following measurement conditions:
LC-MS system: Vanquish Flex UHPLC and Orbitrap exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program 1: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Gradient program 2
Injected sample amount: 2 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

ADC characterization. ADCs were characterized using the following analytical methods. Drug to antibody ratios (DAR) of the ADCs were determined by LCMS method or HIC method. SEC purity of ADCs made were all >95% purity.

LCMS method: LC-MS analysis was carried out under the following measurement conditions:
LC-MS system: Vanquish Flex UHPLC and Orbitrap exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 mi)
Injected sample amount: 1 μg
MS parameters: Intact and denaturing MIS data were acquired in HMR mode at setting of R=15k and deconvolved using the ReSpect™ algorithm and Sliding Window integration in Thermo Scientific™ BioPharma Finder™ 4.0 software.

HIC method: HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 g

SEC method to determine ADC purity: HPLC analysis was carried out under the following measurement conditions:
HPLC system: Waters H-Class UPLC System
Detector: measurement wavelength: 280 nm
Column: ACQUITY UPLC BE1200 SEC 1.7 um 4.6×150 mm, Waters
Column temperature: room temperature
Mobile phase A: 200 mM Phosphate buffer, 250 mM potassium chloride, 15% isopropyl alcohol, pH 7.0
Gradient program: under 10 min isocratic elutions with the flow rate of 0.3 mL/min
Injected sample amount: 20 g

ADC hydrophobicity evaluation: An ADC with a higher hydrophobic property would appear with a later retention time (“RT”) from HIC (hydrophobicity interaction column) chromatography. Results are presented in Tables 2A-2D using the DAR8 peak as a reference.

TABLE 2A
Isotype ADC Prepared with anti-HIV Isotype Antibody as ″Ab″

| ADC | | DAR8 |
|---|---|---|
| No. | ADC Structure | RT (min) |
| Iso- type | 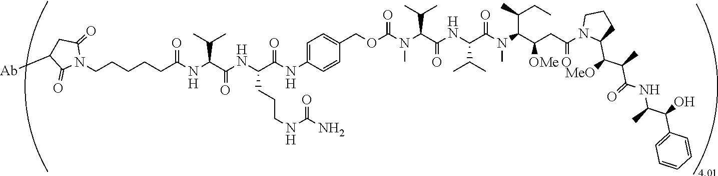 | 14.288 |

TABLE 2B
ADCs Prepared with 6E7 as ″Ab″

| ADC | | DAR8 |
|---|---|---|
| No. | ADC Structure | RT (min) |
| 3-A | 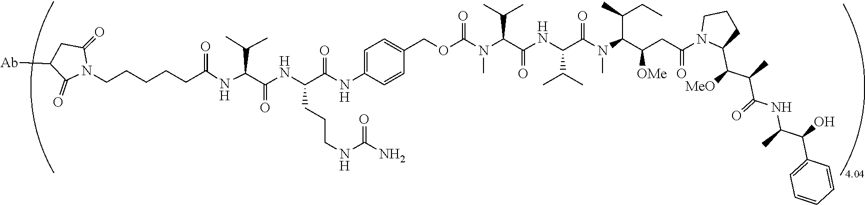 | 14.466 |
| 3-1 | 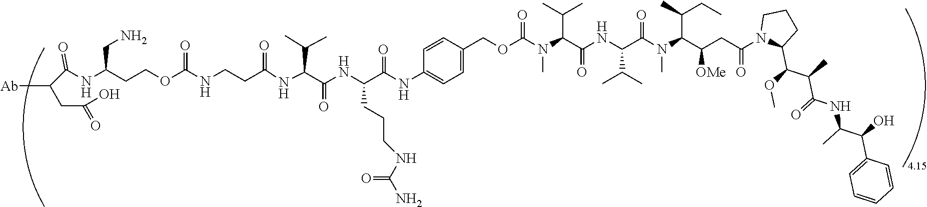 | 14.491 |
| 3-2 | 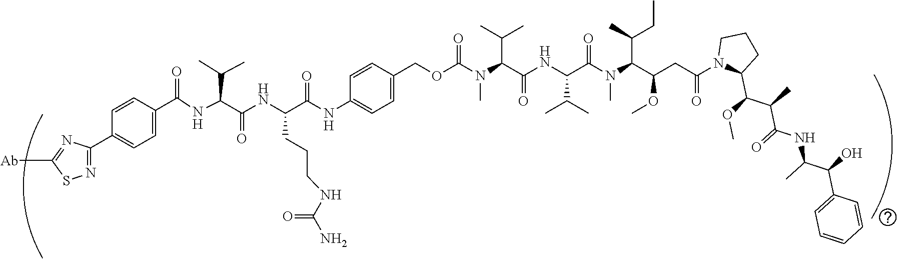 | 14.167 |
| 3-3 | 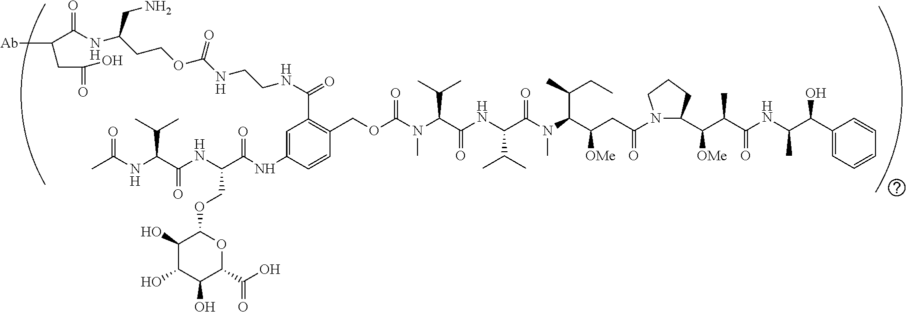 | 12.967 |
| 3-4 | 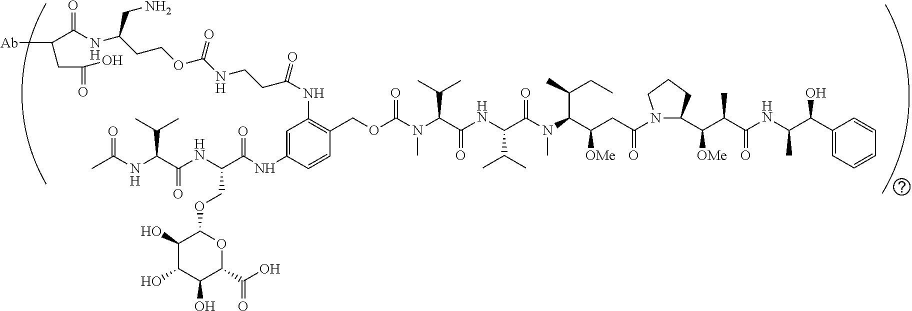 | 12.669 |
| 3-5 | 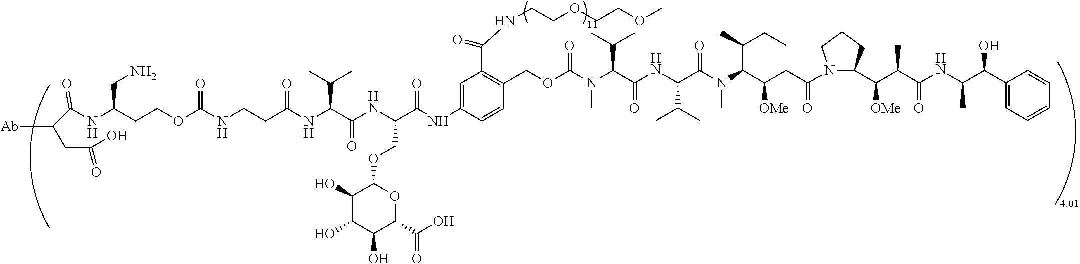 | 12.353 |
| 3-6 | 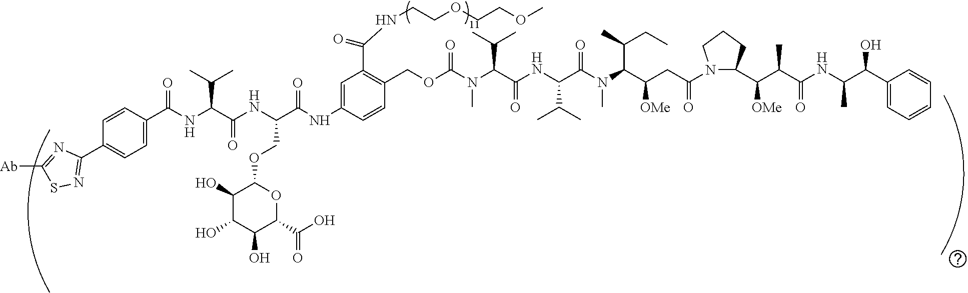 | 12.466 |
| 3-7 | 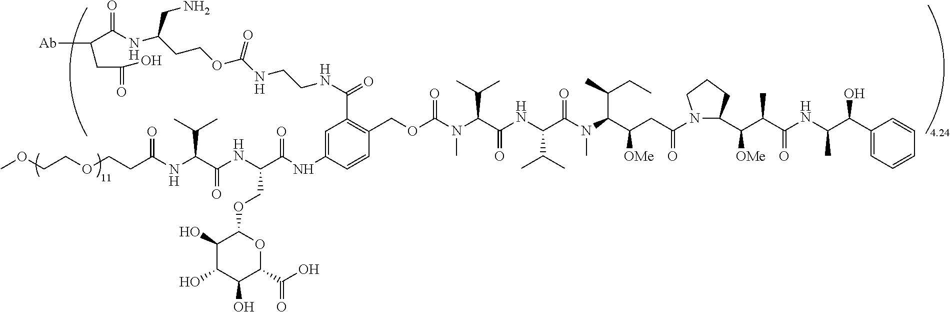 | 11.961 |
| 3-8 | 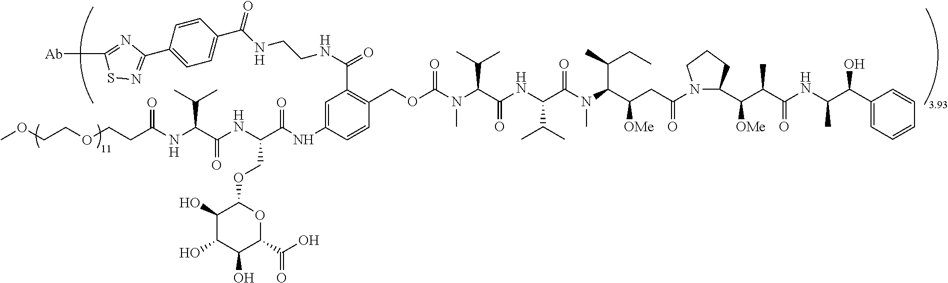 | 11.87 |
| 3-9 | 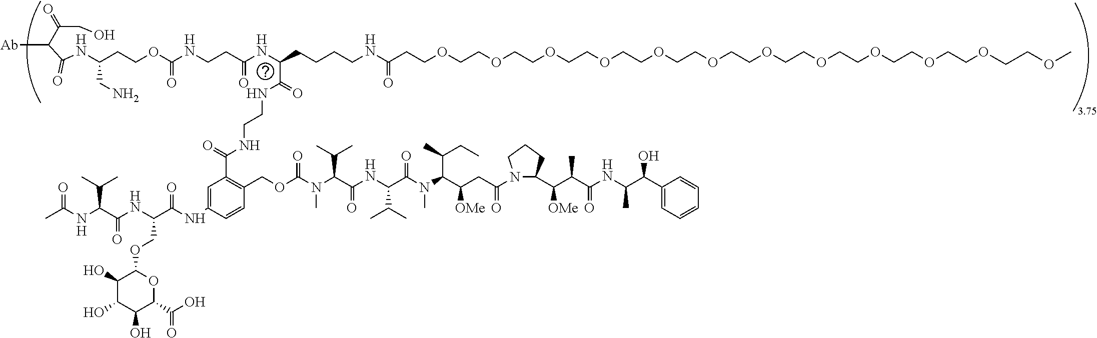 | 13.370 |
| 3-10 | 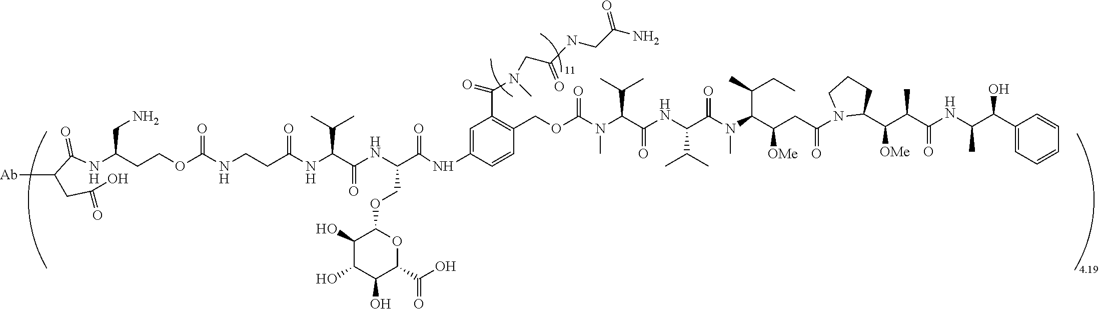 | 11.116 |
| 3-11 | 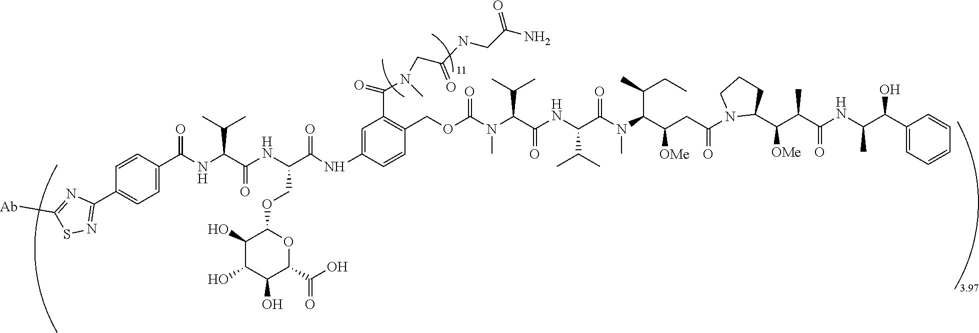 | NoData |
| 3-12 | 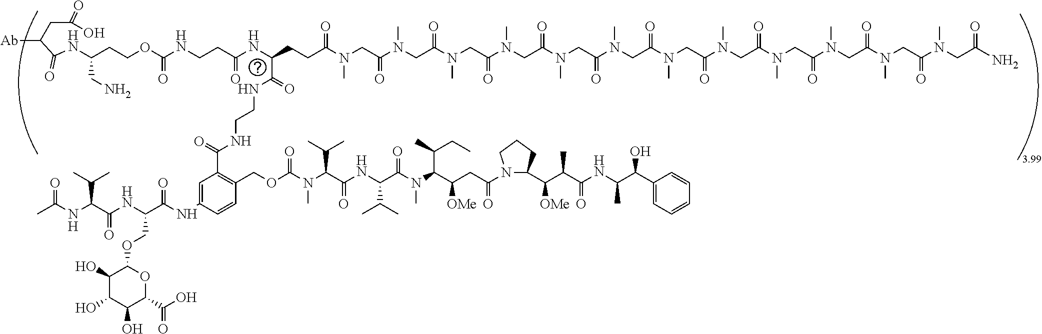 | 12.095 |
| 3-15 | 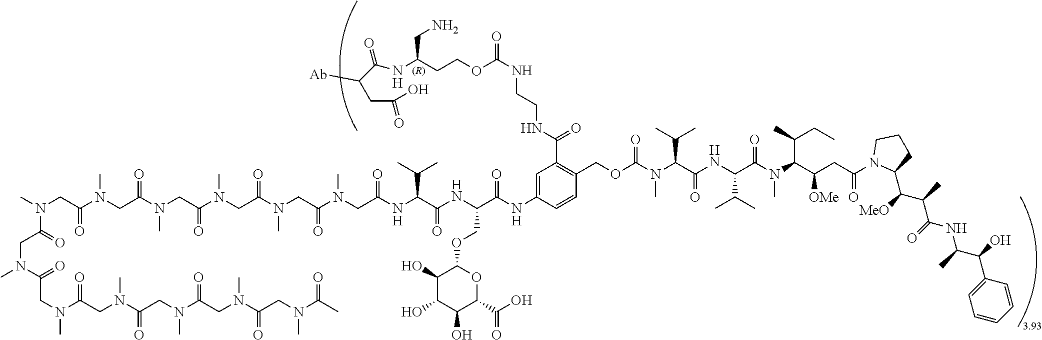 | 10.911 |

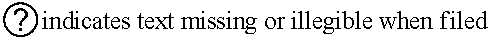

TABLE 2C
ADCs Prepared with Infinatamab as ″Ab″

| ADC | | DAR8 |
|---|---|---|
| No. | ADC Structure | RT (min) |
| 3-13 | 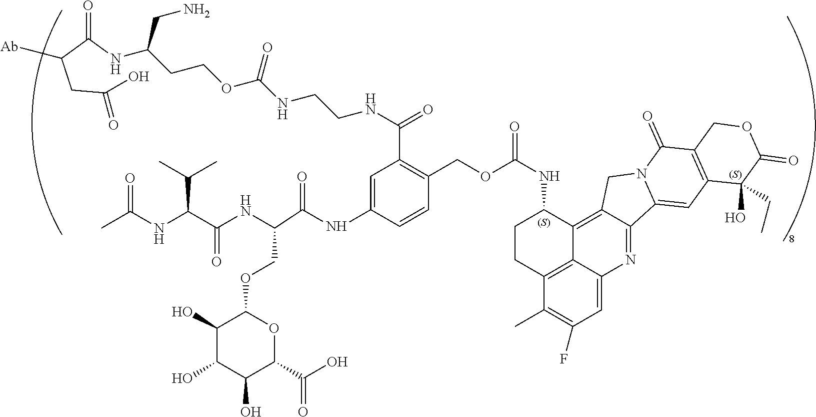 | 9.647 |
| 3-14 | 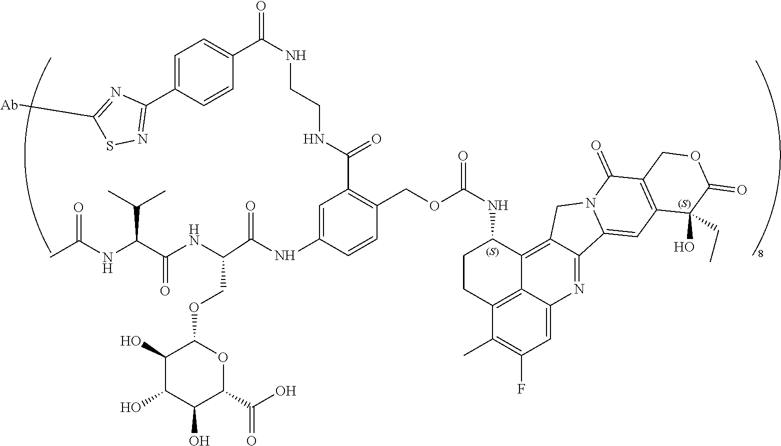 | 9.708 |
| 3-B | 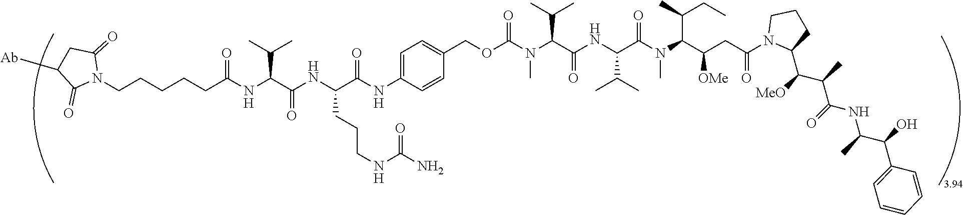 | 14.537 |
| 4-1 | 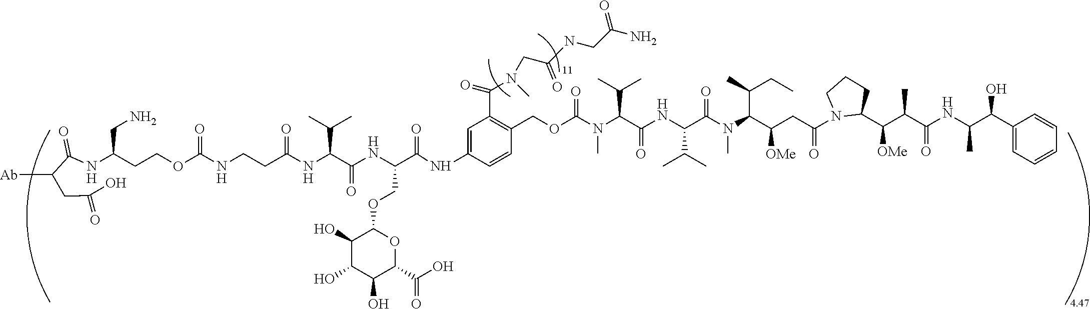 | 11.838 |
| 4-2 | 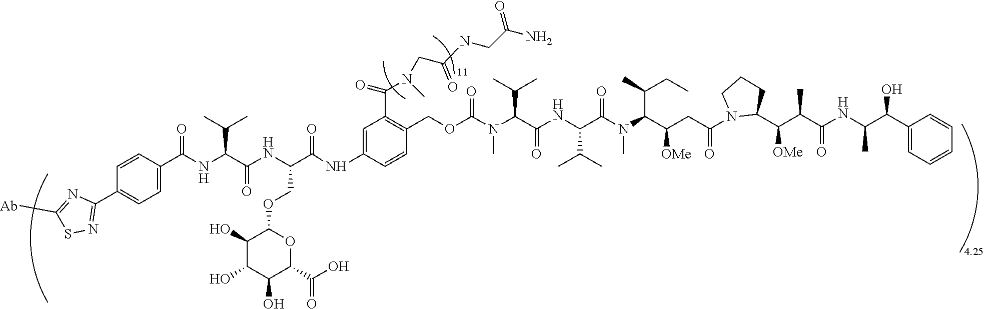 | No data |
| 4-3 | 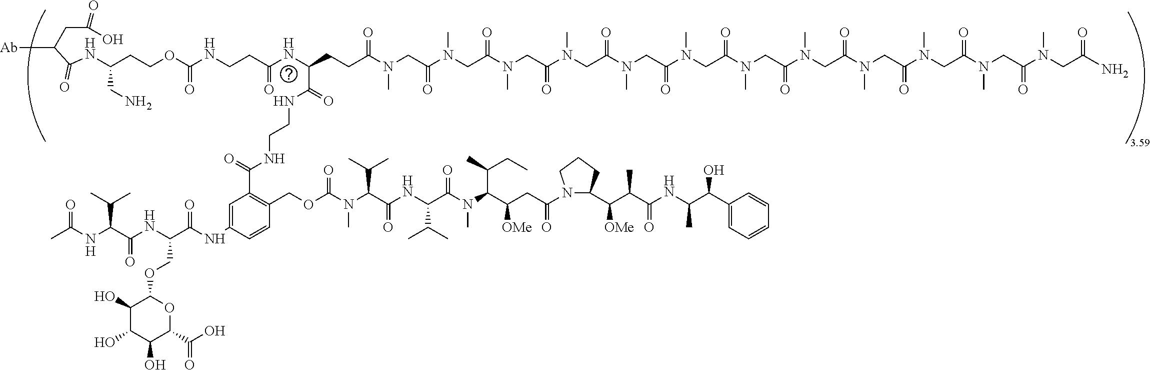 | 12.189 |

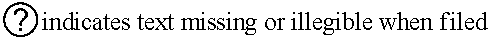

TABLE 2D
ADCs Prepared with Cofetuzumab as ″Ab″

| ADC | | DAR8 |
|---|---|---|
| No. | ADC Structure | RT (min) |
| 5-A | 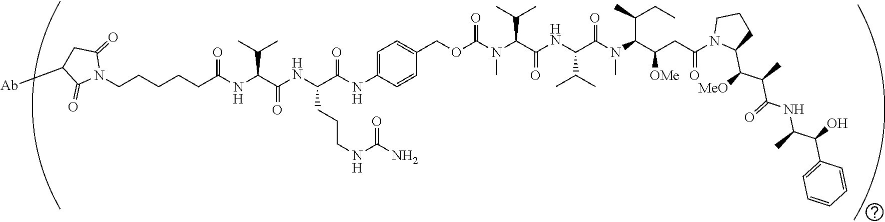 | 13.595 |
| 5-1 | 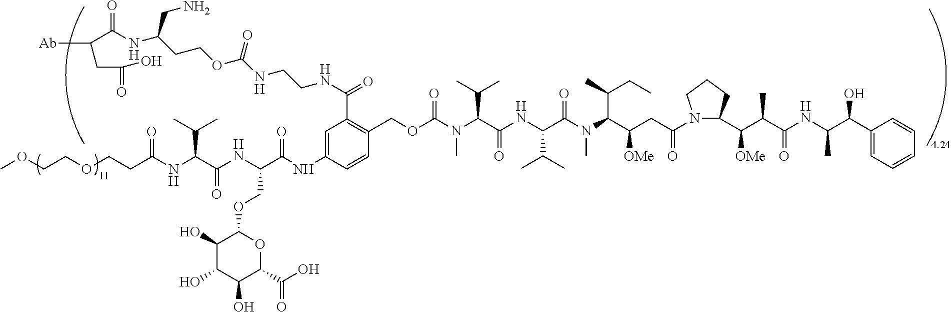 | 12.682 |
| 5-2 | 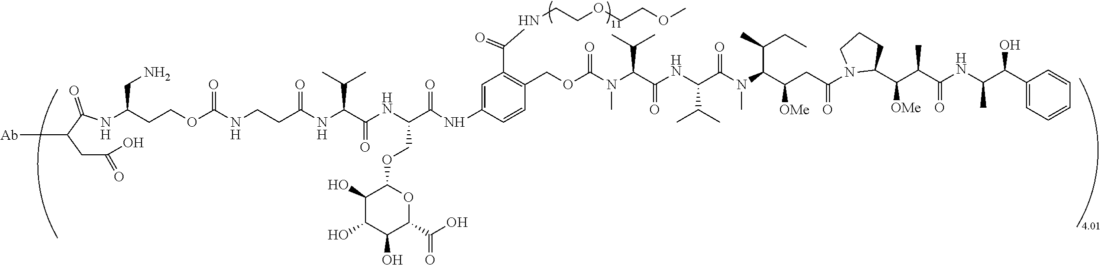 | 12.58 |

### Antibody Information

### Ifinatamab (Anti-B7H3 Antibody)

Light chain sequence
(SEQ ID NO: 1)
EIVLTQSPATLSLSPGERATLSCRASSRLIYMHWYQQKPGQAPRP
LIYATSNLASGIPARFSGSGSGTDFTLTISSLEPEDFAVYYCQQW
NSNPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
Heavy chain sequence
(SEQ ID NO: 2)
QVQLVQSGAEVKKPGSSVKVSCKASGYTFTNYVMHWVRQAPGQGL
EWMGYINPYNDDVKYNEKFKGRVTITADESTSTAYMELSSLRSED
TAVYYCARWGYYGSPLYYFDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKRVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK
6E7 (anti-CLL1 antibody)
Light chain sequence
(SEQ ID NO: 3)
DIQMTQSPSSLSASVGDRVTITCRASQSVSTSSYNYMHWYQQKPG
KPPKLLIKYASNLESGVPSRFSGSGSGTDFTLTISSLQPEDFATY
YCQHSWEIPLTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy chain sequence
(SEQ ID NO: 4)
EVQLVQSGAEVKKPGASVKVSCKASGYSFTDYYMHWVRQAPGQGL
EWIGRINPYAGAAFYSQNFKDRVTLTVDTSTSTAYLELSSLRSED
TAVYYCAIERGADLEGYAMDYWGQGTLVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK

### Cofetuzumab (Anti-PTK7 Antibody)

Light chain sequence
(SEQ ID NO: 5)
DIEMTQSPDSLAVSLGERATINCRSSQSVLYSSSNRNYLAWYQQN
PGQPPKLLIYWASTRESGVPDRFSGSGSGTDFTLTISSLQAEDVA
VYYCQQYYSTPRTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
Heavy chain sequence
(SEQ ID NO: 6)
QVQLVQSGPEVKKPGASVKVSCKASGYTFTDYAVHWVRQAPGKRL
EWIGVISTYNDYTYNNQDFKGRVTMTRDTSASTAYMELSRLRSED
TAVYYCARGNSYFYALDYWGQGTSVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK

### Example 3: ADC Direct Killing in U937, HL60, TF-1, NCI-H358, NCI-H1048, and MDA-MB-453 Cancer Lines

### Cell Lines

U937 (ATCC, CRL-1593.2). U-937 is a cell line exhibiting monocyte morphology that was derived in 1974 from malignant cells obtained from the pleural effusion of a 37-year-old white male with histiocytic lymphoma. U937 was purchased from ATCC. The base medium for U937 is ATCC-formulated RPMI-1640 Medium (ATCC 30-2001). To make the complete growth medium, fetal bovine serum to a final concentration of 10% (Gibco, 10099-141 C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

HL60 (ATCC, CCL-240). HL-60 cells are promyeoloblasts isolated from the peripheral blood by leukopheresis from a 36-year-old white female with acute promyelocytic leukemia. HL60 was purchased from ATCC. The bas' me'ium for HL60 is ‘AT’C-formulated Iscove's Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

TF1 (ATCC, CRL-2003). TF-1 erythroblasts were isolated in 1987 from bone marrow derived from a 35-year-old Asian male with severe pancytopenia. TF-1 was purchased from ATCC. The base medium for TF-1 is ATCC-formulated RPMI-1640 Medium, Catalog No. 30-2001. To make the complete growth medium, fetal bovine serum to a final concentration of 10% (Gibco, 10099-141 C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

NCI-H358 (ATCC, CRL-5807). NCI-H358 cells are epithelial-like cells isolated from the bronchiole of a male patient with bronchioalveolar carcinoma. NCI-H358 was purchased from ATCC. The base medium for NCI-H358 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

NCI-H1048 (ATCC, CRL-5853). NCI-H1048 is a cell line exhibiting epithelial morphology. NCT-H1048 was purchased from ATCC. The base medium for NCI-H1048 is ATCC-formulated DMEM:F12 (Catalog No. 30-2006). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

MDA-MB-453 (SIBS). MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MDA-MB-453 is RPMI 1640 Medium, HEPES (Gibco, 22400105). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAlert™ PLUS Mycoplasma Detection Kit (Lonza, LT07-710).

TABLE 3
CLL1 expression level

| | FACS | |
|---|---|---|
| | (Emax MFI, | Molecular No. |
| Cell line | 100 nM) | (QSC kit) |
| U937 | 1607 | 84858 |
| HL60 | 846 | NA |
| TF-1 | 50 | <5806 |

TABLE 4
B7H3 expression level

| | Molecular No. |
|---|---|
| Cell line | (QSC kit) |
| NCI-H358 | 603012 |
| NCI-H1048 | 100687 |
| MDA-MB-453 | <5806 |

ADC direct killing ADC direct killing was assessed in U937, HL60, and TF1 cancer lines. Cells were seeded (U937 or HL60 (3E3/well) or TF1 (6E3/well)) into 2D 96-well plates (Greiner: 655090), 100 μl/well (including 150 μg/ml Fc blocker), and incubated at 37° C., 5% CO₂, for 1 h. Fresh growth medium was added containing varying concentrations of ADCs, 50 μl/well, and incubated at 37° C., 5% CO₂, for 6 days. The cell viability was detected by Cell Titer-Glo (Promega, G7573), 70 μl/well. The 2D plates were allowed to incubate at room temperature for 10 minutes to stabilize the luminescent signal. The plates were analyzed with a microplate reader.

ADC direct killing was assessed in NCI-H358, NCI-H1048, and MDA-MB-453 cancer lines. Cells were seeded (2E3/well) into 3D 96-well plates (Corning: 4520), 80 μl/well, and incubated at 37° C., 5% CO₂, overnight. Fresh growth medium was added containing varying concentrations of ADCs, 40 μl/well, and incubated at 37° C., 5% CO₂, for 6 days. The cell viability was detected by 3D reagent (Promega, G9683), 100 μl/well. The 3D plates were allowed to incubate at room temperature for 30 minutes to stabilize the luminescent signal. The plates were analyzed with a microplate reader.

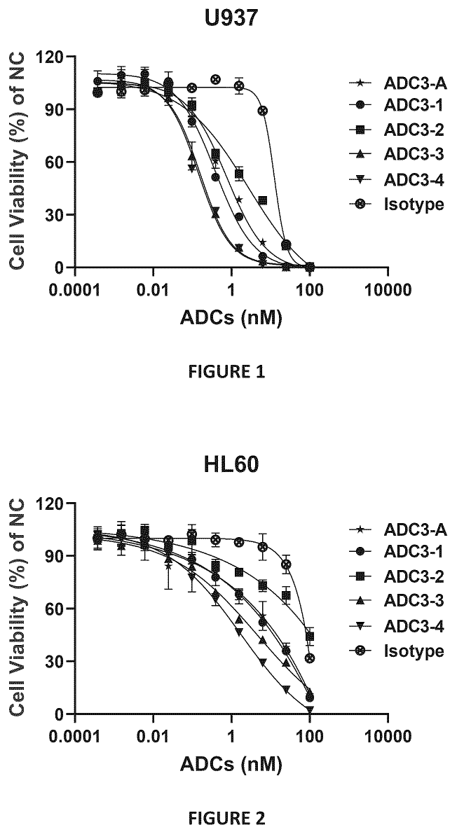

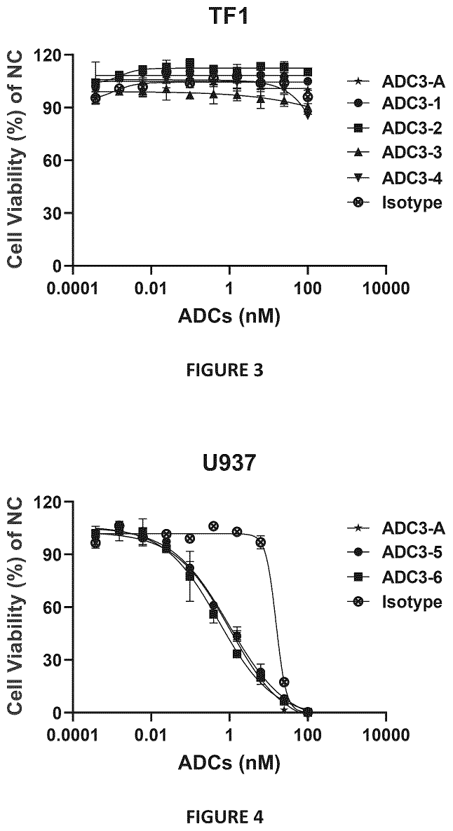

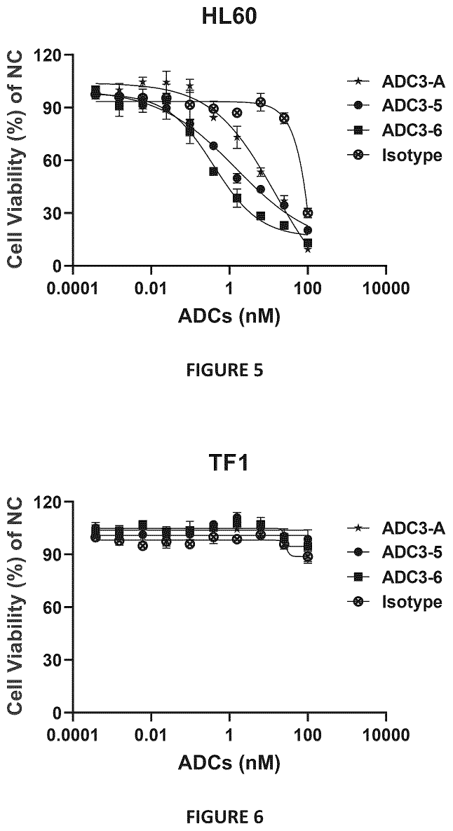

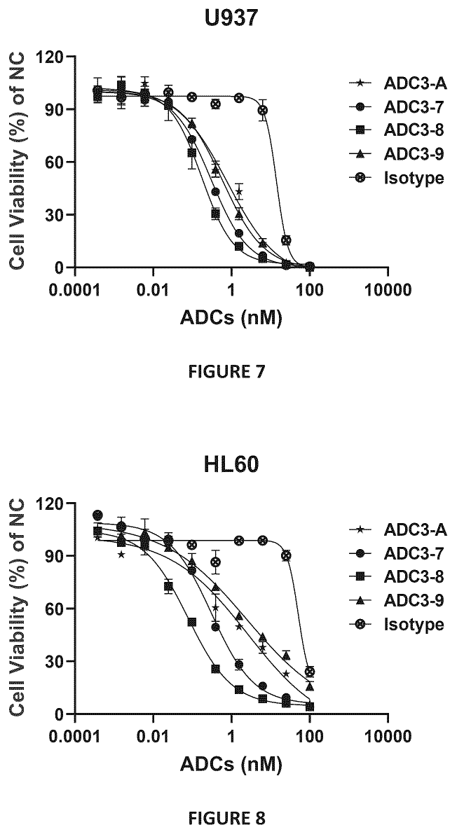

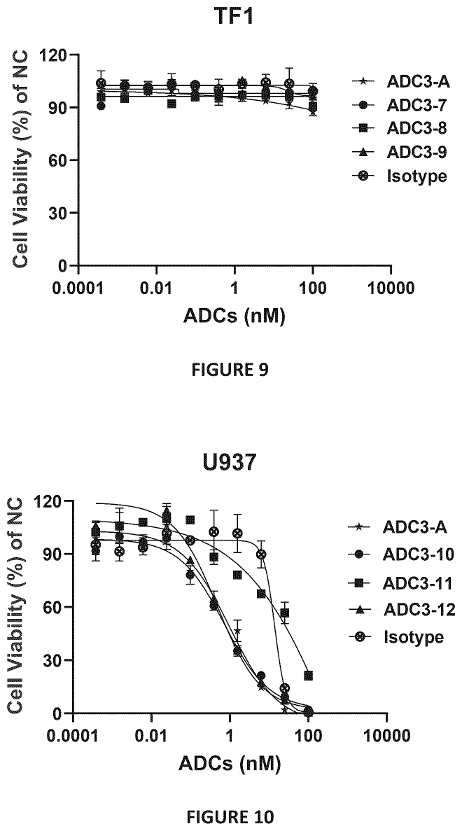

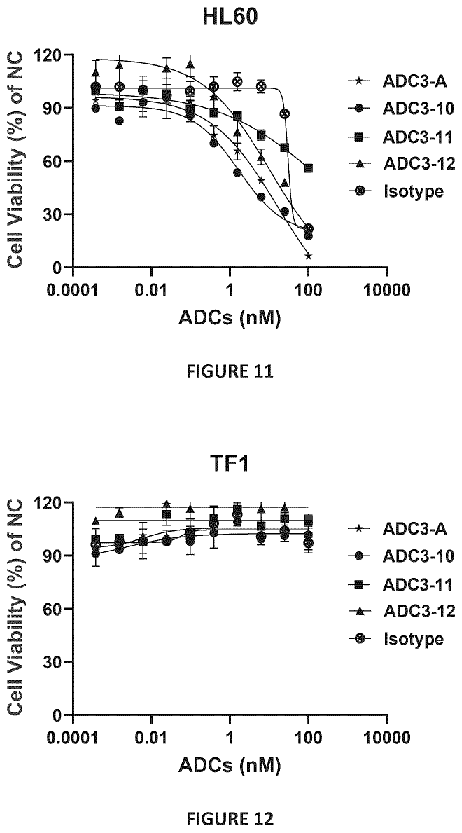

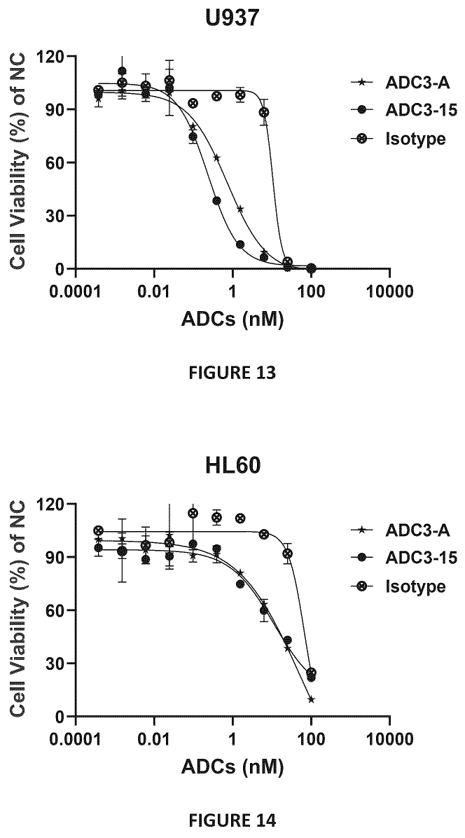

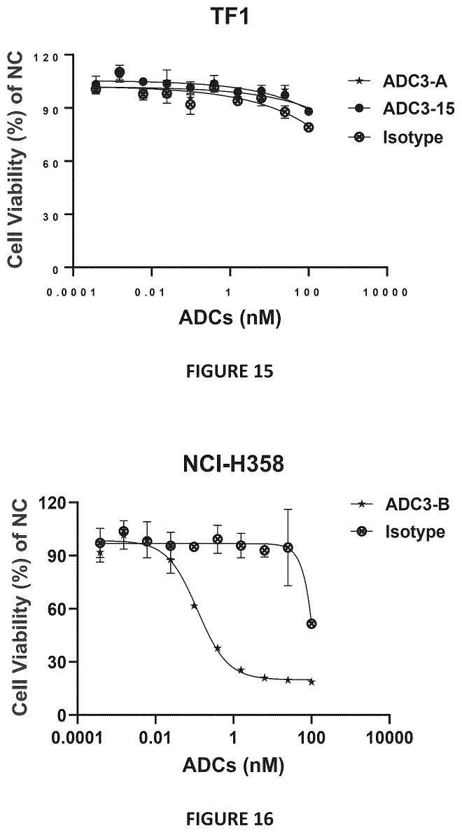

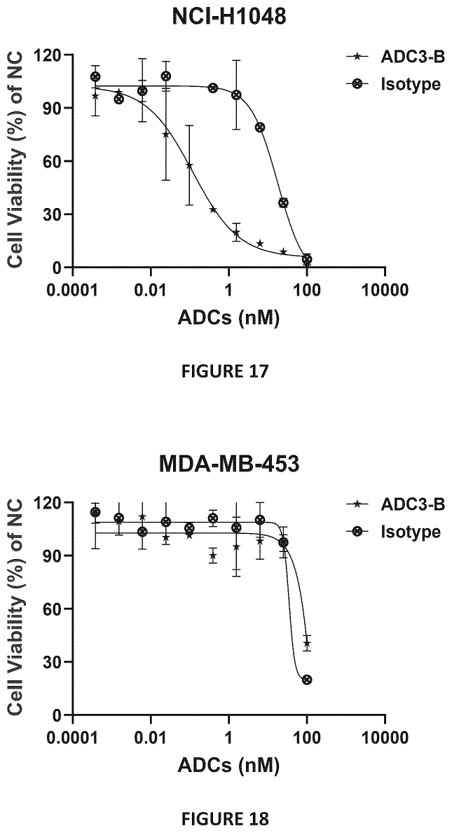

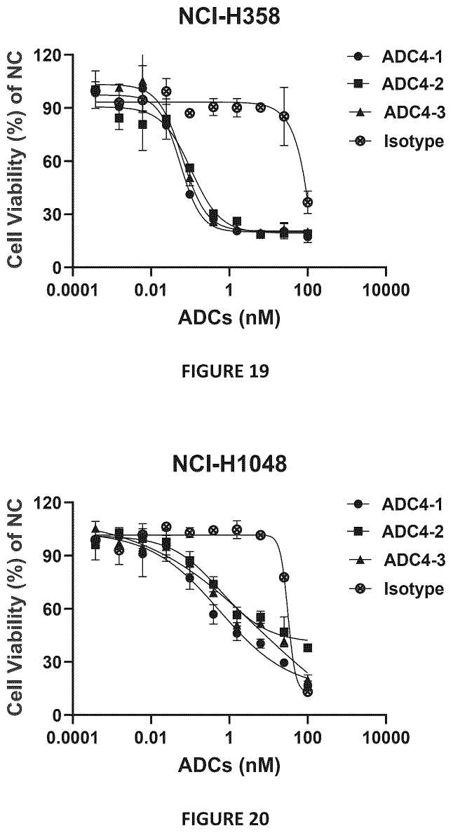

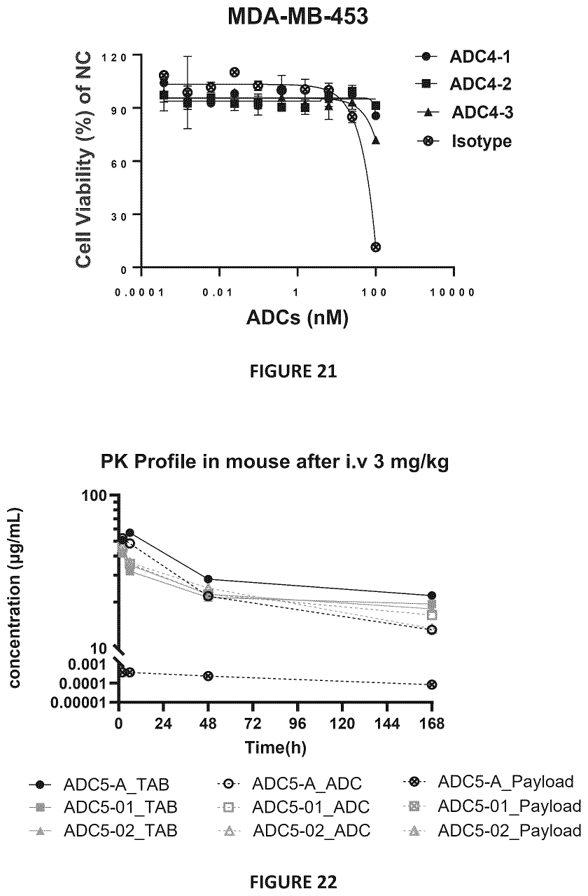

ADC direct cellular killing data are presented in FIGS. 1-15 and Tables 5-9 for U937, HL60, and TF1 cells. ADC direct cellular killing data are presented in FIGS. 16-21 and Tables 10 and 11 for NCI-H358, NCI-H1048, and MDA-MB-453 cells.

TABLE 5
ADC direct cellular killing on CLL1-expressing cells

| | |
|---|---|
| U937 | HL60 |

| | Emax | EC50 | Emax | EC50 |
|---|---|---|---|---|
| ADCs | (%) | (nM) | (%) | (nM) |
| ADC3-A | 99.9 | 0.8 | 89.0 | 6.3 |
| ADC3-1 | 99.9 | 0.5 | 90.8 | 5.7 |
| ADC3-2 | 99.5 | 0.2 | 55.9 | 8.2 |
| ADC3-3 | 99.9 | 0.2 | 87.5 | 1.6 |
| ADC3-4 | 99.9 | 0.2 | 98.0 | 1.1 |

TABLE 6
ADC direct cellular killing on CLL1-expressing cells

| | |
|---|---|
| U937 | HL60 |

| | Emax | EC50 | Emax | EC50 |
|---|---|---|---|---|
| ADCs | (%) | (nM) | (%) | (nM) |
| ADC3-A | 99.9 | 0.9 | 90.7 | 6.5 |
| ADC3-5 | 99.8 | 1.0 | 79.8 | 0.9 |
| ADC3-6 | 99.8 | 0.6 | 86.9 | 0.4 |

TABLE 7
ADC direct cellular killing on CLL1-expressing cells

| | |
|---|---|
| U937 | HL60 |

| | Emax | EC50 | Emax | EC50 |
|---|---|---|---|---|
| ADCs | (%) | (nM) | (%) | (nM) |
| ADC3-A | 99.9 | 0.7 | 94.0 | 1.4 |
| ADC3-7 | 99.9 | 0.3 | 96.0 | 0.4 |
| ADC3-8 | 99.8 | 0.2 | 95.8 | 0.1 |
| ADC3-9 | 99.9 | 0.6 | 84.1 | 1.7 |

TABLE 8
ADC direct cellular killing on CLL1-expressing cells

| | |
|---|---|
| U937 | HL60 |

| | Emax | EC50 | Emax | EC50 |
|---|---|---|---|---|
| ADCs | (%) | (nM) | (%) | (nM) |
| ADC3-A | 99.8 | 0.9 | 93.6 | 4.8 |
| ADC3-10 | 98.1 | 0.7 | 82.2 | 1.2 |
| ADC3-11 | 78.7 | 12.8 | 43.9 | 4.6 |
| ADC3-12 | 99.2 | 0.7 | 81.0 | 8.0 |

TABLE 9
ADC direct cellular killing on CLL1-expressing cells

| | |
|---|---|
| U937 | HL60 |

| | Emax | EC50 | Emax | EC50 |
|---|---|---|---|---|
| ADCs | (%) | (nM) | (%) | (nM) |
| ADC3-A | 99.9 | 0.7 | 90.5 | 10.9 |
| ADC3-15 | 99.9 | 0.3 | 78.1 | 6.5 |

TABLE 10
ADC direct cellular killing on B7H3-expressing cells

| | |
|---|---|
| NCI-H358 | NCI-H1048 |

| | Emax | EC50 | Emax | EC50 |
|---|---|---|---|---|
| ADCs | (%) | (nM) | (%) | (nM) |
| ADC3-B | 81.3 | 0.1 | 98.1 | 0.2 |

TABLE 11
ADC direct cellular killing on B7H3-expressing cells

| | |
|---|---|
| NCI-H358 | NCI-H1048 |

| | Emax | EC50 | Emax | EC50 |
|---|---|---|---|---|
| ADCs | (%) | (nM) | (%) | (nM) |
| ADC4-1 | 82.8 | 0.1 | 83.6 | 0.6 |
| ADC4-2 | 81.0 | 0.1 | 62.0 | 0.7 |
| ADC4-3 | 79.9 | 0.1 | 80.0 | 1.6 |

### Example 4: ADC PK Studies in Mice

Female BALB/c nude mice (n==3 per group) were treated with freshly prepared ADCs (5 or 10 mg/kg) through intravenous administration. At the designated time points after dosing, mice were anesthetized by isoflurane. Whole blood samples were collected from orbital venous sinuses into coagulation tubes (Kangjian, #041-0121) at 0.5 h, 2 h, 6h, 8 h, 24 h, 48h, 72h, and 168 h post-dosing. Blood samples were placed at room temperature for about 30 minutes and processed by centrifugation (4° C., 3000×g, 7 min) to separate plasma. Serum samples were transferred into new 1.5 mL tubes and kept in a −80° C. freezer prior to analysis.

Serum payload concentrations. To determine serum payload concentrations, an aliquot of 15 μL sample was added with 150 μL IS (Verapamil, 10 ng/mL) in ACN/MeOH (1:1). The mixture was vortexed for 1 min and centrifuged at 4000 rpm for 10 min at 4° C. An aliquot of 80 μL supernatant was diluted with 80 μL, water, and the mixed sample was injected into LC-MS/MS.

LC-MS/MS method to determine payload bioanalysis. LC-MS/MS analysis was carried out under the following measurement conditions:
Instrument: LC-MS/MS (Triple Quad 6500 plus)
Monitor: MRM
Column: Advanced Materials Technology, HALO AQ-C18 2.7 μm 90 Å, 50*2.1 mm
Column temperature: 40° C.
canile phase A: H₂O-0.1% FA
Mobile phase B: ACN-0.1% FA
Gradient program for MMAE: 15% B-15% B (0 min-0.4 min), 15% B-30% B (0.4 min-0.8 min), 30% B-30% B (0.8 min-1.8 min), 30% B-90% B (1.8 min-1.9 min), 90% B-90% B (1.9 min-2.4 min), 90% B-15% B (2.4 min-2.5 min), 15% B-15% B (2.5 min-3.0 min)
Injected sample amount: 10 μL (MMAE)

Plasma ADC and total Ab (Tab) concentrations. Plasma ADC and Tab concentrations were determined using an ELISA assay in which the capture reagent was CLL1 or PTK7 (as applicable) ECD (extracellular domain). The detection reagent was anti-payload Ab for ADC concentration and anti-human IgG polyclonal Ab for Tab concentration.

For ADC concentration, microplates were coated with CLL1 or PTK7, as applicable, ECD at 1 μg/mL, 100 μL per well and incubated overnight at 4° C. The microplates were washed 3 times with 0.05% PBST, then 200 μL 2% BSA solution was added per well. The plates were incubated at 37° C. for 1 h followed by washing 3 times with 0.05% PBST. Then 100 μL (of ADC samples or for standard curves) were added to each well. Plates were incubated at 37° C. for 1 h followed by washing 3 times with 0.05% PBST. 100 μL of 1 μg/ml biotin anti-MMAE antibody was added per well. Plates were again incubated at 37° C. for 1 h and then washed 3 times with 0.05% PBST. Then 100 μl of 1:10000 SA-HRP was added per well, incubated at 37° C. for 0.5h, followed by washing 3 times with 0.05% PBST. Lastly, 100 μL TMB was added per well and plates were incubated for 15 mins at room temperature before the addition of 100 μL stop solution per well. Plates were read at OD 450 on an ELISA microplate reader.

For Tab concentration of anti-CLL1 antibodies, the foregoing ADC method was used except for the following changes: 100 μL of anti human lgG Fc HRP (1:10000) was added per well.

For Tab concentration of anti-PTK1 antibodies, the foregoing ADC method was used except for the following changes: 100 μL of 1 μg/ml anti human lgG Fc HRP (1:10000) was added per well.

ADC DAR changes in mouse serum PK samples. ADC stability as reflected in DAR decreases over time was studied using the plasma samples described above. The post-dosing timepoints of the samples tested are indicated in Tables 16 and 17 below.

Human CLL1 or PTK7, as applicable, ECD was biotinylated and immobilized onto Dynabeads M-280 Streptavidin. ADCs were captured from the plasma samples by incubation with an ECD-bead system for 2 hours at 37° C. The captured ADCs were then washed with HBS-EP buffer (10 mM Hepes [pH 7.4], 150 mM NaCl, 3.4 mM ethylenediaminetetraacetic acid [EDTA], 0.005% Surfactant P20) and digested using IdeS enzyme at 37° C. for 1 h. After extensive washing of the beads with HBS-EP, water, and 10% acetonitrile, the ADC analytes were eluted using 30% acetonitrile with 1% formic acid. The samples were then reduced with 100 mM TCEP for 45 min. Liquid chromatography-tandem mass spectrometry (LC-MS/MS) methods were used for the ADC DAR analysis.

Mouse PK parameters of some anti-CLL1 ADCs (ADC3-A, ADC3-3, ADC3-5, ADC3-6, and ADC3-7) are presented in Table 12 below. The results demonstrate that ADC3-3, ADC3-5, ADC3-6, and ADC3-7 exhibited lower payload exposure and lower DAR changes than did ADC3-A.

TABLE 12
Mouse PK parameters of anti-CLL1 ADCs

| | | Tab | ADC | Payload | | |
|---|---|---|---|---|---|---|
| | | AUCₗₐₛₜ | AUCₗₐₛₜ | AUCₗₐₛₜ | | |
| | Dose | (h · | (h · | (h · | | |
| ADC No. | (mg/kg) | μg/mL) | μg/mL) | μg/mL) | DAR | |
| ADC3-A | 5 | 6448 | 4353 | 8.58 | 2.76 | (0.5 h) |
| | | | | | 1.96 | (72 h) |
| | | | | | 1.22 | (168 h) |
| ADC3-3 | 5 | 4923 | 4229 | NA | 3.48 | (2 h) |
| | | | | | 2.82 | (168 h) |
| ADC3-5 | 5 | 8051 | 8380 | NA | 2.94 | (2 h) |
| | | | | | 2.34 | (168 h) |
| ADC3-6 | 5 | 7965 | 6969 | NA | 2.96 | (2 h) |
| | | | | | 2.24 | (168 h) |
| ADC3-7 | 10 | 12900 | 13175 | NA | 3.60 | (2 h) |
| | | | | | 3.36 | (168 h) |

NA: not available (because limited concentration for AUC calculation as payload concentration below quantitation limit (lowest limit of quantitation = 50 pg/mL))

Mouse PK parameters of anti-PTK7 ADCs (ADC5-A, ADC5-1, and ADC5-2) are presented in Table 13 and FIG. 22. The results demonstrate that ADC5-1 and ADC5-2 exhibited lower payload exposure and lower DAR changes than did ADC5-A.

TABLE 13
Mouse PK parameters of anti-PTK7 ADCs

| | | Tab | ADC | Payload | | |
|---|---|---|---|---|---|---|
| | | AUCₗₐₛₜ | AUCₗₐₛₜ | AUCₗₐₛₜ | | |
| | Dose | (h · | (h · | (h · | | |
| ADC No. | (mg/kg) | μg/mL) | μg/mL) | μg/mL) | DAR | |
| ADC5-A | 3 | 5129 | 3890 | 0.033 | 3.38 | (2 h) |
| | | | | | 2.02 | (168 h) |
| ADC5-1 | 3 | 3816 | 3818 | NA | 3.42 | (2 h) |
| | | | | | 2.66 | (168 h) |
| ADC5-2 | 3 | 3903 | 3846 | NA | 2.80 | (2 h) |
| | | | | | 2.42 | (168 h) |

NA: not available (because limited concentration for AUC calculation as payload concentration below quantitation limit (lowest limit of quantitation = 50 pg/mL))

### Example 5: Tumor Growth Inhibition in NCI-H1650 Xenograft Model

NCI-H1650 (ATCC) is a cell line exhibiting epithelial morphology that was isolated in 1987 from the lung tissue of a 27-year-old male smoker with stage 3 bronchoalveolar carcinoma. NCI-H1650 cells were cultured in RPMI-1640 medium supplemented with 10% (v/v) fetal bovine serum, 100 U/mi penicillin and 100 μg/mL streptomycin. On the day of implantation, cultured cells were collected and re-suspended in a cold (4° C.) mixture of PBS and Matrigel (1:1). Cell density was adjusted to 2.5×10⁷ cells/mL and the cells were placed on ice prior to inoculation.

Female BALB/c nude mice were subcutaneously injected with 5×10⁶ NCI-H1650 cells (mixed with Matrigel at a ratio of 1:1) in the right flank. After inoculation, tumor length (a) and width (b) were measured with calipers and tumor volumes were calculated using the formula V=0.5 (a×b²). When tumor volumes reached approximately 200 mm³ in size, mice were randomized into 4 groups with 6 animals in each of a vehicle, ADC5-A, ADC5-1, and ADC5-2 group on Day 0. Animals were intravenously administrated with vehicle, ADC5-A (3 mg/kg), ADC5-1 (3 mg/kg), or ADC5-2 (3 mg/kg) on treatment Day 1. Whole blood was collected at timepoints of 2, 6, 48, and 168 h post-treatment from 3 mice per timepoint per group (samples at 2 and 48 h were collected from one group of 3; samples at 6 and 168 h were collected from another group of 3). Blood was centrifuged at 5000×g for 10 minutes at 4° C. to obtain serum for PK assays. Animal body weight and tumor volume were measured twice weekly. Tumor growth inhibition (TGI) was calculated using the following formula:

$$\% TGI = [ 1 - ( treated {T}_{t} - treated {T}_{0} ) / ( vehicle {T}_{t} - vehicle {T}_{0} ) ] \times 100 \%$$

treated Tₜ=mean tumor volume of a dosing group on Day t
treated T₀=mean tumor volume of a dosing group on Day 0
vehicle Tₜ=mean tumor volume of vehicle group on Day t
vehicle T₀=mean tumor volume of vehicle group on Day 0

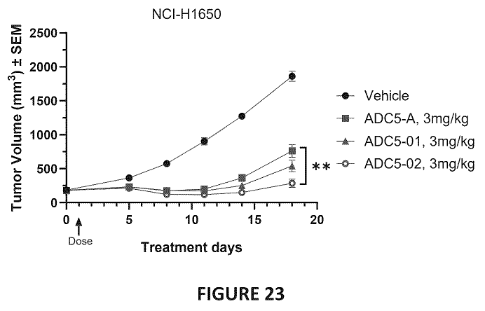

Results of evaluating the anti-tumor effect of ADC5-A, ADC5-1, and ADC5-2 in a subcutaneous NCI-H1650 xenograft model are presented in Table 14 and FIG. 23. Data is presented as mean tumor volume±standard error of the mean (SEM). Single dose treatment with 3 mg/kg ADC5-A, ADC5-1, and ADC5-2 exhibited anti-tumor activity with a TGI of 65% (adjusted p=0.0008), 78% (adjusted p=0.0007) and 94% (adjusted p=0.0012), respectively, on treatment Day 18. ADC5-1 and ADC5-2 showed better efficacy than did ADC5-A at 3 mg/kg. ADCs in all treatment groups were well tolerated and animals in all treatment groups showed no significant body weight decrease or abnormal clinical observation.

TABLE 14
TGI measurement of ADC5-A, ADC5-1,
and ADC5-2 on treatment Day 18

| ADC | TGI (%) on D18 |
|---|---|
| ADC5-A, 3 mg/kg | 65 |
| ADC5-1, 3 mg/kg | 78 |
| ADC5-2, 3 mg/kg | 94 |

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any prior art publication is referred to herein, such reference does not constitute an admission that the publication forms a part of the common general knowledge in the art in any country.

The disclosures of all non-patent publications, patents, patent applications, and published patent applications referred to herein by an identifying citation are hereby incorporated herein by reference in their entireties.

1. A compound of formula (Ia): 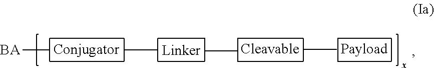
or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III): 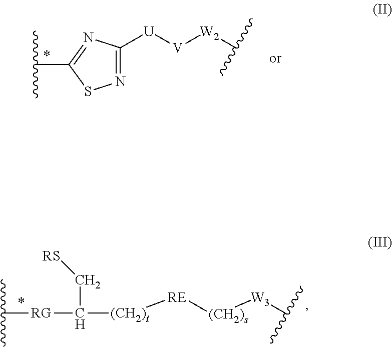
wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is 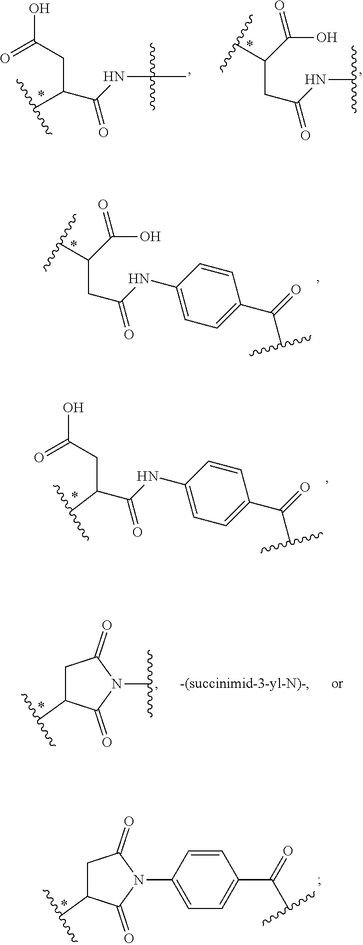 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]
RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 1 or 2; [symbol]
indicates a point of covalent attachment within the compound, and
* marks the bond where Conjugator connects to BA;
Linker has formula (IV): 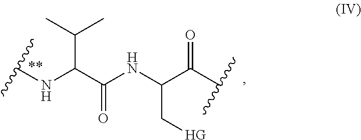
wherein:
HG is a hydrophilic residue, and
** marks the bond where Linker connects to Conjugator;
Cleavable has formula (V): 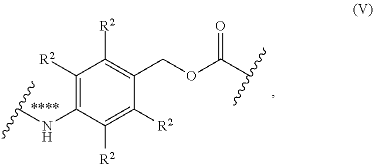
wherein:
each R² is independently hydrogen, a halogen, substituted or unsubstituted C₁₋₄ alkyl, —CN, or —NO₂, and
**** marks the bond where Cleavable connects to Linker;
Payload is a payload residue; and
x is from 1 to 15 inclusive.

2. The compound of claim 1, or a pharmaceutically acceptable salt, tautomer, solvate, or stereoisomer thereof, wherein Cleavable has the following formula:

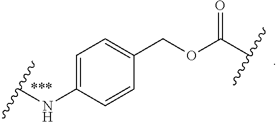 [SMILES: *Nc1ccc(COC(*)=O)cc1]

3. A compound of Formula (Ib): 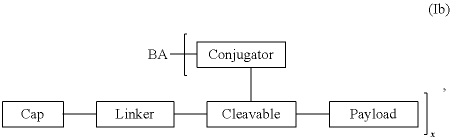
or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III): 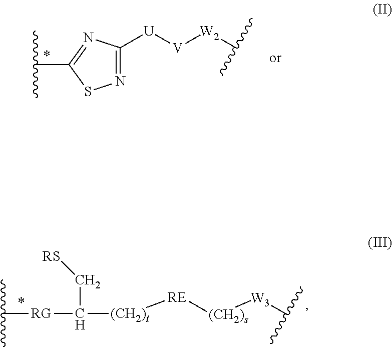
wherein:
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is 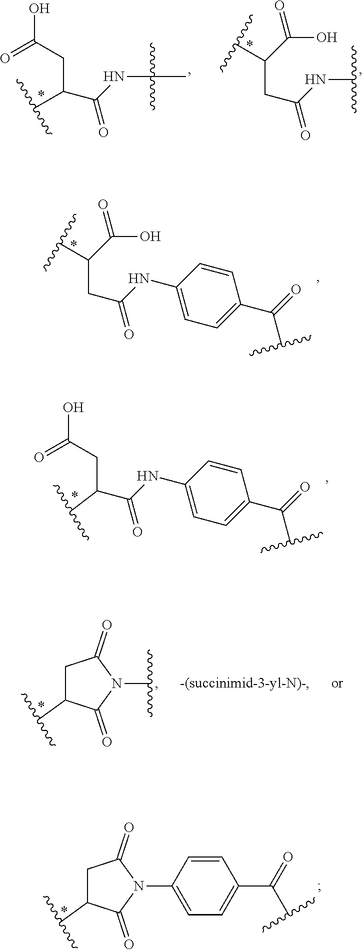 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]
RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Cleavable has formula (Va), (Vb), or (Vc): 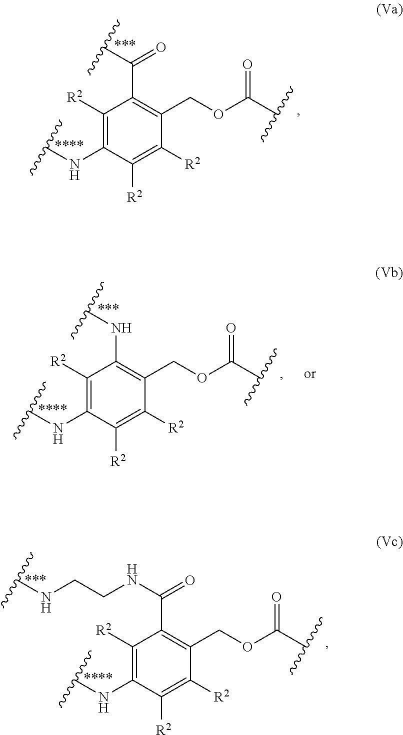
wherein:
each R² is, independently, hydrogen, halogen, substituted or unsubstituted C₁₋₄ alkyl, —CN, or —NO₂,
*** marks the bond where Cleavable connects to Conjugator, and
**** marks the bond where Cleavable connects to Linker;
Linker has formula (IV′): 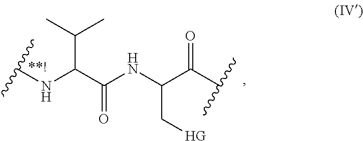
wherein
HG is a hydrophilic residue, and
**! marks the bond where Linker connects to Cap;
Cap is CH₃C(═O)—, CH₃—(CH₂)_{b}—O—(CH₂CH₂O)ₐ—(CH₂CH₂)_{d}—C(═O)—, CH₃—(CH₂)_{b}—O—(CH₂CH₂O)ₐ—(CH₂CH₂)_{d}—NH—C(═O)—, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂, wherein:
a is an integer between 1 and 18 inclusive,
each of b and d is, independently, 0, 1, or 2, and
c is an integer between 1 and 4 inclusive;
Payload is a payload residue; and
x is from 1 to 15 inclusive.

4-6. (canceled)

7. A compound of Formula (Ic): 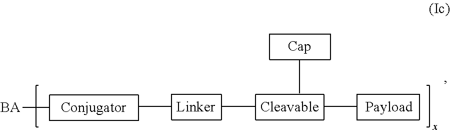
or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III): 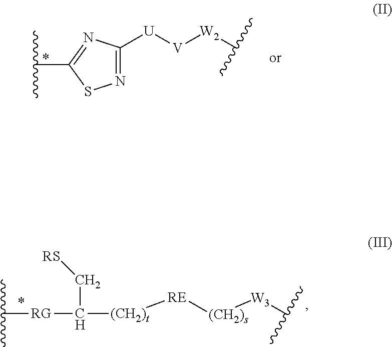
wherein;
U is a bond, heteroarylene, or arylene;
V is a bond or —C≡C—(CH₂)ₙ—;
n is an integer between 0 and 10 inclusive;
W₂ is —C(═O)—, —NH—, or —O—;
RG is 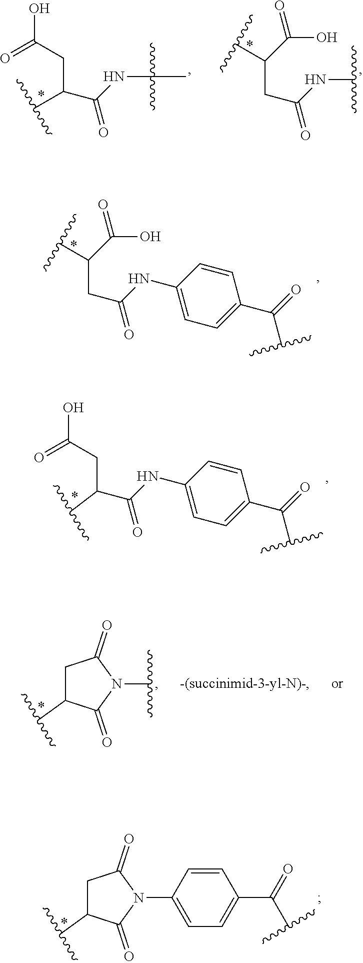 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]
RS is an amino group or —NR^{1a}R^{b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Linker has formula (IV): 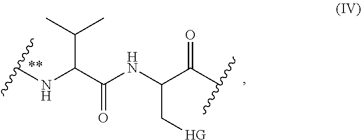
wherein:
HG is a hydrophilic residue, and;
** marks the bond where Linker connects to Conjugator;
Cleavable has formula (Va′), (Vb′), or (Vc′): 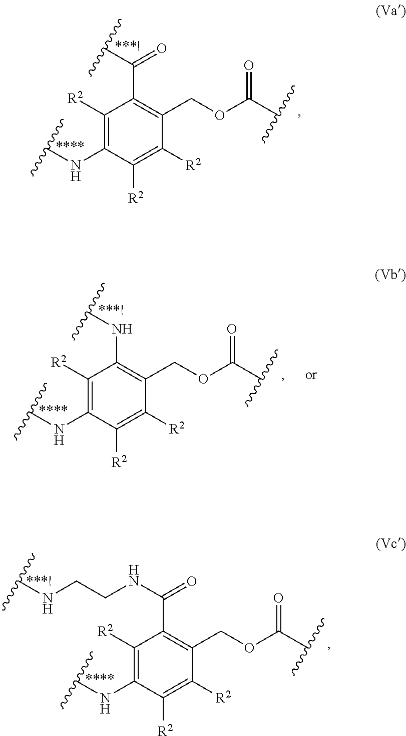
wherein:
each R² is, independently, hydrogen, halogen, substituted or unsubstituted C₁₋₄ alkyl, —CN, or —NO₂,
***! marks the bond where Cleavable connects to Cap, and
**** marks the bond where Cleavable connects to Linker;
Cap is CH₃C(═O)—, CH₃—(CH₂)_{b}—O—(CH₂CH₂O)ₐ—(CH₂CH₂)_{d}—C(═O)—, —C(═O)—(CH₂CH₂O)ₐ—(CH₂)_{b}CH₃, CH₃(C(═O)N(CH₃)CH₂)ₐC(═O)—, —NH—(CH₂CH₂O)ₐ—(CH₂)_{b}CH₃, or —(N(CH₃)(CH₂)_{c}C(═O))ₐNH₂;
a is an integer between 1 and 18 inclusive;
each of b and d, independently, is 0, 1, or 2;
c is an integer between 1 and 4 inclusive;
Payload is a payload residue; and
x is from 1 to 15 inclusive.

8-17. (canceled)

18. The compound of claim 1, or a pharmaceutically acceptable salt, tautomer, solvate, or stereoisomer thereof, wherein Conjugator has formula (II).

19-20. (canceled)

21. The compound of claim 18, or a pharmaceutically acceptable salt, tautomer, solvate, or stereoisomer thereof, wherein U is 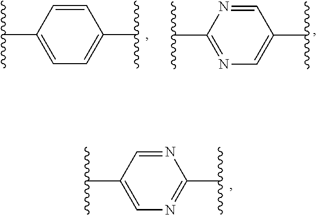
or a bond.

22-25. (canceled)

26. The compound of claim 18, or a pharmaceutically acceptable salt, tautomer, solvate, or stereoisomer thereof, wherein V is a bond or —C≡C—(CH₂)₃—.

27-28. (canceled)

29. The compound of claim 1, or a pharmaceutically acceptable salt, tautomer, solvate, or stereoisomer thereof, wherein W₂ is —C(═O)—.

30. The compound of claim 1, or a pharmaceutically acceptable salt, tautomer, solvate, or stereoisomer thereof, wherein Conjugator has formula (III).

31. The compound of claim 30, or a pharmaceutically acceptable salt, tautomer, solvate, or stereoisomer thereof, wherein RS is —NH₂ or —N(CH₃)₂.

32. (canceled)

33. The compound of claim 30, or a pharmaceutically acceptable salt, tautomer, solvate, or stereoisomer thereof, wherein RE is —OC(═O)NH—.

34. The compound of claim 30, or a pharmaceutically acceptable salt, tautomer, solvate, or stereoisomer thereof, wherein RG is

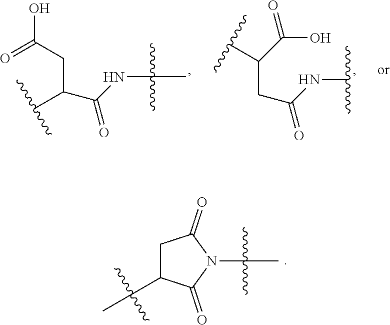 [SMILES: *NC(=O)C(*)CC(=O)O ; *NC(=O)CC(*)C(=O)O ; *C1CC(=O)N(*)C1=O]

35. (canceled)

36. The compound of claim 30, or a pharmaceutically acceptable salt, tautomer, solvate, or stereoisomer thereof, wherein W₃ is —C(═O)—.

37-38. (canceled)

39. The compound of claim 1, or a pharmaceutically acceptable salt, tautomer, solvate, or stereoisomer thereof, wherein HG is 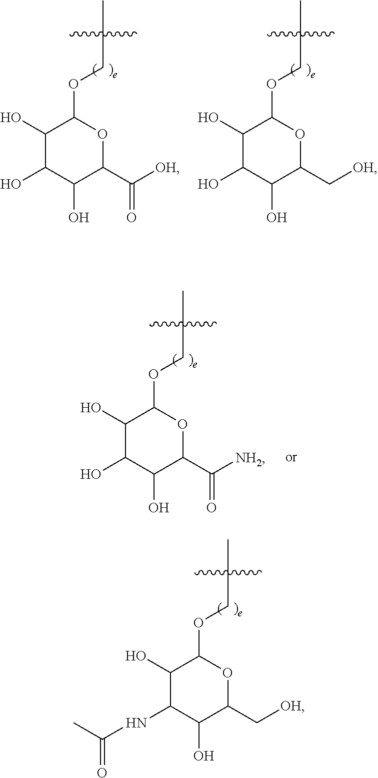
wherein e is 0, 1, or 2; or
HG is —(CH₂CH₂)_{z}—NR^{a}C(O)NR^{b}R^{c}, wherein z is 1, 2, 3, or 4, and each of R^{a}, R^{b}, and R^{c} is, independently, H, or substituted or unsubstituted C₁₋₄ alkyl.

40. (canceled)

41. The compound of claim 1, or a pharmaceutically acceptable salt, tautomer, solvate, or stereoisomer thereof, wherein Payload is a residue of one of the following formulas:

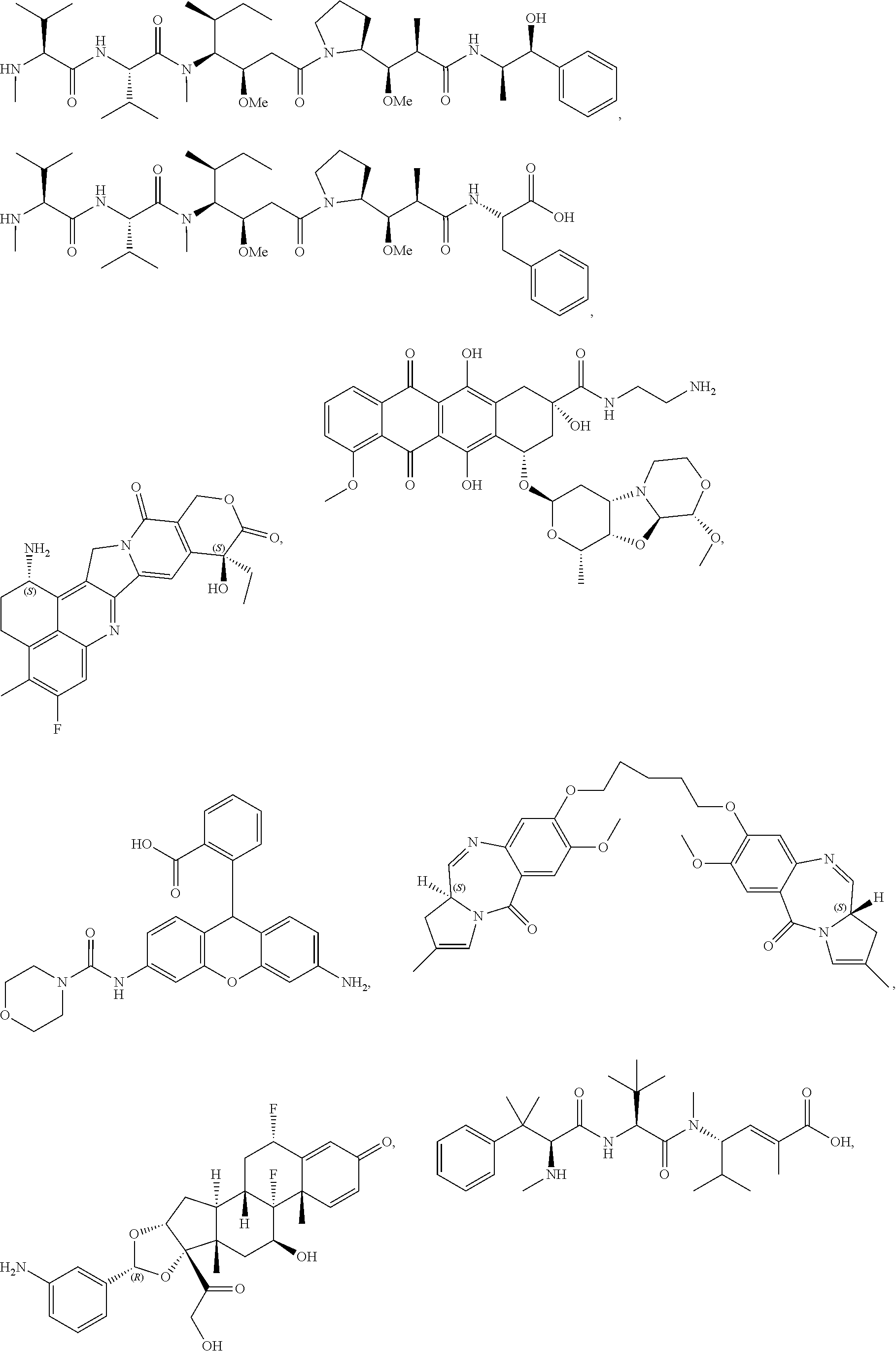

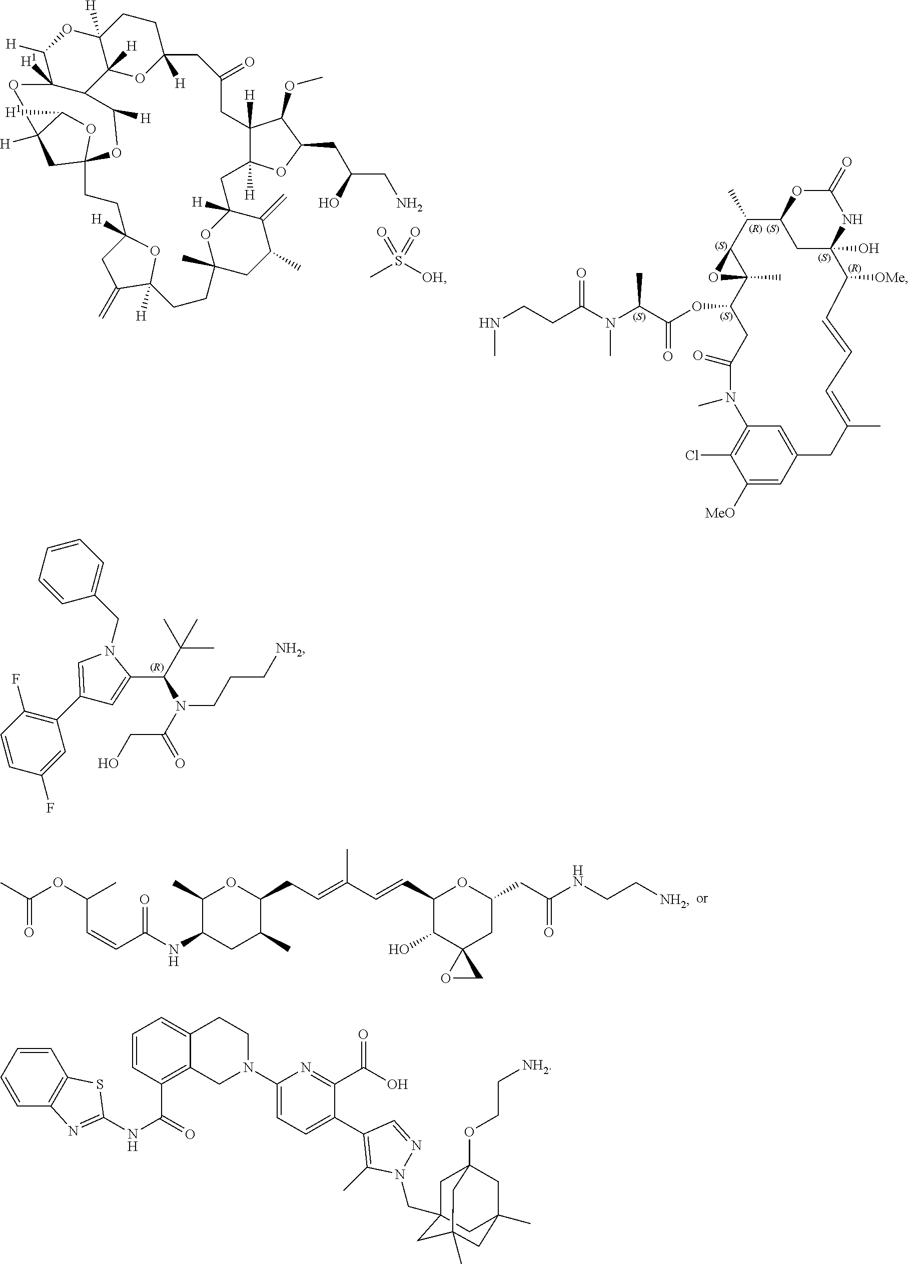

42-43. (canceled)

44. The compound of claim 1, or a pharmaceutically acceptable salt, tautomer, solvate, or stereoisomer thereof, wherein BA is ifinatamab, 6E7, or cofetuzumab, or an antigen-binding fragment of ifinatamab, 6E7, or cofetuzumab.

45. The compound of claim 1, wherein the compound is 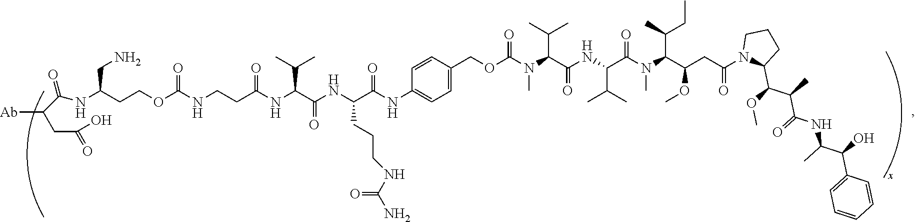
wherein Ab is 6E7 or an antigen-binding fragment thereof; or 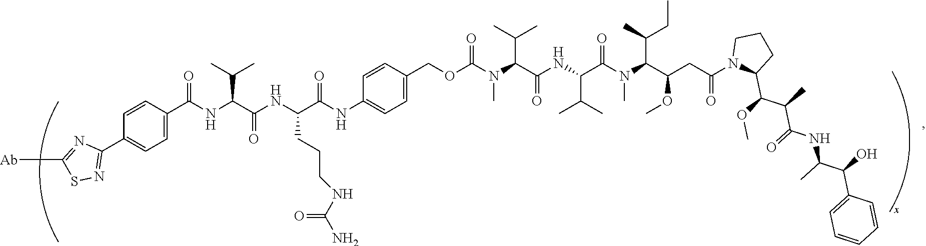
wherein Ab is 6E7 or an antigen-binding fragment thereof,
or a pharmaceutically acceptable salt, tautomer, solvate, or stereoisomer of any of the foregoing.

46. The compound of claim 1, or a pharmaceutically acceptable salt, tautomer, solvate, or stereoisomer thereof, wherein x is about 3.5 to about 4.5.

47. (canceled)

48. The compound of claim 3, wherein the compound is 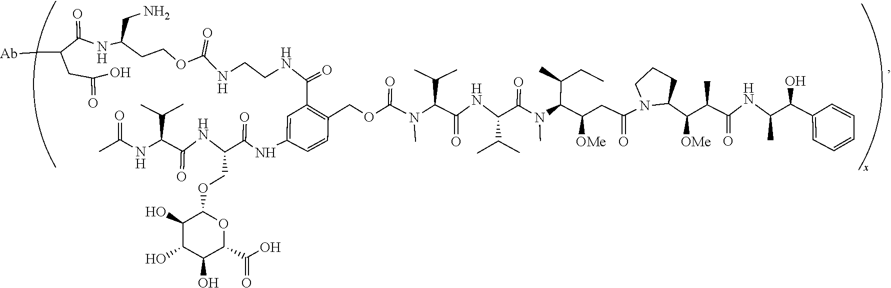
wherein Ab is 6E7 or an 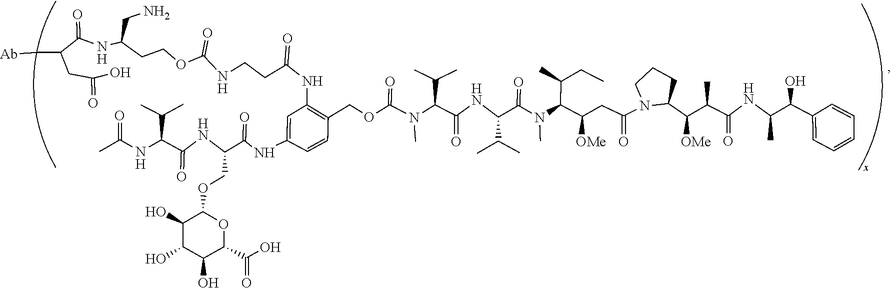
antigen-binding fragment thereof; 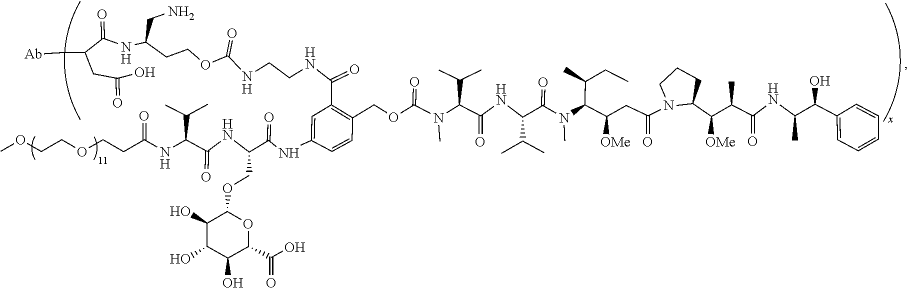
wherein Ab is 6E7 or an antigen-binding fragment thereof; 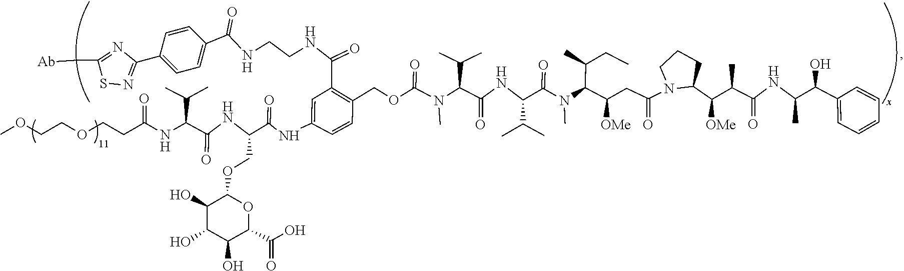
wherein Ab is 6E7 or an antigen-binding fragment thereof; 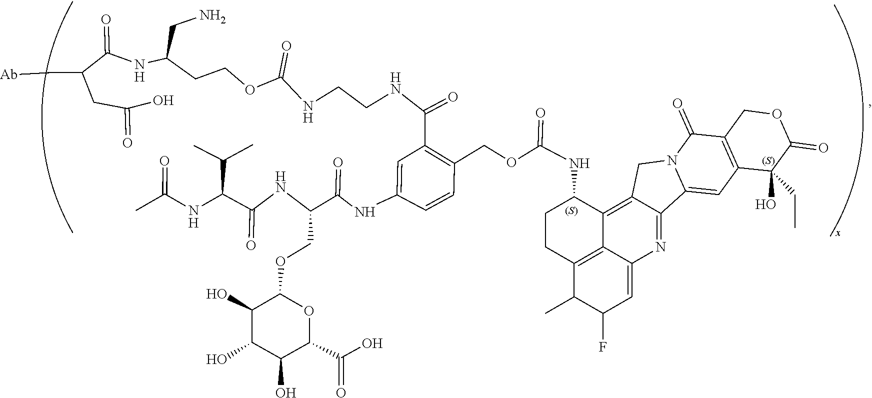
wherein Ab is ifinatamab or an antigen-binding fragment thereof; 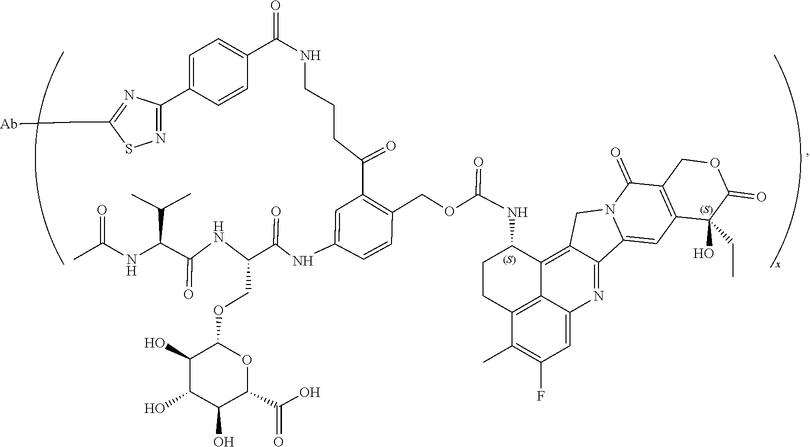
wherein Ab is ifinatamab or an antigen-binding fragment thereof; 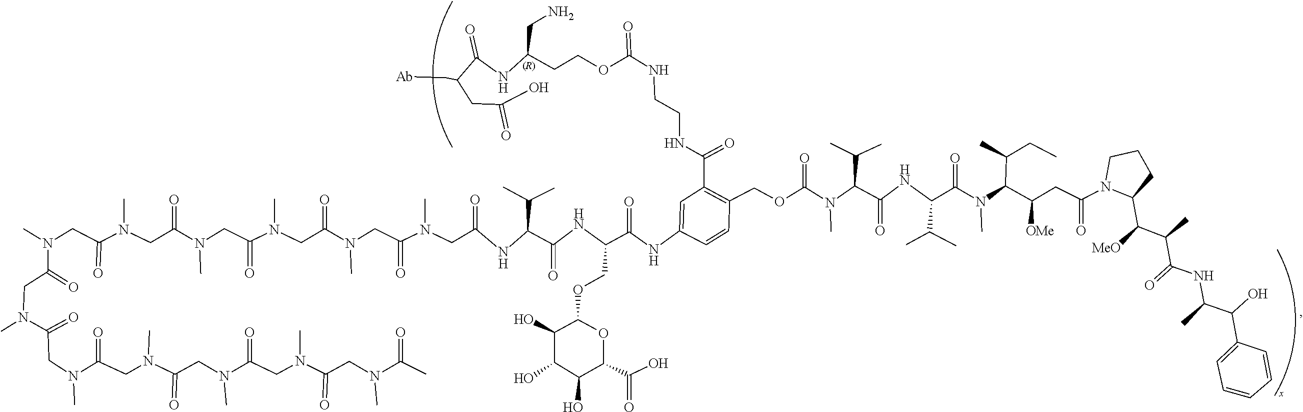
wherein Ab is 6E7 or an antigen-binding fragment thereof; or 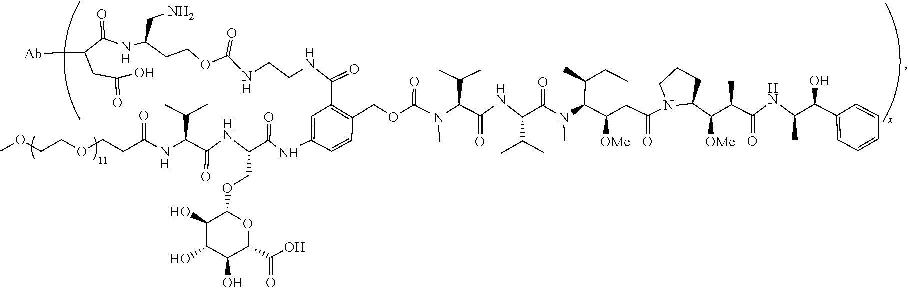
wherein Ab is cofetuzumab or an antigen-binding fragment thereof,
or a pharmaceutically acceptable salt, tautomer, solvate, or stereoisomer of any of the foregoing.

49-50. (canceled)

51. The compound of claim 7, wherein the compound is 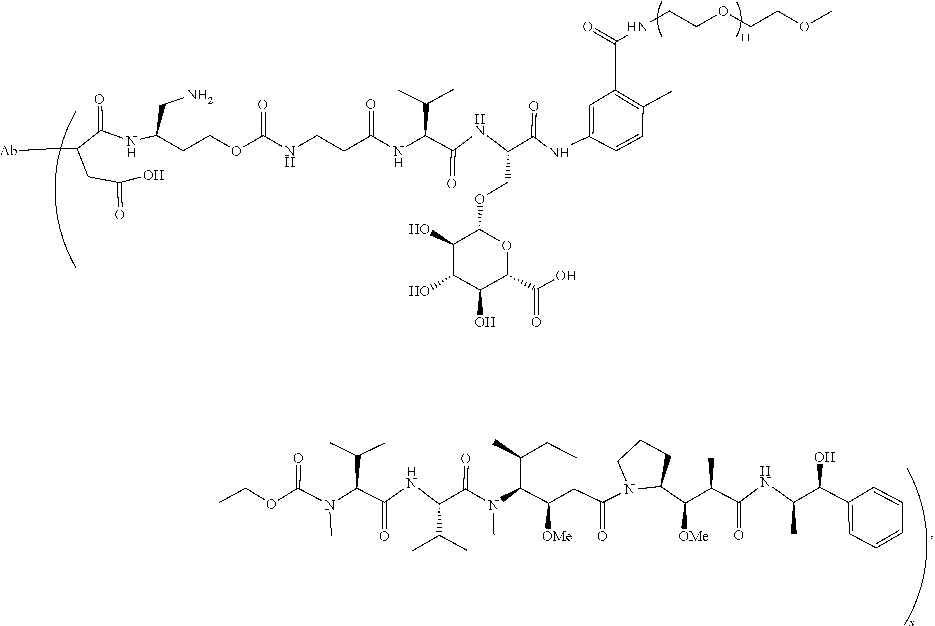
wherein Ab is 6E7 or an 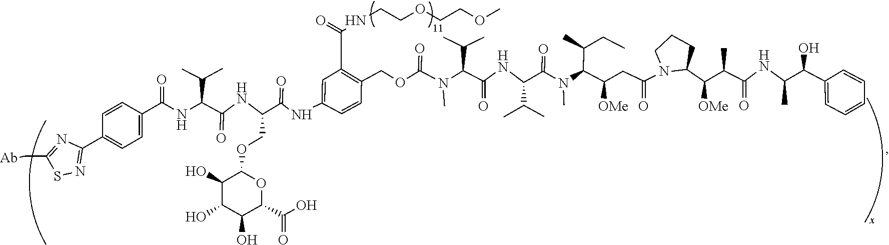
antigen-binding fragment thereof; 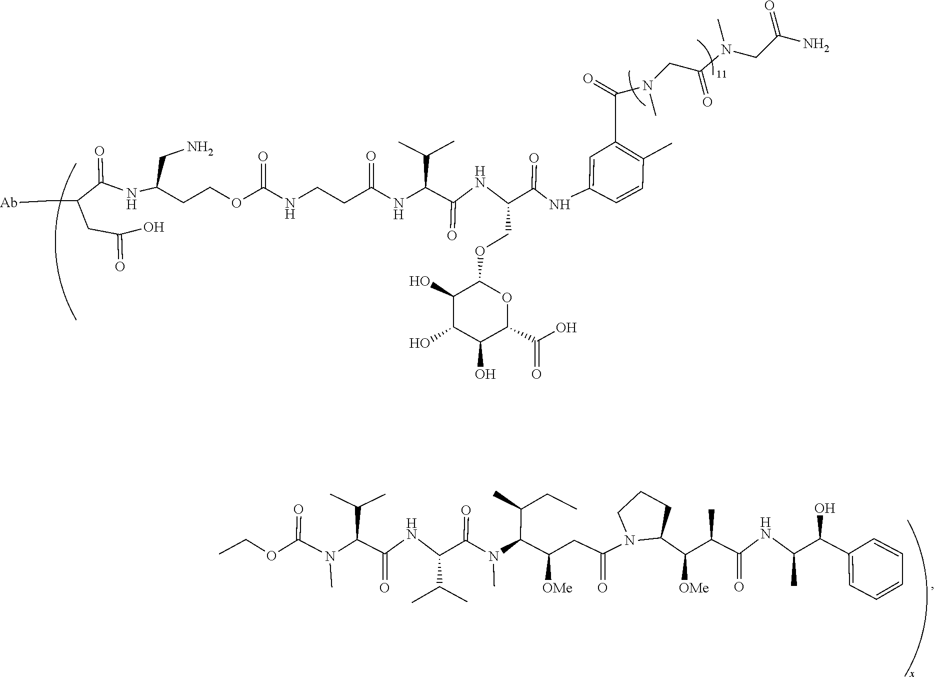
wherein Ab is 6E7 or an antigen-binding fragment thereof; 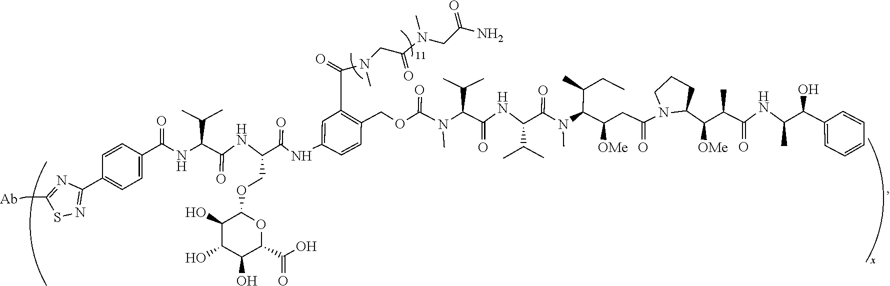
wherein Ab is 6E7 or an antigen-binding fragment thereof; 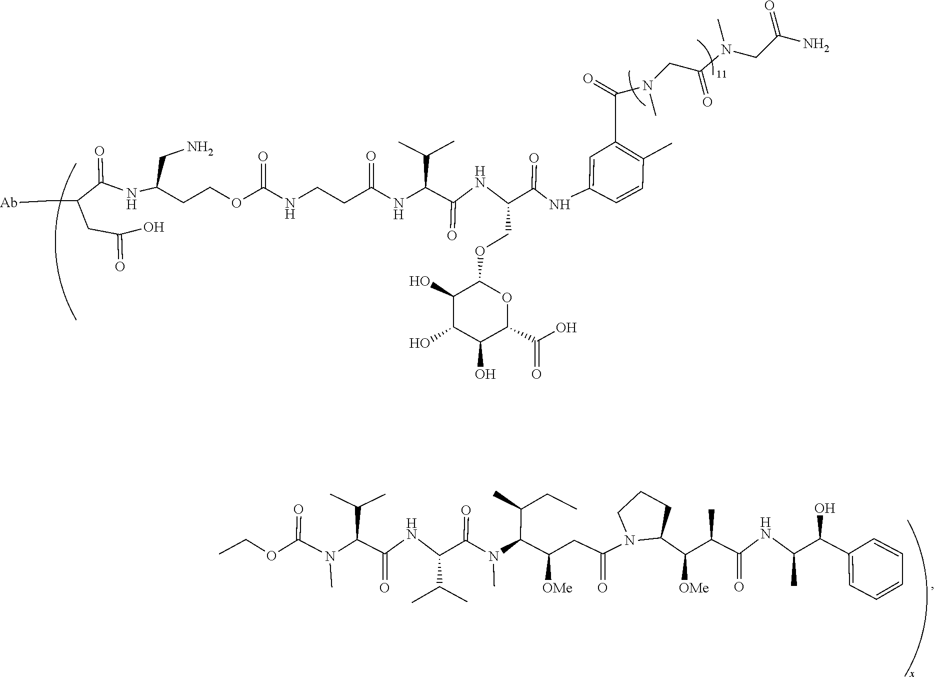
wherein Ab is ifinatamab or an antigen-binding fragment thereof; 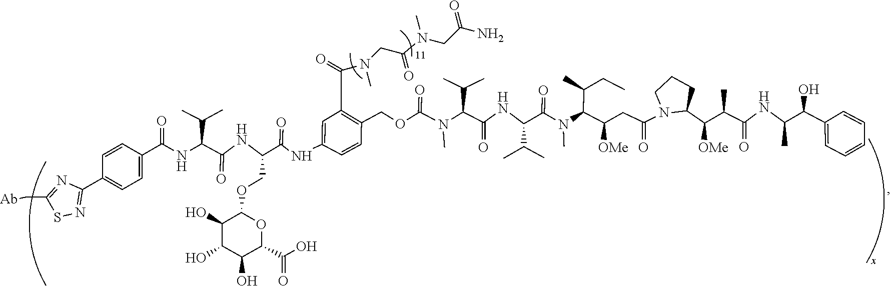
wherein Ab is ifinatamab or an antigen-binding fragment thereof; or 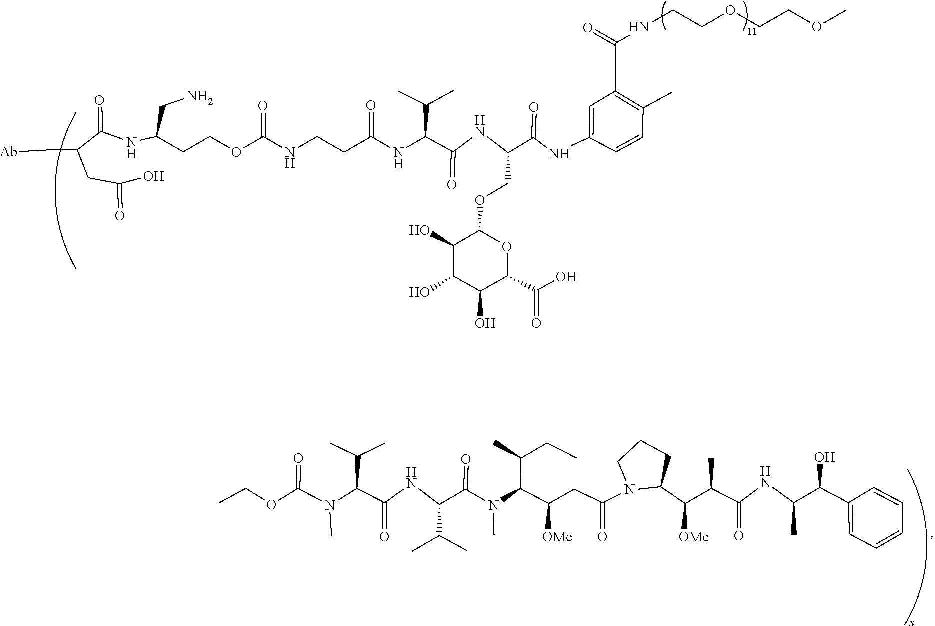
wherein Ab is cofetuzumab or an antigen-binding fragment thereof,
or a pharmaceutically acceptable salt, tautomer, solvate, or stereoisomer thereof.

52-102. (canceled)


SEQUENCE LISTING (6 sequences)

SEQ ID NO: 1 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 218 aa, synthetic construct
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 452 aa, synthetic construct
EVQLVQSGAEVKKPGASVKVSCKASGYSFTDYYMHWVRQAPGQGLEWIGRINPYAGAAFYSQNFKDRVTLTVDTSTSTAYLELSSLRSEDTAVYYCAIERGADLEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK